(19) United States
(12) Patent Application Publication
(10) Pub. No.: US 2022/0033464 A1
(43) Pub. Date: Feb. 3, 2022

(54) METHODS AND COMPOSITONS FOR MODULATIONS OF IMMUNE RESPONSE

(71) Applicants: La Jolla Institute for Immunology, La Jolla, CA (US); University of Southampton, Southampton Hampshire (GB)

(72) Inventors: Pandurangan VIJAYANAND, La Jolla, CA (US); Ferhat AY, La Jolla, CA (US); Divya SINGH, La Jolla, CA (US); Anusha Preethi GANESAN, La Jolla, CA (US); Bharat PANWAR, La Jolla, CA (US); Christian OTTENSMEIER, Southampton Hampshire (GB)

(73) Assignees: La Jolla Institute for Immunology, La Jolla, CA (US); University of Southampton, Southampton Hampshire (GB)

(21) Appl. No.: 17/270,403

(22) PCT Filed: Aug. 23, 2019
(86) PCT No.: PCT/US2019/048036

§ 371 Date: Feb. 22, 2021

Related U.S. Application Data
(60) Provisional application No. 62/722,176, filed on Aug. 24, 2018.

(51) Int. Cl. C07K 14/73; C07K 14/52; C12N 9/64; C07K 14/725; G01N 33/50; A61K 35/17

(52) U.S. Cl. CPC C07K 14/70514; C07K 14/521; C12N 9/6467; C12Y 304/21079; C07K 2319/02; G01N 33/5091; A61K 35/17; C07K 2319/03; C07K 14/7051

(57) ABSTRACT

Disclosed herein are isolated follicular helper T cell (TFH) and engineered follicular helper T cell (TFH) and methods of isolating or engineering such cells. Further disclosed herein are methods of using such cells for treating diseases, such as cancer.

### CROSS-REFERENCE TO RELATED APPLICATIONS

This application claims priority under 35 U.S.C. § 119(e) to U.S. Provisional Application No. 62/722,176, filed Aug. 24, 2018, the content of which is hereby incorporated by reference in its entirety.

### STATEMENT REGARDING FEDERALLY SPONSORED RESEARCH OR DEVELOPMENT

This invention was made with government support under grant numbers P30 CA030199, awarded by the National Institute of Health (NIH) through the National Cancer Institute. The U.S. Government has certain rights in the invention.

### BACKGROUND

Throughout and within this disclosure, technical and patent publications are referenced by an Arabic number that is superscripted, the full citation for which are found immediately preceding the claims. These publications are incorporated by reference in their entireties.

CD8⁺ cytotoxic T cells (CTLs) are vital components of anti-tumor immunity¹. A distinct subset of the CD8⁺ CTLs, tissue resident memory (TRM) T cells, have recently emerged as critical players in mediating robust anti-tumor immune responses^{2, 3, 4, 5}. Cancer immunotherapies to potentiate CD8⁺ CTL responses have led to remarkable clinical success, albeit in a small proportion of patients^{6, 7}. Therapeutic failure is, at least in part, due to an incomplete understanding of the signals and cell types that operate at different stages of the CTL immune response to modulate the magnitude and quality of developing effector or memory CD8⁺ CTLs.

CD4⁺ T helper cells (T_{H}), the central orchestrators of an efficient immune response, play a pivotal role in the activation and maintenance of CTL responses and in facilitating immunological memory^{8,9}. During immunization or infections, CD4⁺ T cells are necessary for robust primary CD8⁺ CTL responses^{10, 11, 12} and memory transition through a multitude of mechanisms involving dendritic cell (DC) licensing and activation^{13, 14}, CD8⁺ CTL recruitment to cognate DC or, in some instances, by direct interactions with CD8⁺ CTLs¹⁵. However, within tumors, it is unknown what properties of CD4⁺ T cells are essential to provide ‘help’ to generate robust CD8⁺ T cell effector and T_{RM} anti-tumor immune responses.

Previous studies of CD4⁺ T cells in cancer have focused on the evaluation of specific CD4⁺ T cell subsets such as regulatory T cells (Treg), CD4⁺ T_{H}1 and CD4⁺ T_{H}17 cells¹⁶. Recently, single-cell sequencing analysis has been applied to tumor cells and immune cells in melanoma and other tumors, which revealed T cell exhaustion signature and their link to T cell activation¹⁷. A similar analysis was performed in tumor infiltrating lymphocytes (TILs) in hepatitis B virus-driven hepatocellular carcinoma where 11 unique T cell subsets were described with a specific focus on exhausted CD8⁺ T cells and CD4⁺ Tregs¹⁸. Whilst these studies have provided valuable insights into specific CD8⁺ T cell and CD4⁺ T cell subsets, the global transcriptional program of tumor-infiltrating CD4⁺ T cells and its association with CD8⁺ T cell effector and T_{RM} anti-tumor immune responses, which are important predictors of improved patient survival, has not been elucidated. Understanding the molecular features of tumor-infiltrating CD4⁺ T cell responses in the context of CD8⁺ CTL responses will reveal novel strategies for bolstering CD4⁺ T cell effector functions in addition to indirectly augmenting CD8⁺ CTL responses within tumors. This disclosure satisfies this need and provides related advantages as well.

### SUMMARY OF THE DISCLOSURE

The properties of human tumor-infiltrating CD4⁺ T cells that provide “help” for generating robust anti-tumor CD8⁺ T cell effector and tissue resident memory (T_{RM}) responses are not known. Applicants performed integrated weighted correlation network analysis (iWGCNA) by merging the transcriptomes of patient-matched, purified CD4⁺ and CD8⁺ T cells present in lung tumors. Applicants found that follicular helper T cell (T_{FH}) program in tumor-infiltrating CD4⁺ T cells was strongly associated with proliferation, cytotoxicity and tissue residency in CD8⁺ T cells within tumors. Single-cell transcriptomic analysis of tumor-infiltrating CD4⁺ T cells confirmed the presence of CXCL13-expressing T_{FH}-like cells, which despite expressing high levels of PDCD1, were enriched for features linked to proliferation, cytotoxicity and CD8⁺ T cell ‘help’, indicative of superior functionality. These findings provide insights into the molecular identity and functional properties of tumor-infiltrating CD4⁺ T cells that are associated with robust anti-tumor CTL and T_{RM} responses, and reveal potential targets for immunotherapy.

Applicants have previously reported on the transcriptomic features of tumor-infiltrating CD8⁺ CTLs in a well-characterized cohort of patients with non-small cell lung cancer (NSCLC)³. To fully characterize the molecular landscape of adaptive immune responses at the tumor site and the differences therein between tumors with or without robust CD8⁺ effector and T_{RM} responses, here, Applicants utilized the transcriptional profiles of patient-matched, purified tumor-infiltrating CD4⁺ T cells from the same cohort of patients to define the molecular interactions between tumor-infiltrating CD4⁺ T cells and CD8⁺ CTLs.

Also disclosed herein is an engineered T-follicular helper (Tfh)-like tumor-infiltrating cell engineered to modulate expression of the surface markers CD4, CXCL13 and CXCR5. In some embodiments, the cell is engineered to express the surface markers CD4 and CXCL13 and lack the surface marker CXCR5. In some embodiments, the cell is further engineered to express GZMB. In some embodiments, the cell is a Tfh-like tumor-infiltrating cell that activates a CD8⁺ CTL response. In some embodiments, the CD8⁺ CTL response is activated in a tumor or tumor microenvironment. In some embodiments, the cell is a Tfh-like tumor-infiltrating cell that activates a CD8⁺ TRM response. In some embodiments, the CD8⁺ TRM response is activated in a tumor or tumor microenvironment.

Disclosed herein is an engineered T-follicular helper-like tumor-infiltrating cell engineered to modulate expression of one or more proteins selected from Table 11. In certain embodiments, the one or more proteins are selected from MAF, SH2D1A (SAP), PDCD1, BTLA, CD200, and BCL6. In some embodiments the cell is engineered to increase expression and/or function of TNFRSF18.

Disclosed herein is an isolated T-follicular helper (Tfh)-like tumor-infiltrating cell expressing the surface markers CD4 and CXCL13 and lacking the surface marker CXCR5. In some embodiments the cell is a cytotoxic Tfh-like tumor-infiltrating cell expressing GZMB.

In some embodiments, any one of the cells disclosed herein is engineered to increase expression and/or function of one or more of: TNFRSF18, TNFRSF4, IFNG, Granzyme B and/or IL21 in the cell.

In some embodiments, any one of the cells disclosed herein is engineered to increase expression and/or function of one or more of the proteins listed in any of Tables 11, 12, and 13 in the cell. In some embodiments, an engineered cell disclosed herein is engineered to increase expression and/or function of one or more of the proteins listed in any of Tables 11, 12, and 13. In some embodiments, an isolated cell disclosed herein is engineered to increase expression and/or function of one or more of the proteins listed in any of Tables 11, 12, and 13. In some embodiments, increasing the expression of one or more of the proteins listed in any of Tables 11, 12, and 13 comprises, consists essentially of, or consists of, contacting an engineered cell with a polynucleotide that encodes for one or more of the proteins listed in any of Tables 11, 12, and 13. In some embodiments, the engineered cell disclosed herein is a cell that is contacted with one or more polynucleotides encoding one or more of the proteins listed in any of Tables 11, 12, and 13. In some embodiments, the isolated cell disclosed herein is contacted with one or more polynucleotides encoding one or more of the proteins listed in any of Tables 11, 12, and 13. In some embodiments, the engineered cell or isolated cell is transfected with one or more polynucleotides encoding one or more of the proteins listed in any of Tables 11, 12, and 13. In some embodiments, the engineered cell or isolated cell is transduced with a polynucleotide encoding a CD4 protein. In some embodiments, the engineered cell or isolated cell is modified to stably express one or more polynucleotides encoding one or more of the proteins listed in any of Tables 11, 12, and 13. In some embodiments, the engineered cell or isolated cell is modified to transiently express one or more polynucleotides encoding one or more of the proteins listed in any of Tables 11, 12, and 13. In some embodiments, the engineered cell or isolated cell is modified to inducibly express one or more polynucleotides encoding one or more of the proteins listed in any of Tables 11, 12, and 13. In some embodiments, the polynucleotide encoding a protein listed in any of Tables 11, 12, and 13 comprises, consists essentially of, or consists of, a polynucleotide sequence listed or referenced in any of Tables 12 and 13. In some embodiments, the polynucleotide encoding a protein listed in any of Tables 11, 12, and 13 comprises, consists essentially of, or consists of, a fragment of the polynucleotide sequence listed or referenced in any of Tables 12 and 13 that encodes for the protein comprising, consisting essentially of, or consisting of, the amino acid sequence listed or referenced in any of Tables 12 and 13. In some embodiments, the protein comprises, consists essentially of, or consists of, the amino acid sequence listed or referenced in Table 12 or 13. In some embodiments, the polynucleotide encoding the protein comprises, consists essentially of, or consists of, a nucleotide sequence that is at least 85%, 90%, 93%, 94%, 95%, 96%, 97%, 98%, 99%, or 100% identical to the nucleotide sequence listed or referenced in Table 12 or 13. In some embodiments, the polynucleotide encoding the protein comprises, consists essentially of, or consists of, a fragment of a nucleotide sequence that is at least 85%, 90%, 93%, 94%, 95%, 96%, 97%, 98%, 99%, or 100% identical to the nucleotide sequence listed or referenced in Table 12 or 13 that encodes for the protein comprising, consisting essentially of, or consisting of, an amino acid sequence that is at least 85%, 90%, 93%, 94%, 95%, 96%, 97%, 98%, 99%, or 100% identical to the amino acid sequence listed in Table 12 or 13. In some embodiments, the polynucleotide encoding the protein comprises, consists essentially of, or consists of, at least 10, 11, 12, 13, 14, 15, 16, 17, 18, 19, 20, 25, 30, 35, 40, 45, 50, 55, 60, 65, 70, 75, 80, 85, 90, 95, 100, 125, 150, 175, 200, 225, 250, 275, or 300 or more contiguous nucleotides of a nucleotide sequence listed or referenced in Table 12 or 13. In some embodiments, the polynucleotide comprises, consists essentially of, or consists of, a nucleotide sequence that differs from the nucleotide sequence listed or referenced in Table 12 or 13 by 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 or more nucleotides. In some embodiments, the protein comprises, consists essentially of, or consists of, an amino acid sequence that is at least 85%, 90%, 93%, 94%, 95%, 96%, 97%, 98%, 99%, or 100% identical to the amino acid sequence listed or referenced in Table 12 or 13. In some embodiments, the protein comprises, consists essentially of, or consists of, at least 10, 11, 12, 13, 14, 15, 16, 17, 18, 19, 20, 25, 30, 35, 40, 45, 50, 55, 60, 65, 70, 75, 80, 85, 90, 95, 100, 125, 150, 175, 200, 225, 250, 275, or 300 or more contiguous amino acids of an amino acid sequence listed or referenced in Table 12 or 13. In some embodiments, the protein comprises, consists essentially of, or consists of, an amino acid sequence that differs from the amino acid sequence listed or referenced in Table 12 or 13 by 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 or more amino acids. In some embodiments, the protein is a mammalian protein. In some embodiments, the protein is a human protein.

In some embodiments, any one of the cells disclosed herein is engineered to express an antigen binding domain that binds at least one tumor antigen. In some embodiments, the tumor antigen comprises, consists essentially of, or consists of, any one of: a CD19, a disialoganglioside-GD2, a c-mesenchymal-epithelial transition (c-Met), a mesothelin, a ROR1, an EGFRvIII, an ephrin type-A receptor 2 (EphA2), an interleukin (IL)-13r alpha 2, an EGFRVIII, aPSMA, anEpCAM, aGD3, afucosyl GM1, a PSCA, a PLAC1, a sarcoma breakpoint, a Wilms Tumor 1 antigen or a combination thereof.

In some embodiments, any one of the cells disclosed herein is engineered to express or expresses an antigen binding domain that binds at least one antigen. For instance, in some embodiments, an engineered cell disclosed herein is engineered to express an antigen binding domain that binds to at least one antigen. Alternatively, an isolated cell disclosed herein naturally expresses an antigen binding domain that binds to at least one antigen. In some embodiments, the antigen is selected from a neo-antigen, tumor-associated antigen, viral antigen, bacterial antigen, and parasitic antigen.

In some embodiments, any one of the cells disclosed herein further comprises, consists essentially of, or consists of, a suicide gene.

In some embodiments, any one of the cells disclosed herein further comprises, consists essentially of, or consists of, a chimeric antigen receptor (CAR). In some embodiments, the chimeric antigen receptor (CAR) comprises, consists essentially of, or consists of: (a) an antigen binding domain; (b) a hinge domain; (c) a transmembrane domain; (d) and an intracellular domain. In some embodiments, the CAR further comprises, consists essentially of, or consists of, a CD3 zeta signaling domain. In some embodiments, the hinge domain is any one of: CD8a or IgG1 hinge domain. In some embodiments, the transmembrane domain is any one of: CD28 or a CD8α transmembrane domain. In some embodiments, the intracellular domain comprises, consists essentially of, or consists of, one or more costimulatory regions selected from a CD28 costimulatory signaling region, a 4-1BB costimulatory signaling region, an ICOS costimulatory signaling region and/or an OX40 costimulatory region.

In some embodiments, the antigen binding domain of the CAR binds a tumor antigen. In some embodiments, the tumor antigen comprises, consists essentially of, or consists of, any one of: a CD19, a disialoganglioside-GD2, a c-mesenchymal-epithelial transition (c-Met), a mesothelin, a ROR1, an EGFRvIII, an ephrin type-A receptor 2 (EphA2), an interleukin (IL)-13r alpha 2, an EGFR VIII, a PSMA, an EpCAM, a GD3, a fucosyl GM1, a PSCA, a PLAC1, a sarcoma breakpoint, a Wilms Tumor 1 antigen, or a combination thereof.

In some embodiments, the CAR further comprises, consists essentially of, or consists of, an inducible or a constitutively active element. In some embodiments, the inducible or the constitutively active element controls the expression of a polynucleotide encoding an immunoregulatory molecule or a cytokine. In some embodiments, the immunoregulatory molecule or cytokine comprises, consists essentially of, or consists of, one or more of B7.1, CCL19, CCL21, CD40L, CD137L, GITRL, GM-CSF, IL-12, IL-2, low-toxicity IL-2, IL-15, IL-18, IL-21, LEC, and/or OX40L. In some embodiments, the immunoregulatory molecule or cytokine comprises, consists essentially of, or consists of, IL-12 and/or GM-CSF; and/or IL-12 and/or one or more of IL-2 and low-toxicity IL-2; and/or IL-12 and/or IL-15; and/or IL-12 and/or IL-21; IL-12 and/or B7.1; and/or IL-12 and/or OX40L; and/or IL-12 and/or CD40L; and/or IL-12 and/or GITRL; and/or IL-12 and/or IL-18; and/or one or more of IL-2 and low-toxicity IL-2 and one or more of CCL19, CCL21, and LEC; and/or IL-15 and one or more of CCL19, CCL21, and LEC; and/or IL-21 and one or more of CCL19, CCL21, and LEC; and/or GM-CSF and one or more of CCL19, CCL21, and LEC; and/or OX40L and one or more of CCL19, CCL21, and LEC; and/or CD137L and one or more of CCL19, CCL21, and LEC; and/or comprises, consists essentially of, or consists of, B7.1 and one or more of CCL19, CCL21, and LEC; and/or CD40L and one or more of CCL19, CCL21, and LEC; and/or GITRL and one or more of CCL19, CCL21, and LEC.

In some embodiments, the antigen binding domain of the CAR comprises, consists essentially of, or consists of, a heavy chain variable region and a light chain variable region.

In some embodiments, the antigen binding domain of the CAR further comprises, consists essentially of, or consists of, a linker polypeptide located between the heavy chain variable region and the light chain variable region. In some embodiments, the linker polypeptide of the CAR comprises, consists essentially of, or consists of, a polypeptide of the sequence (GGGGS)n wherein n is an integer from 1 to 6.

In some embodiments, the CAR further comprises, consists essentially of, or consists of, a detectable marker attached to the CAR.

In some embodiments, the CAR further comprises, consists essentially of, or consists of, a purification marker attached to the CAR.

In some embodiments, any one of the cells disclosed herein comprises, consists essentially of, or consists of, a polynucleotide encoding the CAR.

In some embodiments, the polynucleotide further comprises, consists essentially of, or consists of, a promoter operatively linked to the polynucleotide to express the polynucleotide in the cell.

In some embodiments, the polynucleotide further comprises, consists essentially of, or consists of, a 2A self-cleaving peptide (T2A) encoding polynucleotide sequence located upstream of the polynucleotide encoding the antigen binding domain.

In some embodiments, the polynucleotide further comprises, consists essentially of, or consists of, a polynucleotide encoding a signal peptide located upstream of the polynucleotide encoding the antigen binding domain.

In some embodiments, the polynucleotide further comprises, consists essentially of, or consists of, a vector. In some embodiments, the vector is a plasmid or a viral vector, wherein the viral vector is optionally selected from the group of a retroviral vector, a lentiviral vector, an adenoviral vector, and an adeno-associated viral vector.

Further disclosed herein is a method of producing any one of the cells disclosed herein, comprising, consisting essentially of, or consisting of, reducing or eliminating expression and/or function of CXCR5 and increasing the expression of CD4 and CXCL13 in the cell using one or more of: RNA interference (RNAi), CRISPR, TALEN and/or ZFN.

Further disclosed herein is a method of producing any one of the cells disclosed herein, increasing the expression of one or more of: TNFRSF18, TNFRSF4, IFNG, Granzyme B and/or IL21 in the cell using one or more of: CRISPR, TALEN and/or ZFN. In some embodiments, the method comprises, consists essentially of, or consists of, increasing the expression of 2, 3, or 4 or more of: TNFRSF18, TNFRSF4, IFNG, Granzyme B and IL21 in the cell using one or more of: CRISPR, TALEN and/or ZFN.

Further disclosed herein is a method of isolating any one of the cells disclosed herein, comprising, consisting essentially of, or consisting of, separating Tfh-like tumor-infiltrating cell from a mixed cell population. In some embodiments, the method further comprises, consists essentially of, or consists of, sorting for cells that express the surface markers CD4 and CXCL13 and lack the surface marker CXCR5. In some embodiments, the method further comprises, consists essentially of, or consists of, increasing the expression or function of one or more of: TNFRSF18, TNFRSF4, IFNG, Granzyme B and/or IL21 in the cell using one or more of: CRISPR, TALEN and/or ZFN.

Further disclosed herein is a method of producing any one of the cells disclosed herein, comprising, consisting essentially of, or consisting of, modulating the expression and/or function of one or more proteins selected from Table 11. In certain embodiments, the one or more proteins are selected from MAF, SH2D1A (SAP), PDCD1, BTLA, CD200, and BCL6. In some embodiments, the expression and/or function of the one or more surface markers is modulated by using one or more of CRISPR, TALEN and/or ZFN.

In some embodiments, the method further comprises, consists essentially of, or consists of, increasing the expression or function of one or more of: TNFRSF18, TNFRSF4, IFNG, Granzyme B and/or IL21 in the cell using one or more of: CRISPR, TALEN and/or ZFN.

Further disclosed herein is an engineered cell prepared by any of the methods disclosed herein.

Further disclosed herein is a substantially homogenous population of cells comprising, consisting essentially of, or consisting of, any one of the cells disclosed herein. In some embodiments, at least 80%, 85%, 90%, 95%, or more of the cells in the homogenous population of cells are any one of the cells disclosed herein.

A heterogeneous population of cells of comprising, consisting essentially of, or consisting of, any of the cells disclosed herein. In some embodiments, at least 60%, 65%, 70%, 75%, 80%, 85%, 90%, 95%, or more of the cells in the heterogeneous population of cells are any one of the cells disclosed herein.

Disclosed herein is a method of preparing any of the population of cells disclosed herein, comprising, consisting essentially of, or consisting of, isolating the cells from a subject and culturing the cells ex vivo.

Disclosed herein is a method of preparing any of the population of cells disclosed herein, comprising, consisting essentially of, or consisting of, expanding the cells in vivo and isolating the cells from a subject.

Further disclosed herein is a composition comprising, consisting essentially of, or consisting of, a carrier and one or more of: the cells disclosed herein and/or any of the population of cells disclosed herein.

In some embodiments, the carrier is a pharmaceutically acceptable carrier.

In some embodiments, the composition further comprises, consists essentially of, or consists of, a cryoprotectant.

Further disclosed herein is a method of determining whether a subject will respond to a treatment for cancer, comprising, consisting essentially of, or consisting of, measuring the amount of one or more of: CD4⁺CXCL13⁺CXCR5′ Tfh-like tumor-infiltrating cell, and/or CD4⁺CXCL13⁺CXCR5⁻GZMB⁺ cytotoxic Tfh-like tumor-infiltrating cell in a sample isolated from the subject, wherein higher amounts of the cells indicates that the subject is likely to respond to the treatment and lower amounts of the cells indicates that the subject is not likely to respond to the treatment.

In some embodiments, the treatment for cancer comprises, consists essentially of, or consists of, a checkpoint inhibitor. In some embodiments, the checkpoint inhibitor is selected from the group of an anti-PD-1, anti-PD-L1, anti-CTLA-4, anti-B7-1, and anti-B7-2 immunotherapy treatment.

In some embodiments, the method further comprises, consists essentially of, or consists of, administering to the subject that is likely to respond to the checkpoint inhibitor therapy an effective amount of the checkpoint inhibitor therapy.

In some embodiments, the method further comprises, consists essentially of, or consists of, administering to the subject an effective amount of a cytoreductive therapy. In some embodiments, the cytoreductive therapy comprises, consists essentially of, or consists of, one or more of chemotherapy, immunotherapy, or radiation therapy.

In some embodiments, the method further comprises, consists essentially of, or consists of, administering to the subject an effective amount of one or more of: the cells disclosed herein, the population of cells disclosed herein and/or the compositions disclosed herein.

Further disclosed herein is a method of treating cancer in a subject comprising, consisting essentially of, or consisting of, administering to the subject an effective amount of one or more of: the cells disclosed herein, the population of cells disclosed herein and/or the compositions disclosed herein.

In some embodiments, the subject is selected for treatment by contacting a sample isolated from the subject with an agent that detects the presence of a tumor antigen in the sample and the subject is selected for the treatment if presence of one or more tumor antigen is detected in the sample. In some embodiments, the subject is a mammal. In some embodiments, the subject is a human.

Further disclosed herein is a pharmaceutical composition comprising, consisting essentially of, or consisting of, any of the cells disclosed herein for the treatment of cancer.

Further disclosed herein is a pharmaceutical composition comprising, consisting essentially of, or consisting of, any of the populations of cells disclosed herein for the treatment of cancer.

Further disclosed herein is a pharmaceutical composition comprising, consisting essentially of, or consisting of, any of the compositions disclosed herein for the treatment of cancer.

Use of any of the cells disclosed herein for the treatment of cancer.

Use of any of the populations of cells disclosed herein for the treatment of cancer.

Use of any of the compositions disclosed herein for the treatment of cancer.

Use of the cells disclosed herein in the manufacture of a medicament for the treatment of cancer.

Use of the populations of cells disclosed herein in the manufacture of a medicament for the treatment of cancer.

Use of the compositions disclosed herein in the manufacture of a medicament for the treatment of cancer.

In some embodiments, the cancer is lung cancer.

Further disclosed herein is a kit comprising, consisting essentially of, or consisting of, one or more of: the cells disclosed herein, the populations of cells disclosed herein, and/or the compositions disclosed herein and instructions to carry out the any of the methods disclosed herein.

### BRIEF DESCRIPTION OF THE FIGURES

FIGS. 1A-1D. Core transcriptional profile of CD4⁺ TILs in human lung cancer. (FIG. 1A) Schematic representation of study method; dotted black box indicates previously published CD8⁺ TIL transcriptomic data³. (FIG. 1B) Canonical pathways (horizontal axis; bars in plot) for which CD4⁺ TILs show enrichment, presented as the frequency of differentially expressed genes encoding components of each pathway that are upregulated (key) in CD4⁺ TILs relative to their expression in CD4⁺ N-TILs (left vertical axis), and adjusted P values (right vertical axis; line; Benjamini-Hochberg test). RA, rheumatoid arthritis; GADD45, growth arrest and DNA damage-inducible 45; TNFR2, tumor necrosis factor receptor 2. (FIG. 1C) GSEA of various gene sets (above plots) in the transcriptome of CD4⁺ TILs versus that of CD4⁺ N-TILs from patients with NSCLC, presented as running enrichment score (RES) for the gene set, from most over-represented genes at left to most under-represented at right; values above the plot represent the normalized enrichment score (NES) and false discovery rate (FDR)-corrected significance value; Kolmogorov-Smimov test. (FIG. 1D) Wind rose plot showing GSEA significance (increasing from center, −log₁₀ adjusted P value) of various gene sets tested as in FIG. 1C. See also FIG. 7; Tables 1-3 and 10.

FIGS. 2A-2D. Concordance in immunotherapy target expression between patient-matched CD8⁺ and CD4⁺ TILs. (FIG. 2A) RNA-Seq analysis (row-wise TPM; bottom key) of various transcripts (right margin; one per row) in CD8⁺ TILs (top panel) and CD4⁺ TILs (bottom panel) from patients with NSCLC (one per column); above, patients ordered based on CD8⁺ TIL expression of PDCD1 transcripts; red frame highlights patient ID 34. (FIG. 2B) Correlation of the expression of transcripts in FIG. 2A in CD8⁺ TILs and that of the same transcripts in CD4⁺ TILs in NSCLC. Spearman correlation coefficient (vertical axis); P values (horizontal axis), Spearman correlation. (FIG. 2C) Scatter plot of the ranking order of patients based on expression of PDCD1 (left) and HAVCR2 (right) in CD4⁺ TILs (horizontal axis) and CD8⁺ TILs (vertical axis); red circle shows patient ID 34. (FIG. 2D) RNA-Seq analysis of PDCD1 and HAVCR2 transcripts in patient ID 34 presented as TPM in CD4⁺ TILs and CD8⁺ TILs (key). See also FIG. 8.

FIGS. 3A-3D. Follicular program in CD4⁺ T cells is associated with CD8⁺ T cell proliferation, cytotoxicity and tissue residency within tumors. (FIG. 3A) Schematic representation of iWGCNA of the CD4⁺ and CD8⁺ TIL transcriptomes and generation of modules (left). Barplots (right, below) show module size (number of genes, left margin) and composition of CD4⁺ T cell- and CD8⁺ T cell-transcripts (key above plot) for each module; number below each bar represents the corresponding module ID. Significance of correlation (right, above) of proliferation-related eigengene to CD8⁺ T cell-transcripts within each module represented by symbols (Spearman correlation, left margin); red line denotes significance threshold of Bonferroni adjusted P value=0.001; red symbol denotes modules with significant correlation. Box highlights Module 7. (FIG. 3B) Hierarchical clustering analysis showing Spearman correlation co-expression matrix of the CD8⁺ T cell- (above) and CD4⁺-T cell transcripts (below) in Module 7 (bottom key); black frame within matrix delineates gene clusters; left margin, number of genes in module; right margin, key genes enriched in the clusters. (FIG. 3C) Enrichment of T_{FH} signature genes (above) or cell cycle genes (below) in CD4⁺ TILs within each module; horizontal axis represents module ID; vertical axis represents percentage of genes (symbols); red symbols denote Bonferroni adjusted P value <0.001 (hypergeometric test). Box highlights Module 7. (FIG. 3D) GSEA of T_{FH} (above) or cell cycle signature (below) in the transcriptome of CD4⁺ TILs from T_{RM}^{high} versus T_{RM}^{low} tumors, presented as in FIG. 1C. See also FIG. 9; Tables 1, 5, 6 and 10.

FIGS. 4A-4G. Single-cell transcriptomics reveal that CXCL13-expressing tumor-infiltrating CD4⁺ T cells possess superior functional properties. (FIG. 4A) Sorting strategy for single-cell RNA-Seq assays. Live, singlet gated, CD14⁻CD19⁻CD20⁻CD8⁻CD56⁻CD45⁺CD3⁺CD4⁺ lymphocytes from 6 lung tumors were sorted as CXCR5⁺, CXCR5⁻CD25⁺CD127⁻ and CXCR5 CD25⁻ subsets. (FIG. 4B) Seurat clustering of ˜5300 single cell TIL transcriptomes identifying 9 clusters (left); each symbol represents a cell; circle delineates CXCL13 cluster. tSNE visualization of cells in FIG. 4B (right); each symbol represents a cell; brown color indicates CXCL13 expression in counts per million (CPM). Pie chart represents the percentage of CXCL13-expressing cells among all TILs (far right, above) and relative proportions of each of the sorted subsets that express CXCL13 (far right, below). (FIG. 4C, FIG. 4D) Percentage (left margin) of CXCL13-expressing or CXCL13-non-expressing cells that express the indicated T_{FH}-related genes (left plot) or cell cycles genes (right plot). Below, GSEA of T_{FH} or cell cycle signature in the transcriptome of CXCL13-expressing versus CXCL13-non-expressing cells, presented as in FIG. 1C. (FIG. 4E) Canonical pathways (horizontal axis; bars in plot) for which CXCL13-expressing TILs show enrichment, presented as the frequency of differentially expressed genes encoding components of each pathway that are upregulated (key) in CXCL13-expressing TILs relative to their expression in CXCL13-non-expressing TILs (left vertical axis), and adjusted P values (right vertical axis; line; Benjamini-Hochberg test). (FIG. 4F) Percentage (left margin) of CXCL13-expressing or CXCL13-non-expressing cells that express the indicated CD4⁺ help-related genes (left plot) and violin plots of expression of the same genes in CXCL13-expressing or CXCL13-non-expressing cells (right); shape represents the distribution of expression among cells and color represents expression (log₂(CPM+1)). (FIG. 4G) Percentage (left margin) of CXCL13-expressing or CXCL13-non-expressing cells that express the indicated cytotoxicity-related genes (left plot). Below, GSEA of cytotoxicity signature in the transcriptome of CXCL13-expressing versus CXCL13-non-expressing cells presented as in FIG. 1C. Right, violin plots of expression of cytotoxicity-related genes in CXCL13-expressing or CXCL13-non-expressing cells, presented as in FIG. 4F. See also FIG. 10; Tables 6-8 and 10.

FIG. 5A-5B. Highly functional T_{FH}-like CD4⁺ T cells were CXCR5 negative. (FIG. 5A) Expression of transcripts differentially expressed in CXCL13-expressing versus CXCL13-non-expressing TILs, in various sorted subsets (above heatmap, right key). Each column represents the average expression (CPM) in a particular subset. Left margin, vertical colored lines indicate subset in which the genes are differentially expressed. Right margin, examples of key transcripts expressed uniquely or shared by corresponding subsets. (FIG. 5B) Expression of indicated transcripts (bars) in various sorted subsets (key as in FIG. 5A), represented as CPM (left margin). Percentage of cells (black symbol, right margin) that express the indicated transcript (above plot) in each sorted subset. See also FIG. 11.

FIGS. 6A-6B. T_{FH}-like cells infiltrate tumor and associate with CD8⁺ T_{RM} cells

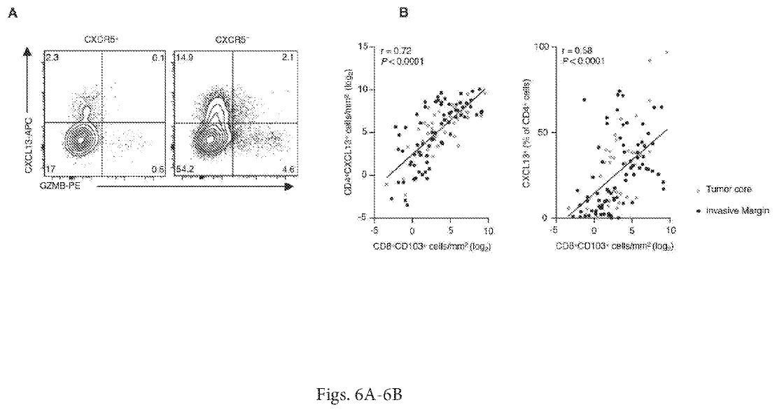

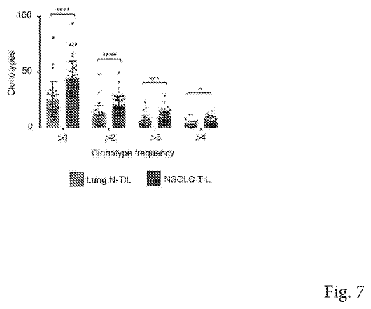

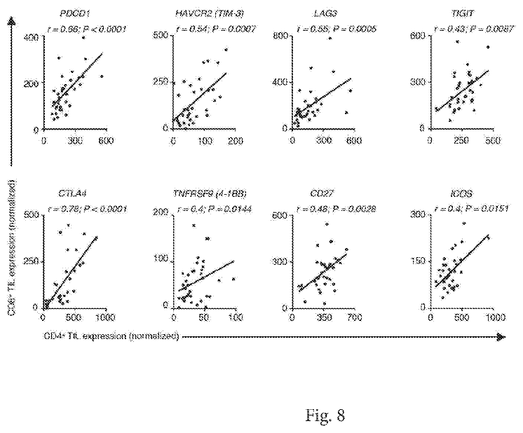

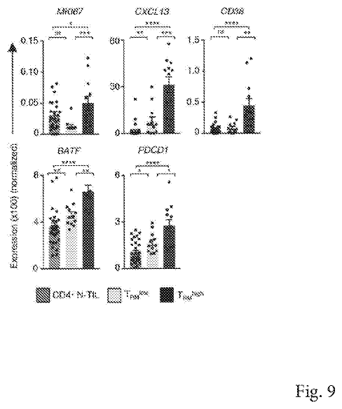

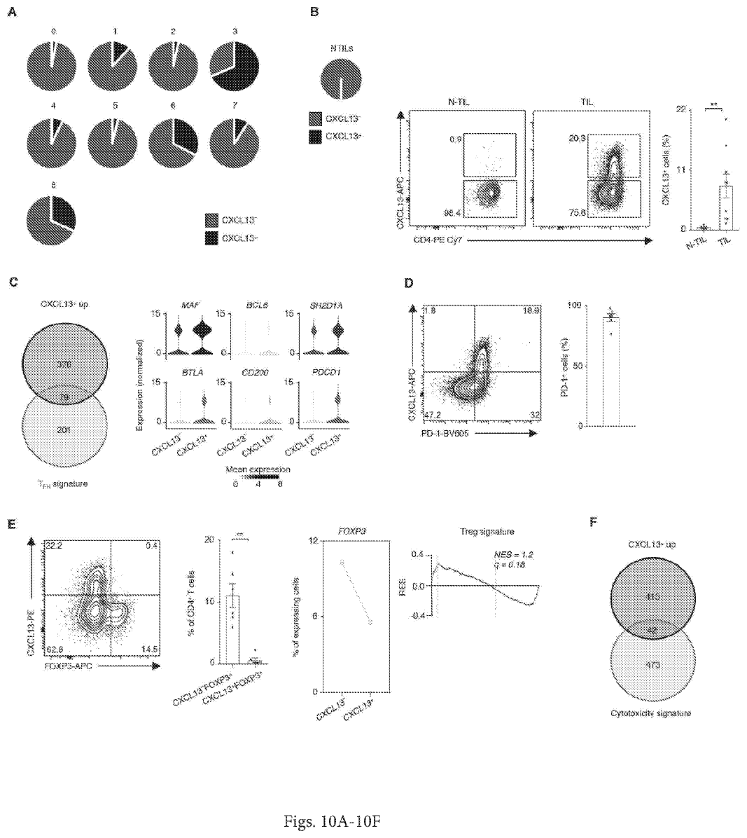

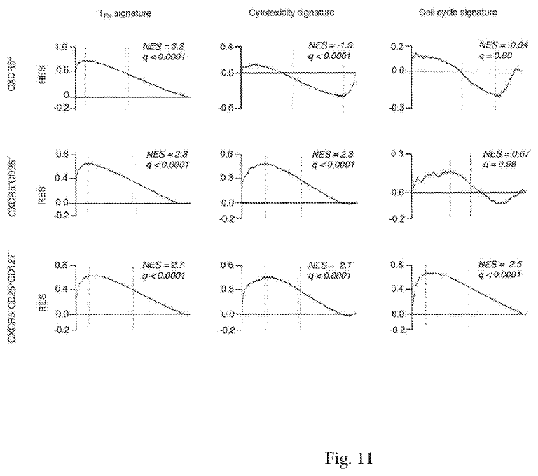

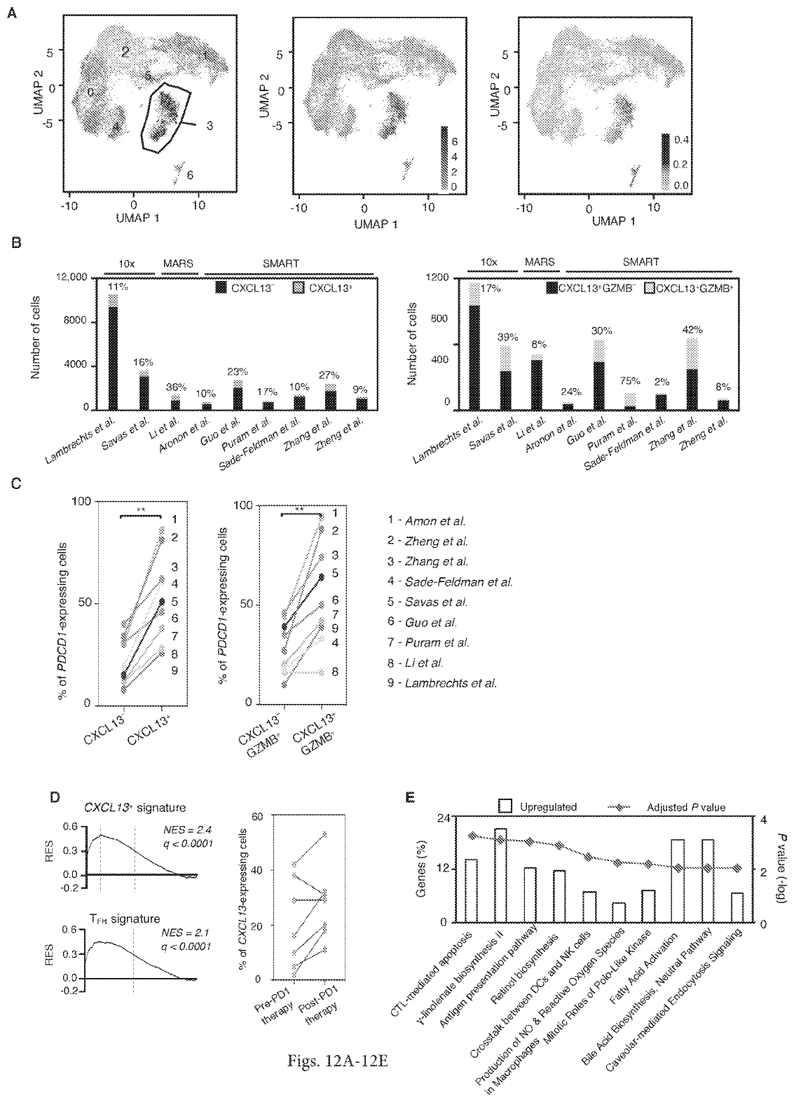

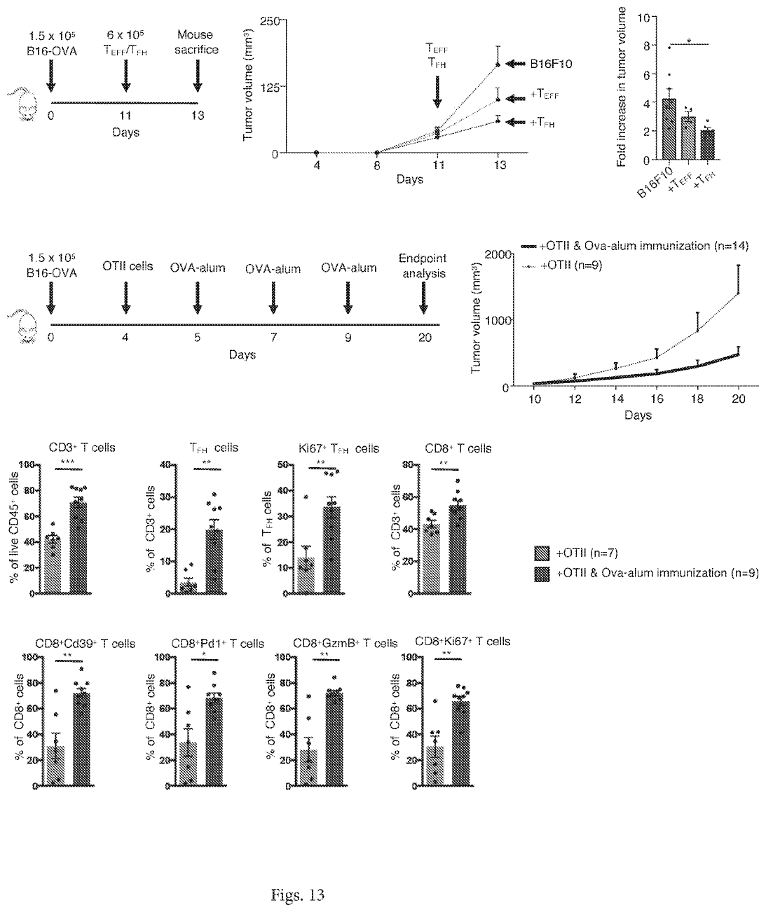

(FIG. 6A) Flow-cytometry analysis shows expression of CXCL13 and granzyme B in CXCR5⁺ and CXCR5⁻ subsets in live, singlet-gated, CD45⁺CD3⁺CD4⁺ TILs; numbers in quadrants indicate percentage of CD4⁺ TILs in each. (FIG. 6B) Correlation of the number of CD4⁺CXCL13⁺ cells and CD8⁺CD103⁺ cells in lung tumors (quantified by IHC) (left). Correlation of the percentage of CXCL13⁺ cells in CD4⁺ cells and the number of CD8⁺CD103⁺ cells in lung tumors (quantified by IHC)(right). Each symbol (key) represents an image; 3 images analyzed per patient (n=41); r values indicates the Spearman correlation coefficient; P values, Spearman correlation,

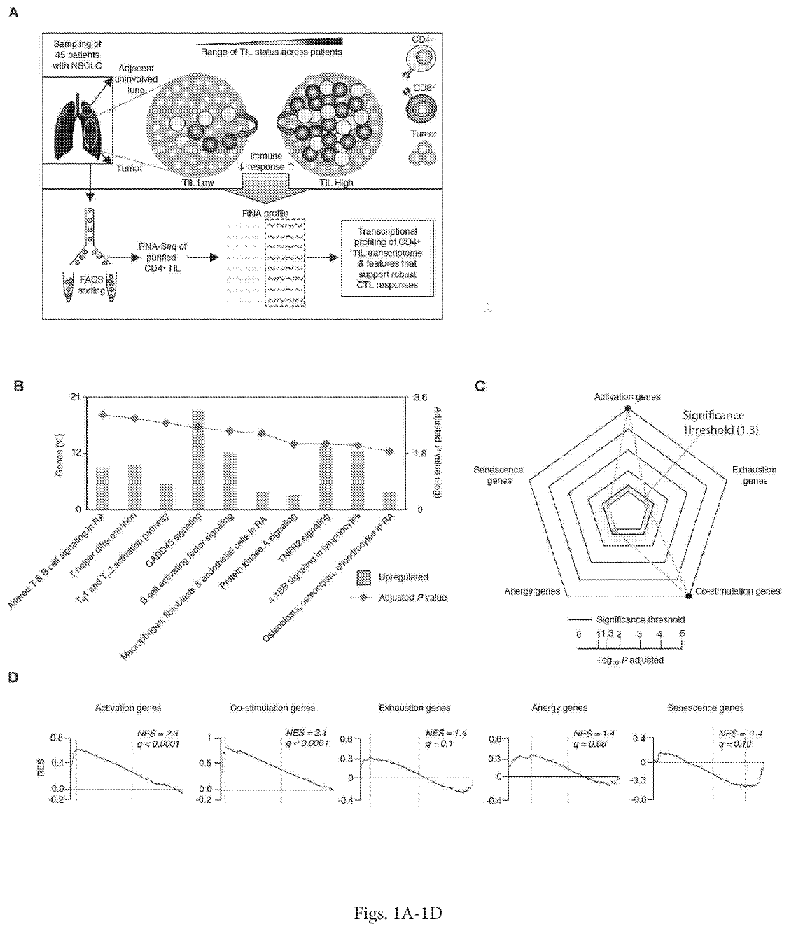

FIG. 7. Core transcriptional profile of CD4⁺ TILs in human lung cancer. Related to FIG. 1; Tables 2 and 4. Quantification of clonotypes (average values) among CD4⁺ N-TILs and NSCLC CD4⁺ TILs (key) according to their frequency in each patient (horizontal axis), derived from RNA-Seq analysis of genes encoding TCR β-chains. Each symbol represents a patient; small horizontal lines indicate the mean (±s.e.m.). *P<0.05; ***P<0.001; ****P<0.0001 (Mann-Whitney test).

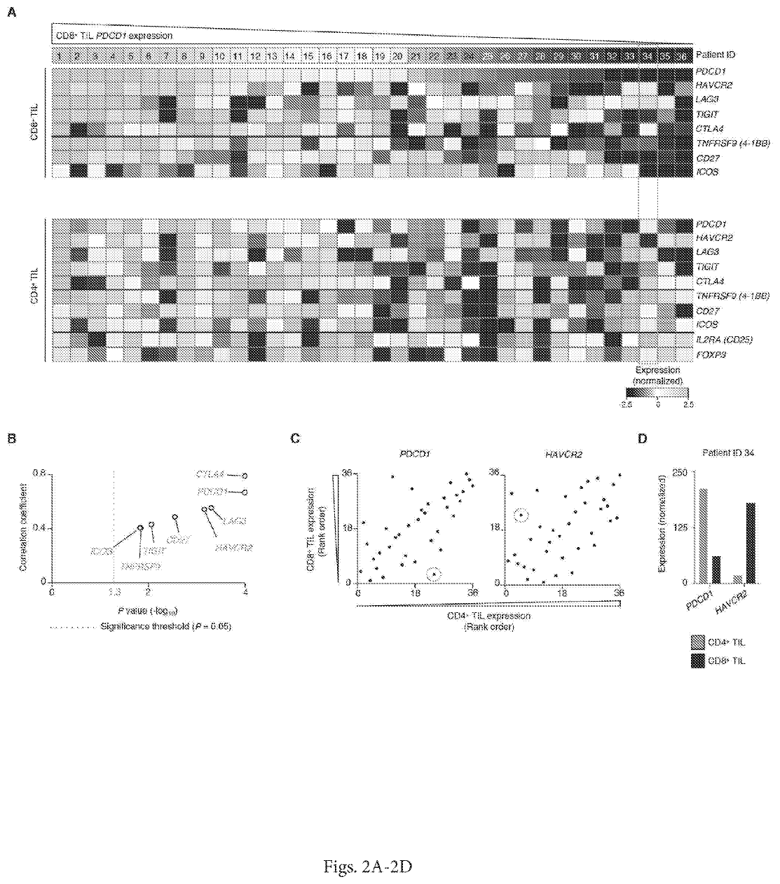

FIG. 8. Concordance in immunotherapy target expression between patient-matched CD8⁺ and CD4⁺ TILs. Related to FIG. 2. Correlation of the expression (TPM) of the indicated transcripts (above plots) in CD8⁺ TILs and that of the same transcripts in CD4⁺ TILs in NSCLC. Each symbol represents a patient (n=36); r values indicate Spearman correlation coefficient; P values, Spearman correlation.

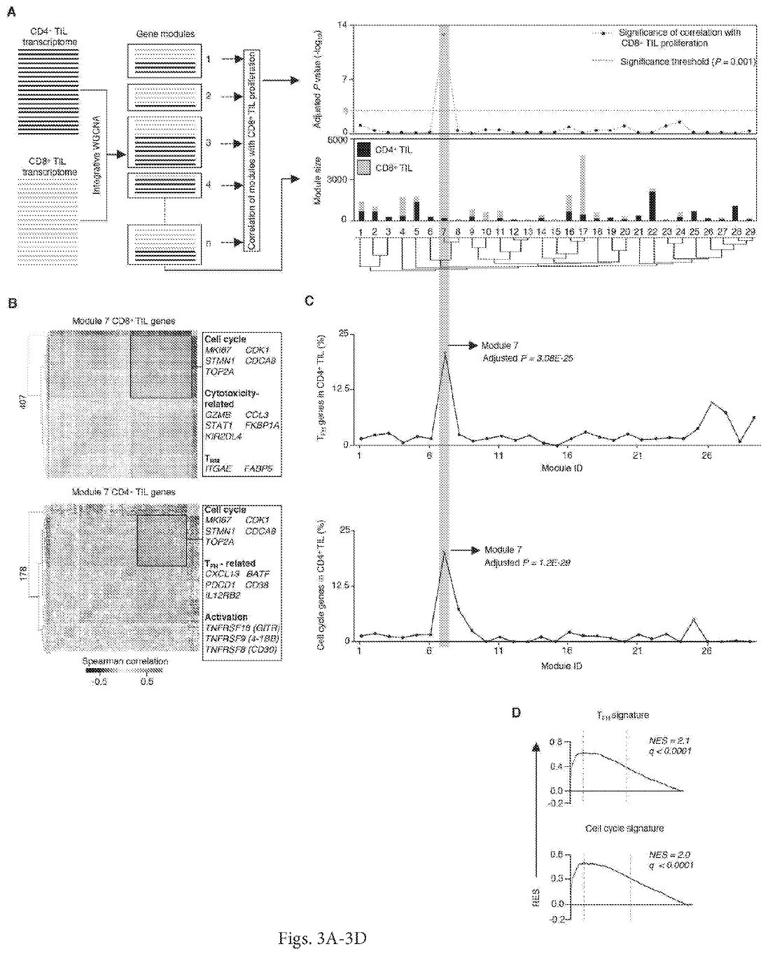

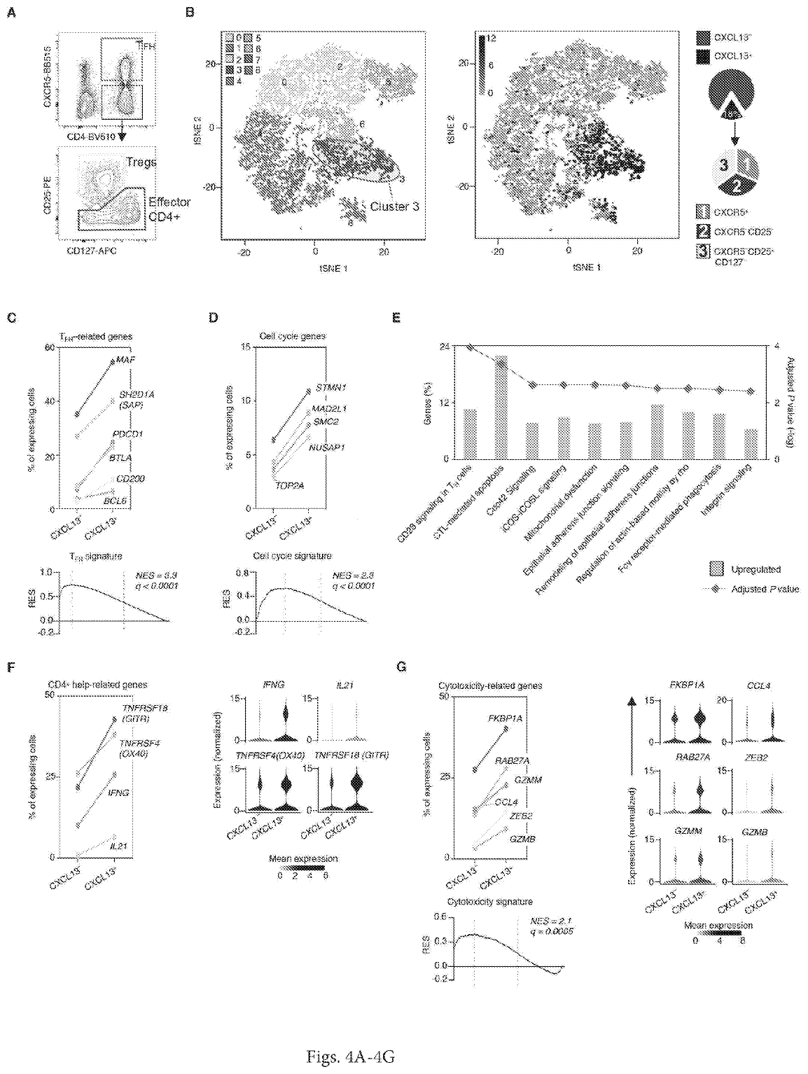

FIG. 9. Follicular program in CD4⁺ T cells is associated with CD8⁺ T cell proliferation, cytotoxicity and tissue residency within tumors. Related to FIG. 3; Table 1. RNA-Seq analysis of the expression (TPM) of indicated genes in CD4⁺ T cells from uninvolved lung or T_{RM}^{low} or T_{RM}^{high} tumors (bottom right key). Each symbol represents an individual patient; small horizontal lines indicate the mean (±s.e.m.). *P<0.05; **P<0.01; ***P<0.001; ****P<0.0001 (Mann-Whitney test).

FIGS. 10A-10F. Single-cell transcriptomics reveal that CXCL13-expressing tumor-infiltrating CD4⁺ T cells possess superior functional properties. Related to FIG. 4; Table 7, 8 and 10. (FIG. 10A) Pie charts represent the proportion of CXCL13-expressing cells (key) in each cluster of CD4⁺ TILs. (FIG. 10B) Pie chart (middle) represents the proportion of CXCL13-expressing cells (key) among all N-TILs. Flow-cytometry analysis shows the expression (right) and percentage (far right) of CXCL13 in live, singlet-gated, CD45⁺CD3⁺CD4⁺ T cells obtained from lung N-TILs (n=4) or NSCLC TILs (n=9) from patients with NSCLC. **P<0.01 (Mann-Whitney test). (FIG. 10C) Venn diagram (left) shows overlap of genes differentially expressed in CXCL13-expressing cells versus non-expressing cells and T_{FH} signature genes. Violin plots (right) of expression of key T_{FH} signature genes in CXCL13-expressing or CXCL13-non-expressing cells; shape represents the distribution of expression among cells and color represents expression (log₂(CPM+1)). (FIG. 10D) Flow-cytometry analysis (left) shows the expression of PD-1 and CXCL13 in live, singlet-gated, CD45⁺CD3⁺CD4⁺ TILs (n=6) from patients with NSCLC. Barplot (right) show's percentage of PD1⁺ cells in CD4⁺CXCL13⁺ TILs. (FIG. 10E) Flow-cytometry analysis shows the expression (left) and percentage (middle) of FOXP3 and CXCL13 in live, singlet-gated, CD45⁺CD3⁺CD4⁺ TILs (n=6) from patients with NSCLC, **P<0.01 (Mann-Whitney test). Percentage (left margin) of CXCL13-expressing or CXCL13-non-expressing cells that express FOXP3 (right). GSEA of Treg signature in the transcriptome of CXCL13-expressing versus CXCL13-non-expressing cells presented as in FIG. 1C (far right). (FIG. 10F) Venn diagram shows overlap of genes differentially expressed in CXCL13-expressing cells versus non-expressing cells and cytotoxicity signature genes.

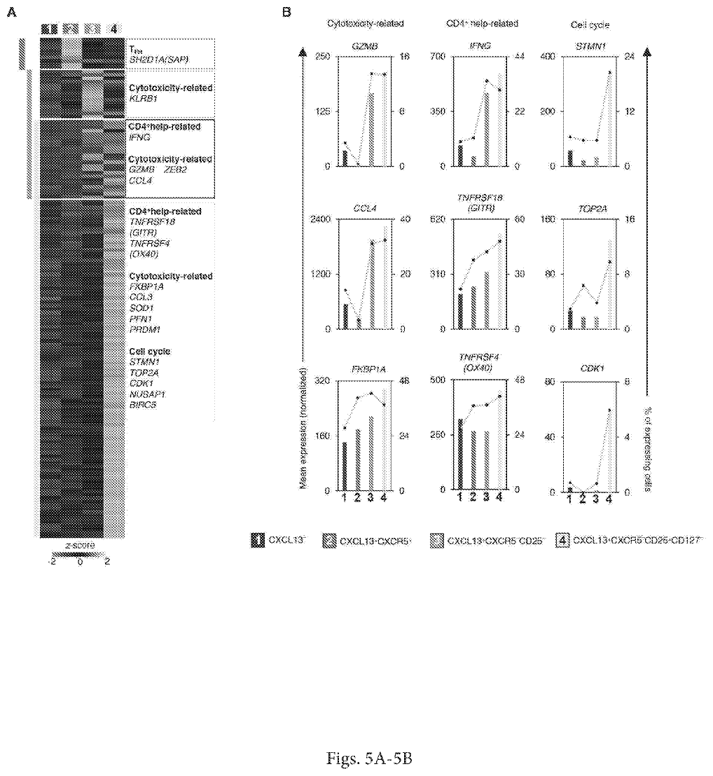

FIG. 11. Highly functional T_{FH}-like CD4⁺ T cells were CXCR5 negative. Related to FIG. 5; Table 10. GSEA of various gene sets (above plots) in the transcriptome of CXCL13-expressing cells in the indicated subsets (left margin) versus all CXCL13-non-expressing cells from CD4⁺ TILs presented as in FIG. 1C; red font indicates significant enrichment.

FIGS. 12A-12E. T_{FH}-like CD4⁺ T cells are present within human tumors and enriched following checkpoint blockade. Analysis of CD4⁺ TIL transcriptomes across a range of human cancers demonstrate that CXCL13-expressing cells are a target of anti-PD1 therapy and contribute to the ensuing anti-tumor immune response.

FIG. 13. Induction of T_{FH} by immunization bolsters CD8⁺ CTL response and impairs tumor growth. T_{FH} cells were increased in tumors of immunized mice relative to unimmunized mice, demonstrating the capacity of T_{FH} cells in lymph nodes to home to tumors. In addition, the proportion of proliferating T_{FH} cells (Ki-67⁺ T_{FH} cells) were increased in tumors of immunized mice compared to unimmunized mice. T_{FH} infiltration was also accompanied by increased frequency of CD8⁺ T cells, higher proportions of which were Cd39⁺ and Pd1⁺ in immunized mice, indicating enhanced activation following antigen-specific engagement. Furthermore, greater number of tumor-infiltrating CD8⁺ T cells from immunized mice expressed granzyme B and Ki-67, implying greater cytotoxic potential and cell proliferation.

### DETAILED DESCRIPTION OF THE DISCLOSURE

Disclosed herein are engineered cells and/or isolated cells that are Tfh-like tumor-infiltrating cells. The engineered cells and/or isolated cells may be Tfh-like cytotoxic T lymphocytes (Tfh-like CTLs). The engineered cells and/or isolated cells may be CD4⁺ Tfh-like tumor infiltrating cells.

Further disclosed herein are methods of producing an engineered cell. Generally the method comprises, consists essentially of, or consists of, modulating the expression and/or function of CXCR5, CD4, and/or CXCL13.

Further disclosed herein are methods of isolating Tfh-like tumor-infiltrating cells and/or Tfh-like CTLs. Generally, the method comprises, consists essentially of, or consists of, contacting a mixed population of cells with an agent that specifically binds to the Tfh-like tumor-infiltrating cell and/or Tfh-like CTL.

Further disclosed herein are methods of determining whether a subject will respond to a treatment for cancer. Generally, the method comprises, consists essentially of, or consists of, measuring the amount of one or more Tfh-like tumor-infiltrating cells and/or Tfh-like CTLs.

Further disclosed herein are methods of treating cancer in a subject in need thereof. Generally, the method comprises, consists essentially of, or consists of, administering a Tfh-like tumor-infiltrating cell and/or Tfh-like CTL to the subject.

### Definitions

Unless otherwise defined, all technical and scientific terms used herein have the same meaning as commonly understood by a person of ordinary skill in the art to which the present disclosure pertains. As used herein, and unless stated otherwise or required otherwise by context, each of the following terms shall have the definition set forth below.

In one embodiment, the methods described herein make use of the measured levels of the cell population of the present disclosure to detect surges, increases, or declines in cell numbers as predictive measures. As used herein, a “surge” or “increase” indicates a statistically significant increase in the level of relevant cells, typically from one measurement to one or more later measurements. In other instances, an increase in the level of relevant cells can be determined from one measure in a subject of interest relative to control (e.g., a value or a range of values for normal, i.e., healthy, individuals). Surges may be a two-fold increase in cell levels (i.e., a doubling of cell counts), a three-fold increase in cell levels (i.e., a tripling of cell numbers), a four-fold increase in cell levels (i.e., an increase by four times the number of cells in a previous measurement), or a five-fold or greater increase. In addition to the marked increase described as a surge, lesser increases in the levels of relevant cells may also have relevance to the methods of the present disclosure. Increases in cell levels may be described in terms of percentages. Surges may also be described in terms of percentages. For example, a surge or increase may be an increase in cell levels of 10%, 20%, 30%, 40%, 50%, 60%, 70%, 80%, 90%, or 100% or more. A “decline” indicates a decrease from one measurement to one or more later measurements. A decline may be a 5%, 10%, 15%, 20%, 25%, 30%, 35%, 40%, 45%, 50%, 55%, 60%, 65%, 70%, 75%, 80%, or 85% or greater decrease in cell levels from one measurement to one or more later measurements. In other instances, a decrease in the level of relevant cells can be determined from one measure in a subject of interest relative to control (e.g., a value or a range of values for normal, i.e., healthy, individuals).

In one embodiment, the surges, increases, or declines in cell numbers can be determined based on a comparison with a reference level derived from samples of at least 20 reference individuals without condition, a non-patient population. The surges or declines in cell numbers in a sample can also refer to a level that is elevated in comparison to the level of the cell numbers reached upon treatment, for example with an anti-cancer compound.

In one embodiment, the term “cancer” refers to a class of diseases in which a group of cells display uncontrolled growth, invasion, and/or metastasis. The term is meant to include, but not limited to, a cancer of the breast, respiratory tract, brain, reproductive organs, digestive tract, urinary tract, eye, liver, skin, head and neck, bone marrow, blood, thyroid, and parathyroid. The cancer may be a solid tumor, a non-solid tumor, or a distant metastasis of a tumor. Some specific examples of cancers include, but are not limited to, non-small cell lung cancer; small cell lung cancer; head and neck squamous cell carcinoma; renal cell carcinoma; bladder cancer; Hodgkin lymphoma; cutaneous squamous cell carcinoma; melanoma; myeloma; leukemia; Merkel-cell carcinoma; lymphomas; multiple myelomas; bone and connective tissue sarcomas; brain tumors; breast cancer; adrenal cancer; thyroid cancer; pancreatic cancer; pituitary cancers; eye cancers; vaginal cancers; cervical cancers; uterine cancers; ovarian cancers; esophageal cancers; stomach cancers; colon cancers; rectal cancers; gastric cancers; liver cancers; bladder cancers; gallbladder cancers; cholangiocarcinoma; lung cancers; testicular cancers; prostate cancers; penile cancers; oral cancers; basal cancers; salivary gland cancers; pharynx cancers; skin cancers; kidney cancers; melanomas or skin cancer, blood cancers such as myeloma, lymphoma, and Wilms' tumor. Examples of solid tumors include solid tumors of the breast, prostate, colon, pancreas, lung, gastric system, bladder, skin, and bone/connective tissue.

As used herein, “relapse” or “recurrence” may include the appearance of at least one new tumor lesion in, or new leukemia cells in the blood or bone marrow of, a subject who previously had cancer but has had no overt evidence of cancer as a result of surgery and/or therapy until relapse. Such recurrence of cancer cells can be local, occurring in the same area as one or more previous tumor lesions or leukemic cells, or distant, occurring in a previously lesion-free or cancer-cell free area, such as lymph nodes or other areas of the body.

As used herein, “response to treatment” may include complete response and partial response to treatment. A “complete response” (CR), in certain embodiments relating to e.g. cancer, is typically understood to include the disappearance of all target lesions and non-target lesions and normalization of tumor marker levels. A “partial response” (PR), in certain embodiments relating to cancer, is typically understood to include an at least 30% decrease in the sum of the diameters of target lesions. Generally speaking, in the context of embodiments relating to e.g. cancer, “response to treatment” may include an at least 30%-100% decrease in the sum of the diameters of target lesions, or disappearance of all target lesions and non-target lesions and normalization of tumor marker levels. “Progression” or “progressive disease” (PD), in certain embodiments relating to e.g. cancer, is typically understood to include an at least 20% increase in the sum of the diameters of target lesions, progression (increase in size) of any existing non-target lesions, and is also typically determined upon appearance of at least one new lesion. Non-CR/non-PD, in certain embodiments relating to e.g. cancer, is typically understood to include the persistence of one or more non-target lesions and/or maintenance of above-normal tumor marker levels. “Stable disease” (SD) is typically understood to include an insufficient increase to qualify for PD, but an insufficient decrease to qualify for PR. While the concepts of CR, PR, PD, and SD have been discussed in the context of cancer, the person of skill will readily understand that these concepts may also apply to other disease/conditions.

In one non-limiting embodiment, the biological sample from the subject which is suspected of including cell populations described herein includes blood or a cell fraction thereof.

In one non-limiting embodiment, the biological sample from the subject which is suspected of including the cell population of the present disclosure includes blood, spleen, tumor tissue, bone marrow or a cell fraction thereof.

As used herein, a “cell fraction” of a biological sample may be obtained using routine clinical cell fractionation techniques, such as gentle centrifugation, e.g., centrifugation at about 300-800×g for about five to about ten minutes or fractionated by other standard methods.

In one non-limiting embodiment, the herein described sample can be obtained by any known technique, for example by drawing, by non-invasive techniques, or from sample collections or banks, etc.

In one non-limiting embodiment, the present disclosure provides a kit which includes reagents that may be useful for implementing at least some of the herein described methods. The herein described kit may include at least one detecting agent which is “packaged”. As used herein, the term “packaged” can refer to the use of a solid matrix or material such as glass, plastic, paper, fiber, foil and the like, capable of holding within fixed limits the at least one detection reagent. Thus, in one non-limiting embodiment, the kit may include the at least one detecting agent “packaged” in a glass vial used to contain microgram or milligram quantities of the at least one detecting agent. In another non-limiting embodiment, the kit may include the at least one detecting agent “packaged” in a microtiter plate well to which microgram quantities of the at least one detecting agent has been operatively affixed. In another non-limiting embodiment, the kit may include the at least one detecting agent coated on microparticles entrapped within a porous membrane or embedded in a test strip or dipstick, etc. In another non-limiting embodiment, the kit may include the at least one detecting agent directly coated onto a membrane, test strip or dipstick, etc. which contacts the sample fluid. Many other possibilities exist and will be readily recognized by those skilled in this art without departing from the disclosure. For example, the kit may include a combination of detecting agent which can be useful for cell sorting the cell populations of the present disclosure, as discussed elsewhere in the present document.

As used herein, a “purified cell population” refers to a cell population which has been processed so as to separate the cell population from other cell populations with which it is normally associated in its naturally occurring state. The purified cell population can, thus, represent an enriched cell population in that the relative concentration of the cell population in a sample can be increased following such processing in comparison to its natural state. In one embodiment, the purified cell population can refer to a cell population which is enriched in a composition in a relative amount of at least 80%, or at least 90%, or at least 95% or 100% in comparison to its natural state. Such purified cell population may, thus, represent a cell preparation which can be further processed so as to obtain commercially viable preparations. For example, in one embodiment, the cell preparation can be prepared for transportation or storage in a serum-based solution containing necessary additives (e.g., DMSO), which can then be stored or transported in a frozen form. In doing so, the person of skill will readily understand that the cell preparation is in a composition that includes a suitable carrier, which composition is significantly different from the natural occurring separate elements. For example, the serum-based preparation may comprise, consist essentially of, or consist of, human serum or fetal bovine serum, which is a structural form that is markedly different from the form of the naturally occurring elements of the preparation. The resulting preparation includes cells that are in dormant state, for example, that may have slowed down or stopped intracellular metabolic reactions and/or that may have structural modifications to its cellular membranes. The resulting preparation includes cells that can, thus, be packaged or shipped while minimizing cell loss which would otherwise occur with the naturally occurring cells. A person skilled in the art would be able to determine a suitable preparation without departing from the present disclosure.

As used herein, the term “about” for example with respect to a value relating to a particular parameter (e.g. concentration, such as “about 100 mM”) relates to the variation, deviation or error (e.g. determined via statistical analysis) associated with a device or method used to measure the parameter. For example, in the case where the value of a parameter is based on a device or method which is capable of measuring the parameter with an error of ±10%, “about” would encompass the range from less than 10% of the value to more than 10% of the value.

As used herein, the singular forms “a”, “an,” and “the” include plural referents unless the context clearly indicates otherwise.

As used herein, the term “comprising” is intended to mean that the compositions or methods include the recited steps or elements, but do not exclude others. “Consisting essentially of” shall mean rendering the claims open only for the inclusion of steps or elements, which do not materially affect the basic and novel characteristics of the claimed compositions and methods. “Consisting of” shall mean excluding any element or step not specified in the claim. Embodiments defined by each of these transition terms are within the scope of this disclosure.

As used herein “a population of cells” intends a collection of more than one cell that is identical (clonal) or non-identical in phenotype and/or genotype.

As used herein, “substantially homogenous” population of cells is a population having at least 70%, or alternatively at least 75%, or alternatively at least 80%, or alternatively at least 85%, or alternatively at least 90%, or alternatively at least 95%, or alternatively at least 98% identical phenotype, as measured by pre-selected markers, phenotypic or genomic traits. In one aspect, the population is a clonal population.

As used herein, “heterogeneous” population of cells is a population having up to 69%, or alternatively up to 60%, or alternatively up to 50%, or alternatively up to 40%, or alternatively up to 30%, or alternatively up to 20%, or alternatively up to 10%, or alternatively up to 5%, or alternatively up to 4%, or alternatively up to 3%, or alternatively up to 2%, or alternatively up to 61%, or alternatively up to 0.5% identical phenotype, as measured by pre-selected markers, phenotypic or genomic traits.

The term “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human, adult, juvenile, children, and infants. In particular, in the context of cancer, the subject can be a mammal who previously had cancer but appears to have recovered as a result of surgery and/or therapy, or who presently has cancer and is undergoing cancer therapy, or has completed a cancer therapeutic regime, or has received no cancer therapy. In some embodiments, a human has or is suspected of having a cancer or neoplastic disorder.

As used herein, the terms “therapeutically effective amount” and “effective amount” are used interchangeably to refer to an amount of a composition of the disclosure that is sufficient to result in the prevention of the development, recurrence, or onset of a disease or condition. For example, in certain embodiments e.g. cancer, these terms refer to an amount of a composition of the disclosure that is sufficient to result in the prevention of the development, recurrence, or onset of cancer stem cells or cancer and one or more symptoms thereof, to enhance or improve the prophylactic effect(s) of another therapy, reduce the severity and duration of cancer, ameliorate one or more symptoms of cancer, prevent the advancement of cancer, cause regression of cancer, and/or enhance or improve the therapeutic effect(s) of additional anticancer treatment(s).

A therapeutically effective amount can be administered to a patient in one or more doses sufficient to palliate, ameliorate, stabilize, reverse or slow the progression of the disease, or otherwise reduce the pathological consequences of the disease, or reduce the symptoms of the disease. The amelioration or reduction need not be permanent but may be for a period of time ranging from at least one hour, at least one day, or at least one week or more. The effective amount is generally determined by the physician on a case-by-case basis and is within the skill of one in the art. Several factors are typically taken into account when determining an appropriate dosage to achieve an effective amount. These factors include age, sex and weight of the patient, the condition being treated, the severity of the condition, as well as the route of administration, dosage form and regimen and the desired result.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Treatments containing the disclosed compositions and methods can be first line, second line, third line, fourth line, fifth line therapy and are intended to be used as a sole therapy or in combination with other appropriate therapies. In one aspect, treatment excludes prophylaxis. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor.

As used herein, the phrase “respond to treatment” or similar phrases refer to the clinical benefit imparted to a patient suffering from a disease or condition, such as cancer, from or as a result of the treatment described herein. A clinical benefit includes a complete remission, a partial remission, a stable disease (without progression), progression-free survival, disease free survival, improvement in the time-to-progression (of the disease), improvement in the time-to-death, or improvement in the overall survival time of the patient from or as a result of the treatment described herein. There are criteria for determining a response to therapy and those criteria allow comparisons of the efficacy to alternative treatments (Slapak and Kufe, Principles of Cancer Therapy, in Harrisons's Principles of Internal Medicine, 13^{th} edition, eds. Isselbacher et al., McGraw-Hill, Inc., 1994). For example, a complete response or complete remission of cancer is the disappearance of all detectable malignant disease. A partial response or partial remission of cancer may be, for example, an approximately 50 percent decrease in the product of the greatest perpendicular diameters of one or more lesions or where there is not an increase in the size of any lesion or the appearance of new lesions.

The term “clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival (DFS), time to tumor recurrence (TTR), time to tumor progression (TTP), relative risk (RR), toxicity or side effects.

A “complete response” (CR) to a therapy defines patients with evaluable but non-measurable disease, whose tumor and all evidence of disease had disappeared.

A “partial response” (PR) to a therapy defines patients with anything less than complete response that were simply categorized as demonstrating partial response.

“Stable disease” (SD) indicates that the patient is stable.

“Progressive disease” (PD) indicates that the tumor has grown (i.e. become larger), spread (i.e. metastasized to another tissue or organ) or the overall cancer has gotten worse following treatment. For example, tumor growth of more than 20 percent since the start of treatment typically indicates progressive disease.

“Disease free survival” (DFS) indicates the length of time after treatment of a cancer or tumor during which a patient survives with no signs of the cancer or tumor.

“Non-response” (NR) to a therapy defines patients whose tumor or evidence of disease has remained constant or has progressed.

“Overall Survival” (OS) intends a prolongation in life expectancy as compared to naïve or untreated individuals or patients.

“Progression free survival” (PFS) or “Time to Tumor Progression” (TTP) indicates the length of time during and after treatment that the cancer does not grow. Progression-free survival includes the amount of time patients have experienced a complete response or a partial response, as well as the amount of time patients have experienced stable disease.

“No Correlation” refers to a statistical analysis showing no relationship between the allelic variant of a polymorphic region or gene expression levels and clinical parameters.

“Tumor Recurrence” as used herein and as defined by the National Cancer Institute is cancer that has recurred (come back), usually after a period of time during which the cancer could not be detected. The cancer may come back to the same place as the original (primary) tumor or to another place in the body. It is also called recurrent cancer.

“Time to Tumor Recurrence” (TTR) is defined as the time from the date of diagnosis of the cancer to the date of first recurrence, death, or until last contact if the patient was free of any tumor recurrence at the time of last contact. If a patient had not recurred, then TTR was censored at the time of death or at the last follow-up.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

As used herein, the terms “stage I cancer,” “stage II cancer,” “stage III cancer,” and “stage IV” refer to the TNM staging classification for cancer. Stage I cancer typically identifies that the primary tumor is limited to the organ of origin. Stage II intends that the primary tumor has spread into surrounding tissue and lymph nodes immediately draining the area of the tumor. Stage III intends that the primary tumor is large, with fixation to deeper structures. Stage IV intends that the primary tumor is large, with fixation to deeper structures. See pages 20 and 21, CANCER BIOLOGY, 2^{nd} Ed., Oxford University Press (1987).

A “tumor” is an abnormal growth of tissue resulting from uncontrolled, progressive multiplication of cells and serving no physiological function. A “tumor” is also known as a neoplasm.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

The term “contacting” means direct or indirect binding or interaction between two or more entities. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

As used herein, the term “administer” and “administering” are used to mean introducing the therapeutic agent (e.g. polynucleotide, vector, cell, modified cell, population) into a subject. The therapeutic administration of this substance serves to attenuate any symptom, or prevent additional symptoms from arising. When administration is for the purposes of preventing or reducing the likelihood of developing cancer, the substance is provided in advance of any visible or detectable symptom or relapse. Routes of administration include, but are not limited to, oral (such as a tablet, capsule or suspension), topical, transdermal, intranasal, vaginal, rectal, subcutaneous intravenous, intraarterial, intramuscular, intraosseous, intraperitoneal, epidural and intrathecal.

As used herein, the term “expression” or “express” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound. The terms “upregulate” and “downregulate” and variations thereof when used in context of gene expression, respectively, refer to the increase and decrease of gene expression relative to a normal or expected threshold expression for cells, in general, or the sub-type of cell, in particular.

As used herein, the term “gene expression profile” refers to measuring the expression level of multiple genes to establish an expression profile for a particular sample.

As used herein, the term “reduce or eliminate expression and/or function of” refers to reducing or eliminating the transcription of said polynucleotides into mRNA, or alternatively reducing or eliminating the translation of said mRNA into peptides, polypeptides, or proteins, or reducing or eliminating the functioning of said peptides, polypeptides, or proteins. In a non-limiting example, the transcription of polynucleotides into mRNA is reduced to at least half of its normal level found in wild type cells.

As used herein, the term “increase expression of” refers to increasing the transcription of said polynucleotides into mRNA, or alternatively increasing the translation of said mRNA into peptides, polypeptides, or proteins, or increasing the functioning of said peptides, polypeptides, or proteins. In a non-limiting example, the transcription of polynucleotides into mRNA is increased to at least twice of its normal level found in wild type cells.

An “an effective amount” or “efficacious amount” is an amount sufficient to achieve the intended purpose, non-limiting examples of such include: initiation of the immune response, modulation of the immune response, suppression of an inflammatory response and modulation of T cell activity or T cell populations. In one aspect, the effective amount is one that functions to achieve a stated therapeutic purpose, e.g., a therapeutically effective amount. As described herein in detail, the effective amount, or dosage, depends on the purpose and the composition, and can be determined according to the present disclosure.

A “composition” typically intends a combination of the active agent, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (I), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The compositions used in accordance with the disclosure, including cells, treatments, therapies, agents, drugs and pharmaceutical formulations can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

The term “isolated” as used herein refers to molecules or biologicals or cellular materials being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, the term “isolated cell” generally refers to a cell that is substantially separated from other cells of a tissue.

As used herein, the term “antibody” (“Ab”) collectively refers to immunoglobulins (or “Ig”) or immunoglobulin-like molecules including but not limited to antibodies of the following isotypes: IgM, IgA, IgD, IgE, IgG, and combinations thereof. Immunoglobulin-like molecules include but are not limited to similar molecules produced during an immune response in a vertebrate, for example, in mammals such as humans, rats, goats, rabbits and mice, as well as non-mammalian species, such as shark immunoglobulins (see Feige, M. et al. Proc. Nat. Ac. Sci. 41(22): 8155-60 (2014)). Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997. One of skill in the art can monitor expression of mRNA using methods such as RNA-sequencing, DNA microarrays, Real-time PCR, or Chromatin immunoprecipitation (ChIP) etc. Protein expression can be monitored using methods such as flow cytometry, Western blotting, 2-D gel electrophoresis or immunoassays etc.

In one aspect, the term “equivalent” or “biological equivalent” of an antibody means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody.

One of skill in the art can use methods such as RNA interference (RNAi), CRISPR, TALEN, ZFN or other methods that target specific sequences to reduce expression and/or increase expression/and or function of CD33.

As used herein, “RNAi” (RNA interference) refers to the method of reducing or eliminating gene expression in a cell by targeting specific mRNA sequences for degradation via introduction of short pieces of double stranded RNA (dsRNA) and small interfering RNA (such as siRNA, shRNA or miRNA etc.) (Agrawal, N. et al.; Microbiol Mol Biol Rev. 2003; 67:657-685, Arenz, C. et al.; Naturwissenschaften. 2003; 90:345-359, Hannon G J.; Nature. 2002; 418:244-251).

As used herein, the term “CRISPR” refers to a technique of sequence specific genetic manipulation relying on the clustered regularly interspaced short palindromic repeats pathway. CRISPR can be used to perform gene editing and/or gene regulation, as well as to simply target proteins to a specific genomic location. “Gene editing” refers to a type of genetic engineering in which the nucleotide sequence of a target polynucleotide is changed through introduction of deletions, insertions, single stranded or double stranded breaks, or base substitutions to the polynucleotide sequence. In some aspects, CRISPR-mediated gene editing utilizes the pathways of non-homologous end-joining (NHEJ) or homologous recombination to perform the edits. Gene regulation refers to increasing or decreasing the production of specific gene products such as protein or RNA.

The term “gRNA” or “guide RNA” as used herein refers to guide RNA sequences used to target specific polynucleotide sequences for gene editing employing the CRISPR technique. Techniques of designing gRNAs and donor therapeutic polynucleotides for target specificity are well known in the art. For example, Doench, J., et al. Nature biotechnology 2014; 32(12): 1262-7, Mohr, S. et al. (2016) FEBS Journal 283: 3232-38, and Graham, D., et al. Genome Biol. 2015; 16: 260. gRNA comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some aspects, a gRNA is synthetic (Kelley, M. et al. (2016) J of Biotechnology 233 (2016) 74-83).

The term “Cas9” refers to a CRISPR associated endonuclease referred to by this name. Non-limiting exemplary Cas9s include Staphylococcus aureus Cas9, nuclease dead Cas9, and orthologs and biological equivalents each thereof. Orthologs include but are not limited to Streptococcus pyogenes Cas9 (“spCas9”), Cas 9 from Streptococcus thermophiles, Legionella pneumophilia, Neisseria lactamica, Neisseria meningitides, Francisella novicida; and Cpf1 (which performs cutting functions analogous to Cas9) from various bacterial species including Acidaminococcus spp. and Francisella novicida UI12.

As used herein, “TALEN” (transcription activator-like effector nucleases) refers to engineered nucleases that comprise a non-specific DNA-cleaving nuclease fused to a TALE DNA-binding domain, which can target DNA sequences and be used for genome editing. Boch (2011) Nature Biotech. 29: 135-6; and Boch et al. (2009) Science 326: 1509-12; Moscou et al. (2009) Science 326: 3501. TALEs are proteins secreted by Xanthomonas bacteria. The DNA binding domain contains a repeated, highly conserved 33-34 amino acid sequence, with the exception of the ¹2th and ¹3th amino acids. These two positions are highly variable, showing a strong correlation with specific nucleotide recognition. They can thus be engineered to bind to a desired DNA sequence. To produce a TALEN, a TALE protein is fused to a nuclease (N), which is a wild-type or mutated Fokl endonuclease. Several mutations to Fokl have been made for its use in TALENs; these, for example, improve cleavage specificity or activity. Cermak et al. (2011) Nucl. Acids Res. 39: e82; Miller et al. (2011) Nature Biotech. 29: 143-8; Hockemeyer et al. (2011) Nature Biotech. 29: 731-734; Wood et al. (2011) Science 333: 307; Doyon et al. (2010) Nature Methods 8: 74-79; Szczepek et al. (2007) Nature Biotech. 25: 786-793; and Guo et al. (2010) J. Mol. Bio. 200: 96. The Fokl domain functions as a dimer, requiring two constructs with unique DNA binding domains for sites in the target genome with proper orientation and spacing. Both the number of amino acid residues between the TALE DNA binding domain and the Fokl cleavage domain and the number of bases between the two individual TALEN binding sites appear to be important parameters for achieving high levels of activity. Miller et al. (2011) Nature Biotech. 29: 143-8. TALENs specific to sequences in immune cells can be constructed using any method known in the art, including various schemes using modular components. Zhang et al. (2011) Nature Biotech. 29: 149-53; Geibler et al. (2011) PLoS ONE 6: e19509.

As used herein, “ZFN” (Zinc Finger Nuclease) refers to engineered nucleases that comprise a non-specific DNA-cleaving nuclease fused to a zinc finger DNA binding domain, which can target DNA sequences and be used for genome editing. Like a TALEN, a ZFN comprises a Fokl nuclease domain (or derivative thereof) fused to a DNA-binding domain. In the case of a ZFN, the DNA-binding domain comprises one or more zinc fingers. Carroll et al. (2011) Genetics Society of America 188: 773-782; and Kim et al. (1996) Proc. Natl. Acad. Sci. USA 93: 1156-1160. A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Like a TALEN, a ZFN must dimerize to cleave DNA. Thus, a pair of ZFNs are required to target non-palindromic DNA sites. The two individual ZFNs must bind opposite strands of the DNA with their nucleases properly spaced apart. Bitinaite et al. (1998) Proc. Natl. Acad. Sci. USA 95: 10570-5. ZFNs specific to sequences in immune cells can be constructed using any method known in the art. See, e.g., Provasi (2011) Nature Med. 18: 807-815; Torikai (2013) Blood 122: 1341-1349; Cathomen et al. (2008) Mol. Ther. 16: 1200-7; Guo et al. (2010) J. Mol. Bioi. 400: 96; U.S. Patent Publication 201110158957; and U.S. Patent Publication 2012/0060230.

The term “culturing” refers to growing cells in a culture medium under conditions that favor expansion and proliferation of the cell. The term “culture medium” or “medium” is recognized in the art, and refers generally to any substance or preparation used for the cultivation of living cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for culture is a medium. Similarly, a powder mixture that when mixed with water or other liquid becomes suitable for cell culture may be termed a “powdered medium.” “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high density culture” is any medium that allows a cell culture to reach an OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. In one aspect, the growth medium may be a complex medium with the necessary growth factors to support the growth and expansion of the cells of the disclosure while maintaining their self-renewal capability. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, McCoy's 5A, Dulbecco's MEM/F-12, RPMI 1640, and Iscove's Modified Dulbecco's Medium (IMDM).

“Cryoprotectants” are known in the art and include without limitation, e.g., sucrose, trehalose, and glycerol. A cryoprotectant exhibiting low toxicity in biological systems is generally used.

“Classical monocyte” intends a monocyte that is (CD14⁺).

“Nonclassical monocyte” intends a monocyte that is (CD14^{lo}CD16⁺).

“Intermediate monocyte” intends a monocyte that is (CD14⁺CD16⁺).

“Checkpoint inhibitor” intends a drug or therapy that blocks certain proteins made by some immune cells, such as T cells and some cancer cells. These proteins assist with immune responses, and keep immune responses in check. When these proteins are blocked, the brakes on the immune system are released and T cell are able to inhibit the growth or kill cancer cells. Non-limiting examples of checkpoint proteins on T cell or cancer cells include PD-1/PD-L1 and CTLA-4/B7-1/B7-2. Checkpoint inhibitors are used to treat cancer alone or in combination with other therapies and treatments. Non-limiting examples of PD-1 inhibits include Pembrolizumab (Keytruda), Nivolumab (Opdivo) and Cemiplimab (Libtayao), which have been shown to treat melanoma, NSCLC, kidney cancer, bladder cancer, head and neck cancers, and Hodgkin lymphoma. Non-limiting examples of PD-L1 inhibitors include Atezolizumab (Tecentriq), Avelumab (Bavencio), and Durvalumab (Imfinzil). These drugs have been shown to treat bladder cancer, NSCLC, Merkel cell skin cancer (Merkel cell carcinoma). Non-limiting examples of drugs that target CTLA-1 include Ipilimumab (Yervoy) which has been used to treat melanoma and other cancers. Additional treatments are under development as described for example in Darvin et al. (2018) Exper. & Mol. Med. 50, Article number 165 and Tang et al. (2018) Nature Reviews Drug Discover 17:854-855. The Cancer Research Institute reports that over 2,250 clinical trials are evaluating PD-1/L1 checkpoint inhibitors and 1,716 trials are assessing regimens that combine PD-1/L1 immune checkpoints with other therapies. 240 drug targets are being evaluated in the current landscape. See cancerresearch.org/news/2018/pd-1-l1-checkpoint-inhibitor-landscape-analysis, last accessed Jun. 5, 2019.

As used herein, the phrase “T-follicular helper (Tfh)-like tumor-infiltrating cell” refers to a cell that is associated with proliferation, cytotoxicity and/or tissue residency in CD8⁺ T cells within tumors. In some embodiments, a Tfh-like tumor-infiltrating cell is a cell that is engineered to express features linked to proliferation, cytotoxicity and/or CD8⁺ T cell ‘help’. Alternatively, a Tfh-like tumor-infiltrating cell is a cell that expresses features linked to proliferation, cytotoxicity and/or CD8⁺ T cell ‘help’.

Engineered Cells and Isolated Cells

The methods, uses, kits, and compositions disclosed herein may comprise, consist essentially of, or consist of, or use one or more engineered cells disclosed herein. In some embodiments, the engineered cell is an engineered T-follicular helper (Tfh)-like tumor-infiltrating cell. In some embodiments, the engineered Tfh-like tumor-infiltrating cell is engineered to modulate expression of the surface markers cluster of differentiation 4 (CD4), chemokine (C-X-C motif) ligand 13 (CXCL13) and C-X-C Motif Chemokine Receptor 5 (CXCR5). In some embodiments, the engineered Tfh-like tumor-infiltrating cell is engineered to express the surface markers CD4 and CXCL13 and lack the surface marker CXCR5. In some embodiments, the engineered Tfh-like tumor-infiltrating cell is further engineered to express granzyme B (GZMB). In some embodiments, the engineered cell is a Tfh-like tumor-infiltrating cell that activates a CD8⁺ CTL response. In some embodiments, the CD8⁺ CTL response is activated in a tumor or tumor microenvironment. In some embodiments the engineered cell is a Tfh-like tumor-infiltrating cell that activates a CD8⁺ T_{RM} response. In some embodiments, the CD8⁺ TRM response is activated in a tumor or tumor microenvironment.

Disclosed herein is an engineered Tfh-like tumor-infiltrating cell engineered to modulate expression of one or more proteins selected from Proto-Oncogene C-Maf (MAF), SH2D1A (SAP), programmed cell death 1 (PDCD1), B And T Lymphocyte Associated (BTLA), CD200, and BCL6. In some embodiments, the cell is engineered to modulate expression of two, three, four, or five, or more proteins selected from Proto-Oncogene C-Maf (MAF), SH2D1A (SAP), programmed cell death 1 (PDCD1), B And T Lymphocyte Associated (BTLA), CD200, and BCL6. In some embodiments, the cell is engineered to increase expression of one, two, three, four, or five, or more proteins selected from Proto-Oncogene C-Maf (MAF), SH2D1A (SAP), programmed cell death 1 (PDCD1), B And T Lymphocyte Associated (BTLA), CD200, and BCL6. In some embodiments, the cell is contacted with one or more polynucleotides encoding one or more proteins selected from Proto-Oncogene C-Maf (MAF), SH2D1A (SAP), programmed cell death 1 (PDCD1), B And T Lymphocyte Associated (BTLA), CD200, and BCL6, thereby increasing expression and/or function of the protein. In some embodiments, the cell is engineered to decrease expression of one, two, three, four, or five, or more proteins selected from Proto-Oncogene C-Maf (MAF), SH2D1A (SAP), programmed cell death 1 (PDCD1), B and T Lymphocyte Associated (BTLA), CD200, and BCL6. In some embodiments, the cell is contacted with one or more oligonucleotides that inhibit expression of one or more proteins selected from Proto-Oncogene C-Maf (MAF), SH2D1A (SAP), programmed cell death 1 (PDCD1), B and T Lymphocyte Associated (BTLA), CD200, and BCL6. In some embodiments, the oligonucleotide that inhibits expression of the protein is an antisense oligonucleotide that targets the protein. In some embodiments, the antisense oligonucleotide is a miRNA, shRNA, or siRNA. In some embodiments, the oligonucleotide that inhibits expression of the protein is a guide RNA that targets the protein. In some embodiments, the cell is engineered to increase expression and/or function of TNF Receptor Superfamily Member 18 (TNFRSF18).

In some embodiments, methods to inhibit expression of gene of interest comprise, consist essentially of, or consist of, delivery of shRNA, such as retroviral transduced CD4 T cells (described in Chen, R., Belanger, S., Frederick, M. A., Li, B., Johnston, R. J., Xiao, N., Liu, Y. C., Sharma, S., Peters, B., Rao, A. and Crotty, S., 2014. In vivo RNA interference screens identify regulators of antiviral CD4+ and CD8+ T cell differentiation. Immunity, 41(T), pp. 325-338., each of which are incorporated by reference in their entirety)

The methods, uses, kits, and compositions disclosed herein may comprise, consist essentially of, or consist of, or use one or more isolated cells disclosed herein. In some embodiments, the isolated Tfh-like tumor-infiltrating cell expresses the surface markers CD4 and CXCL13 and lacks the surface marker CXCR5. In some embodiments, the cell is a cytotoxic Tfh-like tumor-infiltrating cell expressing GZMB.

Further disclosed herein is a substantially homogenous population of cells comprising, consisting essentially of, or consisting of, any one of the cells disclosed herein. In some embodiments, at least 80%, 85%, 90%, 95%, 96%, 97%, 98%, 99%, or more of the cells in the homogenous population of cells are any one of the cells disclosed herein. In some embodiments, at least 80%, 85%, 90%, 95%, 96%, 97%, 98%, 99%, or more of the cells in the homogenous population of cells express CD4 and CXCL13 and lack the surface marker CXCR5. In some embodiments, at least 80%, 85%, 90%, 95%, 96%, 97%, 98%, 99%, or more of the cells in the homogenous population of cells express CD4, CXCL13, GZMB and lack the surface marker CXCR5.

A heterogeneous population of cells of comprising, consisting essentially of, or consisting of, any of the cells disclosed herein. In some embodiments, at least 60%, 65%, 70%, 75%, 80%, 85%, 90%, 95%, or more of the cells in the heterogeneous population of cells are any one of the cells disclosed herein. In some embodiments, at least 60%, 65%, 70%, 75%, 80%, 85%, 90%, 95%, or more of the cells in the heterogeneous population of cells express CD4 and CXCL13 and lack the surface marker CXCR5. In some embodiments, at least 60%, 65%, 70%, 75%, 80%, 85%, 90%, 95%, or more of the cells in the heterogeneous population of cells express CD4, CXCL13, GZMB and lack the surface marker CXCR5.

In some embodiments, any one of the cells disclosed herein is engineered to increase expression and/or function of one, two, three, or four or more of: TNF Receptor Superfamily Member 18 (TNFRSF18), TNF Receptor Superfamily Member 4 (TNFRSF4), interferon gamma (IFNG), Granzyme B and/or IL21 in the cell. In some embodiments, an engineered cell disclosed herein is engineered to increase expression and/or function of one, two, three, or four or more of: TNF Receptor Superfamily Member 18 (TNFRSF18), TNF Receptor Superfamily Member 4 (TNFRSF4), interferon gamma (IFNG), Granzyme B and/or IL21 in the cell. In some embodiments, the engineered cell is contacted with one or more polynucleotides encoding one or more proteins selected from: TNF Receptor Superfamily Member 18 (TNFRSF18), TNF Receptor Superfamily Member 4 (TNFRSF4), interferon gamma (IFNG), Granzyme B and IL21, thereby increasing expression and/or function of the protein. In some embodiments, an isolated cell disclosed herein is engineered to increase expression and/or function of one, two, three, or four or more of: TNF Receptor Superfamily Member 18 (TNFRSF18), TNF Receptor Superfamily Member 4 (TNFRSF4), interferon gamma (IFNG), Granzyme B and/or IL21 in the cell. In some embodiments, the isolated cell is contacted with one or more polynucleotides encoding one or more proteins selected from: TNF Receptor Superfamily Member 18 (TNFRSF18), TNF Receptor Superfamily Member 4 (TNFRSF4), interferon gamma (IFNG), Granzyme B and IL21, thereby increasing expression and/or function of the protein. In some embodiments, an isolated cell disclosed herein has increased expression and/or function of one, two, three, or four more of: TNF Receptor Superfamily Member 18 (TNFRSF18), TNF Receptor Superfamily Member 4 (TNFRSF4), interferon gamma (IFNG), Granzyme B and/or IL21 in the cell.

In some embodiments, the engineered cell disclosed herein is a cell that is contacted with a polynucleotide encoding a CD4 protein. In some embodiments, the isolated cell disclosed herein is contacted with a polynucleotide encoding a CD4 protein. In some embodiments, the polynucleotide encoding the CD4 protein comprises, consists essentially of, or consists of, the nucleotide sequence of any one of SEQ ID NOs: 1, 3, 5, 7, and 9. In some embodiments, the polynucleotide encoding the CD4 protein comprises, consists essentially of, or consists of, a fragment of the nucleotide sequence of any one of SEQ ID NOs: 1, 3, 5, 7, and 9 that encodes for the CD4 protein comprising, consisting essentially of, or consisting of, the amino acid sequence of any one of SEQ ID NOs: 2, 4, 6, 8, and 10. In some embodiments, the CD4 protein comprises, consists essentially of, or consists of, the amino acid sequence of any one of SEQ ID NOs: 2, 4, 6, 8, and 10. In some embodiments, the polynucleotide encoding the CD4 protein comprises, consists essentially of, or consists of, a nucleotide sequence that is at least 85%, 90%, 93%, 94%, 95%, 96%, 97%, 98%, 99%, or 100% identical to the nucleotide sequence of any one of SEQ ID NOs: 1, 3, 5, 7, and 9. In some embodiments, the polynucleotide encoding the CD4 protein comprises, consists essentially of, or consists of, a fragment of a nucleotide sequence that is at least 85%, 90%, 93%, 94%, 95%, 96%, 97%, 98%, 99%, or 100% identical to the nucleotide sequence of any one of SEQ ID NOs: 1, 3, 5, 7, and 9 that encodes for the CD4 protein comprising, consisting essentially of, or consisting of, an amino acid sequence that is at least 85%, 90%, 93%, 94%, 95%, 96%, 97%, 98%, 99%, or 100% identical to the amino acid sequence of any one of SEQ ID NOs: 2, 4, 6, 8, and 10. In some embodiments, the CD4 protein comprises, consists essentially of, or consists of, an amino acid sequence that is at least 85%, 90%, 93%, 94%, 95%, 96%, 97%, 98%, 99%, or 100% identical to the amino acid sequence of any one of SEQ ID NOs: 2, 4, 6, 8, and 10. In some embodiments, the CD4 is a mammalian CD4. In some embodiments, the CD4 is a human CD4.

In some embodiments, the engineered cell disclosed herein is a cell that is contacted with a polynucleotide encoding a CXCL13 protein. In some embodiments, the isolated cell disclosed herein is contacted with a polynucleotide encoding a CXCL13 protein. In some embodiments, the polynucleotide encoding the CXCL13 protein comprises, consists essentially of, or consists of, the nucleotide sequence of any one of SEQ ID NOs: 11 and 13. In some embodiments, the polynucleotide encoding the CXCL13 protein comprises, consists essentially of, or consists of, a fragment of the nucleotide sequence of any one of SEQ ID NOs: 11 and 13 that encodes for the CXCL13 protein comprising, consisting essentially of, or consisting of, the amino acid sequence of any one of SEQ ID NOs: 12 and 14. In some embodiments, the CXCL13 protein comprises, consists essentially of, or consists of, the amino acid sequence of any one of SEQ ID NOs: 12 and 14. In some embodiments, the polynucleotide encoding the CXCL13 protein comprises, consists essentially of, or consists of, a nucleotide sequence that is at least 85%, 90%, 93%, 94%, 95%, 96%, 97%, 98%, 99%, or 100% identical to the nucleotide sequence of any one of SEQ ID NOs: 11 and 13. In some embodiments, the polynucleotide encoding the CXCL13 protein comprises, consists essentially of, or consists of, a fragment of a nucleotide sequence that is at least 85%, 90%, 93%, 94%, 95%, 96%, 97%, 98%, 99%, or 100% identical to the nucleotide sequence of any one of SEQ ID NOs: 11 and 13 that encodes for the CXCL13 protein comprising, consisting essentially of, or consisting of, an amino acid sequence that is at least 85%, 90%, 93%, 94%, 95%, 96%, 97%, 98%, 99%, or 100% identical to the amino acid sequence of any one of SEQ ID NOs: 12 and 14. In some embodiments, the CXCL13 protein comprises, consists essentially of, or consists of, an amino acid sequence that is at least 85%, 90%, 93%, 94%, 95%, 96%, 97%, 98%, 99%, or 100% identical to the amino acid sequence of any one of SEQ ID NOs: 12 and 14. In some embodiments, the CXCL13 is a mammalian CXCL13. In some embodiments, the CXCL13 is a human CXCL13.

In some embodiments, the engineered cell disclosed herein is a cell that is contacted with an oligonucleotide that inhibits expression of CXCR5. In some embodiments, the isolated cell disclosed herein is contacted with an oligonucleotide that inhibits expression of CXCR5. In some embodiments, the oligonucleotide that inhibits expression of CXCR5 is an antisense oligonucleotide that targets a CXCR5 polynucleotide. In some embodiments, the antisense oligonucleotide is a microRNA (miRNA), short hairpin RNA (shRNA), or small interfering RNA (siRNA). In some embodiments, the oligonucleotide that inhibits expression of CXCR5 is a guide RNA (gRNA) that targets a CXCR5 polynucleotide. Examples of oligonucleotides that inhibit expression of CXCR5 are known in the art, and include, but are not limited to, CXCR5 shRNA TL306391 (Origene), CXCR5 siRNA SR300441 (Origene), CXCR5 shRNA TR306391 (Origene), CXCR5 shRNA TL306391V (Origene), CXCR5 CRISPR gRNA ABIN5115520 (Genomics Online), and CXCR5 CRISPR gRNA ABIN5115519 (Genomics Online). In some embodiments, the CXCR5 polynucleotide is a mammalian CXCR5 polynucleotide. In some embodiments, the CXCR5 polynucleotide is a human CXCR5 polynucleotide. In some embodiments, the CXCR5 polynucleotide comprises, consists essentially of, or consists of, the nucleotide sequence of any one of SEQ ID NOs: 15 and 17. In some embodiments, the CXCR5 polynucleotide comprises, consists essentially of, or consists of, a fragment of the nucleotide sequence of any one of SEQ ID NOs: 15 and 17 that encodes for a CXCR5 protein comprising, consisting essentially of, or consisting of, the amino acid sequence of any one of SEQ ID NOs: 16 and 18. In some embodiments, the CXCR5 polynucleotide comprises, consists essentially of, or consists of, a nucleotide sequence that is at least 85%, 90%, 93%, 94%, 95%, 96%, 97%, 98%, 99%, or 100% identical to the nucleotide sequence of any one of SEQ ID NOs: 15 and 17. In some embodiments, the CXCR5 polynucleotide comprises, consists essentially of, or consists of, a fragment of a nucleotide sequence that is at least 85%, 90%, 93%, 94%, 95%, 96%, 97%, 98%, 99%, or 100% identical to the nucleotide sequence of any one of SEQ ID NOs: 15 and 17 that encodes for a CXCR5 protein comprising, consisting essentially of, or consisting of, an amino acid sequence that is at least 85%, 90%, 93%, 94%, 95%, 96%, 97%, 98%, 99%, or 100% identical to the amino acid sequence of any one of SEQ ID NOs: 16 and 18. In some embodiments, the CXCR5 polynucleotide comprises, consists essentially of, or consists of, the nucleotide sequence of any of SEQ ID NOs: 15 and 17. In some embodiments, the CXCR5 polynucleotide comprises, consists essentially of, or consists of, a nucleotide sequence that is at least 85%, 90%, 93%, 94%, 95%, 96%, 97%, 98%, 99%, or 100% identical to the nucleotide sequence of any of SEQ ID NOs: 16 and 18.

In some embodiments, the engineered cell disclosed herein is a cell that is contacted with a polynucleotide encoding a GZMB protein. In some embodiments, the isolated cell disclosed herein is contacted with a polynucleotide encoding a GZMB protein. In some embodiments, the polynucleotide encoding the GZMB protein comprises, consists essentially of, or consists of, the nucleotide sequence of any one of SEQ ID NOs: 19 and 21. In some embodiments, the polynucleotide encoding the GZMB protein comprises, consists essentially of, or consists of, a fragment of the nucleotide sequence of any one of SEQ ID NOs: 19 and 21 that encodes for the GZMB protein comprising, consisting essentially of, or consisting of, the amino acid sequence of any one of SEQ ID NOs: 20 and 22. In some embodiments, the GZMB protein comprises, consists essentially of, or consists of, the amino acid sequence of any one of SEQ ID NOs: 20 and 22. In some embodiments, the polynucleotide encoding the GZMB protein comprises, consists essentially of, or consists of, the nucleotide sequence that is at least 85%, 90%, 93%, 94%, 95%, 96%, 97%, 98%, 99%, or 100% identical to the nucleotide sequence of any one of SEQ ID NOs: 19 and 21. In some embodiments, the polynucleotide encoding the GZMB protein comprises, consists essentially of, or consists of, a fragment of the nucleotide sequence that is at least 85%, 90%, 93%, 94%, 95%, 96%, 97%, 98%, 99%, or 100% identical to the nucleotide sequence of any one of SEQ ID NOs: 19 and 21 that encodes for the GZMB protein comprising, consisting essentially of, or consisting of, an amino acid sequence that is at least 85%, 90%, 93%, 94%, 95%, 96%, 97%, 98%, 99%, or 100% identical to the amino acid sequence of any one of SEQ ID NOs: 20 and 22. In some embodiments, the GZMB protein comprises, consists essentially of, or consists of, an amino acid sequence that is at least 85%, 90%, 93%, 94%, 95%, 96%, 97%, 98%, 99%, or 100% identical to the amino acid sequence of any one of SEQ ID NOs: 20 and 22. In some embodiments, the GZMB is a mammalian GZMB. In some embodiments, the GZMB is a human GZMB.

In some embodiments, the engineered cell disclosed herein is a cell that is contacted with a polynucleotide encoding a TNFRSF18 protein. In some embodiments, the isolated cell disclosed herein is contacted with a polynucleotide encoding a TNFRSF18 protein. In some embodiments, the polynucleotide encoding the TNFRSF18 protein comprises, consists essentially of, or consists of, the nucleotide sequence of any one of SEQ ID NOs: 23, 25 and 27. In some embodiments, the polynucleotide encoding the TNFRSF18 protein comprises, consists essentially of, or consists of, a fragment of the nucleotide sequence of any one of SEQ ID NOs: 23, 25 and 27 that encodes for the TNFRSF18 protein comprising, consisting essentially of, or consisting of, the amino acid sequence of any one of SEQ ID NOs: 24, 26, and 28. In some embodiments, the TNFRSF18 protein comprises, consists essentially of, or consists of, the amino acid sequence of any one of SEQ ID NOs: 24, 26, and 28. In some embodiments, the polynucleotide encoding the TNFRSF18 protein comprises, consists essentially of, or consists of, the nucleotide sequence that is at least 85%, 90%, 93%, 94%, 95%, 96%, 97%, 98%, 99%, or 100% identical to the nucleotide sequence of any one of SEQ ID NOs: 23, 25 and 27. In some embodiments, the polynucleotide encoding the TNFRSF18 protein comprises, consists essentially of, or consists of, a fragment of the nucleotide sequence that is at least 85%, 90%, 93%, 94%, 95%, 96%, 97%, 98%, 99%, or 100% identical to the nucleotide sequence of any one of SEQ ID NOs: 23, 25 and 27 that encodes for the TNFRSF18 protein comprising, consisting essentially of, or consisting of, an amino acid sequence that is at least 85%, 90%, 93%, 94%, 95%, 96%, 97%, 98%, 99%, or 100% identical to the amino acid sequence of any one of SEQ ID NOs: 24, 26, and 28. In some embodiments, the TNFRSF18 protein comprises, consists essentially of, or consists of, an amino acid sequence that is at least 85%, 90%, 93%, 94%, 95%, 96%, 97%, 98%, 99%, or 100% identical to the amino acid sequence of any one of SEQ ID NOs: 24, 26, and 28. In some embodiments, the TNFRSF18 is a mammalian TNFRSF18. In some embodiments, the TNFRSF18 is a human TNFRSF18.

In some embodiments, any one of the cells disclosed herein is engineered to express or expresses an antigen binding domain that binds at least one tumor antigen. In some embodiments, an engineered cell disclosed herein is engineered to express an antigen binding domain that binds at least one tumor antigen. In some embodiments, an isolated cell disclosed herein is engineered to express an antigen binding domain that binds at least one tumor antigen. In some embodiments, an isolated cell disclosed herein is naturally expresses an antigen binding domain that binds at least one tumor antigen. In some embodiments, the engineered cell disclosed herein is a cell that is contacted with a polynucleotide encoding the antigen binding domain. In some embodiments, the isolated cell disclosed herein is contacted with a polynucleotide encoding the binding domain. In some embodiments, the tumor antigen comprises, consists essentially of, or consists of, any one of: a CD19, a disialoganglioside-GD2, a c-mesenchymal-epithelial transition (c-Met), a mesothelin, a Receptor Tyrosine Kinase Like Orphan Receptor 1 (ROR1), an EGFRvIII, an ephrin type-A receptor 2 (EphA2), an interleukin (IL)-13r alpha 2, an EGFR VIII, a PSMA, an EpCAM, a GD3, a fucosyl GM1, a Prostate Stem Cell Antigen (PSCA), a placenta enriched 1 (PLAC1), a sarcoma breakpoint, a Wilms Tumor 1 antigen or a combination thereof.

In some embodiments, any one of the cells disclosed herein is engineered to express or expresses an antigen binding domain that binds at least one antigen. In some embodiments, an engineered cell disclosed herein is engineered to express an antigen binding domain that binds at least one antigen. In some embodiments, an isolated cell disclosed herein is engineered to express an antigen binding domain that binds at least one antigen. In some embodiments, an isolated cell disclosed herein expresses an antigen binding domain that binds at least one antigen. In some embodiments, the engineered cell disclosed herein is a cell that is contacted with a polynucleotide encoding the antigen binding domain. In some embodiments, the isolated cell disclosed herein is contacted with a polynucleotide encoding the binding domain. In some embodiments, the antigen is selected from a neo-antigen, tumor-associated antigen, viral antigen, bacterial antigen, and parasitic antigen.

In some embodiments, any one of the cells disclosed herein further comprises, consists essentially of, or consists of, a suicide gene. In some embodiments, the engineered cell disclosed herein is a cell that is contacted with a polynucleotide encoding a suicide gene. In some embodiments, the isolated cell disclosed herein is contacted with a polynucleotide encoding a suicide gene. As used herein, a suicide gene refers to a gene that, upon expression of the gene, triggers apoptosis in the cell or targets the cell for degradation.

In some embodiments, any one of the cells disclosed herein further comprises, consists essentially of, or consists of, a chimeric antigen receptor (CAR). In some embodiments, the engineered cell disclosed herein is a cell that is contacted with a polynucleotide encoding a CAR. In some embodiments, the isolated cell disclosed herein is contacted with a polynucleotide encoding a CAR. In some embodiments, the chimeric antigen receptor (CAR) comprises, consists essentially of, or consists of: (a) an antigen binding domain; (b) a hinge domain; (c) a transmembrane domain; (d) and an intracellular domain. In some embodiments, the CAR further comprises, consists essentially of, or consists of, a CD3 zeta signaling domain. In some embodiments, the hinge domain is any one of: CD8a or IgG1 hinge domain. In some embodiments, the transmembrane domain is any one of: CD28 or a CD8α transmembrane domain. In some embodiments, the intracellular domain comprises, consists essentially of, or consists of, one or more costimulatory regions selected from a CD28 costimulatory signaling region, a 4-1BB costimulatory signaling region, an ICOS costimulatory signaling region and/or an OX40 costimulatory region.

In some embodiments, the antigen binding domain of the CAR binds a tumor antigen. In some embodiments, the tumor antigen comprises, consists essentially of, or consists of, any one of: a CD19, a disialoganglioside-GD2, a c-mesenchymal-epithelial transition (c-Met), a mesothelin, a ROR1, an EGFRvIII, an ephrin type-A receptor 2 (EphA2), an interleukin (IL)-13r alpha 2, an EGFR VIII, a PSMA, an EpCAM, a GD3, a fucosyl GM1, a PSCA, a PLAC1, a sarcoma breakpoint, a Wilms Tumor 1 antigen, or a combination thereof.

In some embodiments, the CAR further comprises, consists essentially of, or consists of, an inducible or a constitutively active element. In some embodiments, the inducible or the constitutively active element controls the expression of a polynucleotide encoding an immunoregulatory molecule or a cytokine. In some embodiments, the immunoregulatory molecule or cytokine comprises, consists essentially of, or consists of, one or more of B7.1, CCL19, CCL21, CD40L, CD137L, GITRL, GM-CSF, IL-12, IL-2, low-toxicity IL-2, IL-15, IL-18, IL-21, LEC, and/or OX40L. In some embodiments, the immunoregulatory molecule or cytokine comprises, consists essentially of, or consists of, IL-12 and/or GM-CSF; and/or IL-12 and/or one or more of IL-2 and low-toxicity IL-2; and/or IL-12 and/or IL-15; and/or IL-12 and/or IL-21; IL-12 and/or B7.1; and/or IL-12 and/or OX40L; and/or IL-12 and/or CD40L; and/or IL-12 and/or GITRL; and/or IL-12 and/or IL-18; and/or one or more of IL-2 and low-toxicity IL-2 and one or more of CCL19, CCL21, and LEC; and/or IL-15 and one or more of CCL19, CCL21, and LEC; and/or IL-21 and one or more of CCL19, CCL21, and LEC; and/or GM-CSF and one or more of CCL19, CCL21, and LEC; and/or OX40L and one or more of CCL19, CCL21, and LEC; and/or CD137L and one or more of CCL19, CCL21, and LEC; and/or comprises, consists essentially of, or consists of, B7.1 and one or more of CCL19, CCL21, and LEC; and/or CD40L and one or more of CCL19, CCL21, and LEC; and/or GITRL and one or more of CCL19, CCL21, and LEC.

In some embodiments, the antigen binding domain of the CAR comprises, consists essentially of, or consists of, a heavy chain variable region and a light chain variable region of an antibody.

In some embodiments, the antigen binding domain of the CAR further comprises, consists essentially of, or consists of, a linker polypeptide located between the heavy chain variable region and the light chain variable region. In some embodiments, the linker polypeptide of the CAR comprises, consists essentially of, or consists of, a polypeptide of the sequence (GGGGS)n wherein n is an integer from 1 to 6.

In some embodiments, the CAR further comprises, consists essentially of, or consists of, a detectable marker attached to the CAR.

In some embodiments, the CAR further comprises, consists essentially of, or consists of, a purification marker attached to the CAR.

In some embodiments, any one of the cells disclosed herein comprises, consists essentially of, or consists of, a polynucleotide encoding the CAR.

In some embodiments, the polynucleotide further comprises, consists essentially of, or consists of, a promoter operatively linked to the polynucleotide to express the polynucleotide in the cell.

In some embodiments, the polynucleotide further comprises, consists essentially of, or consists of, a 2A self-cleaving peptide (T2A) encoding polynucleotide sequence located upstream of the polynucleotide encoding the antigen binding domain.

In some embodiments, the polynucleotide further comprises, consists essentially of, or consists of, a polynucleotide encoding a signal peptide located upstream of the polynucleotide encoding the antigen binding domain.

In some embodiments, the polynucleotide further comprises, consists essentially of, or consists of, a vector. In some embodiments, the vector is a plasmid or a viral vector, wherein the viral vector is optionally selected from the group of a retroviral vector, a lentiviral vector, an adenoviral vector, and an adeno-associated viral vector.

Any of the cells disclosed herein may be modified to express one or more proteins, antigen binding domains, tumor antigens, CARs, HCVRs, and/or LCVRs disclosed herein. In some embodiments, the engineered cell disclosed herein is a cell that is contacted with one or more polynucleotides that encode for any of the proteins, antigen binding domains, tumor antigens, CARs, HCVRs, and/or LCVRs disclosed herein. In some embodiments, the engineered cell disclosed herein is a cell that is contacted with one or more polynucleotides disclosed herein. In some embodiments, the isolated cell disclosed herein is contacted with one or more polynucleotides that encode for any of the proteins, antigen binding domains, tumor antigens, CARs, HCVRs, and/or LCVRs disclosed herein. In some embodiments, the isolated cell disclosed herein is contacted with one or more polynucleotides disclosed herein.

Methods of Preparing Engineered Cells and Isolated Cells

Further disclosed herein is a method of producing any one of the cells disclosed herein, comprising, consisting essentially of, or consisting of, reducing or eliminating expression and/or function of CXCR5 and/or increasing the expression of CD4 and CXCL13 in the cell.

In some embodiments, reducing or eliminating expression and/or function of CXCR5 comprises, consists essentially of, or consists of, the use of one or more of: RNA interference (RNAi), CRISPR, TALEN and/or ZFN. In some embodiments, reducing or eliminating expression and/or function of CXCR5 comprises, consists essentially of, or consists of, contacting an engineered cell with an oligonucleotide that inhibits expression of CXCR5. In some embodiments, reducing or eliminating expression and/or function of CXCR5 comprises, consists essentially of, or consists of, contacting an isolated cell with an oligonucleotide that inhibits expression of CXCR5. In some embodiments, the engineered cell or isolated cell is transfected with the oligonucleotide. In some embodiments, the engineered cell or isolated cell is transduced with the oligonucleotide. In some embodiments, the engineered cell or isolated cell is modified to stably express the oligonucleotide. In some embodiments, the engineered cell or isolated cell is modified to transiently express the oligonucleotide. In some embodiments, the engineered cell or isolated cell is modified to inducibly express the oligonucleotide. In some embodiments, the oligonucleotide is an antisense oligonucleotide. In some embodiments, the antisense oligonucleotide is a miRNA, shRNa, or siRNA that targets CXCR5. In some embodiments, the oligonucleotide is a guide RNA that targets CXCR5. Examples of oligonucleotides that inhibit expression of CXCR5 are known in the art, and include, but are not limited to, CXCR5 shRNA TL306391 (Origene), CXCR5 siRNA SR300441 (Origene), CXCR5 shRNA TR306391 (Origene), CXCR5 shRNA TL306391V (Origene), CXCR5 CRISPR gRNA ABIN5115520 (Genomics Online), and CXCR5 CRISPR gRNA ABIN5115519 (Genomics Online). In some embodiments, reducing or eliminating expression and/or function of CXCR5 comprises, consists essentially of, or consists of, contacting an engineered cell with a TALEN or ZFN that targets CXCR5. In some embodiments, reducing or eliminating expression and/or function of CXCR5 comprises, consists essentially of, or consists of, contacting an isolated cell with a TALEN or ZFN that targets CXCR5. In some embodiments, the CXCR5 polynucleotide is a mammalian CXCR5 polynucleotide. In some embodiments, the CXCR5 polynucleotide is a human CXCR5 polynucleotide. In some embodiments, the CXCR5 polynucleotide comprises, consists essentially of, or consists of, the nucleotide sequence of any one of SEQ ID NOs: 15 and 17. In some embodiments, the CXCR5 polynucleotide comprises, consists essentially of, or consists of, a fragment of the nucleotide sequence of any one of SEQ ID NOs: 15 and 17 that encodes for a CXCR5 protein comprising, consisting essentially of, or consisting of, the amino acid sequence of any one of SEQ ID NOs: 16 and 18. In some embodiments, the CXCR5 polynucleotide comprises, consists essentially of, or consists of, a nucleotide sequence that is at least 85%, 90%, 93%, 94%, 95%, 96%, 97%, 98%, 99%, or 100% identical to the nucleotide sequence of any one of SEQ ID NOs: 15 and 17. In some embodiments, the CXCR5 polynucleotide comprises, consists essentially of, or consists of, a fragment of a nucleotide sequence that is at least 85%, 90%, 93%, 94%, 95%, 96%, 97%, 98%, 99%, or 100% identical to the nucleotide sequence of any one of SEQ ID NOs: 15 and 17 that encodes for a CXCR5 protein comprising, consisting essentially of, or consisting of, an amino acid sequence that is at least 85%, 90%, 93%, 94%, 95%, 96%, 97%, 98%, 99%, or 100% identical to the amino acid sequence of any one of SEQ ID NOs: 16 and 18. In some embodiments, the CXCR5 polynucleotide comprises, consists essentially of, or consists of, the nucleotide sequence of any of SEQ ID NOs: 15 and 17. In some embodiments, the CXCR5 polynucleotide comprises, consists essentially of, or consists of, a nucleotide sequence that is at least 85%, 90%, 93%, 94%, 95%, 96%, 97%, 98%, 99%, or 100% identical to the nucleotide sequence of any of SEQ ID NOs: 16 and 18.

In some embodiments, increasing the expression of CD4 comprises, consists essentially of, or consists of, contacting an engineered cell with a polynucleotide that encodes for CD4. In some embodiments, the engineered cell disclosed herein is a cell that is contacted with a polynucleotide encoding a CD4 protein. In some embodiments, the isolated cell disclosed herein is contacted with a polynucleotide encoding a CD4 protein. In some embodiments, the engineered cell or isolated cell is transfected with a polynucleotide encoding a CD4 protein. In some embodiments, the engineered cell or isolated cell is transduced with a polynucleotide encoding a CD4 protein. In some embodiments, the engineered cell or isolated cell is modified to stably express a CD4 protein. In some embodiments, the engineered cell or isolated cell is modified to transiently express a CD4 protein. In some embodiments, the engineered cell or isolated cell is modified to inducibly express a CD4 protein. In some embodiments, the polynucleotide encoding the CD4 protein comprises, consists essentially of, or consists of, the nucleotide sequence of any one of SEQ ID NOs: 1, 3, 5, 7, and 9. In some embodiments, the polynucleotide encoding the CD4 protein comprises, consists essentially of, or consists of, a fragment of the nucleotide sequence of any one of SEQ ID NOs: 1, 3, 5, 7, and 9 that encodes for the CD4 protein comprising, consisting essentially of, or consisting of, the amino acid sequence of any one of SEQ ID NOs: 2, 4, 6, 8, and 10. In some embodiments, the CD4 protein comprises, consists essentially of, or consists of, the amino acid sequence of any one of SEQ ID NOs: 2, 4, 6, 8, and 10. In some embodiments, the polynucleotide encoding the CD4 protein comprises, consists essentially of, or consists of, a nucleotide sequence that is at least 85%, 90%, 93%, 94%, 95%, 96%, 97%, 98%, 99%, or 100% identical to the nucleotide sequence of any one of SEQ ID NOs: 1, 3, 5, 7, and 9. In some embodiments, the polynucleotide encoding the CD4 protein comprises, consists essentially of, or consists of, a fragment of a nucleotide sequence that is at least 85%, 90%, 93%, 94%, 95%, 96%, 97%, 98%, 99%, or 100% identical to the nucleotide sequence of any one of SEQ ID NOs: 1, 3, 5, 7, and 9 that encodes for the CD4 protein comprising, consisting essentially of, or consisting of, an amino acid sequence that is at least 85%, 90%, 93%, 94%, 95%, 96%, 97%, 98%, 99%, or 100% identical to the amino acid sequence of any one of SEQ ID NOs: 2, 4, 6, 8, and 10. In some embodiments, the CD4 protein comprises, consists essentially of, or consists of, an amino acid sequence that is at least 85%, 90%, 93%, 94%, 95%, 96%, 97%, 98%, 99%, or 100% identical to the amino acid sequence of any one of SEQ ID NOs: 2, 4, 6, 8, and 10. In some embodiments, the CD4 is a mammalian CD4. In some embodiments, the CD4 is a human CD4.

In some embodiments, increasing the expression of CXCL13 comprises, consists essentially of, or consists of, contacting an engineered cell with a polynucleotide that encodes for CXCL13. In some embodiments, the engineered cell disclosed herein is a cell that is contacted with a polynucleotide encoding a CXCL13 protein. In some embodiments, the isolated cell disclosed herein is contacted with a polynucleotide encoding a CXCL13 protein. In some embodiments, the engineered cell or isolated cell is transduced with a polynucleotide encoding a CXCL13 protein. In some embodiments, the engineered cell or isolated cell is modified to stably express a CXCL13 protein. In some embodiments, the engineered cell or isolated cell is modified to transiently express a CXCL13 protein. In some embodiments, the engineered cell or isolated cell is modified to inducibly express a CXCL13 protein. In some embodiments, the polynucleotide encoding the CXCL13 protein comprises, consists essentially of, or consists of, the nucleotide sequence of any one of SEQ ID NOs: 11 and 13. In some embodiments, the polynucleotide encoding the CXCL13 protein comprises, consists essentially of, or consists of, a fragment of the nucleotide sequence of any one of SEQ ID NOs: 11 and 13 that encodes for the CXCL13 protein comprising, consisting essentially of, or consisting of, the amino acid sequence of any one of SEQ ID NOs: 12 and 14. In some embodiments, the CXCL13 protein comprises, consists essentially of, or consists of, the amino acid sequence of any one of SEQ ID NOs: 12 and 14. In some embodiments, the polynucleotide encoding the CXCL13 protein comprises, consists essentially of, or consists of, a nucleotide sequence that is at least 85%, 90%, 93%, 94%, 95%, 96%, 97%, 98%, 99%, or 100% identical to the nucleotide sequence of any one of SEQ ID NOs: 11 and 13. In some embodiments, the polynucleotide encoding the CXCL13 protein comprises, consists essentially of, or consists of, a fragment of a nucleotide sequence that is at least 85%, 90%, 93%, 94%, 95%, 96%, 97%, 98%, 99%, or 100% identical to the nucleotide sequence of any one of SEQ ID NOs: 11 and 13 that encodes for the CXCL13 protein comprising, consisting essentially of, or consisting of, an amino acid sequence that is at least 85%, 90%, 93%, 94%, 95%, 96%, 97%, 98%, 99%, or 100% identical to the amino acid sequence of any one of SEQ ID NOs: 12 and 14. In some embodiments, the CXCL13 protein comprises, consists essentially of, or consists of, an amino acid sequence that is at least 85%, 90%, 93%, 94%, 95%, 96%, 97%, 98%, 99%, or 100% identical to the amino acid sequence of any one of SEQ ID NOs: 12 and 14. In some embodiments, the CXCL13 is a mammalian CXCL13. In some embodiments, the CXCL13 is a human CXCL13.

In some embodiments, increasing the expression of GZMB comprises, consists essentially of, or consists of, contacting an engineered cell with a polynucleotide that encodes for GZMB. In some embodiments, the engineered cell disclosed herein is a cell that is contacted with a polynucleotide encoding a GZMB protein. In some embodiments, the isolated cell disclosed herein is contacted with a polynucleotide encoding a GZMB protein. In some embodiments, the engineered cell or isolated cell is transduced with a polynucleotide encoding a GZMB protein. In some embodiments, the engineered cell or isolated cell is modified to stably express a GZMB protein. In some embodiments, the engineered cell or isolated cell is modified to transiently express a GZMB protein. In some embodiments, the engineered cell or isolated cell is modified to inducibly express a GZMB protein. In some embodiments, the polynucleotide encoding the GZMB protein comprises, consists essentially of, or consists of, the nucleotide sequence of any one of SEQ ID NOs: 19 and 21. In some embodiments, the polynucleotide encoding the GZMB protein comprises, consists essentially of, or consists of, a fragment of the nucleotide sequence of any one of SEQ ID NOs: 19 and 21 that encodes for the GZMB protein comprising, consisting essentially of, or consisting of, the amino acid sequence of any one of SEQ ID NOs: 20 and 22. In some embodiments, the GZMB protein comprises, consists essentially of, or consists of, the amino acid sequence of any one of SEQ ID NOs: 20 and 22. In some embodiments, the polynucleotide encoding the GZMB protein comprises, consists essentially of, or consists of, the nucleotide sequence that is at least 85%, 90%, 93%, 94%, 95%, 96%, 97%, 98%, 99%, or 100% identical to the nucleotide sequence of any one of SEQ ID NOs: 19 and 21. In some embodiments, the polynucleotide encoding the GZMB protein comprises, consists essentially of, or consists of, a fragment of the nucleotide sequence that is at least 85%, 90%, 93%, 94%, 95%, 96%, 97%, 98%, 99%, or 100% identical to the nucleotide sequence of any one of SEQ ID NOs: 19 and 21 that encodes for the GZMB protein comprising, consisting essentially of, or consisting of, an amino acid sequence that is at least 85%, 90%, 93%, 94%, 95%, 96%, 97%, 98%, 99%, or 100% identical to the amino acid sequence of any one of SEQ ID NOs: 20 and 22. In some embodiments, the GZMB protein comprises, consists essentially of, or consists of, an amino acid sequence that is at least 85%, 90%, 93%, 94%, 95%, 96%, 97%, 98%, 99%, or 100% identical to the amino acid sequence of any one of SEQ ID NOs: 20 and 22. In some embodiments, the GZMB is a mammalian GZMB. In some embodiments, the GZMB is a human GZMB.

In some embodiments, increasing the expression of TNFRSF18 comprises, consists essentially of, or consists of, contacting an engineered cell with a polynucleotide that encodes for TNFRSF18. In some embodiments, the engineered cell disclosed herein is a cell that is contacted with a polynucleotide encoding a TNFRSF18 protein. In some embodiments, the isolated cell disclosed herein is contacted with a polynucleotide encoding a TNFRSF18 protein. In some embodiments, the engineered cell or isolated cell is transduced with a polynucleotide encoding a TNFRSF18 protein. In some embodiments, the engineered cell or isolated cell is modified to stably express a TNFRSF18 protein. In some embodiments, the engineered cell or isolated cell is modified to transiently express a TNFRSF18 protein. In some embodiments, the engineered cell or isolated cell is modified to inducibly express a TNFRSF18 protein. In some embodiments, the polynucleotide encoding the TNFRSF18 protein comprises, consists essentially of, or consists of, the nucleotide sequence of any one of SEQ ID NOs: 23, 25 and 27. In some embodiments, the polynucleotide encoding the TNFRSF18 protein comprises, consists essentially of, or consists of, a fragment of the nucleotide sequence of any one of SEQ ID NOs: 23, 25 and 27 that encodes for the TNFRSF18 protein comprising, consisting essentially of, or consisting of, the amino acid sequence of any one of SEQ ID NOs: 24, 26, and 28. In some embodiments, the TNFRSF18 protein comprises, consists essentially of, or consists of, the amino acid sequence of any one of SEQ ID NOs: 24, 26, and 28. In some embodiments, the polynucleotide encoding the TNFRSF18 protein comprises, consists essentially of, or consists of, the nucleotide sequence that is at least 85%, 90%, 93%, 94%, 95%, 96%, 97%, 98%, 99%, or 100% identical to the nucleotide sequence of any one of SEQ ID NOs: 23, 25 and 27. In some embodiments, the polynucleotide encoding the TNFRSF18 protein comprises, consists essentially of, or consists of, a fragment of the nucleotide sequence that is at least 85%, 90%, 93%, 94%, 95%, 96%, 97%, 98%, 99%, or 100% identical to the nucleotide sequence of any one of SEQ ID NOs: 23, 25 and 27 that encodes for the TNFRSF18 protein comprising, consisting essentially of, or consisting of, an amino acid sequence that is at least 85%, 90%, 93%, 94%, 95%, 96%, 97%, 98%, 99%, or 100% identical to the amino acid sequence of any one of SEQ ID NOs: 24, 26, and 28. In some embodiments, the TNFRSF18 protein comprises, consists essentially of, or consists of, an amino acid sequence that is at least 85%, 90%, 93%, 94%, 95%, 96%, 97%, 98%, 99%, or 100% identical to the amino acid sequence of any one of SEQ ID NOs: 24, 26, and 28. In some embodiments, the TNFRSF18 is a mammalian TNFRSF18. In some embodiments, the TNFRSF18 is a human TNFRSF18.

In some embodiments, producing an engineered cell comprises, consists essentially of, or consists of, increasing expression of one or more proteins selected CD4, CXCL13, GZMB, TNFRSF18, TNFRSF4, IFNG, IL21, and a protein listed in Table 11 in the cell. In some embodiments, increasing the expression of CD4, CXCL13, GZMB, TNFRSF18, TNFRSF4, IFNG, IL21, and a protein listed in Table 11 comprises, consists essentially of, or consists of, contacting an engineered cell with one or more polynucleotides that encode for one or more of CD4, CXCL13, GZMB, TNFRSF18, TNFRSF4, IFNG, IL21, and a protein listed in Table 11. In some embodiments, the engineered cell disclosed herein is a cell that is contacted with one or more polynucleotides encoding one or more of CD4, CXCL13, GZMB, TNFRSF18, TNFRSF4, IFNG, IL21, and a protein listed in Table 11 protein. In some embodiments, the isolated cell disclosed herein is contacted with one or more polynucleotides encoding one or more of CD4, CXCL13, GZMB, TNFRSF18, TNFRSF4, IFNG, IL21, and a protein listed in Table 11 protein. In some embodiments, the engineered cell or isolated cell is transduced with one or more polynucleotides encoding one or more of CD4, CXCL13, GZMB, TNFRSF18, TNFRSF4, IFNG, IL21, and a protein listed in Table 11 protein. In some embodiments, the engineered cell or isolated cell is modified to stably express one or more of CD4, CXCL13, GZMB, TNFRSF18, TNFRSF4, IFNG, IL21, and a protein listed in Table 11 protein. In some embodiments, the engineered cell or isolated cell is modified to transiently express one or more of CD4, CXCL13, GZMB, TNFRSF18, TNFRSF4, IFNG, IL21, and a protein listed in Table 11 protein. In some embodiments, the engineered cell or isolated cell is modified to inducibly express one or more of CD4, CXCL13, GZMB, TNFRSF18, TNFRSF4, IFNG, IL21, and a protein listed in Table 11 protein.

In some embodiments, producing an engineered cell comprises, consists essentially of, or consists of, decreasing expression of one or more proteins selected CXCR5, MAF, SH2D1A (SAP), PDCD1, BTLA, CD200, BCL6, and a protein listed in Table 11 in the cell. In some embodiments, decreasing the expression of CXCR5, MAF, SH2D1A (SAP), PDCD1, BTLA, CD200, BCL6, and a protein listed in Table 11 comprises, consists essentially of, or consists of, contacting an engineered cell with one or more oligonucleotides that inhibit expression of one or more of CXCR5, MAF, SH2D1A (SAP), PDCD1, BTLA, CD200, BCL6, and a protein listed in Table 11. In some embodiments, the engineered cell disclosed herein is a cell that is contacted with one or more oligonucleotides that inhibit expression of one or more of CXCR5, MAF, SH2D1A (SAP), PDCD1, BTLA, CD200, BCL6, and a protein listed in Table 11. In some embodiments, the isolated cell disclosed herein is contacted with one or more oligonucleotides that inhibit expression of one or more of CXCR5, MAF, SH2D1A (SAP), PDCD1, BTLA, CD200, BCL6, and a protein listed in Table 11. In some embodiments, the engineered cell or isolated cell is transduced with one or more oligonucleotides that inhibit expression of one or more of CXCR5, MAF, SH2D1A (SAP), PDCD1, BTLA, CD200, BCL6, and a protein listed in Table 11. In some embodiments, the engineered cell or isolated cell is modified to stably express one or more oligonucleotides that inhibit expression of one or more of CXCR5, MAF, SH2D1A (SAP), PDCD1, BTLA, CD200, BCL6, and a protein listed in Table 11. In some embodiments, the engineered cell or isolated cell is modified to transiently one or more oligonucleotides that inhibit expression of one or more of CXCR5, MAF, SH2D1A (SAP), PDCD1, BTLA, CD200, BCL6, and a protein listed in Table 11. In some embodiments, the engineered cell or isolated cell is modified to inducibly express one or more oligonucleotides that inhibit expression of one or more of CXCR5, MAF, SH2D1A (SAP), PDCD1, BTLA, CD200, BCL6, and a protein listed in Table 11.

In some embodiments, the cell is a mammalian cell. In some embodiments, the mammalian cell is a human cell. In some embodiments the cell is an immune cell. In some embodiments, the immune cell is a lymphocyte. In some embodiments, the lymphocyte is a T cell. In some embodiments, the T cell is a CD4⁺ T cell. In some embodiments, the lymphocyte is an NK cell.

In some embodiments, the Tfh-like cells are engineered from Tfh cells isolated from human peripheral blood mononuclear cells (PBMCs) and expanded and stimulated, before transducing cells to generate the desired genetic profile (c-K, CD4⁺CXCR13⁺CXCR5′, optionally expressing GZMB).

In some embodiments, the Tfh-like cells are engineered from naïve CD4+ T cells isolated from human PBMCs and expanded and stimulated, before transducing cells to generate the desired genetic profile (e.g. CD4⁺CXCR13⁺CXCR5′, optionally expressing GZMB).

Further disclosed herein is a method of producing any one of the cells disclosed herein, increasing the expression of one or more of: TNFRSF18, TNFRSF4, IFNG, Granzyme B and/or IL21 in the cell. In some embodiments, the method comprises, consists essentially of, or consists of, increasing the expression of 2, 3, or 4 or more of: TNFRSF18, TNFRSF4, IFNG, Granzyme B and IL21.

Further disclosed herein is a method of isolating any one of the cells disclosed herein, comprising, consisting essentially of, or consisting of, separating Tfh-like tumor-infiltrating cell from a mixed cell population. In some embodiments, the method further comprises, consists essentially of, or consists of, sorting for cells that express the surface markers CD4 and CXCL13 and lack the surface marker CXCR5. In some embodiments, the method further comprises, consists essentially of, or consists of, increasing the expression or function of one or more of: TNFRSF18, TNFRSF4, IFNG, Granzyme B and/or IL21 in the cell.

In some embodiments, the method of isolating cells comprises, consists essentially of, or consists of, (a) contacting a sample comprising, consisting essentially of, or consisting of, a plurality of cells with one or more agents that bind to CD4 and/or CXCL13; and (b) collecting cells that are bound to the one or more agents. In some embodiments, isolating cells comprises, consists essentially of, or consists of, (a) contacting the sample with an agent that binds to CD4 and an agent that binds to CXCL13; and (b) collecting cells that are bound to both the agent that binds to CD4 and the agent that binds to CXCL13. In some embodiments, the one or more agents are conjugated to a label. In some embodiments, the label that is conjugated to the agent that binds to CD4 is different from the label that is conjugated to the agent that binds to CXCL13. The label may be any of the labels disclosed herein. In some embodiments, the label is a magnetic label. In some embodiments, collecting cells that are bound to the one or more agents comprises, consists essentially of, or consists of, applying a magnetic field to the sample. In some embodiments, the label is a ferrofluid magnetic particle. In some embodiments, collecting cells comprises, consists essentially of, or consists of, the use of ferrofluid technology. In some embodiments, the label is a fluorescent label. In some embodiments, collecting cells comprises, consists essentially of, or consists of, the use of FACS.

In some embodiments, the method of isolating cells further comprises, consists essentially of, or consists of, contacting the sample with an agent that binds to CXCR5 and removing cells from the sample that are bound to the agent that binds to CXCR5. In some embodiments, the agent that binds to CXCR5 is conjugated to a label. The label may be any of the labels disclosed herein. In some embodiments, the label that is conjugated to CXCR5 is different from the label that is conjugated to the agent that binds to CD4 and the agent that binds to CXCL13. For instance, if the label that is conjugated to the agent that binds to CD4 is a green fluorescent protein (GFP), the label that is conjugated to the agent that binds to CXCR5 is not a GFP, but the label may be another fluorescent protein, such as a red fluorescent protein (RFP).

In some embodiments, the method of isolating cells further comprises, consists essentially of, or consists of, contacting the sample with one or more agents that bind to one or more proteins selected from TNFRSF18, TNFRSF4, IFNG, Granzyme B and IL21. In some embodiments, these agents are conjugated to a label. The label may be any of the labels disclosed herein. In some embodiments, the labels that are conjugated to these agents are different from the labels that are conjugated to the agents that bind to CD4, CXCL13, and CXCR5.

Further disclosed herein is a method of producing any one of the cells disclosed herein, comprising, consisting essentially of, or consisting of, modulating the expression and/or function of one or more proteins selected from Table 11. In certain embodiments, the one, two, three, four, or five or more proteins are selected from MAF, SH2D1A (SAP), PDCD1, BTLA, CD200, and BCL6. In some embodiments, the expression and/or function of the one or more surface markers is modulated by using one or more of CRISPR, TALEN and/or ZFN. In some embodiments, the expression and/or function of the one or more surface markers is modulated by using one or more polynucleotides encoding expression of the one or more surface markers.

In some embodiments, the method further comprises, consists essentially of, or consists of, increasing the expression or function of one, two, three, or four or more of: TNFRSF18, TNFRSF4, IFNG, Granzyme B and/or IL21 in the cell using one or more of: CRISPR, TALEN and/or ZFN. In some embodiments, the expression and/or function of one or more of TNFRSF18, TNFRSF4, IFNG, Granzyme B and IL21 is modulated by using one or more polynucleotides encoding expression of the one or more of TNFRSF18, TNFRSF4, IFNG, Granzyme B and IL21.

In some embodiments, the method for isolating the cells use of commercially available products for isolating cells (e.g., T cells) from samples, such as biological fluids (e.g., blood, serum, and plasma) and samples containing other cells (e.g., peripheral blood mononuclear cells (PMBCs)). Examples of products that are commercially available to isolate various T cells from human PBMCs, including Pan T Cell Isolation Kit, human (Miltenyi Biotech, #130-096-535), CD4⁺CD25⁺ Regulatory T Cell Isolation Kit, human (Miltenyi Biotech, #130-091-301), In some embodiments, the method for isolating the cells comprises, consists essentially of or consists of, (a) a cell separation step (e.g. magnetic beads with antibodies that bind specific cell surface proteins); and (b) a flow cytometry and cell analysis method.

In some embodiments, the methods disclosed herein further comprise, consist essentially of, or consist of, expanding the cell (e.g., T cell) in vitro. Methods of expanding human T cells in vitro are commercially available. In some embodiments, the method of expanding the cell comprise, consist essentially of, or consist of, the use of one or more commercially available kits. Examples of commercially available kits for expanding human T cells in vitro include, but are not limited to, T Cell Activation/Expansion Kit, human (Miltenyi Biotech, #130-091-441) and Treg Expansion Kit, human (Miltenyi Biotech, 130-095-3435). In some embodiments, the method for expanding the cells comprise, consist essentially of, or consist of, (a) a ceil separation step (e.g. magnetic beads with antibodies that bind specific cell surface proteins); (b) a flow cytometry and cell analysis method to isolate cell populations of interest; and (c) expansion of the isolated cells.

In some embodiments, the methods disclosed herein further comprise, consist essentially of, or consist of, stimulating and/or expanding engineered cells or isolated cells disclosed herein. Methods for stimulating or expanding cells may comprise, consist essentially of, or consist of, use of one or more commercially available reagents. In some embodiments, the commercially available reagents that are used to stimulate and expand antigen-specific T cells include, but are not limited to, reagents that direct ex vivo characterization of human antigen-specific CD154⁺CD4⁺ T cells, and lyophilized peptides, such as PepTivator BKV VP1 (Miltenyi Biotech, research grade, human, #130-097-272) and PepTivator BKV LT (Miltenyi Biotech, research grade, human, #130-096-504). In some cases, the methods for stimulating and/or expanding the cells further comprise, consist essentially of or consist of (a) cell separation (e.g. magnetic beads with antibodies that bind specific cell surface proteins); (b) flow cytometry and cell analysis methods to isolate cell populations of interest; and (c) expansion of the isolated cells,

Disclosed herein is a method of preparing any of the population of cells disclosed herein, comprising, consisting essentially of, or consisting of, isolating the cells from a subject and culturing the cells ex vivo.

Disclosed herein is a method of preparing any of the population of cells disclosed herein, comprising, consisting essentially of, or consisting of, expanding the cells in vivo and isolating the cells from a subject.

Further disclosed herein is a composition comprising, consisting essentially of, or consisting of, a carrier and one or more of: the cells disclosed herein and/or any of the population of cells disclosed herein.

In some embodiments, the carrier is a pharmaceutically acceptable carrier.

In some embodiments, the composition further comprises, consists essentially of, or consists of, a cryoprotectant.

Determining Responders to Anti-Cancer Therapy

Further disclosed herein is a method of determining whether a subject will respond to a treatment for cancer, comprising, consisting essentially of, or consisting of, measuring the amount of one or more of: CD4⁺CXCL13⁺CXCR5′ Tfh-like tumor-infiltrating cell, and/or CD4⁺CXCL13⁺CXCR5′GZMB⁺ cytotoxic Tfh-like tumor-infiltrating cell in a sample isolated from the subject, wherein higher amounts of the cells indicates that the subject is likely to respond to the treatment and lower amounts of the cells indicates that the subject is not likely to respond to the treatment. The amount of the cells may be measured by any method known in the art for quantifying cells. For instance, measuring the amount of cells may comprise, consist essentially of, or consist of, flow cytometry (e.g., FACS), immunoassays, image analysis, stereologic cell counting, and spectrophotometry. The method may further comprise, consist essentially of, or consist of, isolating or purifying CD4⁺CXCL13⁺CXCR5′ Tfh-like tumor-infiltrating cell and/or CD4⁺CXCL13⁺CXCR5′GZMB⁺ cytotoxic Tfh-like tumor-infiltrating cell from a sample prior to measuring the amount of cells.

In some embodiments, the treatment for cancer comprises, consists essentially of, or consists of, a checkpoint inhibitor. In some embodiments, the checkpoint inhibitor is selected from the group of an anti-PD-1, anti-PD-L1, anti-CTLA-4, anti-B7-1, and anti-B7-2 immunotherapy treatment.

In some embodiments, the method further comprises, consists essentially of, or consists of, administering to the subject that is likely to respond to the checkpoint inhibitor therapy an effective amount of the checkpoint inhibitor therapy.

In some embodiments, the method further comprises, consists essentially of, or consists of, administering to the subject an effective amount of a cytoreductive therapy. In some embodiments, the cytoreductive therapy comprises, consists essentially of, or consists of, one or more of chemotherapy, immunotherapy, or radiation therapy.

In some embodiments, the method further comprises, consists essentially of, or consists of, administering to the subject an effective amount of one or more of: the cells disclosed herein, the population of cells disclosed herein and/or the compositions disclosed herein.

Method of Treatment

Further disclosed herein is a method of treating cancer in a subject comprising, consisting essentially of, or consisting of, administering to the subject an effective amount of one or more of: the cells disclosed herein, the population of cells disclosed herein and/or the compositions disclosed herein.

In some embodiments, the subject is selected for treatment by contacting a sample isolated from the subject with an agent that detects the presence of a tumor antigen in the sample and the subject is selected for the treatment if presence of one or more tumor antigen is detected in the sample. In some embodiments, the subject is a mammal. In some embodiments, the subject is a human.

Further disclosed herein is a pharmaceutical composition comprising, consisting essentially of, or consisting of, any of the cells disclosed herein for the treatment of cancer.

Further disclosed herein is a pharmaceutical composition comprising, consisting essentially of, or consisting of, any of the populations of cells disclosed herein for the treatment of cancer.

Further disclosed herein is a pharmaceutical composition comprising, consisting essentially of, or consisting of, any of the compositions disclosed herein for the treatment of cancer.

Use of any of the cells disclosed herein for the treatment of cancer.

Use of any of the populations of cells disclosed herein for the treatment of cancer.

Use of any of the compositions disclosed herein for the treatment of cancer.

Use of the cells disclosed herein in the manufacture of a medicament for the treatment of cancer.

Use of the populations of cells disclosed herein in the manufacture of a medicament for the treatment of cancer.

Use of the compositions disclosed herein in the manufacture of a medicament for the treatment of cancer.

In one aspect, the cancer or tumor is an epithelial, a head, neck, lung, lung, prostate, colon, pancreas, esophagus, liver, skin, kidney, adrenal gland, brain, or comprises a lymphoma, breast, endometrium, uterus, ovary, testes, lung, prostate, colon, pancreas, esophagus, liver, skin, kidney, adrenal gland and/or brain cancer or tumor, a metastasis or recurring tumor, cancer or neoplasia, a non-small cell lung cancer (NSCLC) and/or head and neck squamous cell cancer (HNSCC). In some embodiments, the cancer is lung cancer.

Compositions and Kits

Disclosed herein is a composition comprising, or consisting essentially of, or yet further consisting of any of the cells disclosed herein. Disclosed herein is a composition comprising, or consisting essentially of, or yet further consisting of any of the engineered cells disclosed herein. Disclosed herein is a composition comprising, or consisting essentially of, or yet further consisting of any of the isolated cells disclosed herein. Further provided herein is a composition comprising, or alternatively consisting essentially of, or yet further consisting of a carrier and one or more of: the engineered cells of this disclosure and/or the population of engineered cells of this disclosure. Further provided herein is a composition comprising, or alternatively consisting essentially of, or yet further consisting of a carrier and one or more of: the isolated cells of this disclosure and/or the population of isolated cells of this disclosure. In one aspect, the population is a substantially homogenous cell population. In another aspect, the population is a heterogeneous population. The composition of the present disclosure also can be bound to many different carriers. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the disclosure. Those skilled in the art will know of other suitable carriers, or will be able to ascertain such, using routine experimentation.

Further disclosed herein is a kit comprising, consisting essentially of, or consisting of, one or more of: the cells disclosed herein, the populations of cells disclosed herein, and/or the compositions disclosed herein and instructions to carry out the any of the methods disclosed herein. In some embodiments, the kit comprises, consists essentially of, or consists of, a CD4⁺CXCL13⁺CXCR5′Tfh-like tumor-infiltrating cell. In some embodiments, the kit comprises, consists essentially of, or consists of, a CD4⁺CXCL13⁺CXCR5′GZMB⁺ cytotoxic Tfh-like tumor-infiltrating cell.

In some embodiments, a kit comprises, consists essentially of, or consists of, one or more of: (i) a polynucleotide encoding one or more proteins selected from: CD4, CXCL13, GZMB, TNFRSF18, TNFRSF4, IFNG, IL21, and a protein listed in Table 11; (ii) an oligonucleotide that inhibits expression a protein selected from CXCR5, MAF, SH2D1A (SAP), PDCD1, BTLA, CD200, BCL6, and a protein listed in Table 11; (iii) an antibody that binds to one or more proteins selected from: CD4, CXCL13, GZMB, TNFRSF18, TNFRSF4, IFNG, IL21, CXCR5, MAF, SH2D1A (SAP), PDCD1, BTLA, CD200, BCL6, and a protein listed in Table 11.

In some embodiments, a kit comprises, consists essentially of, or consists of, (a) a cell; and (b) at least one of (i) a polynucleotide encoding a CD4 protein; (ii) polynucleotide encoding CXCL13 protein; and (iii) an oligonucleotide that inhibits expression of a CXCR5 protein.

In some embodiments, a kit comprises, consists essentially of, or consists of, two or more of (i) a polynucleotide encoding a CD4 protein; (ii) polynucleotide encoding CXCL13 protein; (iii) an oligonucleotide that inhibits expression of a CXCR5 protein; and (iv) a polynucleotide encoding a granzyme B protein.

In some embodiments, a kit comprises, consists essentially of, or consists of: (a) a polynucleotide encoding a CD4 protein; and a polynucleotide encoding a CXCL13 protein.

In some embodiments, a kit comprises, consists essentially of, or consists of: (a) polynucleotide encoding a CD4 protein; and (b) an oligonucleotide that inhibits expression of CXCR5.

In some embodiments, a kit comprises, consists essentially of, or consists of: (a) polynucleotide encoding a CXCL13 protein; and (b) an oligonucleotide that inhibits expression of CXCR5.

In some embodiments, the kits disclosed herein comprise, consist essentially of, or consist of, a polynucleotide encoding a CD4 protein. In some embodiments, the polynucleotide encoding the CD4 protein comprises, consists essentially of, or consists of, the nucleotide sequence of any one of SEQ ID NOs: 1, 3, 5, 7, and 9. In some embodiments, the polynucleotide encoding the CD4 protein comprises, consists essentially of, or consists of, a fragment of the nucleotide sequence of any one of SEQ ID NOs: 1, 3, 5, 7, and 9 that encodes for the CD4 protein comprising, consisting essentially of, or consisting of, the amino acid sequence of any one of SEQ ID NOs: 2, 4, 6, 8, and 10. In some embodiments, the CD4 protein comprises, consists essentially of, or consists of, the amino acid sequence of any one of SEQ ID NOs: 2, 4, 6, 8, and 10. In some embodiments, the polynucleotide encoding the CD4 protein comprises, consists essentially of, or consists of, a nucleotide sequence that is at least 85%, 90%, 93%, 94%, 95%, 96%, 97%, 98%, 99%, or 100% identical to the nucleotide sequence of any one of SEQ ID NOs: 1, 3, 5, 7, and 9. In some embodiments, the polynucleotide encoding the CD4 protein comprises, consists essentially of, or consists of, a fragment of a nucleotide sequence that is at least 85%, 90%, 93%, 94%, 95%, 96%, 97%, 98%, 99%, or 100% identical to the nucleotide sequence of any one of SEQ ID NOs: 1, 3, 5, 7, and 9 that encodes for the CD4 protein comprising, consisting essentially of, or consisting of, an amino acid sequence that is at least 85%, 90%, 93%, 94%, 95%, 96%, 97%, 98%, 99%, or 100% identical to the amino acid sequence of any one of SEQ ID NOs: 2, 4, 6, 8, and 10. In some embodiments, the CD4 protein comprises, consists essentially of, or consists of, an amino acid sequence that is at least 85%, 90%, 93%, 94%, 95%, 96%, 97%, 98%, 99%, or 100% identical to the amino acid sequence of any one of SEQ ID NOs: 2, 4, 6, 8, and 10. In some embodiments, the CD4 is a mammalian CD4. In some embodiments, the CD4 is a human CD4.

In some embodiments, the kits disclosed herein comprise, consist essentially of, or consist of, a polynucleotide encoding a CXCL13 protein. In some embodiments, the polynucleotide encoding the CXCL13 protein comprises, consists essentially of, or consists of, the nucleotide sequence of any one of SEQ ID NOs: 11 and 13. In some embodiments, the polynucleotide encoding the CXCL13 protein comprises, consists essentially of, or consists of, a fragment of the nucleotide sequence of any one of SEQ ID NOs: 11 and 13 that encodes for the CXCL13 protein comprising, consisting essentially of, or consisting of, the amino acid sequence of any one of SEQ ID NOs: 12 and 14. In some embodiments, the CXCL13 protein comprises, consists essentially of, or consists of, the amino acid sequence of any one of SEQ ID NOs: 12 and 14. In some embodiments, the polynucleotide encoding the CXCL13 protein comprises, consists essentially of, or consists of, a nucleotide sequence that is at least 85%, 90%, 93%, 94%, 95%, 96%, 97%, 98%, 99%, or 100% identical to the nucleotide sequence of any one of SEQ ID NOs: 11 and 13. In some embodiments, the polynucleotide encoding the CXCL13 protein comprises, consists essentially of, or consists of, a fragment of a nucleotide sequence that is at least 85%, 90%, 93%, 94%, 95%, 96%, 97%, 98%, 99%, or 100% identical to the nucleotide sequence of any one of SEQ ID NOs: 11 and 13 that encodes for the CXCL13 protein comprising, consisting essentially of, or consisting of, an amino acid sequence that is at least 85%, 90%, 93%, 94%, 95%, 96%, 97%, 98%, 99%, or 100% identical to the amino acid sequence of any one of SEQ ID NOs: 12 and 14. In some embodiments, the CXCL13 protein comprises, consists essentially of, or consists of, an amino acid sequence that is at least 85%, 90%, 93%, 94%, 95%, 96%, 97%, 98%, 99%, or 100% identical to the amino acid sequence of any one of SEQ ID NOs: 12 and 14. In some embodiments, the CXCL13 is a mammalian CXCL13. In some embodiments, the CXCL13 is a human CXCL13.

In some embodiments, the kits disclosed herein comprise, consist essentially of, or consist of, an oligonucleotide that inhibits expression of CXCR5. In some embodiments, the oligonucleotide that inhibits expression of CXCR5 is an antisense oligonucleotide that targets a CXCR5 polynucleotide. In some embodiments, the antisense oligonucleotide is a microRNA (miRNA), short hairpin RNA (shRNA), or small interfering RNA (siRNA). In some embodiments, the oligonucleotide that inhibits expression of CXCR5 is a guide RNA (gRNA) that targets a CXCR5 polynucleotide. Examples of oligonucleotides that inhibit expression of CXCR5 are known in the art, and include, but are not limited to, CXCR5 shRNA TL306391 (Origene), CXCR5 siRNA SR3 00441 (Origene), CXCR5 shRNA TR306391 (Origene), CXCR5 shRNA TL306391V (Origene), CXCR5 CRISPR gRNA ABIN5115520 (Genomics Online), and CXCR5 CRISPR gRNA ABIN5115519 (Genomics Online). In some embodiments, the CXCR5 polynucleotide is a mammalian CXCR5 polynucleotide. In some embodiments, the CXCR5 polynucleotide is a human CXCR5 polynucleotide. In some embodiments, the CXCR5 polynucleotide comprises, consists essentially of, or consists of, the nucleotide sequence of any one of SEQ ID NOs: 15 and 17. In some embodiments, the CXCR5 polynucleotide comprises, consists essentially of, or consists of, a fragment of the nucleotide sequence of any one of SEQ ID NOs: 15 and 17 that encodes for a CXCR5 protein comprising, consisting essentially of, or consisting of, the amino acid sequence of any one of SEQ ID NOs: 16 and 18. In some embodiments, the CXCR5 polynucleotide comprises, consists essentially of, or consists of, a nucleotide sequence that is at least 85%, 90%, 93%, 94%, 95%, 96%, 97%, 98%, 99%, or 100% identical to the nucleotide sequence of any one of SEQ ID NOs: 15 and 17. In some embodiments, the CXCR5 polynucleotide comprises, consists essentially of, or consists of, a fragment of a nucleotide sequence that is at least 85%, 90%, 93%, 94%, 95%, 96%, 97%, 98%, 99%, or 100% identical to the nucleotide sequence of any one of SEQ ID NOs: 15 and 17 that encodes for a CXCR5 protein comprising, consisting essentially of, or consisting of, an amino acid sequence that is at least 85%, 90%, 93%, 94%, 95%, 96%, 97%, 98%, 99%, or 100% identical to the amino acid sequence of any one of SEQ ID NOs: 16 and 18. In some embodiments, the CXCR5 polynucleotide comprises, consists essentially of, or consists of, the nucleotide sequence of any of SEQ ID NOs: 15 and 17. In some embodiments, the CXCR5 polynucleotide comprises, consists essentially of, or consists of, a nucleotide sequence that is at least 85%, 90%, 93%, 94%, 95%, 96%, 97%, 98%, 99%, or 100% identical to the nucleotide sequence of any of SEQ ID NOs: 16 and 18.

In some embodiments, the kits disclosed herein comprise, consist essentially of, or consist of, a polynucleotide encoding a GZMB protein. In some embodiments, the polynucleotide encoding the GZMB protein comprises, consists essentially of, or consists of, the nucleotide sequence of any one of SEQ ID NOs: 19 and 21. In some embodiments, the polynucleotide encoding the GZMB protein comprises, consists essentially of, or consists of, a fragment of the nucleotide sequence of any one of SEQ ID NOs: 19 and 21 that encodes for the GZMB protein comprising, consisting essentially of, or consisting of, the amino acid sequence of any one of SEQ ID NOs: 20 and 22. In some embodiments, the GZMB protein comprises, consists essentially of, or consists of, the amino acid sequence of any one of SEQ ID NOs: 20 and 22. In some embodiments, the polynucleotide encoding the GZMB protein comprises, consists essentially of, or consists of, the nucleotide sequence that is at least 85%, 90%, 93%, 94%, 95%, 96%, 97%, 98%, 99%, or 100% identical to the nucleotide sequence of any one of SEQ ID NOs: 19 and 21. In some embodiments, the polynucleotide encoding the GZMB protein comprises, consists essentially of, or consists of, a fragment of the nucleotide sequence that is at least 85%, 90%, 93%, 94%, 95%, 96%, 97%, 98%, 99%, or 100% identical to the nucleotide sequence of any one of SEQ ID NOs: 19 and 21 that encodes for the GZMB protein comprising, consisting essentially of, or consisting of, an amino acid sequence that is at least 85%, 90%, 93%, 94%, 95%, 96%, 97%, 98%, 99%, or 100% identical to the amino acid sequence of any one of SEQ ID NOs: 20 and 22. In some embodiments, the GZMB protein comprises, consists essentially of, or consists of, an amino acid sequence that is at least 85%, 90%, 93%, 94%, 95%, 96%, 97%, 98%, 99%, or 100% identical to the amino acid sequence of any one of SEQ ID NOs: 20 and 22. In some embodiments, the GZMB is a mammalian GZMB. In some embodiments, the GZMB is a human GZMB.

In some embodiments, the kits disclosed herein comprise, consist essentially of, or consist of, a polynucleotide encoding a TNFRSF18 protein. In some embodiments, the polynucleotide encoding the TNFRSF18 protein comprises, consists essentially of, or consists of, the nucleotide sequence of any one of SEQ ID NOs: 23, 25 and 27. In some embodiments, the polynucleotide encoding the TNFRSF18 protein comprises, consists essentially of, or consists of, a fragment of the nucleotide sequence of any one of SEQ ID NOs: 23, 25 and 27 that encodes for the TNFRSF18 protein comprising, consisting essentially of, or consisting of, the amino acid sequence of any one of SEQ ID NOs: 24, 26, and 28. In some embodiments, the TNFRSF18 protein comprises, consists essentially of, or consists of, the amino acid sequence of any one of SEQ ID NOs: 24, 26, and 28. In some embodiments, the polynucleotide encoding the TNFRSF18 protein comprises, consists essentially of, or consists of, the nucleotide sequence that is at least 85%, 90%, 93%, 94%, 95%, 96%, 97%, 98%, 99%, or 100% identical to the nucleotide sequence of any one of SEQ ID NOs: 23, 25 and 27. In some embodiments, the polynucleotide encoding the TNFRSF18 protein comprises, consists essentially of, or consists of, a fragment of the nucleotide sequence that is at least 85%, 90%, 93%, 94%, 95%, 96%, 97%, 98%, 99%, or 100% identical to the nucleotide sequence of any one of SEQ ID NOs: 23, 25 and 27 that encodes for the TNFRSF18 protein comprising, consisting essentially of, or consisting of, an amino acid sequence that is at least 85%, 90%, 93%, 94%, 95%, 96%, 97%, 98%, 99%, or 100% identical to the amino acid sequence of any one of SEQ ID NOs: 24, 26, and 28. In some embodiments, the TNFRSF18 protein comprises, consists essentially of, or consists of, an amino acid sequence that is at least 85%, 90%, 93%, 94%, 95%, 96%, 97%, 98%, 99%, or 100% identical to the amino acid sequence of any one of SEQ ID NOs: 24, 26, and 28. In some embodiments, the TNFRSF18 is a mammalian TNFRSF18. In some embodiments, the TNFRSF18 is a human TNFRSF18.

In some embodiments, the kits disclosed herein may further comprise, consist essentially of, or consist of, one or more vectors. The vectors may comprise, consist essentially of, or consist of, any of the polynucleotides and/or oligonucleotides disclosed herein. For instance, the vector may comprise, consist essentially of, or consist of, one or more polynucleotides encoding one or more proteins selected from CD4, CXCL13, GZMB, TNFRSF18, TNFRSF4, IFNG, IL21, and a protein listed in Table 11. In some embodiments, the vector comprises, consists essentially of, or consists of, one or more oligonucleotides that inhibit expression of one or more proteins selected from CXCR5, MAF, SH2D1A (SAP), PDCD1, BTLA, CD200, BCL6, and a protein listed in Table 11. In some embodiments, the vector is a mammalian expression vector. In some embodiments, the vector is a plasmid. In some embodiments, the vector is a viral vector selected from the group of a retroviral vector, a lentiviral vector, an adenoviral vector, and an adeno-associated viral vector.

The kits disclosed herein may comprise, consist essentially of, or consist of, one or more cells. In some embodiments the cell is a mammalian cell. In some embodiments, the mammalian cell is a human cell. In some embodiments the cell is an immune cell. In some embodiments, the immune cell is a lymphocyte. In some embodiments, the lymphocyte is a T cell. In some embodiments, the T cell is a CD4⁺ T cell. In some embodiments, the lymphocyte is an NK cell.

The kits disclosed herein may further comprise, consist essentially of, or consist of, one or more agents that bind to one or more proteins selected from CD4, CXCL13, GZMB, TNFRSF18, TNFRSF4, IFNG, IL21, CXCR5, MAF, SH2D1A (SAP), PDCD1, BTLA, CD200, BCL6, and a protein listed in Table 11. The one or more agents may be an antibody or antigen binding fragment thereof. In some embodiments, the kit comprises, consists essentially of, or consists of, (i) an agent that binds to CD4; (ii) an agent that binds to CXCL13, and (iii) an agent that binds to CXCR5. In some embodiments, the kit comprises, consists essentially of, or consists of, (i) an agent that binds to CD4; (ii) an agent that binds to CXCL13, (iii) an agent that binds to CXCR5; and (iv) an agent that binds to GZMB.

In some embodiments, the agents disclosed herein are conjugated to a label. In some embodiments, the one or more agents that bind to different proteins are conjugated to different labels. In some embodiments, the label is a purification marker. A non-exhaustive list of purification markers include His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise, consist essentially of, or consist of, FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

### EXAMPLES

The disclosure is further illustrated by the following non-limiting examples.

### Example 1

Human Subjects

Written informed consent was obtained from all subjects³. Newly diagnosed, untreated patients with non-small cell lung cancer (Table 1), UK between 2014 and 2017 were prospectively recruited. Freshly resected tumor tissue and, where available, matched adjacent non-tumor tissue was obtained from patients with lung cancer following surgical resection.

Method Details

Flow Cytometry of Fresh Samples

Samples were processed as described previously³. For sorting of fresh CD4⁺ TILs for transcriptomic analysis, cells were first incubated with FcR block (Miltenyi Biotec), then stained with a mixture of the following antibodies: anti-CD45-FITC (HI30; Biolegend), anti-CD4-PE (RPA-T4; BD Biosciences), anti-CD3-PE-Cy7 (SK7; Biolegend), anti-CD8a-PerCP-Cy5.5 (SKI; BD Biosciences), anti-HLA-DR-APC (L243; BD Biosciences), anti-CD14-APC-H7 (McpP9; BD Biosciences), anti-CD19-PerCP-Cy5.5 (clone HIB19; Biolegend) and anti-CD20-PerCP-Cy5.5 (clone 2H7; Biolegend) for 30 min at 4° C. Live/dead discrimination was performed by DAPI staining. Stained samples were analyzed using BD FACSAria™ (BD Biosciences) and FlowJo software (Treestar), and CD4⁺ T cells were sorted into ice-cold TRIzol LS reagent (Ambion).

Flow Cytometry of Cryopreserved Samples

For 10× single-cell transcriptomic analysis and phenotypic characterization, tumor and lung samples were first processed and cryopreserved in freezing media (50% complete RMPI (Fisherscientific), 40% human decomplemented AB serum, 10% DMSO (both Sigma). Cryopreserved samples were thawed, incubated with FcR block (Miltenyi Biotec), then stained with a combination of anti-CD45-AlexaFluor700 (HI30; BioLegend); anti-CD3-APC-Cy7 (SK7; Biolegend); anti-CD8A-PerCP-Cy5.5 (SKI; Biolegend); anti-CXCR5-BB515 (RF8B2; BD Biosciences); anti-CD25-PE (MA251; BD Biosciences); anti-CD127-APC (eBioRDR5; eBiosciences); anti-CD19/20-BV421 (HIB19/2H7; Biolegend); anti-CD56-BV570 (HCD56; Biolegend) and anti-CD4-BV510 (OKT4; Biolegend) for flow cytometric analysis and sorting. Live/dead discrimination was performed using propidium iodide (PI). 1500 TILs from each of the three subsets, CD4⁺CXCR5⁺, CD4⁺CXCR5 CD25⁺ and CD4CXCR5CD25CD127⁻ from tumor and 4500 CD4⁺ T cells (N-TILs) from adjacent uninvolved lung of each patient were sorted into 50% ice cold PBS, 50% FBS (Sigma) using BD Aria-III (BD Biosciences).

For intracellular staining for the chemokine CXCL13 and granzyme B, TILs and N-TILs were incubated in RPMI 1640 medium (Life Technologies) containing brefeldin A (5 ug/ul) for 3.5 hrs. TILs and N-TILs were stained using Zombie Aqua fixable viability kit (Biolegend), following which surface staining was performed with a mixture of fluorescently conjugated antibodies: anti-CD45-Alexafluor700 (HI30; Biolegend), anti-CD3-APC-Cy7 (SK3; Biolegend), anti-CD4-PE-Cy7 (RPA-T4; Biolegend), anti-CD8-PerCP-Cy5.5 (SKI; Biolegend), anti-CXCR5-BB515 (RF8B2; BD Biosciences), anti-CD25-BV421 (2A3; BD Biosciences), anti-CD127-PE-Dazzle594 (A019D5; Biolegend), anti-PD1-BV605 (EH12.2H7, Biolegend) for 30 min at 4° C. After fixation (BD Cytofix/Cytoperm) and permeabilization (BD Perm/Wash buffer), intracellular staining was performed with anti-CXCL13-APC (53610, R&D Systems) and anti-GZMB-PE (REA226, Miltenyl Biotec) for 30 min at 4° C. For intracellular staining for T regulatory cells, the following intracellular antibodies and buffers were used: anti-Foxp3-APC (PCH101, ThermoFisher), anti-CXCL13-PE (53610, ThermoFisher) and Foxp3 staining buffer kit (eBioscience). Samples were analyzed on BD LSRII and ImageStream. All FACS data was analyzed in FlowJo 10.4.1.

ImageStream Analysis

Samples were processed as described above for flow cytometry. Images were acquired on a 2-camera ImageStreamX MkII imaging flow cytometer (Amnis, Seattle) at low speed with 40× objective and INSPIRE software version 200.1.620.0. The cytometer passed all ASSIST performance checks prior to image acquisition. BB515 (Ch02, 480-560 nm), PE (Ch03, 560-595 nm) PE-Dazzle594 (Ch04, 595-642 nm), PerCP-Cy5.5 (Ch05, 648-745 nm) and PE-Cy7 (Ch06, 745-780 nm) were excited at 488 nm (40 mW). BV421 (Ch07, 435-505 nm) was excited at 405 nm (20 mW). APC (Chi 1, 640-745 nm) and APC-Cy7 (Ch12, 745-780 nm) were excited at 642 nm (150 mW). The acquisition gate was set to include all single, in-focus, live, CD3⁺ events. Data was compensated and analyzed with IDEAS software version 6.2.64.0 using the default masks and feature set.

Histology and Immunohistochemistry.

Deparaffinisation, rehydration, antigen retrieval and IHC staining was carried out using a Dako PT Link Autostainer. Antigen retrieval was performed using the EnVision FLEX Target Retrieval Solution, High pH (Agilent) for all antibodies. The primary antibodies used for IHC includes anti-CD103 (EPR4166(2); 1:500; Abeam), anti-CXCL13 (polyclonal; 1:100; ThermoFisher Scientific), anti-CD8 (C8/144B; pre-diluted; Agilent Dako), anti-CD4 (4B12; pre-diluted; Agilent Dako), anti-PanCK (AE1/AE3; pre-diluted; Agilent Dako). Primary antibodies were detected using EnVision FLEX HRP (Agilent Dako) and either Rabbit or Mouse Link reagents (Agilent Dako) as appropriate. Chromogenic visualization was completed with either two washes for five minutes in DAB or one wash for thirty minutes in AEC and counterstained with hematoxylin. To analyze multiple markers on single sections, multiplexed IHC staining was performed as described previously⁵⁹. 4 micron tissue sections were stained with anti-PanCK antibody, visualized using DAB chromogenic substrate and scanned using a ZEISS Axio Scan.Z1 with a 20× air immersion objective. Each immune marker was then visualized using AEC chromogenic substrate and scanned. Between each staining iteration, antigen retrieval was performed along with removal of the labile AEC staining and denaturation of the preceding antibodies.

For each tissue section, regions within the tumor core (1 per section) or at the invasive margin (2 per section) were identified by a pathologist (GJT). These regions were exported as ome.tiff files and processed using Fiji image analysis software⁶⁰ as follows. The PanCK alone image was used as a reference for registering each iteration of staining, using the linear stack alignment with SIFT plugin. Color deconvolution for hematoxylin, DAB and AEC staining was performed using a customized vector matrix⁶¹. 8 bit deconvoluted images were then visually inspected to determine a pixel intensity threshold of positive staining for each marker and this value was subtracted from each image to remove non-specific staining. This color deconvolution approach resulted in DAB positive regions also being identified as AEC positive, therefore the PanCK alone image was used to generate a 0/255 pixel intensity binary “DAB mask”, which was then subtracted from each AEC image. Cell simulation and analysis was then performed using Tissue Studio image analysis software (Definiens). A machine learning classifier was trained to recognize epithelial and stromal regions using hematoxylin and PanCK staining. Cells were then identified by nucleus detection and cytoplasmic regions were simulated up to 5 μm. CD4⁺CXCL13⁺ and CD8⁺CD103⁺ cells were then enumerated within the stromal regions of each image. This analysis was performed for 41 patients out of the total 45 patients in the cohort; due to insufficient sample, 4 patients were not analyzed.

Bulk RNA Sequencing

Total RNA was purified using a miRNAeasy micro kit (Qiagen, USA) and quantified as described previously⁶² (on average, ˜8000 CD4⁺ T cells per sample were processed for RNA-Seq analysis). Purified total RNA was amplified following the smart-seq2 protocol^{62, 63}. cDNA was purified using AMPure XP beads (1:1.1 ratio, Beckman Coulter). From this step, 1 ng of cDNA was used to prepare a standard Nextera XT sequencing library (Nextera XT DNA sample preparation kit and index kit, Illumina). Samples were sequenced using HiSeq2500 (Illumina) to obtain 50-bp single-end reads. Quality control steps were included to determine total RNA quality and quantity, optimal number of PCR pre-amplification cycles, and cDNA fragment size⁶². Samples that failed quality control were eliminated from further downstream steps.

10× Single-Cell RNA Sequencing

Samples were processed using 10× v2 chemistry as per manufacturer's recommendations; 11 and 12 cycles were used for cDNA amplification and library preparation respectively⁶⁴. Barcoded RNA was collected and processed following manufacturer recommendations, as described previously. Libraries were sequenced on a HiSeq4000 (Illumina) to obtain 100- and 32-bp paired-end reads using the following read length: read 1, 26 cycles; read 2, 98 cycles; and i7 index, 8 cycles.

Bulk-RNA-Seq Analysis

Bulk RNA-Seq data were mapped against the hg19 reference using TopHat⁶⁵ (v1.4.1 (—library-type fr-unstranded —no-coverage-search) and read counts were calculated using htseq-count -m union -s no -t exon -i gene_id (part of the HTSeq framework, version 0.7.1))⁶⁶. Cutadapt (v1.3) was used to remove adapters. To identify genes expressed differentially by two groups, Applicants performed negative binomial tests for paired comparisons by employing the Bioconductor package DESeq2 (v1.14.1), disabling the default options for independent filtering and Cooks cutoff⁶⁷. Applicants considered genes to be expressed differentially by any comparison when the DESeq2 analysis resulted in a Benjamini-Hochberg-adjusted P value of <0.05 and a fold change of at least 1.5. The Qlucore Omics Explorer 3.2 software package was used for visualization and representation (heat maps, principal component analysis) of RNA-Seq data⁶⁸. The biological relevance of differentially expressed genes identified by DESeq2 analysis was further investigated using the Ingenuity Pathways Analysis platform as reported previously³. Unsupervised hierarchical clustering of samples based on the expression of genes (n=2000) with the highest variance, which accounted for 35% of the total variance, was performed using DESeq2 package and custom scripts on R. For tSNE analysis, the data frame was filtered to genes with >1 TPM expression in at least one condition and visualizations created using the top 2000 most variable genes, as calculated in DESeq2; this allowed for unbiased visualization of the Log₂ (TPM+1) data, using package Rtsne (v0.13). T cell receptor (TCR) sequences were retrieved from CD4⁺ T cell RNA-Seq data sets and the frequency of TCR beta chain clonotypes was determined using default parameters of the MiXCR v2.1.5 package⁶⁹ (Table 4). The CD103 status of TILs was determined as previously described³. GSEA, correlations, and heatmaps were generated as previously described^{3, 68}. Genes used in the GSEA analysis are shown in Table 10. Windrose plot was generated on Microsoft Office Excel suite. Union gene signatures were calculated using the online tool jvenn⁷⁰, of which genes must have common directionality.

Weighted Gene Coexpression Network Analysis

WGCNA was completed using a R package WGCNA (v1.61) from the TPM data matrix²⁷. Expressed genes with TPM>1 in at least 25% of the samples, were used in both CD4⁺ and CD8⁺ TIL data. In the integrated WGCNA approach, highly correlated genes from combined transcriptomes of patient-matched CD4⁺ TILS and CD8⁺ TILS were identified and summarized with a modular eigengene (ME) profile²⁷. Gene modules were generated using blockwiseModules function (parameters: checkMissingData=TRUE, power=6, TOMType=“unsigned”, minModuleSize=50, maxBlockSize=25426, mergeCutHeight=0.40). Module 30, which represented the default ‘grey’ module generated by WGCNA for non-co-expressed genes, was excluded from further analysis. For each gene module, individual MEs were also calculated for CD4⁺ TIL-genes and CD8⁺ TIL-genes separately. As each module by definition is comprised of highly correlated genes, their combined expression may be usefully summarized by eigengene profiles, effectively the first principal component of a given module. A small number of eigengene profiles may therefore effectively ‘summarize’ the principle patterns within the cellular transcriptome with minimal loss of information. This dimensionality-reduction approach also facilitates correlation of ME with traits. Cell cycle signature was used to generate an eigengene vector from CD8⁺ TIL-genes, which was then used as a trait and correlated with MEs. Significance of correlation between this trait and MEs was assessed using linear regression with Bonferroni adjustment to correct for multiple testing.

To visualize co-expression network, Applicants used the function exportNetworkToCytoscape at weighted=true, threshold=0.05. A soft thresholding power was chosen based on the criterion of approximate scale-free topology. Networks were generated in Gephi (v0.92)^{71, 72} using Fruchterman Reingold and Noverlap functions. The size and color were scaled according to the Average Degree as calculated in Gephi, while the edge width was scaled according to the WGCNA edge weight value.

Single-Cell RNA-Seq Analysis

Raw 10× data was processed as previously described, merging multiple sequencing runs using cellranger count function in cell ranger, then merging multiple cell types with cell ranger aggr (V2.0.2). The merged data was transferred to the R statistical environment for analysis using the package Seurat (v2.1)^{64, 73}. Only cells expressing more than 200 genes and genes expressed in at least 3 cells were included in the analysis. The data was then log-normalized and scaled per cell and variable genes were detected. Transcriptomic data from each cell was then further normalized by the number of UMI-detected and mitochondrial genes. A principal component analysis was then run on variable genes, and the first 6 principal components (PCs) were selected for TILs for further analyses based on the standard deviation of PCs, as determined by an elbow plot in Seurat. Cells were clustered using the FindClusters function from Seurat with default settings, resolution=0.6. Clusters with less than 50 cells were excluded from analysis. Seurat software was used to identify cluster-specific differentially expressed gene sets (cutoff used is q<0.05).

Differential expression between two groups was determined by converting the data to CPM and analyzing group-specific differences using MAST (q<0.05, v1.2.1) (FIG. 4; Table 8)^{64, 74, 15}. For differential expression between three groups (FIG. 5A), pairwise comparisons were performed. A gene was considered significantly different (unique to a group), only if the gene was commonly positively enriched in every comparison for a singular group^{64, 68}. A gene was considered shared between two groups if the gene was commonly positively enriched in the two groups compared to the third group. For this analysis in FIG. 5A, only genes differentially expressed in CXCL13-expressing versus CXCL13-non-expressing cells (FIG. 4; Table 8) were used.

The mean CPM and percentage of cells expressing a transcript expressing cells was calculated with custom R scripts. Further visualizations of exported normalized data were generated using the Seurat package and custom R scripts. Cell-state hierarchy maps were generated using Monocle version 2.4.0⁷⁶ and default settings with expressionFamily=negbinomial.size( ), lowerDetectionLimit=0.5 and num_dim=3, including the most variable genes identified in Seurat for consistency. Average expression across a cell cluster was calculated using the AverageExpression function, and downsampling was achieved using the SubsetData function (both in Seurat).

Quantification and Statistical Analysis

Comparison between two groups was assessed with Mann-Whitney test (FIG. 7, S3B, S4C and S4F) using GraphPad Prism 7. Hypergeometric test using phyper function and p.adjust in R was used to calculate adjusted significance values for gene enrichment tests (FIG. 3C). Spearman correlation coefficient (r value) was calculated to assess significance of correlation between any two parameters of interest (FIG. 6B; FIG. 8).

Experimental Results

Tumor-Infiltrating CD4⁺ T Cells Show Features of Activation and Co-Stimulation

Applicants recently reported on the transcriptomes of human tumor-infiltrating CD8⁺ T cells, however, the global gene expression profile of tumor-infiltrating CD4⁺ T cells and their state of activation, differentiation and function within tumors has not been fully characterized^{17, 19, 20, 21}. Specifically, the nature of CD4⁺ T cell responses that are linked to robust anti-tumor CD8⁺ effector and T_{RM} responses within tumors, where priming and induction of T cell responses may occur^{22, 23, 24}, are unknown. To understand the molecular interactions between CD4⁺ and CD8⁺ T cells in the tumor microenvironment that lead to robust anti-tumor CD8⁺ effector and T_{RM} responses, Applicants analyzed the transcriptomes of CD4⁺ T cells present in tumor samples (TILs) and in adjacent uninvolved lung tissue (N-TILs) from 45 treatment-naïve patients with early stage NSCLC, and related these findings to transcriptomic data from patient-matched tumor-infiltrating CD8⁺ T cells (FIG. 1A; Table 1)³.

Over 750 transcripts were differentially expressed by tumor-infiltrating CD4⁺ T cells when compared to lung CD4⁺ T cells (Benjamini-Hochberg adjusted P<0.05 and >1.5 fold change, Methods) (; Table 2), which suggested major changes in the transcriptional landscape of tumor-infiltrating CD4⁺ T cells. Pathway analysis of the transcripts with increased expression in tumor-infiltrating CD4⁺ T cells revealed significant enrichment of T cell and B cell activation and T cell receptor (TCR) engagement pathways (FIG. 1B; Table 3). Applicants next performed gene set enrichment analysis (GSEA) to test for enrichment of transcriptional signatures reflecting various T cell-related phenotypes that may co-exist within tumors. The dominant signatures that emerged were that of T cell activation and co-stimulation but not exhaustion (FIG. 1C and FIG. 1D), which suggested that tumor-infiltrating CD4⁺ T cells were undergoing TCR ligation-mediated activation and differentiation, likely driven by tumor-associated antigens (TAA) presented by antigen presenting cells (APCs) within tumors. In support of this, analysis of TCR repertoire demonstrated significantly greater clonal expansion of tumor-infiltrating CD4⁺ T cells when compared to lung CD4⁺ T cells (FIG. 7; Table 4). These findings indicated that such activated CD4⁺ T cells might have an important functional role in immune responses within lung tumors.

Concordant Expression Pattern of Immunotherapy Target Molecules in Tumor-Infiltrating CD4⁺ and CD8⁺ T Cells

Therapeutic targeting of signaling pathways involved in T cell co-stimulation, co-inhibition as well as immune checkpoints have shown considerable promise in preclinical and human studies^{6, 25, 26}. Although typically CD8⁺ T cells are presumed to be the key cellular targets of such therapies, e.g., anti PD-1 therapy, it is important to determine whether concordant responses can occur in the CD4⁺ T cells present in the tumor, as therapies that simultaneously boost CD8⁺ and CD4⁺ T cell responses may be more beneficial. Applicants therefore evaluated the pattern of expression of some important immunotherapy targets in CD4⁺ T cells and CD8⁺ T cells present in the tumor microenvironment of the same patients. Similar to CD8⁺ T cells, considerable inter-patient heterogeneity was observed in the expression of transcripts encoding the co-stimulatory and co-inhibitory molecules in CD4⁺ T cells (FIG. 2A). Most importantly, the expression of these transcripts was positively correlated between patient-matched tumor-infiltrating CD4⁺ and CD8⁺ T cells (FIG. 2B; FIG. 8). This suggested that biological therapies targeting molecules on CD8⁺ T cells may also activate CD4⁺ T cells present in the tumor. But, there were some exceptions; for example, whilst patient 34 was low for PDCD1 (encoding for PD-1) expression in CD8⁺ TILs, there was substantial PDCD1 expression in matched CD4⁺ TILs; the converse was true for the expression pattern of HAVCR2, encoding for the immune checkpoint molecule TIM-3 (FIG. 2C and FIG. 2D). Thus, a detailed assessment of the global gene expression programs of tumor-infiltrating CD8⁺ and CD4⁺ T cells may provide information to guide the rational choice of combination therapies aimed at activating both cell types.

Follicular Program in CD4⁺ T Cells is Associated with CD8⁺ T Cell Proliferation, Cytotoxicity and Tissue Residency

The general concordance in the expression pattern of potential immune therapy targets between CD4⁺ and CD8⁺ T cells present in the tumor microenvironment suggested TCR engagement and activation of both cell types, presumably in response to TAA. To characterize the molecular interplay between these cells and to define the properties in CD4⁺ T cells that are strongly associated with either robust or poor anti-tumor CD8⁺ effector and T_{RM} responses across these cohort of patients with lung cancer, Applicants performed integrated weighted gene correlation network analysis (iWGCNA). In a complex set of data such as the transcriptome, similar measurements may be grouped together by weighted gene correlation network analysis (WGCNA) to form discrete gene modules that may be correlated with specific traits to determine the gene network module linked to that trait²⁷. Applicants applied this principle in iWGCNA to group transcript expression from different cell types of matched patients to form integrated gene modules to reveal the molecular cross talk between cell types and their relationship to specific traits that are variable across the cohort.

Applicants performed iWGCNA by merging the transcriptomes from patient-matched CD4⁺ and CD8⁺ T cells present in the tumor (n=36) and generated 29 gene network modules, each of which were composed of varying proportions of CD4⁺ T cell- and CD8⁺ T cell-transcripts (FIG. 3A; Table 5). To determine what properties in CD4⁺ T cells were associated with robust CD8⁺ T cell responses, Applicants correlated these gene modules with CD8⁺ T cell proliferation signature as a trait (Methods), as it represented a feature of productive and robust TAA-specific T cell responses within tumors. Module 7 emerged as the most significantly correlated gene module (FIG. 3A) and, as expected, nearly 25% of the CD8⁺ T cell-transcripts in Module 7 were cell cycle-related genes. Clustering analysis of the CD8⁺ T cell-transcripts (n=407) present in Module 7 identified a tightly correlated and co-expressed subset of transcripts (n=171), which included cell cycle genes and several genes encoding products linked to effector and cytotoxic functions such as GZMB, CCL3, STAT1, FKBP1A, KIR2DL4 (FIG. 3B and FIG. 3C; Tables 5 and 6). Remarkably, this highly co-expressed cluster of CD8⁺ T cell-transcripts also contained ITGAE (which encodes for the α_{E} subunit of the integrin molecule α_{E}β₇), a well-established marker of lung CD8⁺ T_{RM} cells^{28, 29}, which Applicants have recently shown to be a critical determinant of survival outcomes in lung cancer (FIG. 3B; Tables 5 and 6)³. Of note, FABP5, a member of this cluster, encodes for fatty-acid-binding protein 5, which plays a crucial role in the maintenance, longevity and function of CD8⁺ T_{RM} cells³⁰. Module 7 also contained NAB 1, which encodes for a transcriptional regulator, which in murine models has been linked to CD4⁺ T cell-mediated ‘help’ to CD8⁺ T cells (Table 5)³¹. Together, these findings indicated that cell proliferation, effector functions and T_{RM} features are highly correlated and interconnected processes in CD8⁺ T cells present within the tumors.

To assess the properties in CD4⁺ T cells associated with these functional features in tumor-infiltrating CD8⁺ T cells, Applicants next analyzed the CD4⁺ T cell-transcripts (n=178) present in Module 7. Applicants observed a tightly correlated and co-expressed cluster of transcripts (n=61), which included several genes linked to follicular helper CD4⁺ T cells³² (T_{FH}) such as CXCL13, BATF, CD38, IL12RB2, PDCD1 and cell proliferation (e.g., MKI67, TOP2A, STMN1, CDK1) (FIG. 3B; Tables 5 and 6). T_{FH} cells, first identified in human tonsils, are the principal CD4⁺ T cell subpopulation that provides essential ‘help’ to B cells promoting antibody affinity maturation in germinal centers (GC)³³. Among the T_{FH}-related transcripts in this cluster, BATF encodes for a transcription factor involved in T_{FH} differentiation³⁴. CXCL13 is a chemokine produced by human T_{FH} cells, but not by their murine counterparts; CXCL13 has been shown to play an important role in the homing of B cells to follicles^{35, 36, 37}. IL12RB2 encodes for the IL-12 receptor beta2 subunit, and IL-12 is a key cytokine that mediates T_{FH} cell formation in humans, in addition to IL-23 and TGF-β³⁸. In further support of a T_{FH}-like transcriptional program in CD4⁺ TILs, Module 7 contained the transcript encoding for the canonical NOTCH signaling mediator, RBPJ, which has been shown to be a critical regulator of T_{FH} development and function (Table 5)³⁹. Furthermore, Module 7 exhibited the highest enrichment for both the T_{FH} and cell cycle signature genes among the 29 total gene modules generated from the iWGCNA (FIG. 3C).

Recently activated T_{FH} cells are marked by the expression of markers such as CD38^{40, 41, 42}. In addition to CD38 transcripts, the Module 7 cluster was also composed of transcripts encoding other T cell activation-related molecules such as TNFRSF9 (which encodes for 4-1BB), TNFRSF18 (which encodes for GITR), and TNFRSF8 (which encodes for CD30) (FIG. 3B; Table 5). CD30 has been shown to be preferentially expressed by activated and memory T_{FH} cells⁴³ whilst GITR-mediated co-stimulation is known to augment T_{FH} cell responses⁴⁴. These results demonstrated that the T_{FH} program was coupled with proliferation and activation of CD4⁺ T cells in the tumor milieu.

Consistent with iWGCNA that indicated the link between T_{FH} program in CD4⁺ T cells and T_{RM} features, the CD4⁺ T cell-transcripts in Module 7 showed increased expression in T_{RM}^{high} tumors relative to their expression in T_{RM}^{low} tumors (FIG. 9; Table 1; Methods). Furthermore, GSEA also showed significant enrichment of proliferation and T_{FH} gene signatures in tumor-infiltrating CD4⁺ T cells from T_{RM}^{high} tumors relative to T_{RM}^{low} tumors (FIG. 3D). Taken together, these results demonstrate that a T_{FH}-like transcriptional program in tumor-infiltrating CD4⁺ T cells was strongly associated with CD8⁺ T cell proliferation, effector function and T_{RM} features in the tumor milieu, all features of a robust immune response.

CXCL13-Ex Pressing Tumor-Infiltrating CD4⁺ T Cells Possess Superior Functional Properties

Applicants next sought to compare tumor-infiltrating CD4⁺ T cells with or without a follicular program to determine the functional properties of T_{FH} cells that render them superior at supporting robust CD8⁺ T cell effector and T_{RM} responses. Conventional GC T_{FH} cells are characterized by the expression of CXCR5, however in the context of tumor and inflammation, T_{FH} cells lacking expression of CXCR5 have also been described^{45, 46}. Therefore, to capture the entire spectrum of CD4⁺ T cells with a follicular program, Applicants performed single-cell RNA-Seq of purified populations of tumor-infiltrating CD4⁺ T cells from an additional 6 patients. The CD4⁺ T cells were flow-sorted as CXCR5+, CXCR5⁻CD25⁺CD127⁻ or CXCR5CD25⁻ subsets to enrich for T_{FH}, Tregs and effector CD4⁺ T cells, respectively (FIG. 4A; Table 1). Given that GC T_{FH} are the major producers of the B cell chemoattractant, CXCL13, involved in the organization of GCs and tertiary lymphoid structures (TLS)³⁵, Applicants utilized CXCL13 expression as a surrogate marker for T_{FH} cells.

Unbiased analysis of the ˜5300 single-cell CD4⁺ TIL transcriptomes revealed 9 clusters (Methods). Cells expressing CXCL13 transcripts were highly enriched in cluster 3 (˜70% of cells expressed CXCL13), which suggested that CAT 7,73-expressing cells likely represented a distinct CD4⁺ T cell subset (FIG. 4B; FIG. 10A; Table 7). In marked contrast, very little CXCL13 expression was seen in lung-infiltrating CD4⁺ T cells (FIG. 10B). These findings were confirmed at the protein level by flow cytometry analysis of CXCL13 expression in TILs and N-TILs (FIG. 10B). Single-cell differential gene expression analysis of the CXCL13-expressing versus CAT 7,73-non-expressing CD4⁺ TILs (Methods) revealed over 1000 differentially expressed transcripts, which indicated a discrete biological identity for the CXCL13-expressing cells (Table 8). GSEA showed significant enrichment of T_{FH} signature genes, nearly one third of which showed increased expression in the CXCL13-expressing cells (FIG. 4C; FIG. 10C). Applicants found both higher expression and higher percentage of cells expressing T_{FH}-related genes (MAP, SH2D1A, PDCD1, BTLA, CD200, BCL6) in CXCL13-expressing cells than in CYCL13-non-expressing cells (FIG. 4C; FIG. 10C). These findings clearly established that the expression of CXCL13 delineated a T_{FH} program, i.e., CXCL13-expressing cells represented T_{FH}-like cells. Notably, Applicants found that CXCL13-expressing cells were present in equal proportions in CXCR5⁺ and the two CXCR5⁻ subsets (FIG. 4B), which indicated that using CXCR5 as a surface marker for T_{FH} cells would have missed several other potentially important T_{FH} subsets.

Consistent with iWGCNA results (FIGS. 3B-3C), GSEA demonstrated enrichment of cell cycle genes in the CXCL13-expressing cells (FIG. 4D). Higher proportions of CXCL13-expressing cells also expressed cell cycle-related transcripts (FIG. 4D), and remarkably, Applicants found that cluster 8 was specifically enriched for cycling T_{FH} cells. Together these results indicated that despite expressing high levels of PDCD1, encoding for the immune checkpoint molecule PD-1 (FIG. 4C; FIG. 10C and FIG. 10D), T_{FH} cells actively proliferated in the tumor microenvironment presumably in response to TAA.

To probe the functional properties of these T_{FH}-like cells, Applicants performed pathway analysis of the transcripts with increased expression in CXCL13-expressing cells relative to CXCL13-non-expressing cells. CXCL13-expressing T_{FH}-like cells showed significant enrichment of pathways linked to helper T cell (T_{H}) co-stimulation (CD28 signaling in T_{H} cells) and ICOS-ICOSL signaling, which is important for T_{FH} activation (FIG. 4E; Table 9)³². As expected of activated and co-stimulated T cells in the tumor, these T_{FH}-like cells showed increased expression of transcripts encoding for cytokines (IFN-gamma and IL21) and co-stimulation molecules (GITR and OX-40), which are all known to play an important role in CD4⁺ T cell-mediated ‘help’ to CD8⁺ T cells (FIG. 4F)^{47, 48, 49, 50}. Notably, the immunosuppressive CD4⁺ T cell subset, i.e., Tregs, was seen mainly within CXCL13-non-expressing CD4⁺ T cells, which also showed differential expression of FOXP3 transcripts (FIG. 10E).

A surprising finding was the enrichment of cytotoxicity pathway in CXCL13-expressing cells relative to CXCL13-non-expressing cells. (FIG. 4E; Table 9). This finding was independently confirmed by GSEA, which also showed significant enrichment of cytotoxicity signature genes, in the CXCL13-expressing cells (FIG. 4G; FIG. 10F). Applicants found higher expression and higher percentage of cells expressing cytotoxicity-related transcripts such as GZMB, GZMM, FKBP1A, RAB27A, CCL4 and ZEB2 in CXCL13-expressing than in CXCL13-non-expressing cells (FIG. 4G), which suggested the presence of cytotoxic T_{FH}-like CD4⁺ T cells in the tumor microenvironment. In summary, these single-cell RNA-Seq studies unraveled the presence and superior functional properties of an important CD4⁺ T cell subset—activated and proliferating T_{FH}-like cells that had the potential for direct cytotoxicity as well as provision of ‘help’ to CD8⁺ CTLs.

Highly Functional T_{FH}-Like CD4⁺ T Cells were CXCR5 Negative

Because these anti-tumor functions were not previously ascribed to T_{FH} cells, which predominantly provide ‘help’ to B cells, Applicants sought to investigate the precise nature of the cells that harbored these functions. Since the CXCL13-expressing T_{FH}-like cells were present in equal proportions in CXCR5⁺ and the two CXCR5⁻ subsets (CD25⁺CD127⁻ and CD25⁺), Applicants first asked whether the superior functional properties were attributes of all or unique to one subset. Pairwise comparisons for differential expression analysis between the three subsets (Methods) showed that T_{FH} signature genes were significantly enriched in all three T_{FH} subsets (FIG. 11), which suggested that all subsets had switched on a T_{FH} molecular program. However, transcripts linked to superior anti-tumor properties, such as cytotoxicity (KLRB1, GZMB, CCL3, CCL4, FKBP1A, SOD1, ZEB2) and provision of CD8⁺ T cell ‘help’ (IFNG, GITR, OX40) were mainly enriched in the CXCR5⁻ T_{FH} subsets (FIG. 5A and FIG. 5B; FIG. 11). Notably, cell cycle-related transcripts were also expressed predominantly in this CXCR5⁻ T_{FH} subset, which together suggested that the highly functional T_{FH} cells that proliferated in the tumor milieu were contained within this subset (FIG. 5A and FIG. 5B; FIG. 11).

T_{FH}-Like Cells Infiltrate Tumor and Associate with CD8⁺ T_{RM} Cells

The enrichment of cytotoxicity pathways and transcripts encoding cytotoxic molecules in CD4⁺ T_{FH} cells was unexpected (FIG. 4E and FIG. 4G). Therefore, Applicants assessed co-expression of CXCL13 and granzyme B in tumor-infiltrating CD4⁺ T cells using three independent approaches: a) intracellular staining by flow cytometry, b) ImageStream imaging cytometry, and c) immunohistochemical (IHC) analyses of human lung tumor samples. Importantly, to directly capture in vivo expression states, tumor-infiltrating CD4⁺ T cells were analyzed without in vitro stimulation in all the three strategies. CXCL13 and granzyme B co-expression was observed by all methods, albeit in a small proportion of tumor-infiltrating CD4⁺ T cells that were indeed predominantly CXCR5⁻ (FIG. 6A).

Applicants next undertook a spatially resolved analysis using multi-parametric immunohistochemistry to gain global insights into the organization of T cells within tumors, and importantly, determine the spatial relationship between tumor cells, T_{FH}-like CD4⁺ T cells and CD8⁺ T_{RM} cells, which has been linked to good survival outcomes^{2, 3, 4, 5}. CXCL13-expressing T_{FH}-like CD4⁺ T cells were localized in TLS as well as in the tumor core and its invasive margins. CD8⁺CD103⁺ T_{RM} cells in the tumor core and invasive margins were seen in close proximity to CXCL13-expressing CD4⁺ T cells, which suggested potential for crosstalk and ‘help’. The high density of cells in TLS rendered precise quantification of individual cell types infeasible, hence Applicants enumerated cells in the tumor core and invasive margins. Applicants found a positive correlation between the absolute number of T_{RM} cells (CD8⁺CD103⁺ cells) and T_{FH} cells (CD4⁺CXCL13⁺ cells) in both tumor core and invasive margins (FIG. 6B). Importantly, the absolute number of T_{RM} cells (CD8⁺CD103⁺ cells) also positively correlated with the proportion of CD4⁺ cells that were CXCL13⁺ (FIG. 6B), indicating a strong association between follicular program in CD4⁺ T cells and CD8⁺ T_{RM} responses, a finding that warrants experimental and functional verification.

Experimental Discussion

Applicants have interrogated the transcriptomes of patient-matched CD4⁺ and CD8⁺ TILs using a novel, integrated gene correlation network analysis-based approach (iWGCNA) to identify molecular features of CD4⁺ TILs linked to robust CD8⁺ anti-tumor immune responses and thus better patient outcomes. Published transcriptional studies of tumor-infiltrating CD4⁺ T cells from patients with cancer have largely focused on the analysis of specific CD4⁺ T cell subtypes or CD4⁺ TILs in isolation without integration with CD8⁺ T cell responses^{16, 17, 18}. Applicants have previously reported on the transcriptional profile of tumor-infiltrating CD8⁺ T cells using a well-characterized cohort of patients with treatment-naïve NSCLC who had a spectrum of TIL responses³. In this study, Applicants surveyed the transcriptomes of CD4⁺ TILs and N-TILs derived from lung tumors and the adjacent uninvolved lung of the same patient cohort in which CD8⁺ TIL transcriptomic analysis was previously performed. This combined TIL transcriptomic dataset generated from patient-matched samples facilitated the capture of in vivo TIL interactions within the tumor microenvironment and functional mapping of CD4⁺ TIL responses with those of CD8⁺ TILs. Such cell specific, context dependent, cross-talk would have otherwise been challenging to decipher in patients.

Applicants revealed a core CD4⁺ TIL transcriptional program comprising ˜750 genes that was shared by tumor subtypes and was distinct from that of N-TILs. This profile suggested extensive molecular reprogramming within the tumor microenvironment and enrichment of presumably TAA-specific cells following TCR engagement, activation and co-stimulation. An additional finding of considerable clinical importance was the striking correlation in the expression of immunotherapy target molecules between CD4⁺ and CD8⁺ TILs within patients despite heterogeneity in levels of expression between patients. This observation suggested the occurrence of coordinated TIL responses in tumors that were “immune hot” and that CD4^{r} TILs may also constitute a critical target, in addition to CD8⁺ TILs, for immune therapies such as anti-PD1 agents. This notion was further supported by recent studies that showed that the gut microbiome modulated responses to PD-1-based therapy, an effect that was dependent on both CD4⁺ and CD8⁺ T cells^{51, 52}. Taken together, these data raise the speculation that in addition to potentially acting as one of the direct mediators of checkpoint blockade-induced responses, CD4⁺ TILs may also indirectly contribute to the observed effects of such therapies by potentiation of CD8⁺ TILs. Applicants propose that evaluation of expression patterns of immune therapy molecules in both CD4⁺ and CD8⁺ TILs may guide rational combination of therapies that beneficially target both cell types for synergistic anti-tumor responses.

Using this cohort of patients with a range of CD8⁺ and T_{RM} TIL densities, Applicants unexpectedly discovered a link between T_{FH} program in CD4⁺ TILs and features of a robust CD8⁺ T cell anti-tumor immune response such as proliferation, cytotoxicity and tissue residency. Furthermore, Applicants found that T_{FH}-like CD4⁺ TILs possessed superior functional properties including proliferation, cytotoxicity and provision of ‘help’ to CD8⁺ CTLs. Conventional GC T_{FH} are known to provide B cell ‘help’ during viral infections by promoting GC development, B cell affinity maturation and class switch recombination³². Consistent with their established role, T_{FH} cells induced B cell responses in breast cancer and were associated with improved survival⁴⁵. However, the association of T_{FH} cells with CD8⁺ T cell ‘help’ and robust CD8⁺ T cell responses has not been described before. Applicants uncovered increased expression of transcripts encoding molecules that mediate CD8⁺ T cell ‘help’ (TNFRSF18, TNFRSF4, IFNG, IL21) in T_{FH}-like CD4⁺ TILs. CD4⁺ helper-derived IL21 has a prominent role in CD8⁺ T cell ‘help’ by inducing the BATF-IRF4 axis to sustain CD8⁺ T cell maintenance and effector response⁴⁹. OX40 and GITR signaling on CD4⁺ T cells critically impacts CD8⁺ T cell priming, accumulation and expansion^{47, 48}. The role of interferon-γ produced by CD4⁺ T cells in helping CD8⁺ T cells and CD8⁺ T_{RM} cells has been well established^{50, 53}. Applicants further showed the co-localization of CD8⁺ T_{RM} cells with CD4⁺CXCL13⁺ T_{FH} cells in tumor invasive margins and tumor core, which lends support to the notion that T_{FH} cells may mediate CD8⁺ T cell ‘help’, a finding that will require experimental confirmation.

This single-cell RNA-Seq studies unraveled another novel finding, the expression of granzyme B by the T_{FH}-like CD4⁺ TILs. The existence of MHC class II-restricted CD4⁺ CTLs has been demonstrated in viral infections where they may play a particularly important role in viral clearance in the face of virus escape strategies to CD8⁺ CTL responses⁵⁴. Although T_{FH} cells with cytotoxic potential has not been reported, it is plausible to hypothesize that a CTL program may also be induced in tumor-infiltrating T_{FH} cells within tumors that have downregulated their MHC I expression. Interestingly, TGF-β signaling has been shown to promote differentiation of both CD4⁺ CTLs^{55, 56, 57} and CD8⁺ T_{RM} cells^{29, 58}, whilst IL2 depletion may induce CXCL13 production and T_{FH} development⁴⁵. Strikingly, both these signaling cues are abundant within tumors that harbor Tregs, suggesting that such CD4⁺ T cell plasticity may have evolved as a mechanism to provide these TILs with survival fitness in such immunosuppressive IL2-deprived environments. Applicants utilized a number of complementary methods and provided a spatially resolved analysis to confirm the presence and location of these GZMB- and CXCL13-expressing CD4⁺ TILs. These results warrant further in vivo studies in mouse models of tumor to elucidate the temporal dynamics of the development of this novel subset and their functional role in tumor elimination.

In further dissecting the molecular profile of the T_{FH}-like CD4 TILs using single cell resolution, Applicants revealed T_{FH} features in both the CXCR5⁺ and CXCR5⁻CD4⁺ T cell subsets. These results are consistent with recent studies, which demonstrated the presence of CXCL13-producing T_{FH} cells that lacked CXCR5 expression both in breast cancer and rheumatoid arthritis^{45, 46}. An additional finding from these studies was that the superior functional properties such as cytotoxicity, provision of CD8⁺‘help’ and proliferation observed in T_{FH}-like CD4⁺ TILs, specifically resided in the CXCR5⁻ subset.

Thus, this study has not only revealed a close link between T_{FH} program in CD4⁺ TILs and robust CD8⁺ CTL and T_{RM} response within tumors, but has also shed light on the distinct functional potential of the T_{FH}-like CXCR5⁻CD4⁺ TILs. Experimental validation of these findings in vivo in murine models will enable further understanding of the mechanisms underlying generation of these cells and their functional significance in anti-tumor immune responses. These findings suggest that eliciting a T_{FH} program in CD4⁺ T cells may be an important component of immunotherapies and vaccination approaches aimed to generate robust and durable CD8⁺ CTL and T_{RM} responses against neo-antigens or shared tumor antigens.

T_{FH}-Like CD4⁺ T Cells are Present within Human Tumors and Enriched Following Checkpoint Blockade

The Applicants validated these findings in NSCLC by performing an integrated analysis of nine published single-cell studies of CD4⁺ TIL transcriptomes (n=25,149) derived from six different cancers. Consistent with the data provided herein, a distinct cluster of CXCL13-expressing cells (cluster 3) was found, reported by some of these studies as “exhausted”, which indeed displayed T_{FH} program (FIG. 12A). Remarkably, cluster 6, which was composed of proliferating cells, also exhibited CXCL13 transcript expression and T_{FH} cell features, indicating that CXCL13-expressing cells were not exhausted. In addition to the presence of CXCL13-expressing cells, expression of GZMB transcripts by a fraction of the CXCL13-expressing cells (FIG. 12B) was also found. Although the CXCL13-expressing cells displayed functional features such as proliferation and cytotoxic potential, higher PDCD1 transcript expression by CXCL13-expressing cells relative to CXCL13-non-expressing cells (FIG. 12C) was discovered, showing that CXCL13-expressing cells may be a potential target of anti-PD1 therapy and may contribute at least in part to the ensuing anti-tumor immune response. In support of this, GSEA showed significant enrichment of T_{FH} gene signatures within tumors following checkpoint blockade and there was a trend for increased proportion of CXCL13-expressing cells in tumors post-PD1 therapy when compared to matched pre-therapy tumors (FIG. 12D). Furthermore, differential gene expression analysis of CXCL13-expressing cells in tumors post-PD1 therapy relative to matched tumors pre-PD1 therapy, showed over 100 upregulated transcripts including that of CXCL13, GZMH and those involved in pathways such as CTL-mediated apoptosis, antigen presentation, cell cycle and fatty acid activation (FIG. 12E). Overall, meta-analysis of large published single cell datasets derived from a range of human cancers confirm the presence of T_{FH}-like cells with unique functional features within tumors and their enrichment following PD1-blockade therapies.

Induction of T_{FH} by Immunization Bolsters CD8⁺ CTL Response and Impairs Tumor Growth

In order to evaluate the functional role of T_{FH} cells in anti-tumor immunity in vivo, the Applicants utilized the B16F10-OVA syngeneic mouse tumor model. The Applicants subcutaneously injected C57BL/6 mice with B16F10-OVA, a melanoma cell line engineered to express the exogenous antigen chicken ovalbumin (OVA). In this widely established model, the aggressive growth of the B16F10-OVA is unhindered by immunotherapy. To assess the impact of T_{FH} responses, OT-II mice were first immunized with OVA-alum, an approach known to generate T_{FH} cells, and adoptively transferred purified OT-II T_{FH} cells or OT-II T_{EFF} cells to tumor-bearing mice at 11 days post-tumor inoculation. Significant impairment of tumor growth immediately following OT-II T_{FH} transfer was found when compared with that following OT-II T_{EFF} transfer or no transfer at all; however, the restraint in tumor growth was not sustained, likely due to the attrition of the transferred OT-II T_{FH} cells (FIG. 13A). The Applicants employed a second strategy to validate previous findings by transferring OT-II cells and immunized tumor-bearing mice with OVA-alum (by footpad and tail-base injection), an approach well established for the induction of T_{FH} cells in secondary lymphoid organs. The Applicants found significant reduction in tumor volumes in the mice that received OT-II transfer and immunization relative to control mice that received OT-II transfer alone (FIG. 13B). The tumors in immunized mice were marked by an increased frequency of infiltrating T cells. T_{FH} cells were increased in tumors of immunized mice relative to unimmunized mice, demonstrating the capacity of T_{FH} cells in lymph nodes to home to tumors. In addition, the proportion of proliferating T_{FH} cells (Ki-67⁺ T_{FH} cells) were increased in tumors of immunized mice compared to unimmunized mice (FIG. 13B). T_{FH} infiltration was also accompanied by increased frequency of CD8⁺ T cells, higher proportions of which were Cd39⁺ and Pd1⁺ in immunized mice, indicating enhanced activation following antigen-specific engagement. Furthermore, greater number of tumor-infiltrating CD8⁺ T cells from immunized mice expressed granzyme B and Ki-67, implying greater cytotoxic potential and cell proliferation (FIG. 13B). Taken together, these results revealed that induction of T_{FH} response functionally bolsters anti-tumor CD8⁺ CTL response and improved tumor control.

### Example 2: Cell Isolation

This example provides an exemplary method for isolating cells using flow cytometry.

A sample comprising a plurality of cells is contacted with the following antibodies: anti-CXCR5-BB515 (RF8B2; BD Biosciences), anti-CXCL13 (MA5-23629; ThermoFisher), and anti-CD4-BV510 (OKT4; Biolegend) for flow cytometric analysis and sorting. Cells that were positive for CXCL13 and CD4, but negative for CXCR5 were isolated from the sample.

### Example 3: Differentiating CXCL13⁺ Th Cells In Vitro

This example provides an exemplary method of differentiating CXCL13-expressing helper T cells in vitro. They culture cells in TGF-β+, IL-2-neutralizing culture conditions that give rise to PD1hiCXCR5-CD4+ T cells that preferentially express CXCL13.

Healthy human naive CD4⁺ T cells were isolated from PBMCs and differentiated under several inflammatory conditions in vitro. TGF-β-positive conditions induced CXCL13-producing CD4⁺ T cells that were highly positive for PD-1 and negative for CXCR5. IL-2-neutralizing antibody was added to the culture conditions, which resulted in a significant upregulation of CXCL13 production by PD-1^{hi}CXCR5⁻CD4⁺ T. Specifically, TGF-β-positive, IL-2-limiting conditions, which are consistent with local inflamed sites in several inflammatory diseases, gave rise to CXCL13-producing PD-1^{hi}CXCR5⁻CD4⁺ T cells in vitro. For additional details, see Yoshitomi, H., Kobayashi, S., Miyagawa-Hayashino, A., Okahata, A., Doi, K., Nishitani, K., Murata, K., Ito, H., Tsuruyama, T., Haga, H. and Matsuda, S., 2018. Human Sox4 facilitates the development of CXCL13-producing helper T cells in inflammatory environments. Nature communications, 9(1), p. 3762, which is incorporated by reference in its entirety.

### Example 4: In Vitro Differentiation of Tfh-Like Cells Expressing CD4, IL-21, PD-1, CXCR5+ and Producing IL-21, IFN-Gamma, but are SAP-Deficient (ShZd1a^{-/-})

This example provides an exemplary method for in vitro differentiation of Tfh-like cells expressing CD4, IL-21, PD-1, CXCR5+ and producing IL-21, IFN-gamma, but are SAP-deficient (Sh2d1a^{-/-})

To evaluate differentiation of a Tfh-like cell population in vitro, sorted naïve CD4⁺ T cells from OT-II mice were stimulated in the presence of antigen presenting cells (e.g. mitomycin-treated T-depleted splenocytes) and neutralizing antibodies against IL-4, IL-12, IFNγ and TGF-β, along with IL-6 and IL-21, cytokines important for Tfh cell differentiation in vivo and IL-21 production in vitro. For additional details, see Lu, K. T., Kanno, Y., Cannons, J. L., Handon, R., Bible, P., Elkahloun, A. G., Anderson, S. M., Wei, L., Sun, H., O'Shea, J. J. and Schwartzberg, P. L., 2011. Functional and epigenetic analyses of in vitro-generated and in vivo-derived interleukin-21-producing follicular T helper-like cells. Immunity, 35(4), p. 622, which is incorporated by reference in its entirety.

### Example 5: Isolation of Tfh Cells from Mice

This example provides an exemplary method for isolating Tfh cells from mice.

CD4⁺ T cells are isolated by anti-CD4 microbeads (Miltenyi Biotec, Bergisch Gladbach, Germany), and CD4⁺CD25⁻CD44⁻CD62L⁺ naïve T cells isolated from pooled spleen and peripheral lymph nodes of naïve C57BL/6 mice. CD4⁺PD-1⁺CXCR5⁺ Tfh cells were isolated from the draining lymph nodes of mice. Treg cells isolated from Foxp3^{RFP} mice using Treg isolation kit (Miltenyi Biotec) were stimulated using Treg expansion kits (Miltenyi Biotec), according to the manufacturer's protocols with a small modification (50 U/ml of mIL-2, instead of 1000 U/ml). Cells were cultured in RPMI 1640 medium (Lonza, Houston, Tex., USA) supplemented with 10% FBS, 55 μM 2-mercaptoethanol, 2 mM L-glutamine, 100 units penicillin-streptomycin (all from Gibco, Carlsbad, Calif., USA), and 10 μg/ml gentamicin (Sigma-Aldrich, St. Louis, Mo., USA). For additional details, see Kim, Y. U., Kim, B. S., Lim, H., Wetsel, R. A. and Chung, Y., 2017. Enforced Expression of CXCR5 Drives T Follicular Regulatory-Like Features in Foxp3+ T Cells. Biomolecules & therapeutics, 25(2), p. 130, which is incorporated by reference in its entirety.

### Example 6: Isolating and Expanding Tregs from Mice, then Genetically Modifying Treg Cells by Retrovirally Introducing Gene of Interest

This example provides an exemplary method for isolating and expanding Tregs from mice, followed by genetic modification of the Treg cells to introduce a gene of interest by retroviral transduction

Mouse Cxcr5 cDNA PCR fragment was ligated into RVKM-IRES-Gfp retroviral vector (RV). 10 μg of pCL-Eco packaging vector with 10 μg of RV-empty vector or RV-Cxcr5 were co-transfected into the 293T cells using calcium phosphate/chloroquine (100 μM, Sigma, St. Louis, Mo., USA) method. Twenty four hours later, stimulated Treg cells were transduced with RV-empty vector or RV-Cxcr5 in the presence of 8 μg/ml of polybrene (Sigma). For additional details, see Kim, Y. U., Kim, B. S., Lim, H., Wetsel, R. A. and Chung, Y., 2017. Enforced Expression of CXCR5 Drives T Follicular Regulatory-Like Features in Foxp3+ T Cells. Biomolecules & therapeutics, 25(2), p. 130, which is incorporated by reference in its entirety.

### Example 7: Generation of Tfh-Like Cells In Vitro

This example provides an exemplary method for generating Tfh-like cells in vitro by co-culturing CD4+ T cells from mouse PBMCs with antigen-specific dendritic cells or B cells expressing a transgenic B cell receptor (BCR) with the antigen of interest. Cells were cultured in R10 medium (RPMI-1640 (Gibco, Gaithersburg, Md.), supplemented with 10% fetal calf serum, 50 1M b-mercaptoethanol, 10 mM HEPES buffer and penicillin-streptomycin). CD4+ T cells were plated and then B cells were added in a ratio of 1:2 (B:T) or DCs were added in a ratio of 1:5 (DC:T) and incubated for 3 or 6 days in the presence of different stimuli. After co-culture, the cells were collected and stained with anti-B220, CD4, CXCR5, GL-7, ICOS, PD-1 and CD40L antibodies for surface expression and with anti-BCL-6, T-bet, GATA3, IL-21, IL-4 and IFN-c antibodies for intracellular proteins. For additional details, see Kolenbrander, A., Grewe, B., Nemazee, D., Uberla, K. and Temchura, V., 2018. Generation of T follicular helper cells in vitro: requirement for B-cell receptor cross-linking and cognate B- and T-cell interaction. Immunology, 153(2), pp. 214-224, which is incorporated by reference in its entirety.

Note that titles or subtitles may be used throughout the present disclosure for convenience of a reader, but in no way should these limit the scope of the disclosure. Moreover, certain theories may be proposed and disclosed herein; however, in no way they, whether they are right or wrong, should limit the scope of the disclosure so long as the disclosure is practiced according to the present disclosure without regard for any particular theory or scheme of action.

All references cited throughout the specification are hereby incorporated by reference in their entirety for all purposes.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this disclosure pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

With respect to ranges of values, the disclosure encompasses the upper and lower limits and each intervening value between the upper and lower limits of the range to at least a tenth of the upper and lower limit's unit, unless the context clearly indicates otherwise. Further, the disclosure encompasses any other stated intervening values.

Although various embodiments of the disclosure have been described and illustrated, it will be apparent to those skilled in the art in light of the present description that numerous modifications and variations can be made. The scope of the disclosure is defined more particularly in the appended claims.

### REFERENCES

1. Bevan M J. Helping the CD8(+) T-cell response. Nature reviews Immunology 2004, 4(8): 595-602.
2. Djenidi F, Adam J, Goubar A, Durgeau A, Meurice G, de Montpreville V, el al. CD8+CD103+ tumor-infiltrating lymphocytes are tumor-specific tissue-resident memory T cells and a prognostic factor for survival in lung cancer patients. Journal of immunology 2015, 194(7): 3475-3486.
3. Ganesan A P, Clarke J, Wood O, Garrido-Martin E M, Chee S J, Mellows T, et al. Tissue-resident memory features are linked to the magnitude of cytotoxic T cell responses in human lung cancer. Nature immunology 2017, 18(8): 940-950.
4. Wang Z Q, Milne K, Derocher H, Webb J R, Nelson B H, Watson P H. CD103 and Intratumoral Immune Response in Breast Cancer. Clinical cancer research; an official journal of the American Association for Cancer Research 2016.
5. Webb J R, Milne K, Watson P, Deleeuw R J, Nelson B H. Tumor-infiltrating lymphocytes expressing the tissue resident memory marker CD103 are associated with increased survival in high-grade serous ovarian cancer. Clinical cancer research: an official journal of the American Association for Cancer Research 2014, 20(2): 434-444.
6. Sharma P, Allison J P. Immune checkpoint targeting in cancer therapy: toward combination strategies with curative potential. Cell 2015, 161(2): 205-214.
7. Drake C G, Lipson E J, Brahmer J R. Breathing new life into immunotherapy: review of melanoma, lung and kidney cancer. Nature reviews Clinical oncology 2014, 11(1): 24-37.
8. den Haan J M, Bevan M J. A novel helper role for CD4 T cells. Proceedings of the National Academy of Sciences of the United States of America 2000, 97(24): 12950-12952.
9. Laidlaw B J, Craft J E, Kaech S M. The multifaceted role of CD4(+) T cells in CD8(+) T cell memory. Nature reviews Immunology 2016, 16(2): 102-111.
10. Schoenberger S P, Toes R E, van der Voort E I, Offringa R, Melief C J. T-cell help for cytotoxic T lymphocytes is mediated by CD40-CD40L interactions. Nature 1998, 393(6684): 480-483.
11. Oh S, Perera L P, Terabe M, Ni L, Waldmann T A, Berzofsky J A. IL-15 as a mediator of CD4+ help for CD8+ T cell longevity and avoidance of TRAIL-mediated apoptosis. Proceedings of the National Academy of Sciences of the United States of America 2008, 105(13): 5201-5206.
12. Hervas-Stubbs S, Olivier A, Boisgerault F, Thieblemont N, Leclerc C. TLR3 ligand stimulates fully functional memory CD8+ T cells in the absence of CD4+ T-cell help. Blood 2007, 109(12): 5318-5326.
13. Castellino F, Huang A Y, Altan-Bonnet G, Stoll S, Scheinecker C, Germain R N. Chemokines enhance immunity by guiding naive CD8+ T cells to sites of CD4+ T cell-dendritic cell interaction. Nature 2006, 440(7086): 890-895.
14. Kumamoto Y, Mattei L M, Sellers S, Payne G W, Iwasaki A. CD4+ T cells support cytotoxic T lymphocyte priming by controlling lymph node input. Proceedings of the National Academy of Sciences of the United States of America 2011, 108(21): 8749-8754.
15. Bourgeois C, Rocha B, Tanchot C. A role for CD40 expression on CD8+ T cells in the generation of CD8+ T cell memory. Science 2002, 297(5589): 2060-2063.
16. De Simone M, Arrigoni A, Rossetti G, Gruarin P, Ranzani V, Politano C, et al. Transcriptional Landscape of Human Tissue Lymphocytes Unveils Uniqueness of Tumor-Infiltrating T Regulatory Cells. Immunity 2016, 45(5): 1135-1147.
17. Tirosh I, Izar B, Prakadan S M, Wadsworth M H, 2nd, Treacy D, Trombetta J J, et al. Dissecting the multicellular ecosystem of metastatic melanoma by single-cell RNA-seq. Science 2016, 352(6282): 189-196.
18. Zheng C, Zheng L, Yoo J K, Guo H, Zhang Y, Guo X, et al. Landscape of Infiltrating T Cells in Liver Cancer Revealed by Single-Cell Sequencing. Cell 2017, 169(7): 1342-1356 e1316.
19. Bindea G, Mlecnik B, Tosolini M, Kirilovsky A, Waldner M, Obenauf A C, et al. Spatiotemporal dynamics of intratumoral immune cells reveal the immune landscape in human cancer. Immunity 2013, 39(4): 782-795.
20. Baitsch L, Baumgaertner P, Devevre E, Raghav S K, Legat A, Barba L, et al. Exhaustion of tumor-specific CD8(+) T cells in metastases from melanoma patients. The Journal of clinical investigation 2011, 121(6): 2350-2360.
21. Collins N, Godec J, Zou L H, Mihm M C, Getz G, Haining W N. Transcriptional Hallmarks Of Tumor Infiltrating Lymphocyte Responses To Melanoma. Blood 2013, 122(21).
22. Schrama D, thor Straten P, Fischer W H, McLellan A D, Brocker E B, Reisfeld R A, et al. Targeting of lymphotoxin-alpha to the tumor elicits an efficient immune response associated with induction of peripheral lymphoid-like tissue. Immunity 2001, 14(2): 111-121.
23. Kirk C J, Hartigan-O'Connor D, Mule J J. The dynamics of the T-cell antitumor response: chemokine-secreting dendritic cells can prime tumor-reactive T cells extranodally. Cancer research 2001, 61(24): 8794-8802.
24. Schrama D, Voigt H, Eggert A O, Xiang R, Zhou H, Schumacher T N, et al. Immunological tumor destruction in a murine melanoma model by targeted LTalpha independent of secondary lymphoid tissue. Cancer immunology, immunotherapy: CII 2008, 57(1): 85-95.
25. Garon E B, Rizvi N A, Hui R, Leighl N, Balmanoukian A S, Eder J P, et al. Pembrolizumab for the treatment of non-small-cell lung cancer. The New England journal of medicine 2015, 372(21): 2018-2028.
26. Pardoll D M. The blockade of immune checkpoints in cancer immunotherapy. Nature reviews Cancer 2012, 12(4): 252-264.
27. Langfelder P, Horvath S. WGCNA: an R package for weighted correlation network analysis. BMC bioinformatics 2008, 9: 559.
28. Gebhardt T, Palendira U, Tscharke D C, Bedoui S. Tissue-resident memory T cells in tissue homeostasis, persistent infection, and cancer surveillance. Immunological reviews 2018, 283(1): 54-76.
29. Mackay L K, Rahimpour A, Ma J Z, Collins N, Stock A T, Hafon M L, et al. The developmental pathway for CD103(+)CD8+ tissue-resident memory T cells of skin. Nature immunology 2013, 14(12): 1294-1301.
30. Pan Y, Tian T, Park C O, Lofftus S Y, Mei S, Liu X, et al. Survival of tissue-resident memory T cells requires exogenous lipid uptake and metabolism. Nature 2017, 543(7644): 252-256.
31. Wolkers M C, Gerlach C, Arens R, Janssen E M, Fitzgerald P, Schumacher T N, el al. Nab2 regulates secondary CD8+ T-cell responses through control of TRAIL expression. Blood 2012, 119(3): 798-804.
32. Crotty S. T follicular helper cell differentiation, function, and roles in disease. Immunity 2014, 41(4): 529-542.
33. Victora G D, Nussenzweig M C. Germinal centers. Annual review of immunology 2012, 30: 429-457.
34. Betz B C, Jordan-Williams K L, Wang C, Kang S G, Liao J, Logan M R, et al. Batf coordinates multiple aspects of B and T cell function required for normal antibody responses. The Journal of experimental medicine 2010, 207(5): 933-942.
35. van de Pavert S A, Olivier B J, Goverse G, Vondenhoff M F, Greuter M, Beke P, et al. Chemokine CXCL13 is essential for lymph node initiation and is induced by retinoic acid and neuronal stimulation. Nature immunology 2009, 10(11): 1193-1199.
36. Kim C H, Lim H W, Kim J R, Rott L, Hillsamer P, Butcher E C. Unique gene expression program of human germinal center T helper cells. Blood 2004, 104(7): 1952-1960.
37. Luther S A, Lopez T, Bai W, Hanahan D, Cyster J G. BLC expression in pancreatic islets causes B cell recruitment and lymphotoxin-dependent lymphoid neogenesis. Immunity 2000, 12(5): 471-481.
38. Schmitt N, Liu Y, Bentebibel S E, Munagala I, Bourdery L, Venuprasad K, et al. The cytokine TGF-beta co-opts signaling via STAT3-STAT4 to promote the differentiation of human TFH cells. Nature immunology 2014, 15(9): 856-865.
39. Auderset F, Schuster S, Fasnacht N, Coutaz M, Charmoy M, Koch U, et al. Notch signaling regulates follicular helper T cell differentiation. Journal of immunology 2013, 191(5): 2344-2350.
40. Ma C S, Deenick E K. The circulating life of a memory T-follicular helper cell. Clinical & translational immunology 2017, 6(5): e141.
41. Herati R S, Reuter M A, Dolfi D V, Mansfield K D, Aung H, Badwan O Z, et al. Circulating CXCR5+PD-1+ response predicts influenza vaccine antibody responses in young adults but not elderly adults. Journal of immunology 2014, 193(7): 3528-3537.
42. Bentebibel S E, Lopez S, Obermoser G, Schmitt N, Mueller C, Harrod C, el al. Induction of ICOS+CXCR3+CXCR5+ TH cells correlates with antibody responses to influenza vaccination. Science translational medicine 2013, 5(176): 176ra132.
43. Guo Y, Sun X, Shibata K, Yamada H, Muta H, Podack E R, et al. CD30 is required for activation of a unique subset of interleukin-17A-producing gammadelta T cells in innate immunity against Mycobacterium bovis Bacillus Calmette-Guerin infection. Infection and immunity 2013, 81(10): 3923-3934.
44. Ma J, Feng D, Wei Y, Tian J, Tang X, Rui K, et al. Blockade of Glucocorticoid-Induced Tumor Necrosis Factor-Receptor-Related Protein Signaling Ameliorates Murine Collagen-Induced Arthritis by Modulating Follicular Helper T Cells. The American journal of pathology 2016, 186(6): 1559-1567.
45. Gu-Trantien C, Migliori E, Buisseret L, de Wind A, Brohee S, Garaud S, et al. CXCL13-producing TFH cells link immune suppression and adaptive memory in human breast cancer. JCI insight 2017, 2(11).
46. Rao D A, Gurish M F, Marshall J L, Slowikowski K, Fonseka C Y, Liu Y, et al. Pathologically expanded peripheral T helper cell subset drives B cells in rheumatoid arthritis. Nature 2017, 542(7639): 110-114.
47. Pascutti M F, Geerman S, Slot E, van Gisbergen K P, Boon L, Arens R, et al. Enhanced CD8 T cell responses through GITR-mediated costimulation resolve chronic viral infection. PLoS pathogens 2015, 11(3): e1004675.
48. Song A, Song J, Tang X, Croft M. Cooperation between CD4 and CD8 T cells for anti-tumor activity is enhanced by OX40 signals. European journal of immunology 2007, 37(5): 1224-1232.
49. Xin G, Schauder D M, Lainez B, Weinstein J S, Dai Z, Chen Y, et al. A Critical Role of IL-21-Induced BATF in Sustaining CD8-T-Cell-Mediated Chronic Viral Control. Cell reports 2015, 13(6): 1118-1124.
50. Laidlaw B J, Zhang N, Marshall H D, Staron M M, Guan T, Hu Y, et al. CD4+ T cell help guides formation of CD103+ lung-resident memory CD8+ T cells during influenza viral infection. Immunity 2014, 41(4): 633-645.
51. Gopalakrishnan V, Spencer C N, Nezi L, Reuben A, Andrews M C, Karpinets T V, et al. Gut microbiome modulates response to anti-PD-1 immunotherapy in melanoma patients. Science 2018, 359(6371): 97-103.
52. Routy B, Le Chatelier E, Derosa L, Duong C P M, Alou M T, Daillere R, et al. Gut microbiome influences efficacy of PD-1-based immunotherapy against epithelial tumors. Science 2018, 359(6371): 91-97.
53. Green A M, Difazio R, Flynn J L. IFN-gamma from CD4 T cells is essential for host survival and enhances CD8 T cell function during Mycobacterium tuberculosis infection. Journal of immunology 2013, 190(1): 270-277.
54. Takeuchi A, Saito T. CD4 CTL, a Cytotoxic Subset of CD4(+) T Cells, Their Differentiation and Function. Frontiers in immunology 2017, 8: 194.
55. Mucida D, Husain M M, Muroi S, van Wijk F, Shinnakasu R, Naoe Y, et al. Transcriptional reprogramming of mature CD4(+) helper T cells generates distinct MHC class II-restricted cytotoxic T lymphocytes. Nature immunology 2013, 14(3): 281-289.
56. Reis B S, Rogoz A, Costa-Pinto F A, Taniuchi I, Mucida D. Mutual expression of the transcription factors Runx3 and ThPOK regulates intestinal CD4(+) T cell immunity. Nature immunology 2013, 14(3): 271-280.
57. Cheroutre H, Husain M M. CD4 CTL: living up to the challenge. Seminars in immunology 2013, 25(4): 273-281.
58. Zhang N, Bevan M J. Transforming growth factor-beta signaling controls the formation and maintenance of gut-resident memory T cells by regulating migration and retention. Immunity 2013, 39(4): 687-696.
59. Remark R, Merghoub T, Grabe N, Litjens G, Damotte D, Wolchok J D, et al. In-depth tissue profiling using multiplexed immunohistochemical consecutive staining on single slide. Science immunology 2016, 1(1): aaf6925.
60. Schindelin J, Arganda-Carreras I, Frise E, Kaynig V, Longair M, Pietzsch T, et al. Fiji: an open-source platform for biological-image analysis. Nature methods 2012, 9(7): 676-682.
61. Ruifrok A C, Johnston D A. Quantification of histochemical staining by color deconvolution. Analytical and quantitative cytology and histology 2001, 23(4): 291-299.
62. Seumois G, Zapardiel-Gonzalo J, White B, Singh D, Schulten V, Dillon M, el al. Transcriptional Profiling of Th2 Cells Identifies Pathogenic Features Associated with Asthma. Journal of immunology 2016, 197(2): 655-664.
63. Rosales S L, Liang S, Engel I, Schmiedel B J, Kronenberg M, Vijayanand P, et al. A Sensitive and Integrated Approach to Profile Messenger RNA from Samples with Low Cell Numbers. Methods in molecular biology 2018, 1799: 275-301.
64. Patil V S, Madrigal A, Schmiedel B J, Clarke J, O'Rourke P, de Silva A D, et al. Precursors of human CD4(+) cytotoxic T lymphocytes identified by single-cell transcriptome analysis. Science immunology 2018, 3(19).
65. Trapnell C, Pachter L, Salzberg S L. TopHat: discovering splice junctions with RNA-Seq. Bioinformatics 2009, 25(9): 1105-1 111.
66. Anders S, Pyl P T, Huber W. HTSeq—a Python framework to work with high-throughput sequencing data. Bioinformatics 2015, 31(2): 166-169.
67. Love M I, Huber W, Anders S. Moderated estimation of fold change and dispersion for RNA-seq data with DESeq2. Genome biology 2014, 15(12): 550.
68. Engel I, Seumois G, Chavez L, Samaniego-Castruita D, White B, Chawla A, et al. Innate-like functions of natural killer T cell subsets result from highly divergent gene programs. Nature immunology 2016, 17(6): 728-739.
69. Bolotin D A, Poslavsky S, Mitrophanov I, Shugay M, Mamedov I Z, Putintseva E V, et al. MiXCR: software for comprehensive adaptive immunity profiling. Nature methods 2015, 12(5): 380-381.
70. Bardou P, Mariette J, Escudie F, Djemiel C, Klopp C. jvenn: an interactive Venn diagram viewer. BMC bioinformatics 2014, 15: 293.
71. Ottensmeier C H, Perry K L, Harden E L, Stasakova J, Jenei V, Fleming J, et al. Upregulated Glucose Metabolism Correlates Inversely with CD8+ T-cell Infiltration and Survival in Squamous Cell Carcinoma. Cancer research 2016, 76(14): 4136-4148.
72. Mellone M, Hanley C J, Thirdborough S, Mellows T, Garcia E, Woo J, et al. Induction of fibroblast senescence generates a non-fibrogenic myofibroblast phenotype that differentially impacts on cancer prognosis. Aging 2016, 9(1): 114-132.
73. Macosko E Z, Basu A, Satija R, Nemesh J, Shekhar K, Goldman M, et al. Highly Parallel Genome-wide Expression Profiling of Individual Cells Using Nanoliter Droplets. Cell 2015, 161(5): 1202-1214.
74. Soneson C, Robinson M D. Bias, robustness and scalability in single-cell differential expression analysis. Nature methods 2018, 15(4): 255-261.
75. Finak G, McDavid A, Yajima M, Deng J, Gersuk V, Shalek A K, et al. MAST: a flexible statistical framework for assessing transcriptional changes and characterizing heterogeneity in single-cell RNA sequencing data. Genome biology 2015, 16: 278.
76. Trapnell C, Cacchiarelli D, Grimsby J, Pokharel P, Li S, Morse M, et al. The dynamics and regulators of cell fate decisions are revealed by pseudotemporal ordering of single cells. Nature biotechnology 2014, 32(4): 381-386.

TABLE 1
Related to FIGS. 1, 3 and 4; FIG. 9
Demographic, clinical and histopathological characteristics of NSCLC patients.
A. Patient cohort used for bulk RNA sequencing

| | | | | Tumor | Nodal | Metastasis |
|---|---|---|---|---|---|---|
| | Age | | | status | status | status |
| Patient ID | (years) | Gender | Stage | (T) | (N) | (M) |
| NSCLC_01 | 72 | F | IIA | 2b | 0 | 0 |
| NSCLC_02 | 84 | F | IIA | 1b | 1 | 0 |
| NSCLC_03 | 72 | F | IIIA | 4 | 0 | 0 |
| NSCLC_04 | 68 | M | IIA | 2b | 0 | 0 |
| NSCLC_05 | 63 | M | IB | 2a | 0 | 0 |
| NSCLC_06 | 77 | F | IIIA | 2a | 2 | 0 |
| NSCLC_07 | 79 | M | IIB | 3 | 0 | 0 |
| NSCLC_08 | 80 | M | IB | 2a | 0 | 0 |
| NSCLC_09 | 51 | F | IB | 2a | 0 | 0 |
| NSCLC_10 | 70 | F | IA | 1b | 0 | 0 |
| NSCLC_11 | 73 | M | IB | 2a | 0 | 0 |
| NSCLC_12 | 69 | F | IB | 2a | 0 | 0 |
| NSCLC_13 | 70 | M | IIIA | 3 | 2 | 0 |
| NSCLC_14 | 76 | M | IA | 1b | 0 | 0 |
| NSCLC_15 | 77 | M | IA | 2b | 2 | 0 |
| NSCLC_16 | 50 | M | IB | 2b | 0 | 0 |
| NSCLC_17 | 87 | M | IA | 1a | 0 | 0 |
| NSCLC_18 | 65 | F | IA | 1a | 0 | 0 |
| NSCLC_19 | 64 | M | IB | 1a/2a | 0 | 0 |
| NSCLC_20 | 74 | M | IIB | 2b | 1 | 0 |
| NSCLC_21 | 60 | F | IA | 1b | 0 | 0 |
| NSCLC_22 | 68 | F | N/A | 1a | 0 | 0 |
| NSCLC_23 | 74 | M | N/A | 2a | 0 | 0 |
| NSCLC_24 | 72 | F | IB | 2a | 0 | 0 |
| NSCLC_25 | 72 | M | IA | 1b | 0 | 0 |
| NSCLC_26 | 67 | M | IB | 2a | 0 | 0 |
| NSCLC_27 | 81 | F | IIIB | 4 | 2 | 0 |
| NSCLC_28 | 72 | M | IV | 1a | 0 | 1B |
| NSCLC_29 | 75 | M | IIIA | 3 | 1 | 0 |
| NSCLC_30 | N/A | N/A | N/A | 3 | 1 | N/A |
| NSCLC_31 | 58 | F | IA | 1a | 0 | 0 |
| NSCLC_32 | 75 | F | IIB | 3 | 0 | 0 |
| NSCLC_33 | 66 | M | IA | 1a | 0 | 0 |
| NSCLC_34 | 77 | F | IB | 2a | 0 | 0 |
| NSCLC_35 | 81 | M | IA | 1a | 0 | 0 |
| NSCLC_36 | 68 | M | IA | 1b | 0 | 0 |
| NSCLC_37 | 68 | F | IIA | 2a | 1 | 0 |
| NSCLC_38 | 69 | M | IB | 1b | 0 | 0 |
| NSCLC_39 | 67 | M | IIB | 3 | 0 | 0 |
| NSCLC_40 | 77 | F | IIA | 2a | 1 | 0 |
| NSCLC_41 | 83 | M | IIA | 2b | 0 | N/A |
| NSCLC_42 | 76 | M | IB | 2a | 0 | 0 |
| NSCLC_43 | 74 | M | IA | 1a | 0 | 0 |
| NSCLC_44 | 81 | F | IIB | 1a | 0 | 0 |
| NSCLC_45 | 74 | M | IIB | 3 | 0 | 0 |

| | | | | ALK | EGFR | |
|---|---|---|---|---|---|---|
| | Performance | Smoking | Asbestos | translocation | mutation | Tumor |
| Patient ID | status | status | exposure | status | status | histology |
| NSCLC_01 | 0 | Never | No | Neg | Neg | adenocarcinoma |
| NSCLC_02 | 0 | Ex | No | Neg | Neg | adenocarcinoma |
| NSCLC_03 | 0 | Current | No | Neg | Neg | adenocarcinoma |
| NSCLC_04 | 0 | Ex | No | Neg | Neg | squamous |
| | | | | | | carcinoma |
| NSCLC_05 | 0 | Ex | No | Neg | Neg | adenocarcinoma |
| NSCLC_06 | 1 | Never | No | Neg | Neg | adenocarcinoma |
| NSCLC_07 | 0 | Ex | No | Neg | Neg | squamous |
| | | | | | | carcinoma |
| NSCLC_08 | 1 | Ex | N/A | Neg | Neg | squamous |
| | | | | | | carcinoma |
| NSCLC_09 | 0 | Never | No | Neg | Pos | adenocarcinoma |
| NSCLC_10 | 1 | Ex | N/A | Neg | Neg | adenocarcinoma |
| NSCLC_11 | 0 | Ex | No | Neg | Neg | adenocarcinoma |
| NSCLC_12 | 0 | Ex | No | Neg | Neg | adenocarcinoma |
| NSCLC_13 | 1 | Ex | Yes | Neg | Neg | adenocarcinoma |
| NSCLC_14 | 0 | Current | No | Neg | Neg | squamous |
| | | | | | | carcinoma |
| NSCLC_15 | 0 | Ex | Yes | Neg | Neg | adenocarcinoma |
| NSCLC_16 | 0 | Current | No | N/A | Neg | adenocarcinoma |
| NSCLC_17 | 0 | Ex | No | Neg | Neg | adenocarcinoma |
| NSCLC_18 | 1 | Ex | No | Neg | Neg | adenocarcinoma |
| NSCLC_19 | 0 | Ex | Yes | Neg | Neg | adenocarcinoma |
| NSCLC_20 | 0 | Ex | No | Neg | Neg | squamous |
| | | | | | | carcinoma |
| NSCLC_21 | 1-2 | Ex | No | Neg | Neg | adenocarcinoma |
| NSCLC_22 | 0 | Ex | No | N/A | N/A | adenocarcinoma |
| NSCLC_23 | 1 | Ex | No | Neg | Neg | adenocarcinoma |
| NSCLC_24 | 0 | Ex | No | Neg | Neg | adenocarcinoma |
| NSCLC_25 | 1 | Ex | No | Neg | Neg | adenocarcinoma |
| NSCLC_26 | 0 | Ex | Yes | Neg | Neg | squamous |
| | | | | | | carcinoma |
| NSCLC_27 | 1-2 | Never | No | Neg | Neg | adenocarcinoma |
| NSCLC_28 | 1 | Ex | No | Neg | Neg | adenocarcinoma |
| NSCLC_29 | 0 | Current | Yes | Neg | Neg | squamous |
| | | | | | | carcinoma |
| NSCLC_30 | N/A | N/A | N/A | N/A | N/A | squamous |
| | | | | | | carcinoma |
| NSCLC_31 | 0 | Current | No | Neg | Neg | adenocarcinoma |
| NSCLC_32 | 1 | Never | No | Neg | Neg | adenocarcinoma |
| NSCLC_33 | 0 | Ex | No | Neg | Neg | adenocarcinoma |
| NSCLC_34 | 1 | Ex | No | Neg | Neg | adenocarcinoma |
| NSCLC_35 | 1 | Ex | Yes | N/A | N/A | squamous |
| | | | | | | carcinoma |
| NSCLC_36 | 0 | Current | No | Neg | Neg | squamous |
| | | | | | | carcinoma |
| NSCLC_37 | 1 | Ex | No | Neg | Neg | adenocarcinoma |
| NSCLC_38 | 0 | Ex | No | N/A | N/A | adenocarcinoma |
| NSCLC_39 | 1 | Current | Yes | Neg | Neg | squamous |
| | | | | | | carcinoma |
| NSCLC_40 | 0 | Ex | No | Neg | Pos | adenocarcinoma |
| NSCLC_41 | N/A | N/A | N/A | N/A | N/A | squamous |
| | | | | | | carcinoma |
| NSCLC_42 | 0 | Ex | No | Neg | Neg | adenocarcinoma |
| NSCLC_43 | 0 | Ex | No | N/A | N/A | adenocarcinoma |
| NSCLC_44 | N/A | N/A | N/A | N/A | N/A | squamous |
| | | | | | | carcinoma |
| NSCLC_45 | N/A | N/A | N/A | N/A | N/A | adenocarcinoma |

| | | | | | | | Rank |
|---|---|---|---|---|---|---|---|
| | | | | | | Matched | order of |
| | Number | | | | | CD8⁺ TIL | patients |
| | of | | | | | RNA-Seq | based on |
| | CD8a⁺ | | | QC | QC | data | CD8⁺ TIL |
| | cells | | | passed | passed | (Ganesan et | PDCD1 |
| | (average | | | TIL | N-TIL | al. Nature | expression |
| | per | TIL | T_{RM} | RNA- | RNA- | Immunology | (related to |
| Patient ID | HPF) | status | status | Seq | Seq | 2017) | FIG. 2) |
| NSCLC_01 | 28.1 | High | Intermediate | Yes | Yes | Yes | 7 |
| NSCLC_02 | 11.4 | Intermediate | High | Yes | No | Yes | 13 |
| NSCLC_03 | 9.9 | Intermediate | Intermediate | Yes | Yes | Yes | 6 |
| NSCLC_04 | 17.5 | High | Low | Yes | Yes | Yes | 21 |
| NSCLC_05 | 21.2 | High | High | Yes | Yes | Yes | 5 |
| NSCLC_06 | 4.8 | Low | Low | Yes | Yes | Yes | 30 |
| NSCLC_07 | 0.3 | Low | N/A | Yes | Yes | N/A | N/A |
| NSCLC_08 | 18.6 | High | High | Yes | Yes | Yes | 27 |
| NSCLC_09 | 9.6 | Intermediate | Intermediate | Yes | Yes | Yes | 18 |
| NSCLC_10 | 12.6 | Intermediate | Intermediate | Yes | Yes | Yes | 17 |
| NSCLC_11 | 3.7 | Low | Intermediate | Yes | Yes | Yes | 24 |
| NSCLC_12 | 6.8 | Low | Intermediate | Yes | Yes | Yes | 32 |
| NSCLC_13 | 4.1 | Low | Low | Yes | Yes | Yes | 20 |
| NSCLC_14 | 2.7 | Low | Intermediate | Yes | Yes | Yes | 23 |
| NSCLC_15 | 28.2 | High | High | Yes | Yes | Yes | 9 |
| NSCLC_16 | 7.1 | Low | High | Yes | No | Yes | 10 |
| NSCLC_17 | 32.7 | High | High | Yes | Yes | Yes | 1 |
| NSCLC_18 | 3.0 | Low | Intermediate | Yes | Yes | Yes | 11 |
| NSCLC_19 | 23.2 | High | High | Yes | Yes | Yes | 2 |
| NSCLC_20 | 8.6 | Intermediate | High | Yes | Yes | Yes | 8 |
| NSCLC_21 | 10.7 | Intermediate | Intermediate | Yes | Yes | Yes | 33 |
| NSCLC_22 | 6.5 | Low | Low | Yes | Yes | Yes | 15 |
| NSCLC_23 | 8.3 | Intermediate | Low | Yes | No | Yes | 14 |
| NSCLC_24 | 38.7 | High | High | Yes | No | Yes | 34 |
| NSCLC_25 | 15.6 | High | High | Yes | No | Yes | 3 |
| NSCLC_26 | 14.7 | High | Intermediate | Yes | No | Yes | 22 |
| NSCLC_27 | 4.3 | Low | Low | Yes | Yes | Yes | 35 |
| NSCLC_28 | 10.3 | Intermediate | Low | Yes | No | Yes | 25 |
| NSCLC_29 | 9.7 | Intermediate | N/A | Yes | Yes | N/A | N/A |
| NSCLC_30 | 15.0 | High | Low | Yes | Yes | Yes | 12 |
| NSCLC_31 | 29.1 | High | N/A | Yes | Yes | N/A | N/A |
| NSCLC_32 | 6.3 | Low | N/A | Yes | No | N/A | N/A |
| NSCLC_33 | 10.1 | Intermediate | Intermediate | Yes | Yes | Yes | 28 |
| NSCLC_34 | 10.8 | Intermediate | Low | Yes | No | Yes | 29 |
| NSCLC_35 | 0.7 | Low | N/A | Yes | Yes | N/A | N/A |
| NSCLC_36 | 2.7 | Low | N/A | Yes | No | N/A | N/A |
| NSCLC_37 | 9.2 | Intermediate | High | Yes | Yes | Yes | 19 |
| NSCLC_38 | 2.8 | Low | N/A | Yes | Yes | N/A | N/A |
| NSCLC_39 | 4.4 | Low | Low | Yes | Yes | Yes | 31 |
| NSCLC_40 | 6.3 | Low | Low | Yes | Yes | Yes | 36 |
| NSCLC_41 | 11.4 | Intermediate | Intermediate | Yes | No | Yes | 16 |
| NSCLC_42 | 6.4 | Low | N/A | Yes | Yes | N/A | N/A |
| NSCLC_43 | 5.0 | Low | N/A | Yes | No | N/A | N/A |
| NSCLC_44 | 10.8 | Intermediate | Intermediate | Yes | No | Yes | 26 |
| NSCLC_45 | 80.3 | High | Intermediate | Yes | No | Yes | 4 |

| | | | | Tumor | Nodal |
|---|---|---|---|---|---|
| | Age | | | status | status |
| Patient ID | (years) | Gender | Stage | (T) | (N) |
| NSCLC_50 | 58 | F | 1A | 1A | 0 |
| NSCLC_51 | 60 | F | 2A | 2B | 0 |
| NSCLC_52 | 75 | M | 1A | 1B | 0 |
| NSCLC_53 | 64 | M | 1B | 2A | 0 |
| NSCLC_54 | 59 | M | 1B | 2A | 0 |
| NSCLC_55 | 64 | M | 1B | 2A | 0 |

| | Metastasis | Performance | Smoking | Asbestos | Tumor |
|---|---|---|---|---|---|
| Patient ID | status (M) | status | status | exposure | histology |
| NSCLC_50 | 0 | 0 | Current | N/A | adenocarcinoma |
| NSCLC_51 | 0 | 1 | Ex | N/A | adenocarcinoma |
| NSCLC_52 | N/A | N/A | Ex | No | adenocarcinoma |
| NSCLC_53 | N/A | 0 | Current | N/A | adenocarcinoma |
| NSCLC_54 | N/A | 0 | Ex | No | squamous |
| | | | | | carcinoma |
| NSCLC_55 | N/A | N/A | Ex | No | adenocarcinoma |

| | Number | | | | |
|---|---|---|---|---|---|
| | of CD8a⁺ | | | | |
| | cells | | | TIL | N-TIL |
| | (average | TIL | T_{RM} | single-cell | single-cell |
| Patient ID | per HPF) | status | status | RNA-Seq | RNA-Seq |
| NSCLC_50 | 34.3 | High | High | Yes | No |
| NSCLC_51 | 32.5 | High | High | Yes | Yes |
| NSCLC_52 | 29.7 | High | Intermediate | Yes | No |
| NSCLC_53 | 25.2 | High | Low | Yes | Yes |
| NSCLC_54 | 30.1 | High | Low | Yes | No |
| NSCLC_55 | 47.1 | High | High | Yes | No |

Data not available is indicated by ‘N/A’.
“ALK translocation status” negative indicates the absence of a translocation involving anaplastic lymphoma kinase gene (ALK)
“EGFR mutation status” positive indicates presence of activating mutations in epidermal growth factor receptor gene (EGFR)

TABLE 2
Related to FIG. 1.
List of differentially expressed genes in CD4⁺ TILs versus N-TILs.

| | DE-Seq statistics | | |
|---|---|---|---|
| | Log₂ fold | | Adjusted |
| Gene name | change | P value | P value |
| AASS | 1.14 | 1.98E−05 | 6.12E−04 |
| ABL2 | −0.78 | 8.21E−04 | 1.30E−02 |
| ACE | −0.81 | 1.25E−03 | 1.80E−02 |
| ACO1 | −0.73 | 4.12E−03 | 4.53E−02 |
| ACOT1 | −0.92 | 1.55E−03 | 2.13E−02 |
| ACOT2 | −0.61 | 2.70E−03 | 3.29E−02 |
| ACOT7 | −0.70 | 1.96E−03 | 2.57E−02 |
| ACTG2 | 1.09 | 5.98E−04 | 1.02E−02 |
| ACTN1 | −0.73 | 2.20E−03 | 2.80E−02 |
| ACVRL1 | −1.56 | 5.28E−07 | 2.65E−05 |
| ADAMTSL4 | −1.87 | 1.33E−09 | 1.15E−07 |
| ADARB1 | −0.76 | 6.41E−04 | 1.08E−02 |
| ADAT2 | 1.20 | 1.89E−08 | 1.27E−06 |
| ADRB2 | −1.49 | 1.94E−08 | 1.30E−06 |
| AGPAT4 | −0.98 | 4.68E−04 | 8.41E−03 |
| AGPAT4-IT1 | −0.90 | 3.69E−03 | 4.17E−02 |
| AGTPBP1 | −0.62 | 1.75E−03 | 2.34E−02 |
| AHI1 | 0.76 | 2.71E−06 | 1.10E−04 |
| AHNAK | −0.85 | 3.73E−16 | 1.31E−13 |
| AIF1 | −1.26 | 1.78E−06 | 7.79E−05 |
| AIM1 | −0.63 | 3.77E−06 | 1.47E−04 |
| AKAP5 | 0.83 | 3.12E−04 | 5.99E−03 |
| AKIRIN2 | −0.80 | 1.65E−06 | 7.39E−05 |
| ALDH2 | −2.67 | 5.64E−21 | 4.57E−18 |
| ALDH3B1 | −1.74 | 2.19E−09 | 1.79E−07 |
| ALDH7A1 | −1.00 | 1.44E−03 | 2.02E−02 |
| ALOX15 | −1.01 | 6.11E−04 | 1.04E−02 |
| ALOX5 | −2.09 | 3.31E−18 | 1.53E−15 |
| AMOTL1 | −0.93 | 1.62E−03 | 2.20E−02 |
| ANKRD28 | −1.18 | 1.15E−12 | 1.90E−10 |
| ANKS1B | 1.72 | 8.20E−10 | 7.52E−08 |
| ANPEP | −1.96 | 5.47E−10 | 5.19E−08 |
| ANXA1 | −1.39 | 8.70E−29 | 2.82E−25 |
| ANXA2 | −1.11 | 7.60E−23 | 1.10E−19 |
| ANXA2P2 | −0.95 | 9.40E−09 | 6.90E−07 |
| ANXA5 | −0.66 | 2.39E−07 | 1.28E−05 |
| AP3D1 | −0.61 | 4.92E−04 | 8.74E−03 |
| AP4E1 | −0.64 | 2.02E−03 | 2.62E−02 |
| APOBEC3A | −0.95 | 1.83E−03 | 2.43E−02 |
| APOBEC3H | −0.64 | 3.16E−03 | 3.74E−02 |
| APOC1 | −1.47 | 1.86E−08 | 1.26E−06 |
| APOE | −1.05 | 1.06E−05 | 3.57E−04 |
| APOL4 | −1.58 | 9.23E−09 | 6.81E−07 |
| APOLD1 | 0.76 | 3.27E−07 | 1.72E−05 |
| AQP9 | −1.15 | 1.21E−04 | 2.78E−03 |
| ARHGAP10 | −0.71 | 8.08E−04 | 1.28E−02 |
| ARHGAP26 | −0.76 | 3.00E−04 | 5.80E−03 |
| ARHGEF10L | −1.02 | 3.13E−04 | 5.99E−03 |
| ARL4A | −0.61 | 2.69E−04 | 5.27E−03 |
| ARRB2 | −0.59 | 5.47E−14 | 1.18E−11 |
| ARRDC4 | −1.66 | 1.14E−07 | 6.51E−06 |
| ARSB | −0.72 | 1.35E−04 | 3.00E−03 |
| ASAH1 | −0.76 | 1.34E−08 | 9.39E−07 |
| ASB2 | 1.12 | 1.73E−06 | 7.69E−05 |
| ATMIN | −1.08 | 6.08E−06 | 2.23E−04 |
| ATP2B1 | −1.05 | 2.36E−12 | 3.61E−10 |
| ATPAF2 | 0.85 | 2.97E−04 | 5.75E−03 |
| AVPI1 | −0.94 | 1.55E−03 | 2.12E−02 |
| AXL | −1.62 | 2.82E−10 | 2.86E−08 |
| B3GNT7 | −1.20 | 9.79E−05 | 2.32E−03 |
| B4GALT5 | −1.14 | 5.64E−06 | 2.09E−04 |
| BACE1 | −0.82 | 1.06E−03 | 1.60E−02 |
| BATF | 0.58 | 6.80E−07 | 3.38E−05 |
| BHLHE40 | −0.64 | 1.69E−06 | 7.52E−05 |
| BHLHE41 | −1.10 | 2.80E−04 | 5.45E−03 |
| BPIFB1 | −1.17 | 9.80E−05 | 2.32E−03 |
| BRI3 | −0.90 | 6.22E−06 | 2.26E−04 |
| BTLA | 1.40 | 9.52E−14 | 1.96E−11 |
| C10orf28 | 0.65 | 4.19E−03 | 4.58E−02 |
| C12orf75 | −0.62 | 1.30E−05 | 4.23E−04 |
| C13orf15 | −0.95 | 2.23E−09 | 1.81E−07 |
| C14orf182 | 0.87 | 9.57E−04 | 1.47E−02 |
| C16orf45 | 1.24 | 2.33E−05 | 7.04E−04 |
| C16orf54 | −0.69 | 2.99E−14 | 6.94E−12 |
| C19orf59 | −2.87 | 4.27E−22 | 5.54E−19 |
| C1orf162 | −0.99 | 3.71E−13 | 6.79E−11 |
| C1orf182 | 0.89 | 4.49E−03 | 4.84E−02 |
| C1orf21 | −0.88 | 3.71E−03 | 4.18E−02 |
| C1orf38 | −1.00 | 1.21E−09 | 1.06E−07 |
| C1QA | −1.96 | 3.35E−13 | 6.21E−11 |
| C1QB | −2.05 | 9.83E−15 | 2.50E−12 |
| C1QC | −1.80 | 4.92E−12 | 7.06E−10 |
| C2 | −1.36 | 1.22E−05 | 4.01E−04 |
| C22orf29 | 0.66 | 3.48E−03 | 4.03E−02 |
| C5AR1 | −1.60 | 2.28E−07 | 1.23E−05 |
| C5orf62 | −0.65 | 4.26E−03 | 4.63E−02 |
| C6orf108 | 1.17 | 1.01E−33 | 6.54E−30 |
| C9orf21 | −1.14 | 2.94E−07 | 1.56E−05 |
| C9orf89 | −0.66 | 2.64E−05 | 7.63E−04 |
| CA5B | −0.96 | 1.72E−08 | 1.18E−06 |
| CADM1 | 1.34 | 6.04E−07 | 3.02E−05 |
| CAPG | −0.77 | 3.81E−07 | 1.97E−05 |
| CAPS | −0.75 | 2.39E−04 | 4.81E−03 |
| CASS4 | −0.95 | 8.51E−05 | 2.06E−03 |
| CCDC112 | −0.97 | 1.23E−03 | 1.78E−02 |
| CCDC50 | 0.86 | 8.32E−06 | 2.90E−04 |
| CCDC84 | 0.72 | 2.76E−04 | 5.38E−03 |
| CCDC88A | −1.25 | 4.19E−07 | 2.14E−05 |
| CCL18 | −1.16 | 9.34E−05 | 2.23E−03 |
| CCL20 | 0.88 | 1.36E−04 | 3.01E−03 |
| CCL22 | 1.55 | 5.69E−09 | 4.43E−07 |
| CCL4 | −0.88 | 3.30E−06 | 1.31E−04 |
| CCL5 | −0.67 | 1.06E−07 | 6.10E−06 |
| CCND1 | 0.96 | 1.63E−03 | 2.21E−02 |
| CCNG2 | 0.85 | 1.04E−03 | 1.59E−02 |
| CCP110 | −0.74 | 1.99E−03 | 2.60E−02 |
| CCR8 | 1.13 | 1.74E−06 | 7.70E−05 |
| CD163 | −1.14 | 2.50E−04 | 4.98E−03 |
| CD177 | 1.43 | 1.95E−06 | 8.41E−05 |
| CD200 | 1.79 | 1.68E−09 | 1.42E−07 |
| CD27 | 1.18 | 1.31E−17 | 5.68E−15 |
| CD300C | −1.49 | 2.71E−06 | 1.10E−04 |
| CD300LF | −1.48 | 2.88E−06 | 1.16E−04 |
| CD36 | −1.34 | 4.27E−06 | 1.65E−04 |
| CD55 | −0.82 | 6.44E−14 | 1.37E−11 |
| CD63 | −0.67 | 1.78E−08 | 1.22E−06 |
| CD68 | −1.89 | 8.01E−14 | 1.68E−11 |
| CD7 | 0.95 | 1.98E−18 | 9.51E−16 |
| CD70 | 0.61 | 3.48E−03 | 4.02E−02 |
| CD79A | 1.02 | 1.30E−03 | 1.86E−02 |
| CD79B | 0.84 | 3.60E−07 | 1.86E−05 |
| CD9 | −0.98 | 4.36E−05 | 1.14E−03 |
| CD97 | −1.09 | 8.13E−19 | 4.23E−16 |
| CDC42BPB | −1.25 | 7.36E−05 | 1.82E−03 |
| CDCP1 | −1.28 | 4.99E−06 | 1.89E−04 |
| CDHR3 | −0.93 | 6.91E−04 | 1.14E−02 |
| CDK2 | 0.68 | 2.66E−03 | 3.25E−02 |
| CDKN2D | −0.74 | 7.92E−05 | 1.93E−03 |
| CDT1 | −0.97 | 5.63E−04 | 9.75E−03 |
| CEACAM5 | 0.95 | 1.47E−03 | 2.05E−02 |
| CEACAM6 | 1.03 | 1.06E−03 | 1.60E−02 |
| CEBPB | −0.89 | 3.17E−10 | 3.12E−08 |
| CEBPD | −0.83 | 1.61E−03 | 2.18E−02 |
| CECR1 | −0.82 | 9.39E−07 | 4.48E−05 |
| CES1 | −2.51 | 9.42E−19 | 4.70E−16 |
| CFD | −1.44 | 1.01E−06 | 4.82E−05 |
| CHN1 | 0.85 | 1.95E−05 | 6.09E−04 |
| CHPT1 | −0.82 | 1.82E−03 | 2.43E−02 |
| CHRNA6 | 1.19 | 1.08E−04 | 2.52E−03 |
| CHST2 | 1.09 | 2.60E−04 | 5.14E−03 |
| CISH | −0.65 | 7.97E−04 | 1.27E−02 |
| CITED4 | −0.84 | 4.00E−03 | 4.42E−02 |
| CKS2 | 0.64 | 1.68E−03 | 2.26E−02 |
| CLEC12A | −0.97 | 2.05E−03 | 2.64E−02 |
| CLEC4A | −0.87 | 3.61E−03 | 4.13E−02 |
| CLEC4E | −0.84 | 3.43E−03 | 3.99E−02 |
| CLU | −1.19 | 3.93E−11 | 4.96E−09 |
| COL9A2 | 1.05 | 1.80E−04 | 3.82E−03 |
| COQ2 | −0.89 | 1.22E−03 | 1.77E−02 |
| CPA5 | 1.13 | 1.81E−04 | 3.82E−03 |
| CPT1A | −0.77 | 5.14E−05 | 1.33E−03 |
| CPVL | −1.27 | 1.99E−05 | 6.17E−04 |
| CRIM1 | −0.91 | 2.41E−05 | 7.24E−04 |
| CRIP1 | −0.84 | 1.19E−15 | 3.86E−13 |
| CRIP2 | −1.35 | 1.86E−09 | 1.55E−07 |
| CSDA | −0.85 | 4.46E−03 | 4.81E−02 |
| CSF1 | 0.79 | 1.75E−06 | 7.72E−05 |
| CST3 | −1.72 | 2.98E−12 | 4.45E−10 |
| CSTA | −1.63 | 1.22E−07 | 6.93E−06 |
| CTBP2 | −0.95 | 1.10E−03 | 1.65E−02 |
| CTDP1 | −0.66 | 1.04E−03 | 1.59E−02 |
| CTLA4 | 0.94 | 2.99E−14 | 6.94E−12 |
| CTSD | −1.01 | 1.48E−09 | 1.27E−07 |
| CTSH | −0.80 | 2.43E−07 | 1.30E−05 |
| CTSS | −0.84 | 1.14E−08 | 8.18E−07 |
| CTSW | −0.99 | 1.88E−06 | 8.20E−05 |
| CTTN | 0.89 | 2.70E−03 | 3.29E−02 |
| CTU1 | −0.94 | 7.76E−04 | 1.25E−02 |
| CUL9 | 0.59 | 1.33E−04 | 2.99E−03 |
| CX3CR1 | −1.06 | 4.65E−04 | 8.38E−03 |
| CXCL13 | 2.59 | 6.50E−20 | 4.45E−17 |
| CXCL16 | −1.55 | 1.67E−09 | 1.42E−07 |
| CXCL2 | −0.95 | 2.71E−03 | 3.30E−02 |
| CXCL3 | −1.43 | 6.62E−06 | 2.39E−04 |
| CXCL5 | −0.95 | 9.33E−04 | 1.44E−02 |
| CXCR2 | −2.44 | 1.54E−15 | 4.56E−13 |
| CXCR5 | 1.18 | 7.01E−06 | 2.51E−04 |
| CYBB | −1.89 | 2.48E−11 | 3.22E−09 |
| CYP27A1 | −2.11 | 5.88E−13 | 1.05E−10 |
| CYP4F22 | −1.05 | 5.77E−04 | 9.93E−03 |
| CYP51A1 | −1.15 | 1.32E−12 | 2.14E−10 |
| CYP7B1 | 1.20 | 8.56E−05 | 2.06E−03 |
| D4S234E | −0.65 | 1.91E−03 | 2.52E−02 |
| DAB2 | −1.36 | 8.15E−06 | 2.85E−04 |
| DDAH2 | −0.66 | 4.14E−03 | 4.54E−02 |
| DENND2D | 0.79 | 1.18E−10 | 1.31E−08 |
| DENND4A | −0.66 | 2.58E−04 | 5.11E−03 |
| DENND5A | −1.30 | 1.97E−05 | 6.12E−04 |
| DGKD | −0.59 | 4.24E−03 | 4.62E−02 |
| DHCR24 | −0.94 | 4.82E−04 | 8.62E−03 |
| DKK3 | 1.07 | 6.84E−04 | 1.13E−02 |
| DNAH8 | 1.52 | 1.06E−06 | 4.98E−05 |
| DNAJA4 | 0.90 | 1.91E−04 | 3.99E−03 |
| DNAJB1 | 1.07 | 6.32E−08 | 3.80E−06 |
| DNAJB4 | 1.02 | 2.32E−04 | 4.71E−03 |
| DOK3 | −0.89 | 3.34E−03 | 3.92E−02 |
| DOPEY2 | −0.62 | 2.40E−03 | 3.00E−02 |
| DPP4 | −0.81 | 1.22E−05 | 4.00E−04 |
| DSC2 | −0.92 | 3.11E−03 | 3.69E−02 |
| DSE | −0.84 | 1.85E−03 | 2.45E−02 |
| DSG2 | 0.84 | 4.04E−03 | 4.46E−02 |
| DTHD1 | 0.96 | 8.63E−04 | 1.35E−02 |
| DUS1L | −0.63 | 1.89E−03 | 2.49E−02 |
| DUSP16 | 0.81 | 2.36E−06 | 9.90E−05 |
| DUSP2 | 0.60 | 3.53E−06 | 1.38E−04 |
| DUSP4 | 0.78 | 3.80E−06 | 1.48E−04 |
| EBI3 | 1.71 | 4.61E−09 | 3.62E−07 |
| EID2 | −0.93 | 9.09E−04 | 1.41E−02 |
| ELK2AP | 1.47 | 1.05E−06 | 4.97E−05 |
| ELOVL6 | −0.90 | 3.27E−03 | 3.84E−02 |
| EMILIN2 | −0.82 | 5.60E−04 | 9.73E−03 |
| EMP1 | −1.05 | 4.84E−04 | 8.63E−03 |
| EMP3 | −0.61 | 8.27E−13 | 1.43E−10 |
| EMR2 | −0.74 | 3.66E−03 | 4.16E−02 |
| ENDOG | −0.88 | 1.42E−03 | 1.99E−02 |
| ENG | −0.82 | 3.98E−04 | 7.34E−03 |
| ENPP4 | −0.85 | 1.14E−03 | 1.68E−02 |
| ENTPD1 | 1.38 | 1.16E−14 | 2.89E−12 |
| EPB41L4A | −1.35 | 1.16E−06 | 5.44E−05 |
| EPHX2 | 1.02 | 4.05E−05 | 1.07E−03 |
| EPSTI1 | 0.61 | 1.79E−04 | 3.81E−03 |
| ETS2 | −0.82 | 6.43E−04 | 1.08E−02 |
| ETV7 | 0.83 | 8.34E−04 | 1.31E−02 |
| F5 | 1.10 | 2.64E−08 | 1.71E−06 |
| FAAH2 | 0.97 | 8.46E−07 | 4.13E−05 |
| FABP3 | −1.23 | 2.45E−05 | 7.33E−04 |
| FABP4 | −3.10 | 8.05E−26 | 1.49E−22 |
| FADS1 | −1.59 | 1.33E−08 | 9.39E−07 |
| FADS3 | −0.83 | 3.99E−03 | 4.42E−02 |
| FAIM2 | 1.37 | 1.44E−05 | 4.64E−04 |
| FAM101B | −0.86 | 6.35E−11 | 7.53E−09 |
| FAM159A | −0.70 | 3.91E−03 | 4.35E−02 |
| FAM174B | 1.26 | 3.33E−05 | 9.16E−04 |
| FAM211A | −1.12 | 3.81E−04 | 7.08E−03 |
| FAM212B | −0.86 | 3.95E−03 | 4.39E−02 |
| FAM65B | −0.63 | 2.46E−06 | 1.03E−04 |
| FAM82A2 | −0.92 | 7.07E−06 | 2.53E−04 |
| FANK1 | 0.80 | 4.00E−03 | 4.42E−02 |
| FAR2 | −0.88 | 3.23E−04 | 6.16E−03 |
| FBLN7 | 1.25 | 1.44E−10 | 1.56E−08 |
| FBP1 | −1.77 | 7.11E−10 | 6.69E−08 |
| FBXL8 | −0.90 | 4.17E−04 | 7.63E−03 |
| FCER1G | −1.58 | 5.97E−09 | 4.57E−07 |
| FCGR1A | −1.12 | 3.69E−04 | 6.89E−03 |
| FCGR2A | −1.63 | 2.60E−08 | 1.70E−06 |
| FCGR3A | −1.94 | 1.71E−12 | 2.71E−10 |
| FCGR3B | −1.26 | 6.61E−05 | 1.65E−03 |
| FCGRT | −1.14 | 8.82E−07 | 4.24E−05 |
| FCN1 | −1.24 | 9.00E−05 | 2.15E−03 |
| FCRL3 | 1.27 | 8.66E−07 | 4.20E−05 |
| FCRL6 | −1.14 | 8.14E−05 | 1.97E−03 |
| FEM1A | −0.67 | 3.58E−03 | 4.11E−02 |
| FGD2 | −1.38 | 1.17E−06 | 5.45E−05 |
| FGD4 | −0.77 | 2.01E−03 | 2.62E−02 |
| FGFBP2 | −1.77 | 8.63E−09 | 6.44E−07 |
| FGR | −1.83 | 2.60E−10 | 2.66E−08 |
| FHAD1 | −1.38 | 2.58E−06 | 1.06E−04 |
| FIG. 4 | −0.59 | 3.79E−03 | 4.24E−02 |
| FLJ35776 | 0.81 | 4.22E−07 | 2.15E−05 |
| FLJ39639 | −0.88 | 3.68E−03 | 4.17E−02 |
| FLNA | −0.66 | 1.14E−20 | 8.73E−18 |
| FLT1 | 1.04 | 7.44E−06 | 2.62E−04 |
| FN1 | −1.96 | 1.32E−11 | 1.81E−09 |
| FOSL2 | −0.71 | 1.68E−03 | 2.27E−02 |
| FOXP3 | 1.59 | 4.98E−20 | 3.59E−17 |
| FPR1 | −1.23 | 1.01E−04 | 2.39E−03 |
| FRMD4A | −0.79 | 3.00E−03 | 3.58E−02 |
| FRMD4B | −0.84 | 4.41E−06 | 1.70E−04 |
| FSD1L | −0.92 | 3.74E−03 | 4.20E−02 |
| FTH1 | −0.67 | 2.56E−10 | 2.64E−08 |
| FTL | −0.85 | 3.79E−08 | 2.37E−06 |
| GAA | −1.02 | 1.30E−04 | 2.92E−03 |
| GAB3 | −0.85 | 2.66E−08 | 1.72E−06 |
| GADD45A | 0.78 | 7.17E−07 | 3.56E−05 |
| GADD45G | 1.18 | 9.35E−07 | 4.48E−05 |
| GALNT6 | −0.83 | 2.08E−04 | 4.31E−03 |
| GAS2L1 | −1.20 | 5.82E−05 | 1.48E−03 |
| GAS7 | −1.16 | 2.58E−06 | 1.06E−04 |
| GCNT2 | 0.91 | 3.48E−03 | 4.02E−02 |
| GEM | 1.76 | 1.02E−08 | 7.37E−07 |
| GFM2 | 0.69 | 2.49E−03 | 3.09E−02 |
| GLB1L | −0.85 | 3.37E−03 | 3.94E−02 |
| GLDN | −1.71 | 4.54E−08 | 2.79E−06 |
| GLIPR2 | −0.74 | 1.32E−10 | 1.44E−08 |
| GLT25D1 | −0.72 | 6.23E−06 | 2.26E−04 |
| GLUL | −1.33 | 1.33E−15 | 4.20E−13 |
| GNG4 | 1.99 | 2.63E−16 | 9.75E−14 |
| GNG8 | 1.17 | 2.23E−04 | 4.57E−03 |
| GNLY | −0.81 | 1.20E−05 | 3.98E−04 |
| GOLGA7B | −0.84 | 4.56E−03 | 4.90E−02 |
| GPA33 | −1.13 | 2.73E−04 | 5.33E−03 |
| GPD1 | −1.38 | 7.08E−06 | 2.53E−04 |
| GPHN | 0.74 | 2.01E−03 | 2.62E−02 |
| GPNMB | −0.96 | 3.34E−04 | 6.33E−03 |
| GPR114 | −1.12 | 6.85E−05 | 1.70E−03 |
| GPR146 | −0.90 | 3.73E−03 | 4.20E−02 |
| GPR174 | 0.88 | 1.88E−07 | 1.03E−05 |
| GPX3 | −0.98 | 1.46E−03 | 2.04E−02 |
| GRAP | 0.64 | 1.74E−06 | 7.69E−05 |
| GRN | −1.50 | 1.53E−12 | 2.45E−10 |
| GSN | −1.19 | 9.14E−06 | 3.11E−04 |
| GZMB | −0.77 | 1.24E−03 | 1.78E−02 |
| GZMH | −1.45 | 1.65E−13 | 3.31E−11 |
| GZMK | 0.74 | 1.21E−05 | 3.99E−04 |
| H1F0 | 0.76 | 1.60E−03 | 2.18E−02 |
| HAVCR2 | 0.86 | 2.29E−05 | 6.98E−04 |
| HBA1 | −1.38 | 1.14E−05 | 3.80E−04 |
| HBA2 | −1.31 | 1.65E−05 | 5.29E−04 |
| HBB | −1.27 | 3.51E−05 | 9.53E−04 |
| HBEGF | −1.29 | 2.66E−05 | 7.63E−04 |
| HCAR2 | −1.08 | 4.00E−04 | 7.36E−03 |
| HCK | −1.94 | 5.25E−11 | 6.43E−09 |
| HCP5 | 0.63 | 3.72E−04 | 6.94E−03 |
| HDGFRP3 | −1.10 | 1.34E−04 | 3.00E−03 |
| HDHD2 | 0.63 | 2.20E−03 | 2.80E−02 |
| HEXB | −0.59 | 1.04E−04 | 2.44E−03 |
| HIATL1 | −0.66 | 2.66E−04 | 5.23E−03 |
| HIST1H2AC | 0.90 | 3.11E−06 | 1.24E−04 |
| HIST1H3H | 0.85 | 3.39E−03 | 3.96E−02 |
| HIST2H2BE | 0.96 | 8.28E−04 | 1.30E−02 |
| HK2 | −0.92 | 1.50E−03 | 2.08E−02 |
| HK3 | −1.94 | 7.78E−10 | 7.27E−08 |
| HLA-DMB | −0.86 | 1.31E−04 | 2.94E−03 |
| HLA-DPA1 | −0.64 | 1.93E−06 | 8.34E−05 |
| HLA-DPB1 | −0.67 | 7.71E−09 | 5.82E−07 |
| HLA-DQA1 | −1.04 | 5.37E−08 | 3.26E−06 |
| HLA-DQA2 | −0.72 | 1.09E−03 | 1.64E−02 |
| HLA-DQB1 | −0.79 | 2.53E−05 | 7.47E−04 |
| HLA-DQB2 | −0.75 | 1.90E−03 | 2.50E−02 |
| HLA-DRA | −1.51 | 6.71E−15 | 1.82E−12 |
| HLA-DRB1 | −1.16 | 1.15E−12 | 1.90E−10 |
| HLA-DRB5 | −1.13 | 5.70E−11 | 6.92E−09 |
| HLA-DRB6 | −1.23 | 7.51E−13 | 1.32E−10 |
| HMG20A | 0.61 | 1.41E−03 | 1.98E−02 |
| HMGCS1 | −0.65 | 2.38E−03 | 2.99E−02 |
| HNMT | −1.22 | 7.32E−05 | 1.81E−03 |
| HOPX | −1.18 | 4.95E−12 | 7.06E−10 |
| HP | −1.49 | 2.33E−06 | 9.78E−05 |
| HSD17B13 | −0.83 | 1.29E−03 | 1.85E−02 |
| HSD3B7 | −1.19 | 1.25E−04 | 2.86E−03 |
| HSPA14 | −0.82 | 3.14E−05 | 8.73E−04 |
| HSPA1A | 1.45 | 2.04E−09 | 1.67E−07 |
| HSPA1B | 2.10 | 1.90E−15 | 5.36E−13 |
| HSPA2 | 1.01 | 1.47E−03 | 2.05E−02 |
| HSPA6 | 1.82 | 4.52E−10 | 4.38E−08 |
| ICA1 | 2.28 | 2.71E−29 | 1.17E−25 |
| ICOS | 0.61 | 1.37E−06 | 6.30E−05 |
| ID3 | 1.15 | 3.36E−06 | 1.33E−04 |
| IFI30 | −1.63 | 7.61E−11 | 8.75E−09 |
| IFIT3 | −0.76 | 2.64E−03 | 3.23E−02 |
| IGF1 | −0.82 | 1.76E−04 | 3.76E−03 |
| IGF2R | −0.64 | 1.41E−03 | 1.98E−02 |
| IGFBP2 | −1.17 | 2.23E−04 | 4.57E−03 |
| IGFL2 | 1.03 | 1.12E−03 | 1.67E−02 |
| IGLL5 | 1.18 | 3.39E−05 | 9.32E−04 |
| IGSF6 | −1.25 | 7.40E−06 | 2.61E−04 |
| IKZF2 | 1.28 | 4.38E−07 | 2.22E−05 |
| IKZF4 | 1.01 | 7.56E−04 | 1.22E−02 |
| IL12RB2 | 0.64 | 1.61E−03 | 2.18E−02 |
| IL13RA1 | −1.12 | 1.28E−04 | 2.91E−03 |
| IL18R1 | 0.82 | 2.14E−06 | 9.05E−05 |
| IL1R2 | 0.92 | 8.27E−04 | 1.30E−02 |
| IL1RN | −0.90 | 3.22E−03 | 3.80E−02 |
| IL21R | 0.70 | 2.48E−04 | 4.96E−03 |
| IL24 | 0.90 | 2.33E−03 | 2.93E−02 |
| IL2RA | 0.83 | 5.28E−08 | 3.23E−06 |
| IL8 | −0.99 | 1.71E−03 | 2.29E−02 |
| IMPDH1 | −0.60 | 1.82E−04 | 3.84E−03 |
| INHBA | −2.13 | 1.39E−11 | 1.88E−09 |
| INPP1 | 0.77 | 2.65E−03 | 3.24E−02 |
| INPP5F | 1.53 | 1.97E−13 | 3.88E−11 |
| IQCC | −0.72 | 4.24E−03 | 4.62E−02 |
| IQSEC2 | −0.92 | 3.02E−03 | 3.60E−02 |
| IRAK3 | −1.20 | 2.58E−05 | 7.54E−04 |
| IRF8 | −1.09 | 4.92E−04 | 8.74E−03 |
| ITGA5 | −0.66 | 6.09E−04 | 1.03E−02 |
| ITGAM | −1.31 | 2.93E−05 | 8.24E−04 |
| ITGAX | −1.15 | 2.36E−05 | 7.12E−04 |
| ITGB1 | −0.68 | 9.21E−10 | 8.37E−08 |
| ITGB7 | −0.73 | 8.16E−15 | 2.12E−12 |
| ITM2A | 0.61 | 3.50E−09 | 2.79E−07 |
| IVNS1ABP | −0.71 | 9.68E−11 | 1.10E−08 |
| KCNE3 | −1.29 | 3.60E−07 | 1.86E−05 |
| KCNK5 | 1.31 | 1.21E−05 | 3.99E−04 |
| KCNN4 | 0.62 | 3.95E−04 | 7.30E−03 |
| KCTD12 | −0.96 | 2.51E−03 | 3.11E−02 |
| KIAA0907 | 0.67 | 1.56E−04 | 3.40E−03 |
| KIAA1324 | 0.81 | 2.95E−04 | 5.72E−03 |
| KIAA1841 | −0.84 | 6.25E−04 | 1.05E−02 |
| KIAA2013 | −0.62 | 2.23E−03 | 2.83E−02 |
| KIF13B | −0.89 | 2.88E−05 | 8.13E−04 |
| KIF21B | −0.74 | 3.20E−05 | 8.86E−04 |
| KLF11 | −0.94 | 2.08E−03 | 2.66E−02 |
| KLF13 | −0.63 | 8.89E−04 | 1.38E−02 |
| KLF2 | −1.09 | 1.15E−08 | 8.18E−07 |
| KLF3 | −0.97 | 9.02E−08 | 5.30E−06 |
| KLF4 | −1.10 | 4.66E−04 | 8.39E−03 |
| KLF6 | −0.74 | 1.99E−12 | 3.11E−10 |
| KLF9 | −0.88 | 5.86E−04 | 1.01E−02 |
| KLRD1 | −0.97 | 2.54E−05 | 7.47E−04 |
| KRT8 | 0.94 | 6.84E−04 | 1.13E−02 |
| KYNU | −0.98 | 1.86E−03 | 2.46E−02 |
| LAG3 | 0.72 | 2.67E−04 | 5.25E−03 |
| LAIR2 | 1.29 | 9.81E−12 | 1.37E−09 |
| LAPTM4B | 1.46 | 1.52E−08 | 1.06E−06 |
| LAT2 | −0.73 | 3.80E−03 | 4.25E−02 |
| LAX1 | 0.66 | 7.14E−08 | 4.25E−06 |
| LAYN | 2.21 | 8.54E−16 | 2.84E−13 |
| LDLR | −1.62 | 1.18E−10 | 1.31E−08 |
| LGALS1 | −0.94 | 5.79E−11 | 6.96E−09 |
| LGALS3 | −0.96 | 1.15E−09 | 1.01E−07 |
| LIF | 0.91 | 4.13E−03 | 4.54E−02 |
| LILRA5 | −1.83 | 3.06E−10 | 3.03E−08 |
| LILRA6 | −1.95 | 2.84E−13 | 5.43E−11 |
| LILRB1 | −1.63 | 2.81E−08 | 1.80E−06 |
| LILRB3 | −1.64 | 2.91E−10 | 2.92E−08 |
| LINC00167 | 0.92 | 2.66E−03 | 3.25E−02 |
| LINC00239 | 0.74 | 1.16E−03 | 1.71E−02 |
| LINC00341 | −0.89 | 1.53E−08 | 1.06E−06 |
| LMCD1 | 1.14 | 1.29E−04 | 2.91E−03 |
| LMF1 | −0.70 | 4.12E−03 | 4.53E−02 |
| LMNA | −1.68 | 1.18E−21 | 1.18E−18 |
| LOC100129196 | −0.82 | 3.17E−03 | 3.75E−02 |
| LOC100131067 | 0.75 | 2.58E−03 | 3.18E−02 |
| LOC100131176 | −1.10 | 4.20E−05 | 1.11E−03 |
| LOC100507582 | 1.37 | 9.57E−09 | 6.98E−07 |
| LOC286442 | 1.99 | 3.66E−11 | 4.67E−09 |
| LOC387723 | −0.84 | 2.68E−04 | 5.26E−03 |
| LOC389641 | 0.80 | 1.11E−04 | 2.57E−03 |
| LOC541471 | 0.86 | 2.38E−09 | 1.92E−07 |
| LOC731424 | −2.19 | 2.34E−14 | 5.74E−12 |
| LPCAT2 | −0.87 | 6.52E−04 | 1.09E−02 |
| LPL | −2.14 | 9.27E−12 | 1.31E−09 |
| LRP1 | −2.01 | 3.95E−12 | 5.83E−10 |
| LRRC2 | 0.85 | 7.52E−04 | 1.22E−02 |
| LRRC25 | −0.82 | 1.24E−03 | 1.79E−02 |
| LRRN3 | −1.02 | 3.55E−04 | 6.65E−03 |
| LSS | −1.12 | 1.93E−06 | 8.34E−05 |
| LST1 | −0.90 | 2.18E−05 | 6.69E−04 |
| LTA | 0.81 | 5.51E−06 | 2.05E−04 |
| LTA4H | −0.84 | 3.99E−05 | 1.06E−03 |
| LY86 | −0.97 | 2.25E−03 | 2.84E−02 |
| LYAR | −0.60 | 3.45E−05 | 9.45E−04 |
| LYN | −0.92 | 3.73E−03 | 4.20E−02 |
| LYZ | −2.05 | 7.45E−19 | 4.03E−16 |
| LZTS1 | −1.04 | 7.76E−04 | 1.25E−02 |
| MACC1 | −0.90 | 2.64E−03 | 3.23E−02 |
| MAGEH1 | 1.27 | 3.61E−10 | 3.52E−08 |
| MAP1LC3A | 1.32 | 8.81E−09 | 6.54E−07 |
| MARCH3 | 1.10 | 2.08E−04 | 4.31E−03 |
| MARCH9 | −1.01 | 5.25E−05 | 1.35E−03 |
| MARCO | −2.51 | 4.32E−19 | 2.44E−16 |
| MATK | −1.01 | 1.49E−05 | 4.78E−04 |
| MCTP2 | −1.04 | 7.13E−05 | 1.77E−03 |
| MEOX1 | 0.89 | 2.70E−03 | 3.29E−02 |
| METTL7A | 0.80 | 3.59E−03 | 4.11E−02 |
| METTL8 | 0.88 | 8.05E−07 | 3.96E−05 |
| MFSD4 | 0.78 | 1.98E−03 | 2.59E−02 |
| MFSD7 | −0.98 | 1.98E−03 | 2.59E−02 |
| MGST3 | −0.59 | 3.07E−07 | 1.63E−05 |
| MICAL2 | 1.02 | 4.63E−09 | 3.62E−07 |
| MILR1 | −1.05 | 4.87E−04 | 8.68E−03 |
| MIR21 | 0.79 | 9.17E−04 | 1.42E−02 |
| MIR210HG | 1.17 | 5.02E−05 | 1.30E−03 |
| MME | −1.32 | 3.33E−05 | 9.16E−04 |
| MMP19 | −1.41 | 8.29E−06 | 2.90E−04 |
| MNDA | −1.49 | 1.61E−06 | 7.24E−05 |
| MOB3B | −1.26 | 7.41E−05 | 1.82E−03 |
| MRC1 | −1.53 | 2.35E−07 | 1.27E−05 |
| MS4A4A | −1.65 | 2.34E−08 | 1.55E−06 |
| MS4A6A | −0.80 | 2.74E−03 | 3.32E−02 |
| MS4A7 | −2.11 | 3.20E−14 | 7.17E−12 |
| MSC | −1.09 | 3.88E−05 | 1.04E−03 |
| MSR1 | −2.11 | 2.13E−13 | 4.12E−11 |
| MT1G | −0.92 | 3.71E−03 | 4.18E−02 |
| MTMR11 | −0.93 | 2.97E−03 | 3.55E−02 |
| MXRA7 | −0.72 | 2.57E−03 | 3.17E−02 |
| MYADM | −1.66 | 1.06E−21 | 1.15E−18 |
| MYBL1 | −0.83 | 1.71E−03 | 2.29E−02 |
| MYL6B | 1.44 | 4.67E−10 | 4.49E−08 |
| MYLIP | 0.79 | 5.71E−05 | 1.45E−03 |
| MYO1G | −0.67 | 6.38E−11 | 7.53E−09 |
| MYO5C | 0.95 | 1.26E−04 | 2.88E−03 |
| NAGLU | −0.80 | 2.34E−03 | 2.94E−02 |
| NAPSA | 0.90 | 2.07E−03 | 2.66E−02 |
| NAPSB | −1.10 | 5.20E−04 | 9.15E−03 |
| NAV2 | 1.07 | 7.22E−04 | 1.18E−02 |
| NCEH1 | −0.98 | 6.73E−04 | 1.12E−02 |
| NCF1 | −1.05 | 2.55E−05 | 7.47E−04 |
| NCF2 | −1.29 | 2.65E−05 | 7.63E−04 |
| NCOR2 | −0.64 | 3.89E−03 | 4.34E−02 |
| NDFIP2 | 0.77 | 1.72E−05 | 5.49E−04 |
| NDST1 | −1.42 | 6.00E−06 | 2.21E−04 |
| NEK6 | −0.85 | 3.09E−03 | 3.67E−02 |
| NELF | −0.87 | 1.44E−03 | 2.01E−02 |
| NFAM1 | −1.54 | 1.04E−09 | 9.30E−08 |
| NFAT5 | 0.78 | 1.17E−04 | 2.69E−03 |
| NFKB2 | 0.73 | 1.97E−06 | 8.48E−05 |
| NFKBID | 0.72 | 1.11E−03 | 1.65E−02 |
| NGFRAP1 | 0.62 | 1.17E−03 | 1.72E−02 |
| NINJ1 | 0.65 | 1.21E−04 | 2.78E−03 |
| NKG7 | −1.05 | 8.14E−10 | 7.52E−08 |
| NLRC4 | −1.04 | 6.16E−04 | 1.04E−02 |
| NMB | 0.89 | 4.29E−05 | 1.13E−03 |
| NOM1 | −0.68 | 4.39E−04 | 7.96E−03 |
| NR1D2 | −0.75 | 1.03E−04 | 2.42E−03 |
| NR1H3 | −0.97 | 1.61E−03 | 2.19E−02 |
| NTRK1 | 1.24 | 3.61E−05 | 9.76E−04 |
| NUDT15 | −0.60 | 4.40E−03 | 4.76E−02 |
| NUP43 | 0.69 | 1.11E−05 | 3.70E−04 |
| NUPR1 | −1.17 | 2.91E−05 | 8.20E−04 |
| O3FAR1 | −1.21 | 6.18E−06 | 2.26E−04 |
| OGFRL1 | −0.84 | 8.62E−04 | 1.35E−02 |
| OLFM2 | 0.89 | 6.37E−04 | 1.07E−02 |
| OLR1 | −2.33 | 2.26E−16 | 8.65E−14 |
| OPN3 | −0.96 | 2.36E−04 | 4.77E−03 |
| OSCAR | −2.21 | 9.43E−13 | 1.61E−10 |
| OSM | −1.16 | 2.89E−06 | 1.16E−04 |
| OXSR1 | −0.62 | 5.99E−04 | 1.02E−02 |
| P2RX5 | 0.65 | 1.13E−03 | 1.68E−02 |
| PAQR5 | −1.35 | 1.95E−05 | 6.09E−04 |
| PASK | 0.71 | 2.45E−05 | 7.32E−04 |
| PCOLCE2 | −1.33 | 2.01E−05 | 6.21E−04 |
| PCSK5 | −0.83 | 3.78E−03 | 4.23E−02 |
| PDCD1 | 1.05 | 2.50E−10 | 2.61E−08 |
| PDCD7 | −0.64 | 4.24E−03 | 4.62E−02 |
| PDE4A | −0.76 | 1.86E−03 | 2.46E−02 |
| PDE4DIP | 0.79 | 1.83E−09 | 1.53E−07 |
| PDE7B | 2.27 | 1.88E−15 | 5.36E−13 |
| PDGFA | 1.04 | 1.80E−04 | 3.82E−03 |
| PDLIM1 | −1.61 | 2.58E−08 | 1.70E−06 |
| PECAM1 | −1.33 | 1.26E−06 | 5.86E−05 |
| PER1 | −0.63 | 1.87E−03 | 2.47E−02 |
| PGD | −0.77 | 3.58E−05 | 9.70E−04 |
| PHLDA2 | 1.14 | 1.28E−05 | 4.17E−04 |
| PHYH | −0.72 | 2.04E−03 | 2.64E−02 |
| PI16 | −1.00 | 1.35E−03 | 1.92E−02 |
| PIGR | −0.99 | 5.11E−04 | 9.00E−03 |
| PIGW | −0.93 | 1.41E−04 | 3.10E−03 |
| PILRA | −1.58 | 5.31E−08 | 3.24E−06 |
| PION | −1.27 | 8.81E−07 | 4.24E−05 |
| PKD2 | −0.99 | 1.62E−04 | 3.50E−03 |
| PKP2 | −1.04 | 3.89E−04 | 7.21E−03 |
| PLAC8 | −0.59 | 6.76E−04 | 1.12E−02 |
| PLAUR | −0.91 | 8.23E−04 | 1.30E−02 |
| PLBD1 | −2.00 | 2.08E−11 | 2.73E−09 |
| PLEKHG3 | −0.77 | 2.94E−03 | 3.53E−02 |
| PLIN2 | −0.59 | 1.23E−07 | 6.95E−06 |
| PLXDC2 | −1.56 | 8.08E−07 | 3.96E−05 |
| PMAIP1 | 1.49 | 5.28E−17 | 2.14E−14 |
| PMCH | 0.99 | 1.16E−03 | 1.72E−02 |
| PNPLA6 | −0.86 | 1.89E−05 | 5.93E−04 |
| POU2AF1 | 1.13 | 6.20E−06 | 2.26E−04 |
| PPARG | −1.29 | 3.06E−06 | 1.22E−04 |
| PPIC | −0.97 | 1.86E−03 | 2.46E−02 |
| PPP3CA | −0.65 | 3.16E−08 | 2.00E−06 |
| PRKX | −0.65 | 8.84E−05 | 2.12E−03 |
| PROCR | −1.13 | 3.25E−04 | 6.18E−03 |
| PROK2 | −1.38 | 9.72E−06 | 3.27E−04 |
| PROS1 | −1.20 | 8.14E−05 | 1.97E−03 |
| PSAP | −0.62 | 8.22E−10 | 7.52E−08 |
| PTAFR | −1.95 | 7.84E−15 | 2.08E−12 |
| PTGER4 | −0.68 | 4.66E−11 | 5.76E−09 |
| PTP4A3 | 0.73 | 1.95E−03 | 2.55E−02 |
| PTPLA | 1.23 | 2.77E−05 | 7.86E−04 |
| PTPN4 | −0.84 | 3.21E−05 | 8.87E−04 |
| PTPN7 | 0.66 | 2.92E−10 | 2.92E−08 |
| PTPRO | −0.89 | 4.28E−03 | 4.65E−02 |
| PVALB | 1.40 | 8.47E−06 | 2.92E−04 |
| PVR | −1.60 | 2.12E−07 | 1.15E−05 |
| PZP | −0.80 | 3.07E−03 | 3.65E−02 |
| RAB31 | −1.23 | 1.78E−05 | 5.65E−04 |
| RAB33A | 0.92 | 5.43E−05 | 1.39E−03 |
| RAB3GAP1 | 0.60 | 1.80E−05 | 5.69E−04 |
| RAP2A | −0.78 | 2.32E−03 | 2.93E−02 |
| RARA | −0.72 | 6.70E−04 | 1.11E−02 |
| RASGEF1B | −1.05 | 2.80E−05 | 7.93E−04 |
| RASGRP2 | −0.76 | 8.74E−08 | 5.16E−06 |
| RBM15B | −0.59 | 3.65E−03 | 4.16E−02 |
| RBM47 | −1.06 | 5.58E−04 | 9.72E−03 |
| RBMS2 | −0.75 | 4.76E−04 | 8.53E−03 |
| RBP4 | −1.66 | 1.65E−07 | 9.11E−06 |
| REREP3 | 1.33 | 7.28E−06 | 2.58E−04 |
| RETN | −1.75 | 3.18E−08 | 2.00E−06 |
| RGS1 | 1.15 | 3.22E−13 | 6.06E−11 |
| RGS2 | 1.18 | 4.05E−11 | 5.05E−09 |
| RHOU | −1.02 | 3.35E−04 | 6.33E−03 |
| RNF130 | −0.82 | 6.50E−04 | 1.09E−02 |
| RNF144B | −0.93 | 1.87E−03 | 2.47E−02 |
| RNF157 | −1.25 | 1.37E−06 | 6.30E−05 |
| RPGR | −0.86 | 1.02E−03 | 1.56E−02 |
| RRAGD | −1.08 | 3.29E−04 | 6.24E−03 |
| RTKN2 | 1.31 | 9.89E−08 | 5.74E−06 |
| RXRA | −1.20 | 2.97E−05 | 8.29E−04 |
| S100A10 | −0.96 | 3.91E−18 | 1.75E−15 |
| S100A11 | −0.69 | 6.46E−11 | 7.56E−09 |
| S100A8 | −1.75 | 8.06E−09 | 6.05E−07 |
| S100A9 | −1.29 | 4.73E−06 | 1.81E−04 |
| S1PR1 | −0.66 | 4.61E−05 | 1.20E−03 |
| S1PR5 | −1.07 | 4.96E−04 | 8.79E−03 |
| SAP30 | −0.88 | 1.73E−03 | 2.31E−02 |
| SARDH | 1.00 | 2.51E−10 | 2.61E−08 |
| SAYSD1 | 0.67 | 5.58E−04 | 9.72E−03 |
| SC5DL | −0.59 | 7.19E−04 | 1.18E−02 |
| SCD | −1.36 | 1.60E−07 | 8.87E−06 |
| SCD5 | −0.64 | 1.56E−03 | 2.14E−02 |
| SCGB1A1 | −2.98 | 5.31E−22 | 6.27E−19 |
| SCGB3A1 | −2.07 | 1.31E−16 | 5.15E−14 |
| SCGB3A2 | −0.95 | 2.44E−03 | 3.04E−02 |
| SCPEP1 | −1.10 | 1.96E−05 | 6.09E−04 |
| SDC4 | 0.71 | 7.38E−05 | 1.82E−03 |
| SDCBP | −0.66 | 2.42E−10 | 2.58E−08 |
| SDSL | −0.95 | 7.13E−04 | 1.17E−02 |
| SEC61A2 | 0.78 | 9.37E−04 | 1.44E−02 |
| SEMA3C | −1.06 | 5.53E−04 | 9.66E−03 |
| SEMA5A | −1.25 | 4.05E−06 | 1.57E−04 |
| SEPT10 | −1.06 | 8.21E−04 | 1.30E−02 |
| SEPT11 | −0.86 | 2.35E−08 | 1.55E−06 |
| SERPINA1 | −2.24 | 1.73E−17 | 7.23E−15 |
| SERPINB6 | −0.70 | 3.42E−03 | 3.99E−02 |
| SERPING1 | −2.07 | 1.19E−11 | 1.65E−09 |
| SESN3 | 0.82 | 3.53E−03 | 4.06E−02 |
| SFTPA1 | −1.06 | 3.81E−04 | 7.08E−03 |
| SFTPC | −2.68 | 4.78E−21 | 4.14E−18 |
| SGMS2 | −1.52 | 1.54E−06 | 6.95E−05 |
| SGPP2 | 1.67 | 5.29E−10 | 5.06E−08 |
| SH2B3 | −0.83 | 4.22E−04 | 7.72E−03 |
| SH3BP5 | −0.90 | 1.26E−04 | 2.88E−03 |
| SIDT2 | −1.02 | 7.13E−06 | 2.53E−04 |
| SIGLEC10 | −0.81 | 8.77E−04 | 1.37E−02 |
| SIGLEC14 | −1.73 | 1.06E−09 | 9.40E−08 |
| SIRPA | −1.92 | 2.45E−10 | 2.58E−08 |
| SIRPB1 | −1.18 | 3.85E−05 | 1.03E−03 |
| SIRPB2 | −1.21 | 2.31E−05 | 7.02E−04 |
| SIRPG | 1.51 | 6.59E−23 | 1.07E−19 |
| SLAMF8 | −0.90 | 3.68E−03 | 4.17E−02 |
| SLC11A1 | −2.57 | 2.16E−19 | 1.28E−16 |
| SLC12A2 | −0.76 | 7.63E−04 | 1.23E−02 |
| SLC15A3 | −1.03 | 1.18E−03 | 1.73E−02 |
| SLC16A6 | −0.89 | 3.27E−03 | 3.84E−02 |
| SLC19A3 | −2.12 | 1.76E−11 | 2.34E−09 |
| SLC25A24 | −0.83 | 8.56E−05 | 2.06E−03 |
| SLC27A3 | −1.00 | 1.46E−04 | 3.20E−03 |
| SLC2A6 | −0.84 | 3.96E−03 | 4.40E−02 |
| SLC2A9 | −0.94 | 2.98E−03 | 3.56E−02 |
| SLC31A1 | −0.64 | 6.22E−04 | 1.05E−02 |
| SLC31A2 | −0.92 | 6.08E−04 | 1.03E−02 |
| SLC44A4 | −1.02 | 1.12E−03 | 1.67E−02 |
| SLC47A1 | −1.17 | 2.24E−04 | 4.57E−03 |
| SLC4A10 | −0.99 | 9.18E−04 | 1.42E−02 |
| SLC7A7 | −1.32 | 2.69E−05 | 7.71E−04 |
| SLCO2B1 | −1.73 | 1.21E−08 | 8.61E−07 |
| SLCO3A1 | −0.89 | 6.25E−05 | 1.58E−03 |
| SLFN11 | −0.69 | 7.97E−04 | 1.27E−02 |
| SLPI | −1.38 | 5.86E−06 | 2.17E−04 |
| SMAD1 | 1.05 | 8.43E−04 | 1.32E−02 |
| SNORA4 | 0.89 | 4.35E−04 | 7.93E−03 |
| SNORA63 | 0.69 | 3.81E−03 | 4.26E−02 |
| SNORD2 | 0.82 | 3.55E−03 | 4.08E−02 |
| SNORD50A | 0.77 | 4.63E−04 | 8.35E−03 |
| SNORD50B | 0.71 | 1.16E−03 | 1.72E−02 |
| SNTA1 | −0.72 | 4.66E−03 | 4.99E−02 |
| SNTB1 | −0.73 | 4.14E−03 | 4.54E−02 |
| SNTB2 | −0.62 | 3.35E−03 | 3.93E−02 |
| SNTN | −1.52 | 4.78E−07 | 2.41E−05 |
| SNX10 | −0.96 | 4.54E−08 | 2.79E−06 |
| SOCS2 | −1.17 | 8.71E−06 | 2.99E−04 |
| SORBS3 | −0.77 | 1.85E−04 | 3.89E−03 |
| SORT1 | −1.21 | 1.01E−04 | 2.39E−03 |
| SOS1 | −0.67 | 4.04E−03 | 4.46E−02 |
| SOWAHC | −1.27 | 2.72E−05 | 7.77E−04 |
| SOX4 | 1.39 | 9.70E−08 | 5.65E−06 |
| SPAG6 | −0.95 | 2.08E−03 | 2.66E−02 |
| SPARC | −1.03 | 1.19E−03 | 1.74E−02 |
| SPI1 | −2.13 | 4.82E−14 | 1.06E−11 |
| SPIRE1 | −1.34 | 2.50E−05 | 7.41E−04 |
| SPON2 | −1.34 | 2.11E−06 | 8.96E−05 |
| SPP1 | 2.24 | 2.74E−14 | 6.59E−12 |
| SPRY1 | 0.98 | 1.21E−03 | 1.76E−02 |
| SSBP4 | −0.85 | 1.20E−07 | 6.85E−06 |
| ST6GALNAC2 | −0.94 | 2.87E−03 | 3.46E−02 |
| ST8SIA1 | 0.84 | 1.29E−03 | 1.86E−02 |
| STAC | −1.21 | 9.05E−05 | 2.16E−03 |
| STAG3 | 1.12 | 1.66E−04 | 3.58E−03 |
| STAM | 1.00 | 9.30E−08 | 5.44E−06 |
| STAMBPL1 | 0.92 | 1.53E−09 | 1.30E−07 |
| STX16 | 0.59 | 3.48E−05 | 9.49E−04 |
| STX3 | −1.63 | 5.82E−09 | 4.50E−07 |
| STX6 | 0.62 | 6.71E−06 | 2.42E−04 |
| SUOX | 0.94 | 2.51E−03 | 3.11E−02 |
| SVIL | −0.81 | 4.45E−06 | 1.70E−04 |
| SYK | −1.26 | 2.56E−06 | 1.06E−04 |
| TAGLN2 | −0.67 | 1.02E−12 | 1.73E−10 |
| TBC1D2 | −0.65 | 4.10E−03 | 4.51E−02 |
| TBC1D4 | 1.28 | 4.06E−15 | 1.12E−12 |
| TBC1D8 | 0.95 | 7.82E−04 | 1.25E−02 |
| TBX21 | −0.65 | 2.90E−03 | 3.49E−02 |
| TCEA3 | −1.00 | 7.19E−04 | 1.18E−02 |
| TCF7L2 | −1.16 | 2.37E−04 | 4.78E−03 |
| TGFB1 | −0.86 | 3.67E−16 | 1.31E−13 |
| TGFBI | −0.84 | 2.71E−04 | 5.30E−03 |
| TGFBR3 | −0.76 | 7.40E−05 | 1.82E−03 |
| TGM2 | −1.45 | 2.61E−06 | 1.07E−04 |
| THADA | 1.04 | 2.89E−06 | 1.16E−04 |
| THBD | −1.17 | 2.01E−04 | 4.20E−03 |
| THBS1 | −1.31 | 1.87E−05 | 5.88E−04 |
| TIAM1 | 1.15 | 6.92E−11 | 8.03E−09 |
| TIGD5 | −0.90 | 1.70E−03 | 2.28E−02 |
| TIGIT | 1.82 | 1.82E−35 | 2.37E−31 |
| TKT | −0.65 | 8.66E−05 | 2.08E−03 |
| TLE4 | −1.01 | 2.51E−06 | 1.04E−04 |
| TLR4 | −1.00 | 1.49E−03 | 2.06E−02 |
| TLR7 | −1.05 | 5.20E−05 | 1.34E−03 |
| TM6SF1 | −1.22 | 1.26E−04 | 2.88E−03 |
| TMEM102 | −0.61 | 3.70E−03 | 4.18E−02 |
| TMEM136 | 0.84 | 2.03E−03 | 2.63E−02 |
| TMEM170B | −1.12 | 8.03E−05 | 1.95E−03 |
| TMEM19 | −0.85 | 2.61E−04 | 5.15E−03 |
| TMEM220 | −0.94 | 2.05E−03 | 2.64E−02 |
| TMEM8A | −0.74 | 1.37E−03 | 1.94E−02 |
| TMPRSS3 | 1.02 | 1.30E−04 | 2.92E−03 |
| TNFAIP1 | −0.78 | 1.46E−03 | 2.04E−02 |
| TNFAIP2 | −1.31 | 2.22E−05 | 6.77E−04 |
| TNFRSF18 | 1.51 | 3.77E−28 | 9.79E−25 |
| TNFRSF4 | 1.44 | 4.05E−26 | 8.77E−23 |
| TNFRSF9 | 1.37 | 4.56E−12 | 6.66E−10 |
| TNFSF13 | −1.73 | 9.66E−09 | 7.01E−07 |
| TNFSF13B | 0.96 | 1.42E−06 | 6.50E−05 |
| TNIP3 | 0.84 | 1.36E−03 | 1.93E−02 |
| TOX | 0.71 | 9.32E−06 | 3.16E−04 |
| TOX2 | 1.18 | 7.17E−08 | 4.25E−06 |
| TP53INP1 | 0.62 | 3.41E−07 | 1.78E−05 |
| TPCN1 | 0.61 | 2.01E−03 | 2.62E−02 |
| TPPP | −1.51 | 1.53E−06 | 6.95E−05 |
| TPPP3 | −1.08 | 6.46E−04 | 1.08E−02 |
| TRAF1 | 1.11 | 1.49E−21 | 1.39E−18 |
| TREM1 | −2.32 | 1.41E−15 | 4.34E−13 |
| TREM2 | −1.11 | 1.10E−04 | 2.56E−03 |
| TRIM16 | 0.90 | 1.23E−05 | 4.01E−04 |
| TSPAN13 | 1.05 | 7.83E−04 | 1.25E−02 |
| TSPAN2 | −1.23 | 5.28E−06 | 1.98E−04 |
| TSPAN5 | 0.59 | 2.50E−03 | 3.10E−02 |
| TTPAL | 0.60 | 1.11E−03 | 1.66E−02 |
| TIYH2 | −0.91 | 3.66E−03 | 4.16E−02 |
| TUBB6 | −1.37 | 1.49E−05 | 4.78E−04 |
| TULP3 | −0.93 | 2.14E−03 | 2.73E−02 |
| TULP4 | 0.74 | 8.38E−06 | 2.90E−04 |
| TYROBP | −1.98 | 1.51E−15 | 4.55E−13 |
| TYW5 | 0.62 | 6.11E−04 | 1.04E−02 |
| UBE2E2 | −1.27 | 3.51E−05 | 9.53E−04 |
| UNC93B1 | −0.69 | 1.61E−06 | 7.24E−05 |
| UNQ6494 | 1.74 | 5.98E−09 | 4.57E−07 |
| URI1 | −0.59 | 4.25E−03 | 4.62E−02 |
| VAT1 | −0.66 | 5.35E−04 | 9.38E−03 |
| VDR | 0.98 | 2.17E−04 | 4.47E−03 |
| VIM | −0.92 | 5.63E−16 | 1.93E−13 |
| VMO1 | −1.16 | 2.44E−04 | 4.89E−03 |
| VPS37B | −0.78 | 5.19E−05 | 1.34E−03 |
| VSIG4 | −2.51 | 2.03E−19 | 1.26E−16 |
| VTRNA1-3 | 0.82 | 1.06E−03 | 1.60E−02 |
| WNT10A | 0.90 | 7.12E−04 | 1.17E−02 |
| XBP1 | −0.85 | 9.56E−20 | 6.21E−17 |
| XIST | 0.67 | 3.46E−04 | 6.49E−03 |
| ZBED2 | 1.41 | 6.92E−08 | 4.14E−06 |
| ZBED4 | −1.07 | 8.39E−06 | 2.90E−04 |
| ZBTB7A | −0.81 | 5.44E−06 | 2.03E−04 |
| ZC3H12D | 0.78 | 7.11E−06 | 2.53E−04 |
| ZC3H7A | 0.64 | 6.20E−08 | 3.74E−06 |
| ZDHHC11 | −1.21 | 6.42E−05 | 1.62E−03 |
| ZDHHC8 | −0.70 | 4.02E−03 | 4.44E−02 |
| ZEB2 | −1.06 | 2.94E−05 | 8.24E−04 |
| ZFP36L1 | 0.66 | 1.29E−13 | 2.61E−11 |
| ZFYVE28 | −0.68 | 6.94E−04 | 1.14E−02 |
| ZMAT3 | 0.61 | 1.18E−03 | 1.73E−02 |
| ZMYND10 | −0.81 | 2.07E−03 | 2.66E−02 |
| ZNF180 | −0.93 | 1.71E−03 | 2.30E−02 |
| ZNF232 | 0.72 | 1.94E−03 | 2.55E−02 |
| ZNF580 | 0.76 | 6.72E−05 | 1.68E−03 |
| ZNF683 | −1.41 | 3.30E−07 | 1.73E−05 |
| ZNF708 | 0.76 | 6.30E−04 | 1.06E−02 |
| ZNF80 | 1.39 | 1.56E−07 | 8.71E−06 |
| ZNRF1 | 0.78 | 3.16E−05 | 8.76E−04 |

TABLE 3
Related to FIG. 1.
Pathway analysis of differentially expressed genes in CD4⁺ TILs relative to N-TILs.

| Ingenuity | | Number of | Number of | Fraction of | |
|---|---|---|---|---|---|
| canonical | -log(B-H | genes in | DEGs in | upregulated | |
| pathways | p-value) | pathway | pathway | genes | DEGS in the pathway |
| Altered T Cell | 3.02 | 90 | 8 | 0.0889 | SPP1, CD79B, CXCL13, |
| and B Cell | | | | | CSF1, LTA, CD79A, NFKB2, |
| Signaling in | | | | | TNFSF13B |
| Rheumatoid | | | | | |
| Arthritis | | | | | |
| T Helper Cell | 2.92 | 73 | 7 | 0.0959 | IL21R, ICOS, FOXP3, |
| Differentiation | | | | | IL12RB2, IL2RA, CXCR5, |
| | | | | | IL18R1 |
| Th1 and Th2 | 2.77 | 185 | 10 | 0.0541 | TNFRSF4, HAVCR2, LTA, |
| Activation | | | | | ICOS, CCR8, IL12RB2, |
| Pathway | | | | | IL2RA, IL24, mir-21, |
| | | | | | IL18R1 |
| GADD45 | 2.62 | 19 | 4 | 0.211 | GADD45A, GADD45G, |
| Signaling | | | | | CCND1, CDK2 |
| B Cell | 2.51 | 41 | 5 | 0.122 | NFKBID, NFAT5, NFKB2, |
| Activating | | | | | TNFSF13B, TRAF1 |
| Factor | | | | | |
| Signaling | | | | | |
| Role of | 2.44 | 312 | 12 | 0.0385 | IL1R2, NFKBID, NFAT5, |
| Macrophages, | | | | | WNT10A, PDGFA, CSF1, |
| Fibroblasts and | | | | | DKK3, LTA, CCND1, |
| Endothelial | | | | | TNFSF13B, IL18R1, TRAF1 |
| Cells in | | | | | |
| Rheumatoid | | | | | |
| Arthritis | | | | | |
| Protein Kinase | 2.11 | 400 | 13 | 0.0325 | GNG4, AKAP5, PTPN7, |
| A Signaling | | | | | MYL6B, NFKB2, DUSP2, |
| | | | | | NFKBID, NFAT5, PDE7B, |
| | | | | | DUSP4, H1F0, EBI3, |
| | | | | | DUSP16 |
| TNFR2 | 2.11 | 30 | 4 | 0.133 | NFKBID, LTA, NFKB2, |
| Signaling | | | | | TRAF1 |
| 4-1BB | 2.05 | 32 | 4 | 0.125 | NFKBID, TNFRSF9, |
| Signaling in T | | | | | NFKB2, TRAF1 |
| Lymphocytes | | | | | |
| Role of | 1.86 | 233 | 9 | 0.0386 | IL1R2, NFKBID, SPP1, |
| Osteoblasts, | | | | | NFAT5, WNT10A, CSF1, |
| Osteoclasts | | | | | DKK3, SMAD1, IL18R1 |
| and | | | | | |
| Chondrocytes | | | | | |
| in Rheumatoid | | | | | |
| Arthritis | | | | | |
| April Mediated | 1.86 | 39 | 4 | 0.103 | NFKBID, NFAT5, |
| Signaling | | | | | NFKB2, TRAF1 |
| D-myo- | 1.86 | 144 | 7 | 0.0486 | PTPN7, INPP5F, PDCD1, |
| inositol(1,4,5,6)- | | | | | EPHX2, SGPP2, DUSP2, |
| Tetrakisphosphate | | | | | DUSP16 |
| Biosynthesis | | | | | |
| D-myo- | 1.86 | 144 | 7 | 0.0486 | PTPN7, INPP5F, PDCD1, |
| inositol(3,4,5,6)- | | | | | EPHX2, SGPP2, DUSP2, |
| tetrakisphosphate | | | | | DUSP16 |
| Biosynthesis | | | | | |
| Th2 Pathway | 1.79 | 150 | 7 | 0.0467 | TNFRSF4, ICOS, CCR8, |
| | | | | | IL12RB2, IL2RA, IL24, |
| | | | | | mir-21 |
| p53 Signaling | 1.78 | 111 | 6 | 0.0541 | PMAIP1, TP53INP1, |
| | | | | | GADD45A, GADD45G, |
| | | | | | CCND1, CDK2 |
| 3- | 1.78 | 200 | 8 | 0.04 | PTPN7, INPP5F, PDCD1, |
| phosphoinositide | | | | | EPHX2, SGPP2, ICOS, |
| Biosynthesis | | | | | DUSP2, DUSP16 |
| 3- | 1.74 | 158 | 7 | 0.0443 | PTPN7, INPP5F, PDCD1, |
| phosphoinositide | | | | | EPHX2, SGPP2, DUSP2, |
| Degradation | | | | | DUSP16 |
| D-myo- | 1.7 | 162 | 7 | 0.0432 | PTPN7, INPP5F, PDCD1, |
| inositol-5- | | | | | EPHX2, SGPP2, DUSP2, |
| phosphate | | | | | DUSP16 |
| Metabolism | | | | | |
| Small Cell | 1.61 | 85 | 5 | 0.0588 | NFKBID, NFKB2, CCND1, |
| Lung Cancer | | | | | CDK2, TRAF1 |
| Signaling | | | | | |
| CD27 | 1.55 | 53 | 4 | 0.0755 | NFKBID, CD70, NFKB2, |
| Signaling in | | | | | CD27 |
| Lymphocytes | | | | | |
| NF-κB | 1.5 | 181 | 7 | 0.0387 | IL1R2, NFKBID, FLT1, |
| Signaling | | | | | LTA, NTRK1, NFKB2, |
| | | | | | TNFSF13B |
| Th1 Pathway | 1.5 | 135 | 6 | 0.0444 | HAVCR2, LTA, ICOS, |
| | | | | | IL12RB2, mir-21, IL18R1 |
| Superpathway | 1.49 | 235 | 8 | 0.034 | PTPN7, INPP5F, PDCD1, |
| of Inositol | | | | | EPHX2, SGPP2, ICOS, |
| Phosphate | | | | | DUSP2, DUSP16 |
| Compounds | | | | | |
| Role of NFAT | 1.49 | 186 | 7 | 0.0376 | GNG4, AKAP5, NFKBID, |
| in Regulation | | | | | NFAT5, CD79B, CD79A, |
| of the Immune | | | | | NFKB2 |
| Response | | | | | |
| Hepatic | 1.49 | 187 | 7 | 0.0374 | IL1R2, FLT1, PDGFA, |
| Fibrosis/ | | | | | CSF1, MYL6B, NFKB2, |
| Hepatic | | | | | COL9A2 |
| Stellate Cell | | | | | |
| Activation | | | | | |
| Lymphotoxin | 1.3 | 67 | 4 | 0.0597 | NFKBID, LTA, NFKB2, |
| β Receptor | | | | | TRAF1 |
| Signaling | | | | | |

TABLE 4
Related to FIG. 7.
Analysis of TCR beta chain sequences from RNA-Seq data of CD4⁺ TILs versus N-TILs.

| | CD4⁺ | CD4⁺ | CD4⁺ | CD4⁺ | CD4⁺ | CD4⁺ | CD4⁺ | CD4⁺ |
|---|---|---|---|---|---|---|---|---|
| | N-TILs | TILs | N-TILs | TILs | N-TILs | TILs | N-TILs | TILs |
| Patient ID | >1 | >1 | >2 | >2 | >3 | >3 | >4 | >4 |
| NSCLC_01 | 36 | 37 | 12 | 20 | 2 | 12 | 0 | 5 |
| NSCLC_02 | N/A | 58 | N/A | 29 | N/A | 14 | N/A | 11 |
| NSCLC_03 | 29 | 38 | 12 | 13 | 5 | 6 | 2 | 0 |
| NSCLC_04 | 13 | 51 | 4 | 17 | 1 | 4 | 1 | 2 |
| NSCLC_05 | 23 | 60 | 12 | 26 | 6 | 10 | 3 | 4 |
| NSCLC_06 | 57 | 41 | 22 | 16 | 16 | 6 | 5 | 2 |
| NSCLC_07 | 30 | 43 | 13 | 19 | 4 | 7 | 1 | 3 |
| NSCLC_08 | 30 | 94 | 14 | 41 | 9 | 19 | 8 | 10 |
| NSCLC_09 | 31 | 30 | 7 | 9 | 3 | 4 | 2 | 2 |
| NSCLC_10 | 30 | 56 | 8 | 26 | 4 | 12 | 3 | 7 |
| NSCLC_11 | 6 | 29 | 3 | 15 | 2 | 7 | 2 | 6 |
| NSCLC_12 | 81 | 74 | 48 | 28 | 23 | 11 | 12 | 5 |
| NSCLC_13 | 23 | 33 | 7 | 9 | 3 | 3 | 2 | 2 |
| NSCLC_14 | 21 | 36 | 12 | 10 | 7 | 2 | 4 | 1 |
| NSCLC_15 | 14 | 40 | 5 | 11 | 1 | 3 | 1 | 3 |
| NSCLC_16 | N/A | 36 | N/A | 20 | N/A | 11 | N/A | 6 |
| NSCLC_17 | 15 | 39 | 3 | 21 | 2 | 9 | 0 | 7 |
| NSCLC_18 | 16 | 25 | 7 | 13 | 3 | 6 | 1 | 2 |
| NSCLC_19 | 33 | 54 | 11 | 25 | 7 | 9 | 6 | 5 |
| NSCLC_20 | 12 | 44 | 8 | 18 | 4 | 10 | 3 | 5 |
| NSCLC_21 | 16 | 26 | 5 | 18 | 1 | 8 | 0 | 6 |
| NSCLC_22 | 19 | 52 | 12 | 17 | 9 | 5 | 4 | 2 |
| NSCLC_23 | N/A | 46 | N/A | 15 | N/A | 7 | N/A | 3 |
| NSCLC_24 | N/A | 45 | N/A | 27 | N/A | 23 | N/A | 13 |
| NSCLC_25 | N/A | 31 | N/A | 12 | N/A | 6 | N/A | 5 |
| NSCLC_26 | N/A | 53 | N/A | 19 | N/A | 10 | N/A | 4 |
| NSCLC_27 | 32 | 67 | 11 | 25 | 3 | 15 | 1 | 9 |
| NSCLC_28 | N/A | 34 | N/A | 16 | N/A | 11 | N/A | 7 |
| NSCLC_29 | 20 | 36 | 9 | 17 | 7 | 12 | 3 | 10 |
| NSCLC_30 | 16 | 47 | 13 | 13 | 8 | 3 | 6 | 1 |
| NSCLC_31 | 10 | 17 | 4 | 7 | 4 | 4 | 3 | 3 |
| NSCLC_32 | N/A | 34 | N/A | 29 | N/A | 14 | N/A | 10 |
| NSCLC_33 | 18 | 47 | 7 | 13 | 1 | 4 | 1 | 2 |
| NSCLC_34 | N/A | 48 | N/A | 24 | N/A | 13 | N/A | 6 |
| NSCLC_35 | 34 | 34 | 10 | 18 | 7 | 6 | 6 | 3 |
| NSCLC_36 | N/A | 37 | N/A | 21 | N/A | 14 | N/A | 8 |
| NSCLC_37 | 26 | 26 | 16 | 18 | 10 | 14 | 7 | 8 |
| NSCLC_38 | 8 | 34 | 2 | 15 | 2 | 7 | 2 | 3 |
| NSCLC_39 | 54 | 75 | 32 | 50 | 18 | 29 | 12 | 15 |
| NSCLC_40 | 17 | 33 | 5 | 16 | 3 | 5 | 1 | 1 |
| NSCLC_41 | N/A | 75 | N/A | 36 | N/A | 17 | N/A | 11 |
| NSCLC_42 | 29 | 30 | 14 | 12 | 7 | 5 | 4 | 1 |
| NSCLC_43 | N/A | 28 | N/A | 7 | N/A | 3 | N/A | 1 |
| NSCLC_44 | N/A | 64 | N/A | 32 | N/A | 17 | N/A | 9 |
| NSCLC_45 | N/A | 50 | N/A | 25 | N/A | 16 | N/A | 10 |

TABLE 5A
Related to FIG. 3 and FIG. 9.
A. List of CD4+ T cell-transcripts in Module 7 in order of clustering.

| Gene name | Cluster number |
|---|---|
| GPSM3 | Cluster 1 |
| CERS2 | Cluster 1 |
| LY9 | Cluster 1 |
| DEGS1 | Cluster 1 |
| SLC46A3 | Cluster 1 |
| SLC2A1 | Cluster 1 |
| EMP3 | Cluster 1 |
| AKTIP | Cluster 1 |
| NUDCD3 | Cluster 1 |
| DHRS3 | Cluster 1 |
| RBM23 | Cluster 1 |
| PWP2 | Cluster 1 |
| PSAT1 | Cluster 1 |
| GDPD5 | Cluster 1 |
| SPATS2L | Cluster 2 |
| LOC541471 | Cluster 2 |
| IL12RB2 | Cluster 2 |
| STMN1 | Cluster 2 |
| BATF | Cluster 2 |
| TNIP3 | Cluster 2 |
| CD38 | Cluster 2 |
| GBP2 | Cluster 2 |
| C9orf16 | Cluster 2 |
| TNFRSF18 | Cluster 2 |
| LINC00152 | Cluster 2 |
| NME1 | Cluster 2 |
| CXCL13 | Cluster 2 |
| PDCD1 | Cluster 2 |
| UCP2 | Cluster 2 |
| CD7 | Cluster 2 |
| TYMP | Cluster 2 |
| TK1 | Cluster 2 |
| PKM2 | Cluster 2 |
| ENO1 | Cluster 2 |
| RRM2 | Cluster 2 |
| TPI1 | Cluster 2 |
| GAPDH | Cluster 2 |
| TRIM69 | Cluster 2 |
| IL1R2 | Cluster 2 |
| ACTG2 | Cluster 2 |
| SOD1 | Cluster 2 |
| NCAPG | Cluster 2 |
| ZWINT | Cluster 2 |
| H2AFV | Cluster 2 |
| MCM4 | Cluster 2 |
| TNFRSF9 | Cluster 2 |
| GINS2 | Cluster 2 |
| FAM96A | Cluster 2 |
| KIF2C | Cluster 2 |
| LDHA | Cluster 2 |
| MYBL2 | Cluster 2 |
| LMNB1 | Cluster 2 |
| SLC1A4 | Cluster 2 |
| TNFRSF8 | Cluster 2 |
| CDCA5 | Cluster 2 |
| CKAP2L | Cluster 2 |
| MELK | Cluster 2 |
| UHRF1 | Cluster 2 |
| CEP55 | Cluster 2 |
| SMC4 | Cluster 2 |
| ARL6IP1 | Cluster 2 |
| TOP2A | Cluster 2 |
| MTHFD1 | Cluster 2 |
| TPX2 | Cluster 2 |
| MKI67 | Cluster 2 |
| MLF1IP | Cluster 2 |
| FAM111B | Cluster 2 |
| DLGAP5 | Cluster 2 |
| FKBP1A | Cluster 2 |
| BIRC5 | Cluster 2 |
| CDCA8 | Cluster 2 |
| KIAA0101 | Cluster 2 |
| CDK1 | Cluster 2 |
| UBE2C | Cluster 2 |
| DTL | Cluster 2 |
| GLMN | Cluster 3 |
| CDKN2C | Cluster 3 |
| ARL15 | Cluster 3 |
| GBP4 | Cluster 3 |
| EED | Cluster 3 |
| PARP9 | Cluster 3 |
| SYT11 | Cluster 3 |
| STAT1 | Cluster 3 |
| GBP1 | Cluster 3 |
| ASB2 | Cluster 3 |
| UBE2L6 | Cluster 3 |
| PSMB9 | Cluster 3 |
| GBP5 | Cluster 3 |
| SPCS1 | Cluster 3 |
| HIST1H2AH | Cluster 3 |
| GBP1P1 | Cluster 3 |
| CCZ1B | Cluster 3 |
| FAM166B | Cluster 3 |
| HIST1H2BJ | Cluster 3 |
| NEK2 | Cluster 3 |
| CAMK1 | Cluster 3 |
| BTN2A2 | Cluster 3 |
| DEPDC1 | Cluster 3 |
| CCNB1 | Cluster 3 |
| ALDOC | Cluster 3 |
| SLBP | Cluster 3 |
| RAC3 | Cluster 3 |
| IDH2 | Cluster 3 |
| SLC27A2 | Cluster 3 |
| TYMS | Cluster 3 |
| TPM3 | Cluster 3 |
| CDC45 | Cluster 3 |
| CALM3 | Cluster 3 |
| LEMD1 | Cluster 3 |
| BUB1 | Cluster 3 |
| KIF15 | Cluster 3 |
| MCM10 | Cluster 3 |
| AURKA | Cluster 3 |
| DHFR | Cluster 3 |
| ASAH2B | Cluster 3 |
| MYO5C | Cluster 3 |
| SLC3A2 | Cluster 3 |
| HNRPLL | Cluster 3 |
| H2AFY | Cluster 3 |
| SP140 | Cluster 3 |
| TRPC4AP | Cluster 3 |
| RBPJ | Cluster 3 |
| HMGB1 | Cluster 3 |
| SNORD70 | Cluster 3 |
| C11orf82 | Cluster 3 |
| RAB27A | Cluster 3 |
| LYST | Cluster 3 |
| NCAPH | Cluster 3 |
| RFC5 | Cluster 3 |
| ZNF593 | Cluster 3 |
| TMSB10 | Cluster 3 |
| COX8A | Cluster 3 |
| SUB1 | Cluster 3 |
| CKS1B | Cluster 3 |
| EPSTI1 | Cluster 3 |
| TUBB | Cluster 3 |
| RANBP1 | Cluster 3 |
| LMCD1 | Cluster 3 |
| LOC10050752 | Cluster 3 |
| TIGIT | Cluster 3 |
| SIRPG | Cluster 3 |
| MAP1LC3A | Cluster 3 |
| WARS | Cluster 3 |
| ZBED2 | Cluster 3 |
| SLC44A3 | Cluster 4 |
| RARRES3 | Cluster 4 |
| IDO1 | Cluster 5 |
| COPS2 | Cluster 5 |
| SLC35A3 | Cluster 5 |
| HELLS | Cluster 5 |
| NEBL | Cluster 5 |

TABLE 5B
List of CD8+ T cell-transcripts in Module 7 in order of clustering

| Gene name | Cluster number |
|---|---|
| SCARNA17 | Cluster 1 |
| SH2B3 | Cluster 1 |
| PITPNC1 | Cluster 1 |
| XPO6 | Cluster 1 |
| LSR | Cluster 1 |
| TRLM44 | Cluster 1 |
| FXYD2 | Cluster 1 |
| C1orf21 | Cluster 1 |
| TCF7 | Cluster 1 |
| ICAM2 | Cluster 1 |
| LTB | Cluster 1 |
| C20orf3 | Cluster 1 |
| ZDHHC2 | Cluster 1 |
| LYAR | Cluster 1 |
| RAB9A | Cluster 1 |
| TP73 | Cluster 2 |
| KIF18B | Cluster 2 |
| MND1 | Cluster 2 |
| CDC20 | Cluster 2 |
| UBE2L6 | Cluster 2 |
| TK1 | Cluster 2 |
| CDKN3 | Cluster 2 |
| FKBP1A | Cluster 2 |
| GAPDH | Cluster 2 |
| PGAM1 | Cluster 2 |
| TUBB | Cluster 2 |
| HMGN2 | Cluster 2 |
| MCM4 | Cluster 2 |
| ASF1B | Cluster 2 |
| TPI1 | Cluster 2 |
| CD38 | Cluster 2 |
| HIST1H2AH | Cluster 2 |
| KIF15 | Cluster 2 |
| MCM6 | Cluster 2 |
| MCM2 | Cluster 2 |
| CDC45 | Cluster 2 |
| CCNE2 | Cluster 2 |
| CKAP2L | Cluster 2 |
| FEN1 | Cluster 2 |
| KIR2DL4 | Cluster 2 |
| TNS3 | Cluster 2 |
| ZWINT | Cluster 2 |
| ETV7 | Cluster 2 |
| KPNA2 | Cluster 2 |
| HAVCR2 | Cluster 2 |
| RRM2 | Cluster 2 |
| STMN1 | Cluster 2 |
| KIAA0101 | Cluster 2 |
| DLGAP5 | Cluster 2 |
| BIRC5 | Cluster 2 |
| AURKB | Cluster 2 |
| CDCA2 | Cluster 2 |
| DTL | Cluster 2 |
| PKMYT1 | Cluster 2 |
| TYMS | Cluster 2 |
| TOP2A | Cluster 2 |
| LOC100507600 | Cluster 2 |
| TPX2 | Cluster 2 |
| RACGAP1 | Cluster 2 |
| PLK1 | Cluster 2 |
| CCNA2 | Cluster 2 |
| NUSAP1 | Cluster 2 |
| KIF23 | Cluster 2 |
| FBXO5 | Cluster 2 |
| BUB1 | Cluster 2 |
| GINS2 | Cluster 2 |
| SLC25A5 | Cluster 2 |
| NEIL3 | Cluster 2 |
| TOMM34 | Cluster 2 |
| MELK | Cluster 2 |
| HIST1H3G | Cluster 2 |
| HIST1H2AJ | Cluster 2 |
| EPSTI1 | Cluster 2 |
| ANXA5 | Cluster 2 |
| CASC5 | Cluster 2 |
| UHRF1 | Cluster 2 |
| CLSPN | Cluster 2 |
| RANBP1 | Cluster 2 |
| HJURP | Cluster 2 |
| MYBL2 | Cluster 2 |
| HIST1H2AM | Cluster 2 |
| FABP5 | Cluster 2 |
| MLF1IP | Cluster 2 |
| GZMB | Cluster 2 |
| MAD2L1 | Cluster 2 |
| PSMC3 | Cluster 2 |
| RAN | Cluster 2 |
| PRDX6 | Cluster 2 |
| NCAPG | Cluster 2 |
| CENPF | Cluster 2 |
| MKI67 | Cluster 2 |
| MTHFD2 | Cluster 2 |
| UBE2T | Cluster 2 |
| HIST1H4C | Cluster 2 |
| HIST1H1B | Cluster 2 |
| ESCO2 | Cluster 2 |
| CDCA7 | Cluster 2 |
| VDR | Cluster 2 |
| GTSE1 | Cluster 2 |
| PIF1 | Cluster 2 |
| CDCA8 | Cluster 2 |
| CCNB2 | Cluster 2 |
| TROAP | Cluster 2 |
| LOC541471 | Cluster 2 |
| GEM | Cluster 2 |
| FANCI | Cluster 2 |
| CCNB1 | Cluster 2 |
| WDHD1 | Cluster 2 |
| RFC2 | Cluster 2 |
| PDIA6 | Cluster 2 |
| ENO1 | Cluster 2 |
| CD82 | Cluster 2 |
| CHMP4A | Cluster 2 |
| CSF1 | Cluster 2 |
| PTPN7 | Cluster 2 |
| PRKAG1 | Cluster 2 |
| C3orf14 | Cluster 2 |
| GBP4 | Cluster 2 |
| STAT1 | Cluster 2 |
| GBP1 | Cluster 2 |
| PTMA | Cluster 2 |
| RRM1 | Cluster 2 |
| HPRT1 | Cluster 2 |
| ARPC2 | Cluster 2 |
| PGK1 | Cluster 2 |
| LDHA | Cluster 2 |
| KIF11 | Cluster 2 |
| GALNT2 | Cluster 2 |
| PKM2 | Cluster 2 |
| MCM5 | Cluster 2 |
| GBP2 | Cluster 2 |
| ITGAE | Cluster 2 |
| SMC2 | Cluster 2 |
| COTL1 | Cluster 2 |
| COX5A | Cluster 2 |
| DUT | Cluster 2 |
| TUBA1B | Cluster 2 |
| ACOT7 | Cluster 2 |
| TALDO1 | Cluster 2 |
| WARS | Cluster 2 |
| CHEK1 | Cluster 2 |
| NDFIP2 | Cluster 2 |
| WDR34 | Cluster 2 |
| BST2 | Cluster 2 |
| PSME2 | Cluster 2 |
| CALM3 | Cluster 2 |
| TOX2 | Cluster 2 |
| SHMT2 | Cluster 2 |
| TRAFD1 | Cluster 2 |
| ID3 | Cluster 2 |
| SARDH | Cluster 2 |
| HAPLN3 | Cluster 2 |
| IGFLR1 | Cluster 2 |
| PSMD8 | Cluster 2 |
| IFI35 | Cluster 2 |
| GBP5 | Cluster 2 |
| OBFC2B | Cluster 2 |
| ANKRD35 | Cluster 2 |
| E2F2 | Cluster 2 |
| PSMB9 | Cluster 2 |
| SNRPB | Cluster 2 |
| FDPS | Cluster 2 |
| SPC24 | Cluster 2 |
| MAD2L2 | Cluster 2 |
| CDK1 | Cluster 2 |
| CKS1B | Cluster 2 |
| SNAP47 | Cluster 2 |
| CXCR6 | Cluster 2 |
| P2RY1 | Cluster 2 |
| CXorf69 | Cluster 2 |
| CCL3 | Cluster 2 |
| C16orf59 | Cluster 2 |
| PBK | Cluster 2 |
| EXO1 | Cluster 2 |
| CCDC74A | Cluster 2 |
| PCNA | Cluster 2 |
| KIF2C | Cluster 2 |
| PARK7 | Cluster 2 |
| SLC27A2 | Cluster 2 |
| AKAP5 | Cluster 2 |
| GLDC | Cluster 2 |
| SIRPG | Cluster 2 |
| TNFRSF9 | Cluster 2 |
| SCCPDH | Cluster 2 |
| LIMK1 | Cluster 2 |
| TSPAN17 | Cluster 2 |
| NXT2 | Cluster 3 |
| CHST14 | Cluster 3 |
| RASD1 | Cluster 3 |
| MAOB | Cluster 3 |
| ACAT2 | Cluster 3 |
| KLRC2 | Cluster 3 |
| IL21 | Cluster 3 |
| LOC729234 | Cluster 3 |
| IFNG | Cluster 3 |
| SLC50A1 | Cluster 3 |
| HIST1H2BM | Cluster 3 |
| CCDC121 | Cluster 3 |
| HIST1H4L | Cluster 3 |
| TMEM97 | Cluster 3 |
| HIST1H3J | Cluster 3 |
| PRRG4 | Cluster 3 |
| H1F0 | Cluster 3 |
| HIST1H3F | Cluster 3 |
| HIST1H3H | Cluster 3 |
| HIST1H2AE | Cluster 3 |
| PIK3AP1 | Cluster 3 |
| SPRY2 | Cluster 3 |
| CHAF1A | Cluster 3 |
| IRF7 | Cluster 3 |
| FAM3C | Cluster 3 |
| MYO1E | Cluster 3 |
| KCNK1 | Cluster 3 |
| WDR65 | Cluster 3 |
| UQCRC2 | Cluster 4 |
| LOC254559 | Cluster 4 |
| LOC100506274 | Cluster 4 |
| SLC7A5 | Cluster 4 |
| GALNT1 | Cluster 4 |
| POC1A | Cluster 4 |
| FKBP4 | Cluster 4 |
| H2AFV | Cluster 4 |
| FAM64A | Cluster 4 |
| GSTZ1 | Cluster 4 |
| CCDC86 | Cluster 4 |
| POLE2 | Cluster 4 |
| PTMS | Cluster 4 |
| GPR19 | Cluster 4 |
| AMZ1 | Cluster 4 |
| MAPK12 | Cluster 4 |
| PMVK | Cluster 4 |
| CHAC2 | Cluster 4 |
| HIST1H2AL | Cluster 4 |
| E2F1 | Cluster 4 |
| NDC80 | Cluster 4 |
| HIST1H2BF | Cluster 4 |
| SCUBE1 | Cluster 4 |
| DPF3 | Cluster 4 |
| TMEM206 | Cluster 4 |
| HMGB3 | Cluster 4 |
| CDCA5 | Cluster 4 |
| CENPH | Cluster 4 |
| PAQR4 | Cluster 4 |
| NCAPH | Cluster 4 |
| WDR76 | Cluster 4 |
| TRAIP | Cluster 4 |
| PARP2 | Cluster 4 |
| HIST1H3C | Cluster 4 |
| STIL | Cluster 4 |
| EME1 | Cluster 4 |
| NUF2 | Cluster 4 |
| CENPN | Cluster 4 |
| HADH | Cluster 4 |
| GBP1P1 | Cluster 4 |
| YWHAE | Cluster 4 |
| KIF4A | Cluster 4 |
| APOBEC3B | Cluster 4 |
| FOXM1 | Cluster 4 |
| E2F7 | Cluster 4 |
| PRDX3 | Cluster 4 |
| CDT1 | Cluster 4 |
| DHFR | Cluster 4 |
| SHCBP1 | Cluster 4 |
| RAD51 | Cluster 4 |
| CISD1 | Cluster 4 |
| UBB | Cluster 4 |
| CKS2 | Cluster 4 |
| FAM111B | Cluster 4 |
| DEPDC1 | Cluster 4 |
| TMEM106C | Cluster 4 |
| C11orf75 | Cluster 4 |
| UBE2C | Cluster 4 |
| RAD51AP1 | Cluster 4 |
| CEP55 | Cluster 4 |
| ORC6 | Cluster 4 |
| HMGB2 | Cluster 4 |
| DIAPH3 | Cluster 4 |
| KIFC1 | Cluster 4 |
| HMMR | Cluster 4 |
| CENPW | Cluster 4 |
| CENPM | Cluster 4 |
| CEND1 | Cluster 4 |
| SPAG5 | Cluster 4 |
| ANLN | Cluster 4 |
| E2F8 | Cluster 4 |
| PHGDH | Cluster 4 |
| MCM10 | Cluster 4 |
| SKA3 | Cluster 4 |
| NEK2 | Cluster 4 |
| PPA1 | Cluster 4 |
| CMC2 | Cluster 4 |
| CDCA3 | Cluster 4 |
| CENPA | Cluster 4 |
| SAE1 | Cluster 4 |
| ENSA | Cluster 4 |
| DNAJB11 | Cluster 4 |
| ECH1 | Cluster 4 |
| DONSON | Cluster 4 |
| PDLIM7 | Cluster 4 |
| ID2 | Cluster 4 |
| GOLIM4 | Cluster 4 |
| CTNNAL1 | Cluster 4 |
| GMNN | Cluster 4 |
| CACYBP | Cluster 4 |
| COMMD7 | Cluster 4 |
| TRIP13 | Cluster 4 |
| CCZ1B | Cluster 4 |
| PTPRK | Cluster 4 |
| PPAP2A | Cluster 4 |
| ETV1 | Cluster 4 |
| TNFSF4 | Cluster 4 |
| LINC00158 | Cluster 4 |
| INPP5F | Cluster 4 |
| ZBED2 | Cluster 4 |
| PDCD1 | Cluster 4 |
| PHEX | Cluster 4 |
| IFITM10 | Cluster 4 |
| CAMK1 | Cluster 4 |
| LAYN | Cluster 4 |
| NAB1 | Cluster 4 |
| PDLIM4 | Cluster 4 |
| PET112 | Cluster 4 |
| WIPF3 | Cluster 4 |
| AFAP1L2 | Cluster 4 |
| TWF2 | Cluster 4 |
| SEMA4A | Cluster 4 |
| LOC100506668 | Cluster 4 |
| MCM7 | Cluster 4 |
| ZNF367 | Cluster 4 |
| DDX49 | Cluster 4 |
| SMTN | Cluster 4 |
| REEP2 | Cluster 4 |
| TTYH3 | Cluster 4 |
| ORC1 | Cluster 4 |
| KIF20A | Cluster 4 |
| KCNK5 | Cluster 4 |
| MYO5B | Cluster 4 |
| SLC4A2 | Cluster 4 |
| KRT86 | Cluster 5 |
| TIGIT | Cluster 5 |
| UBE2F | Cluster 5 |
| TSHZ2 | Cluster 5 |
| VOPP1 | Cluster 5 |
| NOTCH1 | Cluster 5 |
| GPR25 | Cluster 5 |
| PKIA | Cluster 5 |
| CALR | Cluster 5 |
| CD160 | Cluster 5 |
| MXD3 | Cluster 5 |
| SPIN4 | Cluster 5 |
| LRRC61 | Cluster 5 |
| CLIP3 | Cluster 5 |
| MAP1LC3A | Cluster 5 |
| FASN | Cluster 5 |
| OAZ1 | Cluster 5 |
| ATP5B | Cluster 5 |
| HNRNPF | Cluster 5 |
| CCT3 | Cluster 5 |
| FAM110A | Cluster 5 |
| IDH2 | Cluster 5 |
| MDH2 | Cluster 5 |
| CPNE7 | Cluster 5 |
| TNFSF10 | Cluster 5 |
| TREX1 | Cluster 5 |
| NKG7 | Cluster 5 |
| IL32 | Cluster 5 |
| PSMB10 | Cluster 5 |
| FIBP | Cluster 5 |
| ELOF1 | Cluster 5 |
| COPE | Cluster 5 |
| LAG3 | Cluster 5 |
| PFN1 | Cluster 5 |
| PSME1 | Cluster 5 |
| LSM2 | Cluster 5 |
| ASNA1 | Cluster 5 |
| CCL5 | Cluster 5 |
| PPIB | Cluster 5 |
| C18orf56 | Cluster 5 |
| MVD | Cluster 5 |
| C9orf16 | Cluster 5 |
| SDHC | Cluster 5 |
| KIAA1671 | Cluster 5 |
| CCRL2 | Cluster 5 |
| PSMB7 | Cluster 5 |
| GSTM4 | Cluster 5 |
| GPR34 | Cluster 5 |
| CSNK2B | Cluster 5 |
| ECHS1 | Cluster 5 |
| UBL7 | Cluster 5 |
| EIF3I | Cluster 5 |
| RALY | Cluster 5 |
| FBXO6 | Cluster 5 |
| YBX1 | Cluster 5 |
| MCM3 | Cluster 5 |
| GTF3C6 | Cluster 5 |
| POLD1 | Cluster 5 |
| H2AFX | Cluster 5 |
| PMF1 | Cluster 5 |
| BARD1 | Cluster 5 |
| PHF19 | Cluster 5 |
| TFDP1 | Cluster 5 |
| RDM1 | Cluster 5 |
| SFXN2 | Cluster 5 |
| DAPK2 | Cluster 5 |
| DCPS | Cluster 5 |
| FDFT1 | Cluster 5 |
| SUMO2 | Cluster 5 |
| KIAA1524 | Cluster 5 |
| RFC3 | Cluster 5 |
| TBK1 | Cluster 5 |
| RAD17 | Cluster 5 |
| NETO2 | Cluster 5 |
| SNORD74 | Cluster 5 |
| CHCHD4 | Cluster 5 |
| GPSM2 | Cluster 5 |
| LOC284581 | Cluster 5 |
| CDT1 | Cluster 5 |
| AK4 | Cluster 5 |
| TUBA1B | Cluster 5 |
| JAKMIP1 | Cluster 5 |
| SKA2 | Cluster 5 |
| ABI1 | Cluster 5 |
| COX4NB | Cluster 5 |
| SAMD9L | Cluster 5 |
| CHST11 | Cluster 5 |
| VDAC2 | Cluster 5 |
| SEPT2 | Cluster 5 |
| ATAD5 | Cluster 5 |
| KIF20B | Cluster 5 |
| FAM193A | Cluster 5 |
| MRC2 | Cluster 5 |
| RDH10 | Cluster 5 |
| PRR19 | Cluster 5 |
| CTLA4 | Cluster 5 |

TABLE 6
Related to FIG. 3.
List of nodes for network analysis in Module 7.

| Node | Cell type |
|---|---|
| AK4 | CD4+ TIL |
| BATF | CD4+ TIL |
| BIRC5 | CD4+ TIL |
| BUB1 | CD4+ TIL |
| C11orf82 | CD4+ TIL |
| CAMK1 | CD4+ TIL |
| CD38 | CD4+ TIL |
| CDCA5 | CD4+ TIL |
| CDK1 | CD4+ TIL |
| CDT1 | CD4+ TIL |
| CEP55 | CD4+ TIL |
| CKAP2L | CD4+ TIL |
| CXCL13 | CD4+ TIL |
| DLGAP5 | CD4+ TIL |
| EPSTI1 | CD4+ TIL |
| FAM111B | CD4+ TIL |
| GAPDH | CD4+ TIL |
| GBP1P1 | CD4+ TIL |
| GBP4 | CD4+ TIL |
| GINS2 | CD4+ TIL |
| H2AFV | CD4+ TIL |
| HIST1H2AH | CD4+ TIL |
| KIAA0101 | CD4+ TIL |
| KIF15 | CD4+ TIL |
| KIF2C | CD4+ TIL |
| LOC541471 | CD4+ TIL |
| MCM10 | CD4+ TIL |
| MCM4 | CD4+ TIL |
| MELK | CD4+ TIL |
| MKI67 | CD4+ TIL |
| MLF1IP | CD4+ TIL |
| MRC2 | CD4+ TIL |
| MTHFD1 | CD4+ TIL |
| MYBL2 | CD4+ TIL |
| NCAPG | CD4+ TIL |
| NUDCD3 | CD4+ TIL |
| PSMB9 | CD4+ TIL |
| RANBP1 | CD4+ TIL |
| RRM2 | CD4+ TIL |
| SNORD70 | CD4+ TIL |
| SNORD74 | CD4+ TIL |
| SPATS2L | CD4+ TIL |
| STMN1 | CD4+ TIL |
| TNIP3 | CD4+ TIL |
| TOP2A | CD4+ TIL |
| TPX2 | CD4+ TIL |
| TRIM69 | CD4+ TIL |
| UBE2C | CD4+ TIL |
| UHRF1 | CD4+ TIL |
| ZWINT | CD4+ TIL |
| ACOT7 | CD8+ TIL |
| ANLN | CD8+ TIL |
| ANXA5 | CD8+ TIL |
| APOBEC3B | CD8+ TIL |
| ARPC2 | CD8+ TIL |
| ASF1B | CD8+ TIL |
| ATP5B | CD8+ TIL |
| AURKB | CD8+ TIL |
| BIRC5 | CD8+ TIL |
| BST2 | CD8+ TIL |
| BUB1 | CD8+ TIL |
| C11orf75 | CD8+ TIL |
| C9orf16 | CD8+ TIL |
| CACYBP | CD8+ TIL |
| CALM3 | CD8+ TIL |
| CAMK1 | CD8+ TIL |
| CASC5 | CD8+ TIL |
| CCDC74A | CD8+ TIL |
| CCL3 | CD8+ TIL |
| CCNA2 | CD8+ TIL |
| CCNB1 | CD8+ TIL |
| CCNB2 | CD8+ TIL |
| CCNE2 | CD8+ TIL |
| CD38 | CD8+ TIL |
| CD82 | CD8+ TIL |
| CDC20 | CD8+ TIL |
| CDC45 | CD8+ TIL |
| CDCA2 | CD8+ TIL |
| CDCA3 | CD8+ TIL |
| CDCA5 | CD8+ TIL |
| CDCA7 | CD8+ TIL |
| CDCA8 | CD8+ TIL |
| CDK1 | CD8+ TIL |
| CDKN3 | CD8+ TIL |
| CDT1 | CD8+ TIL |
| CENPA | CD8+ TIL |
| CENPF | CD8+ TIL |
| CENPM | CD8+ TIL |
| CENPN | CD8+ TIL |
| CENPW | CD8+ TIL |
| CEP55 | CD8+ TIL |
| CHEK1 | CD8+ TIL |
| CHST14 | CD8+ TIL |
| CKAP2L | CD8+ TIL |
| CKS1B | CD8+ TIL |
| CKS2 | CD8+ TIL |
| CLIP3 | CD8+ TIL |
| CLSPN | CD8+ TIL |
| COTL1 | CD8+ TIL |
| COX5A | CD8+ TIL |
| CSF1 | CD8+ TIL |
| CTNNAL1 | CD8+ TIL |
| CXorf69 | CD8+ TIL |
| DEPDC1 | CD8+ TIL |
| DHFR | CD8+ TIL |
| DIAPH3 | CD8+ TIL |
| DLGAP5 | CD8+ TIL |
| DPF3 | CD8+ TIL |
| DTL | CD8+ TIL |
| DUT | CD8+ TIL |
| E2F2 | CD8+ TIL |
| E2F7 | CD8+ TIL |
| E2F8 | CD8+ TIL |
| EME1 | CD8+ TIL |
| ENO1 | CD8+ TIL |
| EPSTI1 | CD8+ TIL |
| ETV1 | CD8+ TIL |
| ETV7 | CD8+ TIL |
| EXO1 | CD8+ TIL |
| FABP5 | CD8+ TIL |
| FAM111B | CD8+ TIL |
| FAM3C | CD8+ TIL |
| FAM64A | CD8+ TIL |
| FANCI | CD8+ TIL |
| FBXO5 | CD8+ TIL |
| FBXO6 | CD8+ TIL |
| FDFT1 | CD8+ TIL |
| FEN1 | CD8+ TIL |
| FKBP1A | CD8+ TIL |
| FOXM1 | CD8+ TIL |
| GAPDH | CD8+ TIL |
| GBP1 | CD8+ TIL |
| GBP1P1 | CD8+ TIL |
| GBP2 | CD8+ TIL |
| GBP4 | CD8+ TIL |
| GBP5 | CD8+ TIL |
| GEM | CD8+ TIL |
| GINS2 | CD8+ TIL |
| GMNN | CD8+ TIL |
| GSTM4 | CD8+ TIL |
| GTSE1 | CD8+ TIL |
| GZMB | CD8+ TIL |
| H2AFX | CD8+ TIL |
| HADH | CD8+ TIL |
| HAPLN3 | CD8+ TIL |
| HAVCR2 | CD8+ TIL |
| HIST1H1B | CD8+ TIL |
| HIST1H2AH | CD8+ TIL |
| HIST1H2AJ | CD8+ TIL |
| HIST1H2AM | CD8+ TIL |
| HIST1H2BF | CD8+ TIL |
| HIST1H2BM | CD8+ TIL |
| HIST1H3C | CD8+ TIL |
| HIST1H3G | CD8+ TIL |
| HIST1H3H | CD8+ TIL |
| HJURP | CD8+ TIL |
| HMGB2 | CD8+ TIL |
| HMGB3 | CD8+ TIL |
| HMGN2 | CD8+ TIL |
| HMMR | CD8+ TIL |
| ICAM2 | CD8+ TIL |
| IDH2 | CD8+ TIL |
| IFI35 | CD8+ TIL |
| IFITM10 | CD8+ TIL |
| IGFLR1 | CD8+ TIL |
| ITGAE | CD8+ TIL |
| KIAA0101 | CD8+ TIL |
| KIF11 | CD8+ TIL |
| KIF15 | CD8+ TIL |
| KIF18B | CD8+ TIL |
| KIF20A | CD8+ TIL |
| KIF23 | CD8+ TIL |
| KIF4A | CD8+ TIL |
| KIFC1 | CD8+ TIL |
| KPNA2 | CD8+ TIL |
| LAG3 | CD8+ TIL |
| LDHA | CD8+ TIL |
| LIMK1 | CD8+ TIL |
| LOC541471 | CD8+ TIL |
| MAD2L1 | CD8+ TIL |
| MAD2L2 | CD8+ TIL |
| MAPK12 | CD8+ TIL |
| MCM10 | CD8+ TIL |
| MCM2 | CD8+ TIL |
| MCM4 | CD8+ TIL |
| MCM5 | CD8+ TIL |
| MCM7 | CD8+ TIL |
| MELK | CD8+ TIL |
| MKI67 | CD8+ TIL |
| MLF1IP | CD8+ TIL |
| MND1 | CD8+ TIL |
| MXD3 | CD8+ TIL |
| NCAPG | CD8+ TIL |
| NCAPH | CD8+ TIL |
| NDC80 | CD8+ TIL |
| NEIL3 | CD8+ TIL |
| NEK2 | CD8+ TIL |
| NUF2 | CD8+ TIL |
| NUSAP1 | CD8+ TIL |
| OAZ1 | CD8+ TIL |
| P2RY1 | CD8+ TIL |
| PBK | CD8+ TIL |
| PCNA | CD8+ TIL |
| PDIA6 | CD8+ TIL |
| PDLIM4 | CD8+ TIL |
| PFN1 | CD8+ TIL |
| PGAM1 | CD8+ TIL |
| PGK1 | CD8+ TIL |
| PHEX | CD8+ TIL |
| PHGDH | CD8+ TIL |
| PIF1 | CD8+ TIL |
| PKM2 | CD8+ TIL |
| PKMYT1 | CD8+ TIL |
| PLK1 | CD8+ TIL |
| PPA1 | CD8+ TIL |
| PRDX6 | CD8+ TIL |
| PRKAG1 | CD8+ TIL |
| PSMB7 | CD8+ TIL |
| PSMC3 | CD8+ TIL |
| PSME1 | CD8+ TIL |
| PSME2 | CD8+ TIL |
| PTMA | CD8+ TIL |
| PTMS | CD8+ TIL |
| PTPN7 | CD8+ TIL |
| RACGAP1 | CD8+ TIL |
| RAD51AP1 | CD8+ TIL |
| RAD51 | CD8+ TIL |
| RANBP1 | CD8+ TIL |
| RAN | CD8+ TIL |
| REEP2 | CD8+ TIL |
| RFC2 | CD8+ TIL |
| RRM2 | CD8+ TIL |
| SCARNA17 | CD8+ TIL |
| SCCPDH | CD8+ TIL |
| SEMA4A | CD8+ TIL |
| SHCBP1 | CD8+ TIL |
| SHMT2 | CD8+ TIL |
| SMC2 | CD8+ TIL |
| SNRPB | CD8+ TIL |
| SPAG5 | CD8+ TIL |
| SPC24 | CD8+ TIL |
| STAT1 | CD8+ TIL |
| STIL | CD8+ TIL |
| STMN1 | CD8+ TIL |
| TCF7 | CD8+ TIL |
| TIGIT | CD8+ TIL |
| TK1 | CD8+ TIL |
| TNFRSF9 | CD8+ TIL |
| TNS3 | CD8+ TIL |
| TOP2A | CD8+ TIL |
| TOX2 | CD8+ TIL |
| TP73 | CD8+ TIL |
| TPI1 | CD8+ TIL |
| TPX2 | CD8+ TIL |
| TRAIP | CD8+ TIL |
| TRIP13 | CD8+ TIL |
| TROAP | CD8+ TIL |
| TSPAN17 | CD8+ TIL |
| TUBA1B | CD8+ TIL |
| TUBB | CD8+ TIL |
| TYMS | CD8+ TIL |
| UBB | CD8+ TIL |
| UBE2C | CD8+ TIL |
| UBE2L6 | CD8+ TIL |
| UBE2T | CD8+ TIL |
| UHRF1 | CD8+ TIL |
| VDR | CD8+ TIL |
| WARS | CD8+ TIL |
| ZBED2 | CD8+ TIL |
| ZWINT | CD8+ TIL |

TABLE 7
Related to FIG. 4 and FIG. 10.
List of Seurat cluster-specific genes for CD4⁺ TILs.

| Gene | Average log₂ fold change | Adjusted P value |
|---|---|---|
| CXCL13 | 2.59 | 3.53E−279 |
| AC092580.4 | 1.07 | 2.52E−76 |
| CCL4 | 0.96 | 7.14E−10 |
| NR3C1 | 0.91 | 4.60E−123 |
| FKBP5 | 0.86 | 8.16E−93 |
| RBPJ | 0.81 | 4.62E−72 |
| CHN1 | 0.78 | 1.22E−50 |
| KLRB1 | 0.75 | 6.73E−51 |
| IFNG | 0.71 | 2.84E−25 |
| ALOX5AP | 0.70 | 1.07E−55 |
| BTLA | 0.69 | 5.99E−48 |
| SRGN | 0.68 | 8.19E−107 |
| MAF | 0.68 | 5.61E−50 |
| IL6ST | 0.61 | 1.42E−35 |
| RNF19A | 0.59 | 9.80E−48 |
| ITM2A | 0.59 | 5.92E−41 |
| TNFRSF18 | 0.58 | 6.41E−49 |
| SNX9 | 0.57 | 8.16E−38 |
| CD7 | 0.56 | 1.33E−32 |
| NAP1L4 | 0.56 | 2.03E−36 |
| ICA1 | 0.54 | 8.29E−24 |
| PDCD1 | 0.54 | 3.07E−34 |
| TSHZ2 | 0.54 | 2.44E−34 |
| FABP5 | 0.54 | 1.32E−27 |
| RP5-1028K7.2 | 0.52 | 5.60E−29 |
| SLA | 0.51 | 4.90E−31 |
| UCP2 | 0.49 | 6.65E−22 |
| CTSD | 0.49 | 4.66E−22 |
| ANKRD28 | 0.49 | 1.90E−13 |
| YWHAQ | 0.48 | 1.36E−24 |
| ID2 | 0.48 | 2.77E−22 |
| MT2A | 0.48 | 2.30E−17 |
| PKM | 0.48 | 1.71E−22 |
| LIMS1 | 0.48 | 9.52E−25 |
| BST2 | 0.47 | 3.35E−19 |
| PGAM1 | 0.47 | 8.38E−23 |
| ELMO1 | 0.46 | 6.06E−22 |
| GAPDH | 0.45 | 1.18E−45 |
| RAB27A | 0.44 | 5.49E−23 |
| TPI1 | 0.44 | 3.54E−22 |
| COTL1 | 0.43 | 8.17E−25 |
| DUSP4 | 0.43 | 1.51E−19 |
| ENTPD1 | 0.42 | 2.48E−17 |
| RGS2 | 0.40 | 2.68E−13 |
| SH2D1A | 0.40 | 1.79E−15 |
| CD2 | 0.39 | 8.45E−30 |
| LAPTM5 | 0.39 | 6.87E−22 |
| CD82 | 0.38 | 6.78E−16 |
| LYST | 0.38 | 1.58E−12 |
| PPP1CC | 0.37 | 1.76E−15 |
| IQGAP1 | 0.37 | 1.86E−14 |
| FKBP1A | 0.36 | 4.27E−13 |
| C9orf16 | 0.36 | 3.17E−14 |
| SEC11A | 0.35 | 3.72E−13 |
| CD3D | 0.35 | 4.07E−27 |
| LBH | 0.35 | 5.99E−11 |
| TMEM167A | 0.34 | 3.42E−11 |
| H2AFZ | 0.34 | 1.37E−09 |
| CKLF | 0.33 | 1.63E−11 |
| RPL7L1 | 0.33 | 9.22E−11 |
| VOPP1 | 0.33 | 3.06E−13 |
| MSI2 | 0.33 | 3.65E−10 |
| SMAP2 | 0.33 | 6.03E−09 |
| ITGA4 | 0.33 | 3.15E−11 |
| CTLA4 | 0.33 | 6.92E−27 |
| CITED2 | 0.33 | 2.13E−05 |
| SPOCK2 | 0.33 | 4.98E−12 |
| RILPL2 | 0.33 | 1.16E−08 |
| METTL8 | 0.33 | 2.57E−10 |
| RHOA | 0.32 | 9.62E−12 |
| TMBIM6 | 0.32 | 1.67E−13 |
| ANXA5 | 0.32 | 1.35E−08 |
| CD247 | 0.32 | 1.50E−09 |
| LY6E | 0.31 | 3.04E−07 |
| CD84 | 0.31 | 1.30E−08 |
| HMGB1 | 0.31 | 2.09E−23 |
| HMGN1 | 0.31 | 4.09E−13 |
| ECH1 | 0.31 | 8.97E−10 |
| PHPT1 | 0.31 | 3.53E−09 |
| FAM107B | 0.30 | 5.53E−09 |
| HIF1A | 0.30 | 1.12E−06 |
| ISCU | 0.30 | 4.36E−09 |
| TSPO | 0.30 | 5.56E−08 |
| PDIA6 | 0.30 | 1.63E−06 |
| SOD1 | 0.30 | 2.16E−11 |
| GADD45G | 0.30 | 2.85E−05 |
| CD40LG | 0.30 | 4.56E−06 |
| ACTB | 0.30 | 1.17E−31 |
| SHFM1 | 0.30 | 1.32E−11 |
| SPCS2 | 0.29 | 2.97E−10 |
| PARK7 | 0.29 | 1.12E−09 |
| ARAP2 | 0.29 | 8.56E−07 |
| WDR83OS | 0.29 | 3.89E−09 |
| SERF2 | 0.29 | 6.86E−23 |
| HMGN3 | 0.29 | 7.04E−07 |
| NDUFV2 | 0.29 | 1.15E−07 |
| RGS1 | 0.29 | 5.35E−19 |
| SH3BGRL3 | 0.28 | 2.90E−12 |
| HP1BP3 | 0.28 | 3.09E−09 |
| RTFDC1 | 0.28 | 2.16E−08 |
| TANK | 0.28 | 2.27E−06 |
| CCND3 | 0.28 | 8.78E−08 |
| OXNAD1 | 0.28 | 6.25E−06 |
| DNPH1 | 0.28 | 1.57E−07 |
| PAG1 | 0.28 | 6.72E−06 |
| TCEB2 | 0.28 | 2.28E−10 |
| SEPT7 | 0.27 | 1.28E−09 |
| GNG5 | 0.27 | 3.46E−06 |
| MYCBP2 | 0.27 | 6.73E−07 |
| PDIA3 | 0.27 | 2.06E−07 |
| PTPRC | 0.27 | 4.29E−24 |
| VDAC1 | 0.27 | 9.56E−08 |
| PTPN7 | 0.27 | 2.59E−06 |
| COX8A | 0.27 | 7.66E−09 |
| TIGIT | 0.27 | 2.51E−16 |
| CTSB | 0.27 | 2.31E−10 |
| ANAPC16 | 0.27 | 1.09E−07 |
| EID1 | 0.26 | 1.28E−05 |
| SPCS1 | 0.26 | 4.45E−07 |
| SAMSN1 | 0.26 | 6.83E−07 |
| S100A11 | 0.26 | 1.49E−05 |
| LSP1 | 0.26 | 8.51E−06 |
| ARPC4 | 0.26 | 1.09E−06 |
| PPP1R7 | 0.26 | 1.37E−05 |
| TMEM50A | 0.26 | 4.96E−06 |
| ITGB1 | 0.26 | 0.001340903 |
| GALM | 0.26 | 0.000119486 |
| BSG | 0.26 | 2.76E−06 |
| C4orf48 | 0.26 | 1.27E−05 |
| NDUFA13 | 0.26 | 6.64E−07 |
| CNOT6L | 0.26 | 0.000101892 |
| CCDC167 | 0.25 | 5.51E−06 |
| TMEM173 | 0.25 | 0.003201356 |
| ARPC1B | 0.25 | 2.87E−07 |
| BRK1 | 0.25 | 0.000124877 |
| PHACTR2 | 0.25 | 0.000101417 |
| H3F3A | 0.25 | 2.14E−11 |
| GPX4 | 0.25 | 6.84E−05 |

TABLE 8
Related to FIG. 4.
List of differentially expressed genes in CXCL13-expressing versus CXCL13-non-
expressing CD4⁺ TILs.

| | | | Mean CPM (counts | | Percentage of | |
|---|---|---|---|---|---|---|
| | Log₂ fold | Adjusted | per million) | | expressing cells | |
| Gene name | change | P value | CXCL13⁻ | CXCL13⁺ | CXCL13⁻ | CXCL13⁺ |
| AC006129.4 | 0.33 | 1.01E−08 | 11.05 | 36.39 | 1.88 | 7.43 |
| AC018816.3 | 0.24 | 1.13E−02 | 15.10 | 35.36 | 3.28 | 7.33 |
| AC069363.1 | 0.45 | 7.52E−19 | 4.76 | 36.50 | 0.66 | 6.18 |
| AC092580.4 | 1.74 | 1.48E−36 | 164.44 | 487.27 | 19.26 | 37.91 |
| AC104820.2 | 0.48 | 2.07E−05 | 33.49 | 73.16 | 7.27 | 15.29 |
| AC112721.2 | 0.07 | 1.28E−02 | 1.23 | 5.36 | 0.25 | 1.68 |
| ACADVL | 0.32 | 3.31E−02 | 49.76 | 71.73 | 10.82 | 17.49 |
| ACSL3 | 0.06 | 4.55E−02 | 19.29 | 20.56 | 4.26 | 6.28 |
| ACTB | 0.46 | 3.40E−26 | 3954.80 | 5231.59 | 97.48 | 98.43 |
| ACTG1 | 0.63 | 3.68E−10 | 968.16 | 1238.11 | 77.74 | 84.71 |
| AF165138.7 | 0.03 | 3.80E−02 | 0.00 | 3.55 | 0.00 | 0.52 |
| AGFG1 | 0.41 | 8.32E−07 | 18.45 | 50.70 | 4.52 | 12.04 |
| AGPAT5 | 0.16 | 3.70E−02 | 10.12 | 26.76 | 2.50 | 5.34 |
| AHI1 | 0.81 | 4.40E−17 | 27.33 | 87.29 | 6.21 | 18.64 |
| AKT3 | 0.13 | 3.87E−02 | 14.84 | 20.60 | 3.51 | 5.97 |
| ALOX5AP | 1.29 | 4.00E−29 | 209.29 | 428.10 | 33.65 | 49.42 |
| ANKRD28 | 0.58 | 2.66E−06 | 89.43 | 172.74 | 13.92 | 21.36 |
| ANKRD35 | 0.37 | 2.12E−11 | 6.56 | 27.63 | 1.28 | 6.39 |
| ANKRD55 | 0.28 | 1.76E−07 | 4.57 | 19.47 | 1.12 | 5.03 |
| ANXA5 | 0.55 | 5.55E−04 | 96.70 | 163.91 | 19.07 | 29.53 |
| APOBEC3C | 0.67 | 6.94E−05 | 103.16 | 189.18 | 16.21 | 25.13 |
| ARL3 | 0.61 | 6.10E−13 | 19.28 | 70.61 | 4.33 | 13.40 |
| ARMC12 | 0.06 | 4.62E−03 | 0.32 | 3.67 | 0.07 | 1.05 |
| ARPC1B | 0.67 | 3.46E−03 | 386.32 | 505.48 | 53.09 | 64.29 |
| ARPC2 | 0.37 | 3.46E−10 | 504.17 | 648.22 | 63.80 | 70.26 |
| ARPC3 | 0.19 | 4.10E−02 | 395.55 | 464.60 | 57.77 | 64.19 |
| ASCL2 | 0.08 | 3.95E−02 | 1.30 | 6.88 | 0.34 | 1.78 |
| ATF7IP | 0.08 | 1.26E−02 | 94.73 | 96.85 | 19.42 | 24.08 |
| ATP5E | 0.36 | 4.43E−11 | 1640.73 | 1940.26 | 91.04 | 94.14 |
| ATP5G2 | 0.24 | 5.67E−04 | 479.92 | 570.77 | 63.20 | 68.90 |
| ATP5I | 0.40 | 2.24E−02 | 361.20 | 448.87 | 54.72 | 62.93 |
| ATP5J2 | 0.54 | 6.02E−04 | 218.90 | 319.81 | 38.88 | 49.32 |
| ATP5L | 0.05 | 5.04E−06 | 643.50 | 741.95 | 73.15 | 75.81 |
| ATP9A | 0.11 | 2.12E−03 | 1.27 | 7.91 | 0.30 | 1.88 |
| AURKA | 0.10 | 3.16E−02 | 4.46 | 11.11 | 0.66 | 2.30 |
| B2M | 0.08 | 4.97E−02 | 16940.03 | 17928.38 | 99.93 | 100.00 |
| BAD | 0.32 | 8.41E−03 | 26.38 | 51.83 | 6.30 | 12.25 |
| BASP1 | 0.10 | 1.13E−02 | 2.95 | 11.41 | 0.66 | 2.93 |
| BATF | 0.54 | 7.95E−03 | 256.71 | 303.14 | 31.61 | 42.51 |
| BCAS3 | 0.42 | 1.28E−07 | 17.56 | 48.24 | 3.69 | 10.47 |
| BCAT1 | 0.25 | 7.34E−11 | 2.64 | 23.56 | 0.60 | 5.03 |
| BHLHE40-AS1 | 0.43 | 6.67E−14 | 8.50 | 47.75 | 1.86 | 9.42 |
| BIRC5 | 0.16 | 1.26E−02 | 8.94 | 24.26 | 0.94 | 2.93 |
| BNIP3L | 0.24 | 2.59E−02 | 36.46 | 51.30 | 8.16 | 13.61 |
| BRD4 | 0.44 | 2.60E−02 | 71.66 | 116.94 | 14.63 | 22.30 |
| BST2 | 0.64 | 1.89E−04 | 115.45 | 199.81 | 21.46 | 32.77 |
| BTLA | 0.99 | 2.09E−19 | 42.32 | 130.16 | 8.41 | 23.04 |
| C14orf2 | 0.18 | 2.26E−02 | 365.16 | 438.45 | 54.68 | 60.73 |
| C16orf45 | 0.19 | 9.32E−04 | 7.04 | 19.41 | 1.63 | 5.13 |
| C16orf87 | 0.40 | 2.01E−03 | 53.93 | 88.94 | 10.75 | 19.27 |
| C17orf58 | 0.10 | 2.44E−02 | 1.68 | 9.48 | 0.41 | 1.88 |
| C1orf228 | 0.47 | 2.24E−09 | 14.05 | 45.67 | 3.39 | 10.37 |
| C1orf52 | 0.21 | 2.03E−03 | 31.89 | 42.44 | 7.22 | 11.62 |
| C7orf55-LUC7L2 | 0.37 | 2.91E−03 | 43.28 | 70.64 | 9.93 | 17.49 |
| C9orf16 | 0.68 | 3.05E−06 | 187.46 | 288.07 | 33.31 | 45.65 |
| CALM2 | 0.25 | 9.48E−03 | 512.25 | 617.77 | 64.17 | 69.95 |
| CAMK1 | 0.29 | 5.68E−05 | 13.44 | 40.10 | 2.89 | 8.38 |
| CAV1 | 0.16 | 9.12E−09 | 1.92 | 12.46 | 0.21 | 2.72 |
| CBLB | 0.50 | 5.34E−04 | 55.36 | 89.95 | 11.33 | 19.79 |
| CCDC50 | 0.64 | 1.23E−12 | 26.20 | 70.10 | 5.18 | 15.18 |
| CCDC6 | 0.63 | 4.74E−09 | 35.34 | 87.46 | 8.12 | 18.95 |
| CCL3 | 0.75 | 4.60E−19 | 52.13 | 273.08 | 2.25 | 9.21 |
| CCL4 | 1.03 | 8.77E−11 | 536.21 | 1517.09 | 14.24 | 22.83 |
| CCL4L1 | 0.08 | 3.61E−02 | 1.76 | 12.39 | 0.16 | 1.05 |
| CD109 | 0.15 | 2.95E−05 | 2.27 | 11.98 | 0.41 | 2.72 |
| CD2 | 0.41 | 2.70E−09 | 1464.42 | 1886.19 | 86.75 | 90.26 |
| CD200 | 0.55 | 2.43E−15 | 13.63 | 52.31 | 2.52 | 10.79 |
| CD247 | 0.53 | 2.94E−03 | 152.11 | 227.27 | 28.50 | 38.74 |
| CD2BP2 | 0.33 | 1.59E−02 | 35.83 | 63.33 | 7.89 | 14.45 |
| CD3D | 0.56 | 2.51E−20 | 1063.16 | 1446.52 | 84.71 | 89.32 |
| CD3G | 0.54 | 5.82E−03 | 437.69 | 552.05 | 58.57 | 67.64 |
| CD7 | 1.15 | 8.14E−12 | 347.34 | 576.45 | 42.11 | 56.86 |
| CD82 | 0.75 | 7.30E−06 | 93.79 | 166.29 | 18.36 | 31.31 |
| CD84 | 0.81 | 3.15E−08 | 72.97 | 136.26 | 14.65 | 26.81 |
| CDK1 | 0.14 | 2.36E−03 | 3.92 | 20.84 | 0.71 | 2.30 |
| CDO1 | 0.05 | 3.56E−02 | 0.20 | 3.04 | 0.05 | 0.73 |
| CFL1 | 0.20 | 9.12E−05 | 833.84 | 987.52 | 78.18 | 81.36 |
| CGGBP1 | 0.13 | 3.41E−02 | 78.84 | 86.95 | 16.69 | 21.78 |
| CH25H | 0.34 | 5.13E−05 | 18.26 | 57.35 | 2.93 | 7.85 |
| CHN1 | 1.15 | 2.92E−22 | 57.82 | 167.39 | 8.41 | 23.56 |
| CHORDC1 | 0.23 | 1.06E−02 | 82.71 | 97.96 | 15.75 | 21.68 |
| CKLF | 0.51 | 9.93E−05 | 350.52 | 483.05 | 48.07 | 56.96 |
| CLECL1 | 0.44 | 6.05E−10 | 12.89 | 38.79 | 2.09 | 7.33 |
| COL6A3 | 0.26 | 3.28E−08 | 5.12 | 25.09 | 1.01 | 5.34 |
| COL9A2 | 0.13 | 1.46E−02 | 7.65 | 26.73 | 1.99 | 4.82 |
| CORO1C | 0.18 | 1.49E−03 | 6.29 | 21.58 | 1.26 | 4.40 |
| COTL1 | 1.61 | 4.76E−26 | 311.81 | 554.64 | 45.05 | 63.87 |
| COX6C | 0.38 | 5.86E−05 | 435.90 | 559.39 | 60.00 | 67.43 |
| COX8A | 0.79 | 2.72E−05 | 268.23 | 376.75 | 43.92 | 56.44 |
| CPD | 0.27 | 4.63E−03 | 18.34 | 38.34 | 4.20 | 9.21 |
| CPM | 0.45 | 3.88E−11 | 13.15 | 49.55 | 2.75 | 10.26 |
| CRTAM | 0.17 | 2.48E−02 | 9.70 | 29.78 | 1.31 | 3.25 |
| CSF2 | 0.30 | 9.84E−07 | 12.92 | 55.29 | 1.03 | 4.29 |
| CSGALNACT1 | 0.36 | 5.40E−04 | 25.47 | 55.00 | 5.36 | 11.41 |
| CTA-293F17.1 | 0.15 | 7.15E−05 | 2.51 | 10.78 | 0.50 | 2.83 |
| CTHRC1 | 0.09 | 2.33E−04 | 0.76 | 8.00 | 0.09 | 1.47 |
| CTLA4 | 1.22 | 3.58E−10 | 289.33 | 420.92 | 30.81 | 47.33 |
| CTNNB1 | 0.54 | 1.72E−03 | 118.51 | 188.39 | 16.39 | 22.20 |
| CTSB | 0.48 | 8.21E−03 | 74.46 | 118.00 | 15.04 | 24.50 |
| CTSD | 0.78 | 3.23E−07 | 93.80 | 180.04 | 17.95 | 29.84 |
| CTTN | 0.14 | 1.68E−05 | 2.20 | 9.96 | 0.44 | 2.93 |
| CXCL13 | 11.02 | 0.00E+00 | 0.00 | 4695.42 | 0.00 | 100.00 |
| CYP7B1 | 0.13 | 6.64E−03 | 3.31 | 12.59 | 0.83 | 3.25 |
| CYSLTR1 | 0.52 | 1.99E−04 | 55.07 | 99.73 | 10.02 | 17.38 |
| DAPK2 | 0.43 | 1.17E−12 | 8.27 | 35.42 | 1.67 | 7.85 |
| DBI | 0.48 | 2.97E−03 | 168.21 | 243.63 | 30.28 | 40.10 |
| DCAF7 | 0.03 | 2.06E−02 | 35.73 | 38.41 | 8.32 | 11.31 |
| DTHD1 | 0.61 | 6.81E−17 | 16.86 | 63.61 | 2.96 | 11.52 |
| DTYMK | 0.20 | 2.83E−02 | 17.61 | 40.82 | 3.87 | 7.64 |
| DUSP4 | 0.92 | 1.94E−06 | 280.55 | 430.96 | 32.69 | 46.18 |
| DYNLL1 | 0.72 | 1.38E−03 | 187.00 | 270.67 | 31.96 | 44.82 |
| ECHS1 | 0.30 | 3.64E−02 | 44.13 | 80.94 | 10.50 | 17.07 |
| EED | 0.39 | 4.45E−03 | 48.99 | 79.37 | 9.97 | 17.49 |
| EGOT | 0.14 | 2.97E−03 | 3.45 | 13.87 | 0.73 | 3.04 |
| ELMO1 | 0.88 | 7.51E−13 | 56.83 | 134.27 | 12.20 | 24.82 |
| ELMO2 | 0.13 | 2.80E−02 | 6.83 | 13.34 | 1.56 | 3.87 |
| ENTPD1 | 0.55 | 3.94E−04 | 102.23 | 153.90 | 13.73 | 22.83 |
| ESCO2 | 0.11 | 9.48E−03 | 1.97 | 12.28 | 0.32 | 1.68 |
| ETFA | 0.03 | 3.56E−02 | 42.72 | 48.33 | 9.83 | 13.93 |
| ETV7 | 0.37 | 7.47E−06 | 19.86 | 53.85 | 4.10 | 11.10 |
| EVI5 | 0.13 | 1.87E−02 | 3.70 | 12.08 | 0.80 | 2.83 |
| FABP5 | 1.38 | 5.15E−31 | 55.27 | 191.04 | 8.85 | 26.70 |
| FAIM2 | 0.09 | 1.54E−02 | 2.07 | 8.99 | 0.50 | 2.51 |
| FAM107B | 0.56 | 4.68E−07 | 257.64 | 359.83 | 43.08 | 53.19 |
| FAM13A | 0.18 | 1.96E−02 | 10.65 | 28.56 | 2.43 | 5.34 |
| FAM166B | 0.04 | 1.45E−02 | 0.11 | 3.86 | 0.02 | 0.84 |
| FAM174A | 0.01 | 4.13E−02 | 10.34 | 10.65 | 2.52 | 3.66 |
| FAM3C | 0.52 | 2.70E−11 | 21.56 | 76.15 | 4.93 | 13.30 |
| FAM43A | 0.20 | 1.25E−02 | 24.48 | 38.15 | 4.86 | 9.21 |
| FBLN5 | 0.05 | 2.92E−04 | 14.46 | 13.99 | 3.35 | 4.82 |
| FBLN7 | 0.15 | 1.45E−02 | 38.20 | 47.86 | 8.25 | 12.36 |
| FDXR | 0.17 | 5.26E−03 | 5.52 | 20.59 | 1.40 | 4.40 |
| FKBP1A | 0.67 | 9.76E−06 | 141.36 | 233.05 | 27.53 | 40.21 |
| FKBP5 | 2.37 | 4.55E−55 | 159.57 | 423.25 | 27.40 | 54.55 |
| FLT1 | 0.07 | 4.83E−02 | 1.18 | 9.20 | 0.34 | 1.47 |
| FRYL | 0.07 | 4.01E−02 | 55.59 | 61.10 | 12.20 | 16.23 |
| FYB | 0.20 | 3.55E−03 | 470.04 | 590.03 | 58.71 | 64.50 |
| FZD3 | 0.15 | 9.17E−04 | 3.74 | 11.22 | 0.78 | 2.93 |
| FZD6 | 0.12 | 6.15E−04 | 0.99 | 9.34 | 0.28 | 1.99 |
| G0S2 | 0.20 | 6.63E−07 | 6.98 | 22.76 | 0.57 | 3.35 |
| GADD45G | 0.74 | 9.98E−06 | 110.76 | 178.76 | 17.15 | 28.38 |
| GALNT1 | 0.40 | 2.83E−04 | 36.50 | 66.57 | 8.09 | 16.02 |
| GALNT2 | 0.22 | 1.44E−03 | 14.41 | 30.06 | 3.32 | 8.17 |
| GAPDH | 1.18 | 1.56E−47 | 1130.85 | 1862.47 | 80.22 | 89.32 |
| GATAD2B | 0.03 | 3.86E−02 | 24.61 | 25.53 | 5.57 | 7.43 |
| GFI1 | 0.21 | 2.72E−02 | 13.95 | 31.78 | 3.21 | 7.33 |
| GIMAP6 | 0.27 | 1.66E−02 | 24.92 | 42.33 | 6.03 | 11.41 |
| GLCCI1 | 0.46 | 4.08E−04 | 59.31 | 97.69 | 12.45 | 21.99 |
| GLDC | 0.05 | 3.35E−02 | 0.18 | 3.63 | 0.05 | 0.84 |
| GNG4 | 0.54 | 1.34E−34 | 3.03 | 48.77 | 0.46 | 9.32 |
| GNG5 | 0.62 | 2.95E−03 | 123.37 | 191.20 | 23.98 | 35.71 |
| GOLIM4 | 0.36 | 1.85E−11 | 5.09 | 35.05 | 0.92 | 5.45 |
| GPR56 | 0.41 | 4.74E−16 | 4.48 | 37.34 | 0.78 | 6.28 |
| GSKIP | 0.10 | 2.12E−02 | 19.66 | 25.04 | 4.36 | 7.43 |
| GSTT1 | 0.09 | 3.99E−02 | 7.67 | 11.71 | 1.81 | 3.66 |
| GTDC1 | 0.21 | 2.12E−03 | 13.92 | 26.15 | 3.16 | 6.91 |
| GZMB | 0.54 | 2.88E−10 | 35.40 | 131.68 | 3.37 | 9.32 |
| GZMM | 0.43 | 2.44E−02 | 70.10 | 111.31 | 15.27 | 22.83 |
| H2AFZ | 1.18 | 1.20E−20 | 226.07 | 450.26 | 36.63 | 52.04 |
| H3F3A | 0.37 | 3.96E−08 | 797.18 | 981.92 | 76.52 | 81.15 |
| HAVCR2 | 0.50 | 2.12E−11 | 17.67 | 68.04 | 3.48 | 11.20 |
| HBS1L | 0.43 | 9.93E−05 | 47.80 | 75.59 | 9.47 | 17.17 |
| HIF1A | 0.49 | 8.24E−04 | 109.59 | 173.81 | 22.24 | 31.73 |
| HINT1 | 0.29 | 2.05E−02 | 579.35 | 689.64 | 68.82 | 74.76 |
| HIST1H2BD | 0.13 | 1.50E−03 | 15.82 | 20.03 | 3.12 | 5.55 |
| HIST1H2BN | 0.03 | 1.97E−02 | 14.61 | 13.30 | 2.77 | 3.87 |
| HLA-A | 0.20 | 2.43E−05 | 2936.05 | 3298.89 | 97.36 | 98.64 |
| HLA-B | 0.16 | 1.17E−03 | 3069.64 | 3427.32 | 97.34 | 98.32 |
| HMGB1 | 0.86 | 1.72E−23 | 901.09 | 1322.99 | 78.93 | 87.23 |
| HMGB2 | 0.23 | 7.98E−03 | 263.10 | 358.47 | 39.52 | 44.92 |
| HMGN1 | 0.94 | 1.24E−13 | 396.81 | 586.22 | 55.91 | 68.06 |
| HMGN2 | 0.12 | 3.60E−02 | 399.34 | 499.62 | 54.72 | 59.79 |
| HMGN3 | 0.52 | 5.35E−04 | 70.51 | 129.57 | 15.29 | 25.45 |
| HMHB1 | 0.17 | 9.21E−08 | 1.83 | 13.33 | 0.32 | 3.14 |
| HMOX1 | 0.09 | 3.31E−02 | 2.27 | 11.33 | 0.53 | 2.30 |
| HMSD | 0.04 | 4.66E−02 | 0.41 | 3.47 | 0.11 | 1.15 |
| HNRNPA1 | 0.12 | 1.14E−04 | 1007.12 | 1155.52 | 83.20 | 84.82 |
| HNRNPA2B1 | 0.23 | 9.48E−03 | 663.38 | 778.42 | 72.03 | 76.54 |
| HNRNPA3 | 0.37 | 5.36E−03 | 290.93 | 380.98 | 45.09 | 53.82 |
| HNRNPH1 | 0.43 | 1.21E−02 | 403.78 | 511.58 | 45.94 | 49.74 |
| HNRNPK | 0.40 | 2.33E−06 | 408.91 | 526.03 | 58.09 | 65.86 |
| HNRNPL | 0.50 | 1.50E−02 | 103.93 | 153.72 | 16.76 | 23.04 |
| HNRNPLL | 0.41 | 2.08E−02 | 67.60 | 100.09 | 13.39 | 20.52 |
| HSD11B1 | 0.10 | 1.82E−03 | 0.95 | 8.31 | 0.18 | 1.57 |
| HSPB1 | 0.62 | 1.91E−02 | 221.37 | 304.94 | 29.25 | 39.37 |
| HTRA1 | 0.22 | 5.29E−08 | 1.92 | 17.08 | 0.50 | 3.66 |
| ICA1 | 1.24 | 8.83E−17 | 109.77 | 232.98 | 15.52 | 32.67 |
| ID2 | 1.45 | 1.06E−18 | 495.71 | 827.36 | 48.07 | 64.19 |
| IFI16 | 0.73 | 1.29E−07 | 444.74 | 601.59 | 58.41 | 68.69 |
| IFI6 | 0.50 | 1.46E−07 | 223.83 | 480.28 | 23.89 | 29.74 |
| IFITM10 | 0.12 | 1.64E−04 | 0.95 | 9.03 | 0.16 | 1.68 |
| IFNG | 1.49 | 1.42E−27 | 132.90 | 385.90 | 9.95 | 25.76 |
| IGFBP4 | 0.23 | 3.49E−04 | 12.68 | 25.87 | 2.84 | 7.23 |
| IGFL2 | 0.62 | 1.13E−34 | 3.37 | 59.67 | 0.46 | 8.90 |
| IL21 | 0.38 | 4.08E−15 | 5.29 | 35.69 | 0.85 | 6.49 |
| IL21-AS1 | 0.13 | 4.16E−05 | 1.19 | 10.69 | 0.23 | 2.09 |
| IL26 | 0.09 | 3.12E−02 | 2.94 | 12.25 | 0.60 | 1.68 |
| IL6ST | 1.40 | 6.56E−25 | 90.04 | 226.65 | 17.26 | 35.81 |
| IPP | 0.13 | 1.06E−02 | 6.79 | 13.51 | 1.63 | 3.98 |
| IQGAP1 | 0.56 | 1.04E−03 | 169.39 | 248.13 | 31.16 | 41.88 |
| ISCU | 0.66 | 1.59E−04 | 179.16 | 262.32 | 33.36 | 45.03 |
| ITGA2 | 0.13 | 1.21E−05 | 3.13 | 10.01 | 0.48 | 2.83 |
| ITM2A | 1.27 | 6.35E−18 | 253.56 | 456.16 | 38.15 | 54.45 |
| ITPR1 | 0.55 | 8.67E−10 | 22.94 | 69.32 | 5.07 | 13.40 |
| ITPRIPL2 | 0.14 | 5.25E−06 | 1.27 | 13.27 | 0.39 | 3.14 |
| JARID2 | 0.35 | 2.00E−02 | 40.83 | 75.05 | 8.80 | 15.71 |
| KCNK5 | 0.20 | 2.39E−05 | 4.05 | 16.58 | 0.99 | 4.29 |
| KIAA0101 | 0.18 | 2.55E−02 | 15.20 | 39.85 | 1.81 | 4.08 |
| KIAA0247 | 0.29 | 8.21E−03 | 24.38 | 47.52 | 5.04 | 10.05 |
| KIAA0319L | 0.44 | 5.66E−05 | 30.47 | 65.71 | 6.46 | 13.51 |
| KIAA1432 | 0.00 | 7.37E−03 | 9.33 | 8.12 | 2.25 | 3.04 |
| KIAA1671 | 0.12 | 1.22E−02 | 6.14 | 11.25 | 1.26 | 3.25 |
| KLF7 | 0.18 | 8.23E−03 | 5.58 | 15.89 | 1.35 | 3.87 |
| KLRB1 | 1.79 | 5.94E−18 | 612.43 | 1030.23 | 36.38 | 55.50 |
| KRT86 | 0.20 | 2.39E−08 | 2.46 | 22.57 | 0.28 | 3.04 |
| LAG3 | 0.70 | 6.79E−13 | 39.72 | 139.74 | 5.20 | 14.66 |
| LAMTOR3 | 0.20 | 3.19E−02 | 22.53 | 36.84 | 5.32 | 10.16 |
| LAPTM5 | 0.72 | 2.05E−07 | 352.32 | 486.54 | 52.91 | 63.56 |
| LBH | 0.59 | 2.33E−03 | 209.58 | 302.97 | 35.21 | 46.07 |
| LHFP | 0.40 | 1.21E−21 | 2.62 | 28.86 | 0.34 | 5.55 |
| LIF | 0.05 | 4.82E−02 | 0.40 | 4.04 | 0.16 | 1.15 |
| LIMA1 | 0.51 | 1.46E−04 | 53.43 | 99.52 | 10.68 | 20.84 |
| LIMS1 | 0.89 | 1.89E−08 | 95.80 | 176.66 | 19.65 | 34.24 |
| LINC00116 | 0.20 | 2.24E−02 | 53.61 | 65.47 | 11.10 | 16.02 |
| LINC00158 | 0.11 | 8.49E−04 | 1.26 | 8.77 | 0.18 | 1.57 |
| LMO4 | 0.67 | 9.96E−13 | 35.29 | 109.38 | 6.99 | 15.92 |
| LRMP | 0.58 | 2.53E−08 | 29.57 | 70.82 | 6.65 | 15.50 |
| LRRC8D | 0.21 | 8.69E−03 | 9.55 | 24.95 | 2.52 | 6.39 |
| LY6E | 0.20 | 1.58E−06 | 195.52 | 301.83 | 33.72 | 40.10 |
| LYST | 0.70 | 1.32E−06 | 78.92 | 147.32 | 15.20 | 26.07 |
| MAD2L1 | 0.25 | 1.28E−02 | 21.39 | 42.25 | 4.31 | 8.90 |
| MAD2L2 | 0.25 | 1.25E−02 | 20.70 | 37.85 | 4.86 | 9.63 |
| MAF | 1.58 | 8.51E−20 | 255.32 | 462.00 | 35.10 | 54.55 |
| MAGEF1 | 0.14 | 4.03E−02 | 9.86 | 17.78 | 2.34 | 5.03 |
| MAML2 | 0.26 | 1.52E−04 | 39.18 | 50.18 | 8.46 | 13.30 |
| MB | 0.07 | 3.63E−03 | 0.22 | 8.23 | 0.02 | 0.84 |
| METTL8 | 0.47 | 1.22E−02 | 76.63 | 127.50 | 14.44 | 22.93 |
| MIF | 0.28 | 1.94E−02 | 216.56 | 286.08 | 39.34 | 46.81 |
| MIR155HG | 0.34 | 4.44E−04 | 25.86 | 55.74 | 4.01 | 9.01 |
| MIR4435-1HG | 0.33 | 1.80E−02 | 195.61 | 223.31 | 25.63 | 34.03 |
| MIS18BP1 | 0.41 | 1.02E−02 | 63.86 | 98.32 | 13.41 | 21.78 |
| MKL1 | 0.04 | 1.94E−02 | 7.95 | 9.40 | 1.90 | 3.14 |
| MORF4L1 | 0.25 | 3.83E−02 | 358.76 | 435.01 | 54.59 | 61.68 |
| MPST | 0.17 | 1.30E−03 | 16.40 | 26.66 | 3.58 | 7.85 |
| MRPL51 | 0.44 | 4.73E−02 | 80.80 | 131.76 | 17.06 | 26.60 |
| MS4A6A | 0.22 | 3.86E−06 | 3.92 | 17.95 | 0.71 | 3.56 |
| MSI2 | 0.64 | 4.16E−05 | 76.08 | 134.42 | 15.77 | 26.81 |
| MT1E | 0.42 | 1.20E−06 | 22.23 | 60.12 | 4.36 | 11.52 |
| MT2A | 1.12 | 5.12E−11 | 299.90 | 514.98 | 35.72 | 49.95 |
| MTUS1 | 0.12 | 2.46E−02 | 4.28 | 9.52 | 0.92 | 2.62 |
| MTX3 | 0.17 | 1.07E−03 | 10.77 | 18.35 | 2.25 | 5.13 |
| MX1 | 0.07 | 9.16E−03 | 49.41 | 85.61 | 10.41 | 13.40 |
| MXD3 | 0.07 | 2.60E−02 | 2.24 | 4.74 | 0.30 | 1.26 |
| MYB | 0.10 | 1.51E−02 | 5.36 | 11.37 | 1.15 | 3.46 |
| MYL6 | 0.24 | 8.53E−09 | 919.32 | 1125.89 | 79.46 | 82.30 |
| MYO6 | 0.17 | 4.19E−03 | 5.81 | 15.02 | 1.28 | 3.87 |
| MYO7A | 0.29 | 7.96E−13 | 3.21 | 22.92 | 0.73 | 5.97 |
| NAB1 | 0.39 | 6.05E−05 | 25.81 | 57.21 | 5.55 | 12.57 |
| NAP1L4 | 1.34 | 2.22E−18 | 163.12 | 308.73 | 29.14 | 47.02 |
| NCALD | 0.23 | 9.86E−03 | 15.23 | 34.50 | 3.21 | 7.54 |
| NCL | 0.13 | 3.16E−02 | 447.80 | 524.93 | 58.21 | 63.46 |
| NCOA7 | 0.42 | 4.67E−02 | 73.34 | 114.01 | 15.15 | 22.51 |
| NDFIP2 | 0.41 | 9.98E−04 | 37.97 | 75.73 | 8.23 | 16.13 |
| NDUFA1 | 0.16 | 1.10E−07 | 304.52 | 389.93 | 50.76 | 56.96 |
| NDUFA13 | 0.50 | 3.50E−02 | 234.20 | 310.04 | 41.29 | 51.52 |
| NEBL | 0.18 | 9.66E−10 | 2.38 | 14.68 | 0.30 | 3.25 |
| NELL2 | 0.40 | 8.36E−06 | 21.88 | 53.86 | 5.09 | 11.20 |
| NENF | 0.44 | 4.00E−03 | 48.69 | 89.01 | 11.21 | 19.06 |
| NFIA | 0.13 | 2.60E−02 | 9.45 | 14.98 | 2.09 | 4.50 |
| NFYA | 0.02 | 4.59E−02 | 6.69 | 6.01 | 1.44 | 2.09 |
| NMB | 1.04 | 3.84E−32 | 24.82 | 160.87 | 3.83 | 17.59 |
| NPDC1 | 0.38 | 3.57E−05 | 24.65 | 55.89 | 5.55 | 13.19 |
| NR3C1 | 2.28 | 9.75E−52 | 251.34 | 573.27 | 36.91 | 61.68 |
| NSMAF | 0.13 | 4.42E−02 | 15.90 | 22.35 | 3.78 | 6.60 |
| NUCKS1 | 0.57 | 2.06E−02 | 194.15 | 263.59 | 33.04 | 42.72 |
| NUDT16 | 0.39 | 1.93E−07 | 13.14 | 38.80 | 3.12 | 9.42 |
| NUDT7 | 0.17 | 3.30E−03 | 4.65 | 16.92 | 1.17 | 3.77 |
| NUSAP1 | 0.23 | 1.98E−02 | 20.25 | 47.76 | 3.05 | 6.60 |
| OAZ1 | 0.52 | 8.78E−10 | 698.76 | 870.80 | 73.57 | 80.10 |
| ORMDL3 | 0.20 | 4.97E−02 | 36.36 | 53.16 | 8.14 | 13.19 |
| OSBPL2 | 0.09 | 2.57E−02 | 15.36 | 18.20 | 3.51 | 5.55 |
| PAM | 0.16 | 4.19E−02 | 15.55 | 28.02 | 3.51 | 7.12 |
| PARK7 | 0.61 | 1.11E−03 | 239.58 | 334.38 | 39.45 | 51.10 |
| PCBP3 | 0.07 | 3.14E−02 | 1.50 | 4.90 | 0.37 | 1.57 |
| PCNA | 0.32 | 2.16E−02 | 35.76 | 68.78 | 6.67 | 12.15 |
| PCNXL2 | 0.29 | 2.03E−03 | 21.09 | 38.26 | 4.42 | 8.80 |
| PDCD1 | 1.29 | 1.25E−32 | 35.90 | 129.05 | 7.11 | 24.50 |
| PDE7B | 0.84 | 8.15E−42 | 5.44 | 55.53 | 1.10 | 12.57 |
| PDIA6 | 0.49 | 2.58E−02 | 116.10 | 186.33 | 22.63 | 33.51 |
| PEAK1 | 0.12 | 2.31E−02 | 4.43 | 11.55 | 0.99 | 3.04 |
| PFN1 | 0.50 | 8.10E−11 | 988.20 | 1236.23 | 80.67 | 85.76 |
| PGAM1 | 0.91 | 7.97E−10 | 106.63 | 222.41 | 20.47 | 35.71 |
| PGM2L1 | 0.32 | 2.55E−03 | 25.19 | 52.76 | 5.34 | 11.41 |
| PHACTR2 | 0.53 | 2.29E−03 | 76.86 | 124.37 | 15.22 | 24.92 |
| PHEX | 0.30 | 8.22E−13 | 3.23 | 24.97 | 0.64 | 5.45 |
| PIN4 | 0.30 | 2.01E−02 | 59.15 | 79.23 | 12.61 | 19.27 |
| PKM | 1.47 | 2.79E−17 | 213.12 | 392.78 | 30.72 | 49.95 |
| PLEKHF1 | 0.19 | 3.44E−02 | 14.98 | 29.79 | 3.71 | 8.06 |
| PLS3 | 0.07 | 6.95E−03 | 0.32 | 5.78 | 0.09 | 1.05 |
| POLR2G | 0.33 | 4.64E−02 | 75.84 | 122.38 | 16.51 | 23.77 |
| PON2 | 0.12 | 2.24E−03 | 3.00 | 10.33 | 0.71 | 3.04 |
| POU2AF1 | 0.11 | 7.98E−03 | 2.69 | 10.44 | 0.57 | 2.51 |
| PPARG | 0.23 | 2.97E−09 | 3.17 | 16.24 | 0.50 | 3.77 |
| PPIA | 0.10 | 2.90E−04 | 563.31 | 664.31 | 67.06 | 71.10 |
| PPP1CC | 0.90 | 3.62E−08 | 99.71 | 171.94 | 20.56 | 34.97 |
| PPP4C | 0.48 | 8.23E−03 | 72.96 | 126.65 | 16.37 | 26.49 |
| PRDM1 | 0.02 | 6.59E−03 | 315.49 | 370.78 | 39.02 | 40.84 |
| PSMA7 | 0.82 | 3.90E−09 | 342.26 | 488.28 | 49.47 | 61.05 |
| PTMS | 0.76 | 4.80E−18 | 25.16 | 93.87 | 4.49 | 15.81 |
| PTPN11 | 0.40 | 1.96E−04 | 28.12 | 63.71 | 6.46 | 13.93 |
| PTPN13 | 0.75 | 9.94E−30 | 9.82 | 55.42 | 2.04 | 13.19 |
| PTPRC | 0.14 | 1.74E−11 | 1509.53 | 1759.96 | 89.61 | 88.80 |
| PTPRN2 | 0.30 | 4.97E−04 | 15.13 | 38.86 | 3.55 | 8.80 |
| PVALB | 0.61 | 2.86E−24 | 6.36 | 45.90 | 1.15 | 8.90 |
| RAB12 | 0.13 | 1.12E−02 | 13.50 | 31.25 | 1.99 | 3.35 |
| RAB27A | 0.92 | 1.37E−12 | 62.81 | 141.98 | 13.82 | 27.96 |
| RANBP1 | 0.39 | 4.01E−03 | 151.91 | 238.09 | 27.26 | 35.71 |
| RBPJ | 2.12 | 2.66E−44 | 194.44 | 495.48 | 30.24 | 54.35 |
| RDH10 | 0.34 | 2.77E−15 | 5.21 | 28.78 | 0.89 | 6.81 |
| REEP2 | 0.07 | 6.46E−03 | 1.06 | 5.33 | 0.21 | 1.57 |
| RGS1 | 0.79 | 2.11E−06 | 2539.03 | 3037.52 | 79.62 | 86.39 |
| RGS2 | 1.29 | 4.58E−12 | 334.89 | 526.03 | 37.48 | 53.30 |
| RHOA | 0.43 | 2.43E−04 | 213.50 | 299.48 | 37.99 | 47.85 |
| RNF19A | 1.12 | 3.36E−13 | 326.15 | 517.93 | 44.70 | 59.58 |
| ROMO1 | 0.69 | 1.26E−03 | 138.67 | 208.16 | 26.11 | 38.43 |
| RP11-132N15.3 | 0.04 | 1.22E−02 | 0.16 | 3.66 | 0.05 | 1.05 |
| RP11-25K19.1 | 0.40 | 4.45E−10 | 10.77 | 34.56 | 2.48 | 9.01 |
| RP11-265P11.2 | 0.38 | 3.54E−08 | 16.99 | 45.00 | 3.60 | 10.99 |
| RP11-279F6.3 | 1.00 | 2.04E−41 | 12.01 | 93.20 | 2.32 | 16.75 |
| RP11-316P17.2 | 0.22 | 1.79E−04 | 8.03 | 30.18 | 1.65 | 5.76 |
| RP11-345M22.1 | 0.31 | 4.07E−08 | 11.99 | 33.79 | 1.70 | 6.28 |
| RP11-431M7.3 | 0.08 | 3.36E−02 | 1.03 | 6.27 | 0.28 | 1.47 |
| RP11-444D3.1 | 0.06 | 2.55E−02 | 0.84 | 3.59 | 0.18 | 1.15 |
| RP11-553L6.2 | 0.19 | 4.32E−02 | 21.27 | 31.45 | 4.49 | 7.85 |
| RP11-65J3.1 | 0.06 | 1.36E−03 | 0.36 | 3.18 | 0.05 | 1.05 |
| RP11-94L15.2 | 0.47 | 1.82E−02 | 83.51 | 129.82 | 16.44 | 24.40 |
| RP13-143G15.4 | 0.30 | 2.64E−14 | 1.91 | 18.69 | 0.41 | 4.71 |
| RP5-1028K7.2 | 1.27 | 1.26E−31 | 36.62 | 130.95 | 7.47 | 24.71 |
| RP6-109B7.3 | 0.29 | 1.27E−03 | 36.47 | 52.97 | 7.27 | 12.77 |
| RP6-91H8.3 | 0.23 | 1.46E−04 | 7.39 | 29.06 | 1.56 | 5.24 |
| RPA3 | 0.49 | 1.31E−02 | 81.92 | 132.62 | 17.10 | 26.81 |
| RPL7L1 | 0.45 | 1.44E−02 | 72.85 | 116.15 | 15.08 | 23.46 |
| RYR2 | 0.06 | 2.76E−04 | 0.00 | 3.17 | 0.00 | 0.94 |
| SAE1 | 0.29 | 3.66E−02 | 30.85 | 60.45 | 7.15 | 13.40 |
| SARDH | 0.40 | 2.02E−09 | 10.87 | 37.38 | 2.71 | 9.63 |
| SCAMP4 | 0.14 | 3.50E−02 | 13.44 | 20.19 | 2.93 | 5.45 |
| SEC11A | 0.89 | 2.04E−07 | 118.04 | 201.74 | 23.73 | 37.80 |
| SEC14L2 | 0.25 | 5.53E−06 | 5.04 | 20.44 | 1.12 | 4.61 |
| SEC22C | 0.14 | 3.60E−02 | 23.88 | 30.73 | 5.55 | 8.90 |
| SEPT7 | 0.34 | 3.92E−05 | 372.90 | 475.72 | 54.79 | 62.20 |
| SERF2 | 0.52 | 8.31E−28 | 902.93 | 1210.48 | 81.84 | 85.86 |
| SERPINE2 | 0.10 | 2.40E−02 | 4.56 | 18.88 | 0.92 | 3.46 |
| SESN1 | 0.33 | 1.86E−02 | 39.86 | 70.86 | 7.79 | 13.30 |
| SET | 0.59 | 1.53E−02 | 292.73 | 387.00 | 41.66 | 50.26 |
| SFXN1 | 0.46 | 1.59E−02 | 77.83 | 122.84 | 15.98 | 24.50 |
| SGK1 | 0.42 | 1.77E−05 | 59.10 | 80.20 | 10.25 | 17.70 |
| SH2D1A | 0.77 | 5.15E−05 | 149.92 | 235.73 | 26.87 | 40.00 |
| SH3BGRL3 | 0.03 | 8.31E−09 | 610.64 | 761.92 | 68.02 | 70.99 |
| SHCBP1 | 0.08 | 6.64E−03 | 0.71 | 9.64 | 0.18 | 1.36 |
| SHFM1 | 0.75 | 2.25E−04 | 212.76 | 300.26 | 36.57 | 49.11 |
| SIRPG | 0.61 | 6.40E−05 | 76.17 | 127.70 | 14.86 | 26.07 |
| SLA | 1.11 | 1.53E−12 | 278.90 | 464.51 | 40.99 | 55.92 |
| SLAMF6 | 0.29 | 4.66E−04 | 23.02 | 40.07 | 5.18 | 10.58 |
| SLC25A46 | 0.21 | 5.68E−04 | 15.71 | 26.89 | 3.69 | 7.96 |
| SLC27A2 | 0.19 | 4.76E−04 | 6.34 | 23.81 | 1.49 | 5.34 |
| SLC28A3 | 0.04 | 8.28E−04 | 0.00 | 3.88 | 0.00 | 0.94 |
| SLC9A9 | 0.63 | 8.31E−09 | 38.84 | 78.26 | 8.46 | 18.43 |
| SMAD1 | 0.09 | 1.51E−02 | 1.97 | 8.82 | 0.46 | 2.30 |
| SMARCA2 | 0.67 | 1.70E−08 | 47.17 | 105.42 | 10.55 | 21.36 |
| SMC2 | 0.20 | 2.46E−02 | 17.41 | 33.77 | 3.65 | 7.75 |
| SMCO4 | 0.76 | 5.08E−17 | 25.95 | 78.92 | 5.48 | 17.38 |
| SMOX | 0.10 | 1.68E−02 | 2.44 | 7.80 | 0.44 | 1.88 |
| SMPDL3A | 0.10 | 7.58E−05 | 0.62 | 6.99 | 0.11 | 1.57 |
| SNRPF | 0.37 | 4.43E−02 | 154.63 | 217.16 | 30.19 | 38.74 |
| SNX14 | 0.15 | 4.43E−02 | 25.20 | 33.58 | 5.62 | 9.21 |
| SNX9 | 0.71 | 2.81E−08 | 56.70 | 119.48 | 11.23 | 23.77 |
| SOD1 | 1.01 | 5.10E−12 | 504.92 | 701.94 | 59.58 | 72.04 |
| SON | 0.57 | 4.98E−07 | 572.88 | 702.04 | 69.19 | 76.86 |
| SPC24 | 0.12 | 2.55E−02 | 3.70 | 18.18 | 0.50 | 1.78 |
| SPC25 | 0.09 | 9.48E−03 | 1.16 | 12.06 | 0.18 | 1.36 |
| SPCS2 | 0.60 | 2.05E−02 | 177.71 | 243.72 | 33.63 | 44.92 |
| SPOCK2 | 0.26 | 1.68E−02 | 295.06 | 390.83 | 45.05 | 52.88 |
| SPRY1 | 0.49 | 2.53E−07 | 28.36 | 61.19 | 4.56 | 10.89 |
| SRGAP3 | 0.37 | 8.15E−05 | 23.33 | 59.39 | 4.84 | 10.47 |
| SRGN | 1.08 | 2.32E−56 | 1366.39 | 2268.95 | 84.89 | 92.77 |
| SRP14 | 0.42 | 1.24E−08 | 603.96 | 756.95 | 70.93 | 77.07 |
| ST6GALNAC3 | 0.09 | 5.00E−03 | 1.20 | 4.93 | 0.25 | 1.47 |
| STMN1 | 0.41 | 1.52E−04 | 57.60 | 140.99 | 6.37 | 10.89 |
| STON1 | 0.09 | 4.00E−03 | 1.06 | 6.15 | 0.21 | 1.57 |
| STRIP2 | 0.09 | 2.12E−03 | 2.45 | 6.37 | 0.32 | 1.57 |
| SUB1 | 0.47 | 2.61E−04 | 499.25 | 630.70 | 61.76 | 69.84 |
| SUMO2 | 0.26 | 5.42E−03 | 382.12 | 461.56 | 56.37 | 63.46 |
| SYT11 | 0.40 | 1.14E−04 | 28.71 | 61.07 | 6.17 | 13.61 |
| TBC1D4 | 0.28 | 5.12E−04 | 158.64 | 175.09 | 23.41 | 31.73 |
| TBCEL | 0.11 | 6.76E−03 | 4.10 | 10.50 | 0.96 | 3.46 |
| TBXAS1 | 0.26 | 1.07E−05 | 8.06 | 21.59 | 1.65 | 5.45 |
| TCEB2 | 0.42 | 8.98E−03 | 332.84 | 421.03 | 51.86 | 60.52 |
| TGIF1 | 0.56 | 5.32E−05 | 51.47 | 95.64 | 10.55 | 18.85 |
| THADA | 0.92 | 2.14E−13 | 60.94 | 149.59 | 10.66 | 23.66 |
| TIGIT | 1.29 | 2.33E−13 | 214.01 | 319.73 | 28.29 | 46.60 |
| TIMM17B | 0.06 | 1.09E−02 | 29.30 | 31.35 | 6.58 | 8.90 |
| TMA7 | 0.20 | 8.92E−03 | 1036.36 | 1151.74 | 84.89 | 87.54 |
| TMBIM6 | 0.47 | 4.19E−02 | 279.43 | 368.31 | 46.79 | 56.65 |
| TMEM128 | 0.24 | 1.70E−02 | 24.18 | 40.19 | 5.41 | 9.74 |
| TMEM167A | 0.52 | 6.52E−04 | 63.60 | 115.09 | 14.03 | 23.77 |
| TMEM245 | 0.12 | 2.12E−02 | 24.26 | 30.32 | 5.78 | 9.01 |
| TMSB10 | 0.15 | 2.91E−03 | 4459.94 | 5059.17 | 98.62 | 99.48 |
| TMSB4X | 0.10 | 6.79E−03 | 16960.05 | 18155.01 | 99.91 | 100.00 |
| TNFRSF18 | 1.61 | 6.95E−21 | 198.22 | 372.72 | 21.92 | 42.83 |
| TNFRSF4 | 0.69 | 6.66E−06 | 322.44 | 330.75 | 26.23 | 38.22 |
| TNFSF8 | 0.36 | 1.98E−03 | 27.88 | 55.21 | 5.73 | 10.68 |
| TOMM34 | 0.12 | 2.76E−03 | 4.81 | 12.87 | 1.15 | 3.66 |
| TOP2A | 0.31 | 5.83E−04 | 26.22 | 57.18 | 2.89 | 6.70 |
| TOPORS | 0.17 | 1.90E−02 | 34.09 | 44.98 | 7.52 | 12.04 |
| TOX | 0.95 | 7.84E−21 | 35.08 | 96.10 | 6.83 | 20.21 |
| TOX2 | 0.85 | 1.14E−15 | 39.94 | 103.14 | 8.21 | 22.20 |
| TP53TG1 | 0.00 | 5.70E−03 | 31.07 | 30.92 | 7.15 | 8.80 |
| TP73 | 0.07 | 1.22E−02 | 1.68 | 5.37 | 0.21 | 1.15 |
| TPI1 | 1.45 | 1.78E−23 | 197.01 | 392.90 | 34.41 | 53.09 |
| TPM3 | 0.41 | 2.86E−05 | 364.76 | 476.86 | 52.36 | 60.73 |
| TRPS1 | 0.57 | 1.44E−06 | 42.02 | 84.17 | 8.83 | 18.12 |
| TSHZ2 | 1.73 | 3.40E−31 | 101.70 | 255.74 | 16.67 | 38.32 |
| TSPAN13 | 0.11 | 7.61E−04 | 3.80 | 11.07 | 0.60 | 2.83 |
| TSPAN5 | 0.02 | 1.38E−02 | 49.42 | 55.46 | 10.22 | 14.66 |
| TSPO | 0.64 | 5.13E−05 | 154.63 | 232.89 | 30.12 | 41.26 |
| TTC38 | 0.11 | 1.43E−02 | 3.46 | 10.20 | 0.83 | 2.93 |
| TUBA3D | 0.09 | 7.41E−03 | 1.08 | 7.94 | 0.21 | 1.47 |
| TWIST1 | 0.19 | 1.25E−03 | 6.90 | 23.17 | 0.96 | 3.35 |
| TXNDC17 | 0.10 | 9.45E−03 | 57.56 | 71.08 | 11.74 | 17.28 |
| TXNRD2 | 0.10 | 1.40E−02 | 8.12 | 11.81 | 1.70 | 3.46 |
| TYMS | 0.14 | 1.22E−02 | 8.12 | 29.87 | 0.96 | 2.41 |
| TYW5 | 0.18 | 8.58E−03 | 10.82 | 22.43 | 2.41 | 5.76 |
| UBB | 0.33 | 1.68E−02 | 762.41 | 888.86 | 73.34 | 78.85 |
| UBE2T | 0.16 | 2.33E−02 | 6.92 | 22.31 | 1.38 | 3.66 |
| UBL5 | 0.28 | 3.93E−06 | 435.79 | 536.11 | 61.51 | 67.64 |
| UBXN10-AS1 | 0.13 | 4.71E−02 | 5.80 | 14.40 | 1.40 | 3.87 |
| UCP2 | 0.98 | 1.08E−07 | 148.94 | 259.01 | 24.23 | 39.37 |
| UQCR11.1 | 0.50 | 7.29E−04 | 477.27 | 587.85 | 64.60 | 72.88 |
| UROS | 0.10 | 1.23E−03 | 29.18 | 33.52 | 6.85 | 10.68 |
| USMG5 | 0.36 | 1.34E−02 | 296.87 | 380.22 | 48.44 | 56.86 |
| VDAC1 | 0.58 | 7.76E−04 | 114.83 | 190.97 | 22.97 | 34.97 |
| VOPP1 | 0.67 | 4.12E−06 | 64.42 | 123.26 | 14.14 | 26.49 |
| WARS | 0.18 | 8.70E−03 | 9.69 | 27.67 | 2.48 | 7.02 |
| WSB2 | 0.21 | 3.32E−03 | 10.02 | 26.59 | 1.83 | 5.03 |
| XXYLT1 | 0.12 | 1.64E−02 | 7.16 | 14.02 | 1.33 | 3.56 |
| YWHAQ | 0.90 | 1.23E−11 | 108.74 | 213.51 | 22.51 | 37.07 |
| ZBED2 | 0.74 | 2.16E−23 | 19.22 | 101.87 | 2.66 | 13.51 |
| ZEB2 | 0.75 | 1.20E−17 | 22.16 | 86.00 | 4.33 | 14.66 |
| ZMYM2 | 0.42 | 3.06E−02 | 67.82 | 103.15 | 14.40 | 21.68 |
| ZNF280D | 0.27 | 8.97E−03 | 30.18 | 47.18 | 6.46 | 11.62 |
| ZNF281 | 0.36 | 1.45E−03 | 40.07 | 68.83 | 8.14 | 15.50 |
| ZNF700 | 0.06 | 4.96E−03 | 7.05 | 7.35 | 1.44 | 2.72 |
| ZNF789 | 0.02 | 2.44E−02 | 4.33 | 10.91 | 1.15 | 1.47 |
| ZNF831 | 0.08 | 3.19E−02 | 13.11 | 16.53 | 2.87 | 4.50 |
| ZNRF1 | 0.39 | 3.01E−04 | 32.31 | 66.27 | 7.36 | 15.50 |

TABLE 9
Related to FIG. 4.
Pathway analysis of differentially expressed genes in CXCL13-expressing
versus CXCL13-non-expressing CD4⁺ TILs.

| Ingenuity | | Number of | Number of | Fraction of | |
|---|---|---|---|---|---|
| canonical | -log(B-H | genes in | DEGs in | upregulated | |
| pathways | p-value) | pathway | pathway | genes | DEGS in the pathway |
| CD28 | 3.94 | 132 | 14 | 0.11 | CD247, ARPC1B, HLA-A, |
| Signaling | | | | | HLA-B, ITPR1, CTLA4, |
| in T Helper | | | | | CD3D, PTPRC, CALM1 |
| Cells | | | | | (includes others), |
| | | | | | CD3G, PTPN11, ARPC2, |
| | | | | | AKT3, ARPC3 |
| Cytotoxic T | 3.36 | 32 | 7 | 0.22 | CD247, B2M, CD3G, |
| Lymphocyte- | | | | | GZMB, HLA-A, HLA-B, |
| mediated | | | | | CD3D |
| Apoptosis of | | | | | |
| Target Cells | | | | | |
| Cdc42 | 2.62 | 167 | 13 | 0.08 | CD247, B2M, ARPC1B, |
| Signaling | | | | | MYL6, CFL1, HLA-A, |
| | | | | | ITGA2, HLA-B, IQGAP1, |
| | | | | | CD3D, CD3G, ARPC2, |
| | | | | | ARPC3 |
| iCOS-iCOSL | 2.62 | 123 | 11 | 0.09 | CD247, PTPRC, CD3G, |
| Signaling | | | | | CALM1 |
| in T Helper | | | | | (includes others), |
| Cells | | | | | BAD, PTPN11, HLA-A, |
| | | | | | HLA-B, AKT3, ITPR1, |
| | | | | | CD3D |
| Mitochondrial | 2.62 | 171 | 13 | 0.08 | ATP5MC2, UCP2, PARK7, |
| Dysfunction | | | | | COX6C, COX8A, ATP5MF, |
| | | | | | ATP5MG, VDAC1, TXNRD2, |
| | | | | | ATP5ME, NDUFA1, NDUFA13, |
| | | | | | ATP5F1E |
| Epithelial | 2.61 | 150 | 12 | 0.08 | ARPC1B, MYL6, RHOA, |
| Adherens | | | | | MYO7A, ACTB, ARPC2, |
| Junction | | | | | TUBA3C/TUBA3D, ARPC3, |
| Signaling | | | | | AKT3, CTNNB1, IQGAP1, |
| | | | | | ACTG1 |
| Remodeling of | 2.51 | 69 | 8 | 0.12 | ARPC1B, ACTB, ARPC2, |
| Epithelial | | | | | TUBA3C/TUBA3D, ARPC3, |
| Adherens | | | | | CTNNB1, IQGAP1, ACTG1 |
| Junctions | | | | | |
| Regulation of | 2.51 | 90 | 9 | 0.10 | PFN1, ARPC1B, CFL1, |
| Actin- based | | | | | MYL6, RHOA, ACTB, |
| Motility by | | | | | ARPC2, ITGA2, ARPC3 |
| Rho | | | | | |
| Fcγ | 2.45 | 93 | 9 | 0.10 | HMOX1, ARPC1B, ACTB, |
| Receptor- | | | | | ARPC2, ARPC3, AKT3, |
| mediated | | | | | FYB1, CSF2, ACTG1 |
| Phagocytosis | | | | | |
| in Macrophages | | | | | |
| and Monocytes | | | | | |
| Integrin | 2.4 | 219 | 14 | 0.06 | TSPAN5, PFN1, ARPC1B, |
| Signaling | | | | | ACTB, ITGA2, PTPN11, |
| | | | | | LIMS1, RHOA, ARPC2, |
| | | | | | CAV1, AKT3, ARPC3, |
| | | | | | CTTN, ACTG1 |
| CTLA4 | 2.32 | 99 | 9 | 0.09 | CD247, B2M, CD3G, |
| Signaling in | | | | | PTPN11, HLA-A, HLA-B, |
| Cytotoxic T | | | | | AKT3, CTLA4, CD3D |
| Lymphocytes | | | | | |
| Sirtuin | 2.18 | 292 | 16 | 0.05 | PPARG, UCP2, GADD45G, |
| Signaling | | | | | TP73, TIMM17B, HIF1A, |
| Pathway | | | | | SOD1, NDUFA1, ATP5F1E, |
| | | | | | NDUFA13, H3F3A/H3F3B, |
| | | | | | PGA M1, TOMM34, TUBA3C/ |
| | | | | | TUBA3D, TSPO, VDAC1 |
| Oxidative | 2.09 | 109 | 9 | 0.08 | ATP5MC2, COX6C, COX8A, |
| Phosphorylation | | | | | ATP5MF, ATP5MG, ATP5ME, |
| | | | | | NDUFA1, NDUFA13, ATP5F1E |
| Calcium- | 2.09 | 66 | 7 | 0.11 | CD247, CD3G, CALM1 |
| induced T | | | | | (includes others), |
| Lymphocyte | | | | | HLA-A, HLA-B, ITPR1, |
| Apoptosis | | | | | CD3D |
| p53 | 2.08 | 111 | 9 | 0.08 | PCNA, PTPN11, GADD45G, |
| Signaling | | | | | TP73, AKT3, HIF1A, |
| | | | | | CTNNB1, BIRC5, SERPINE2 |
| Mouse | 2.08 | 112 | 9 | 0.08 | IL6ST, ID2, LIF, |
| Embryonic | | | | | PTPN11, FZD3, FZD6, |
| Stem Cell | | | | | AKT3, CTNNB1, SMAD1 |
| Pluripotency | | | | | |
| Signaling | 2 | 252 | 14 | 0.06 | GNG4, ARPC1B, MYL6, |
| by Rho Family | | | | | CFL1, ACTB, ITGA2, |
| GTPases | | | | | IQGAP1, STMN1, PTPN11, |
| | | | | | RHOA, ARPC2, ARPC3, |
| | | | | | GNG5, ACTG1 |
| Caveolar- | 2 | 71 | 7 | 0.10 | B2M, HLA-A, ACTB, |
| mediated | | | | | HLA-B, ITGA2, CAV1, |
| Endocytosis | | | | | ACTG1 |
| Signaling | | | | | |
| CCR5 | 1.96 | 95 | 8 | 0.08 | CD247, GNG4, CD3G, |
| Signaling in | | | | | CALM1 |
| Macrophages | | | | | (includes others), |
| | | | | | CCL4, GNG5, CCL3, |
| | | | | | CD3D |
| Actin | 1.91 | 233 | 13 | 0.06 | PFN1, ARPC1B, MYL6, |
| Cytoskeleton | | | | | CFL1, ACTB, ITGA2, |
| Signaling | | | | | IQGAP1, PTPN11, RHOA, |
| | | | | | ARPC2, ARPC3, TMSB10/ |
| | | | | | TMSB4X, ACTG1 |
| RhoGDI | 1.91 | 177 | 11 | 0.06 | GNG4, ARPC1B, CFL1, |
| Signaling | | | | | MYL6, RHOA, ACTB, |
| | | | | | ARPC2, ITGA2, ARPC3, |
| | | | | | GNG5, ACTG1 |
| Th2 Pathway | 1.91 | 150 | 10 | 0.07 | CD247, CD3G, IFNG, |
| | | | | | TNFRSF4, PTPN11, HLA-A, |
| | | | | | MAF, HLA-B, GFI1, |
| | | | | | CD3D |
| RhoA | 1.91 | 124 | 9 | 0.07 | PFN1, ARPC1B, CFL1, |
| Signaling | | | | | MYL6, RHOA, ACTB, |
| | | | | | ARPC2, ARPC3, ACTG1 |
| Ephrin | 1.9 | 179 | 11 | 0.06 | GNG4, ARPC1B, PTPN11, |
| Receptor | | | | | CFL1, PTPN13, RHOA, |
| Signaling | | | | | ARPC2, ITGA2, ARPC3, |
| | | | | | AKT3, GNG5 |
| Protein | 1.85 | 401 | 18 | 0.04 | SMPDL3A, GNG4, BAD, |
| Kinase A | | | | | MYL6, PTPN13, RYR2, |
| Signaling | | | | | ITPR1, PTPRC, YWHAQ, |
| | | | | | CALM1 |
| | | | | | (includes others), |
| | | | | | PPP1CC, H3F3A/H3F3B, |
| | | | | | PTPN11, PDE7B, RHOA, |
| | | | | | DUSP4, CTNNB1, GNG5 |
| Differential | 1.84 | 23 | 4 | 0.17 | IFNG, CCL4, CSF2, |
| Regulation | | | | | CCL3 |
| of Cytokine | | | | | |
| Production in | | | | | |
| Intestinal | | | | | |
| Epithelial | | | | | |
| Cells by | | | | | |
| IL- 17A and | | | | | |
| IL-17F | | | | | |
| Nur77 | 1.84 | 59 | 6 | 0.10 | CD247, CD3G, CALM1 |
| Signaling in | | | | | (includes others), |
| T Lymphocytes | | | | | HLA-A, HLA-B, CD3D |
| Th1 and Th2 | 1.84 | 185 | 11 | 0.06 | CD247, CD3G, IFNG, |
| Activation | | | | | TNFRSF4, PTPN11, HLA-A, |
| Pathway | | | | | HAVCR2, MAF, HLA-B, |
| | | | | | GFI1, CD3D |
| Glucocorticoid | 1.78 | 345 | 16 | 0.05 | CD247, IFNG, SGK1, |
| Receptor | | | | | ACTB, CCL3, CD3D, |
| Signaling | | | | | NR3C1, KRT86, POLR2G, |
| | | | | | HMGB1, CD3G, PTPN11, |
| | | | | | SMARCA2, AKT3, CSF2, |
| | | | | | FKBP5 |
| Role of NFAT | 1.74 | 192 | 11 | 0.06 | CD247, GNG4, CD3G, |
| in Regulation | | | | | CALM1 |
| of the Immune | | | | | (includes others), |
| Response | | | | | PTPN11, HLA-A, HLA-B, |
| | | | | | AKT3, ITPR1, GNG5, |
| | | | | | CD3D |
| Type I | 1.73 | 111 | 8 | 0.07 | CD247, CD3G, IFNG, |
| Diabetes | | | | | ICA1, GZMB, HLA-A, |
| Mellitus | | | | | HLA-B, CD3D |
| Signaling | | | | | |
| Glycolysis I | 1.73 | 26 | 4 | 0.15 | TPI1, PGAM1, PKM, |
| | | | | | GAPDH |
| Regulation | 1.73 | 195 | 11 | 0.06 | ID2, PTPN11, FZD3, |
| of the | | | | | RHOA, ZEB2, TWIST1, |
| Epithelial- | | | | | FZD6, AKT3, RBPJ, |
| Mesenchymal | | | | | HIF1A, CTNNB1 |
| Transition | | | | | |
| Pathway | | | | | |
| ILK | 1.71 | 197 | 11 | 0.06 | PTPN11, CFL1, MYL6, |
| Signaling | | | | | LIMS1, RHOA, ACTB, |
| | | | | | AKT3, HIF1A, TMSB10/ |
| | | | | | TMSB4X, CTNNB1, ACTG1 |
| Virus | 1.66 | 116 | 8 | 0.07 | B2M, PTPN11, HLA-A, |
| Entry via | | | | | ACTB, HLA-B, ITGA2, |
| Endocytic | | | | | CAV1, ACTG1 |
| Pathways | | | | | |
| OX40 | 1.66 | 91 | 7 | 0.08 | CD247, B2M, CD3G, |
| Signaling | | | | | TNFRSF4, HLA-A, HLA-B, |
| Pathway | | | | | CD3D |
| Hematopoiesis | 1.6 | 48 | 5 | 0.10 | CD247, CD3G, LIF, |
| from | | | | | CSF2, CD3D |
| Pluripotent | | | | | |
| Stem Cells | | | | | |
| Communication | 1.58 | 95 | 7 | 0.07 | B2M, IFNG, CCL4, |
| between Innate | | | | | HLA-A, HLA-B, CSF2, |
| and Adaptive | | | | | CCL3 |
| Immune Cells | | | | | |
| Germ Cell- | 1.57 | 179 | 10 | 0.06 | PTPN11, CFL1, HOA, |
| Sertoli Cell | | | | | MYO7A, ACTB, ITGA2, |
| Junction | | | | | TUBA3C/TUBA3D, CTNNB1, |
| Signaling | | | | | IQGAP1, ACTG1 |
| ERK5 | 1.57 | 72 | 6 | 0.08 | IL6ST, YWHAQ, LIF, |
| Signaling | | | | | BAD, PTPN11, SGK1 |
| Rac | 1.57 | 123 | 8 | 0.07 | ARPC1B, PTPN11, CFL1, |
| Signaling | | | | | RHOA, ARPC2, ITGA2, |
| | | | | | ARPC3, IQGAP1 |
| Breast | 1.57 | 211 | 11 | 0.05 | STMN1, GNG4, CALM1 |
| Cancer | | | | | (includes others), |
| Regulation by | | | | | PPP1CC, CAMK1, PTPN11, |
| Stathmin 1 | | | | | RHOA, TUBA3C/TUBA3D, |
| | | | | | ITPR1, GNG5, CDK1 |
| Ephrin B | 1.57 | 73 | 6 | 0.08 | GNG4, CFL1, RHOA, |
| Signaling | | | | | CTNNB1, GNG5, HNRNPK |
| Osteoarthritis | 1.57 | 212 | 11 | 0.05 | PPARG, HMGB1, FZD3, |
| Pathway | | | | | ANXA5, ITGA2, FZD6, |
| | | | | | RBPJ, HIF1A, HTRA1, |
| | | | | | CTNNB1, SMAD1 |
| Role of | 1.5 | 128 | 8 | 0.06 | IL6ST, LIF, PTPN11, |
| NANOG in | | | | | FZD3, FZD6, AKT3, |
| Mammalian | | | | | CTNNB1, SMAD1 |
| Embryonic | | | | | |
| Stem Cell | | | | | |
| Pluripotency | | | | | |
| fMLP | 1.49 | 129 | 8 | 0.06 | GNG4, CALM1 |
| Signaling in | | | | | (includes others), |
| Neutrophils | | | | | ARPC1B, PTPN11, ARPC2, |
| | | | | | ARPC3, ITPR1, GNG5 |
| Colorectal Cancer | 1.45 | 254 | 12 | 0.05 | IL6ST, GNG4, IFNG, |
| Metastasis | | | | | BAD, PTPN11, FZD3, |
| Signaling | | | | | RHOA, FZD6, AKT3, |
| | | | | | CTNNB1, GNG5, BIRC5 |
| Gαq | 1.45 | 161 | 9 | 0.06 | GNG4, HMOX1, CALM1 |
| Signaling | | | | | (includes others), |
| | | | | | RGS2, PTPN11, RHOA, |
| | | | | | AKT3, ITPR1, GNG5 |
| Differential | 1.41 | 18 | 3 | 0.17 | CCL4, CSF2, CCL3 |
| Regulation | | | | | |
| of Cytokine | | | | | |
| Production in | | | | | |
| Macrophages | | | | | |
| and T Helper | | | | | |
| Cells by | | | | | |
| IL-17A | | | | | |
| and IL-17F | | | | | |
| Th1 Pathway | 1.41 | 135 | 8 | 0.06 | CD247, CD3G, IFNG, |
| | | | | | PTPN11, HLA-A, HAVCR2, |
| | | | | | HLA-B, CD3D |
| VEGF | 1.37 | 109 | 7 | 0.06 | BAD, FLT1, PTPN11, |
| Signaling | | | | | ACTB, AKT3, HIF1A, |
| | | | | | ACTG1 |
| GADD45 | 1.36 | 19 | 3 | 0.16 | PCNA, GADD45G, CDK1 |
| Signaling | | | | | |
| Systemic | 1.35 | 233 | 11 | 0.05 | CD247, PTPRC, CD3G, |
| Lupus | | | | | CD2BP2, PTPN11, HLA-A, |
| Erythematosus | | | | | SNRPF, HNRNPA2B1, |
| Signaling | | | | | HLA-B, AKT3, CD3D |
| Role of | 1.35 | 233 | 11 | 0.05 | CALM1 |
| Osteoblasts, | | | | | (includes others), |
| Osteoclasts | | | | | IFNG, BAD, PTPN11, |
| and | | | | | FZD3, ITGA2, FZD6, |
| Chondrocytes | | | | | AKT3, CTNNB1, CSF2, |
| in Rheumatoid | | | | | SMAD1 |
| Arthritis | | | | | |
| Antigen | 1.35 | 38 | 4 | 0.11 | B2M, IFNG, HLA-A, |
| Presentation | | | | | HLA-B |
| Pathway | | | | | |
| CXCR4 | 1.35 | 171 | 9 | 0.05 | GNG4, PTPN11, MYL6, |
| Signaling | | | | | RHOA, AKT3, ITPR1, |
| | | | | | ELMO2, GNG5, ELMO1 |
| Nitric Oxide | 1.35 | 113 | 7 | 0.06 | CALM1 |
| Signaling | | | | | (includes others), |
| in the | | | | | FLT1, PTPN11, RYR2, |
| Cardiovascular | | | | | CAV1, AKT3, ITPR1 |
| System | | | | | |
| PCP pathway | 1.35 | 61 | 5 | 0.08 | PFN1, FZD3, RHOA, |
| | | | | | FZD6, CTHRC1 |
| ERK/MAPK | 1.35 | 204 | 10 | 0.05 | PPARG, YWHAQ, LAMTOR3, |
| Signaling | | | | | PPP1CC, H3F3A/H3F3B, |
| | | | | | BAD, PTPN11, ITGA2, |
| | | | | | DUSP4, HSPB1 |
| Actin | 1.34 | 62 | 5 | 0.08 | ARPC1B, RHOA, ARPC2, |
| Nucleation | | | | | ITGA2, ARPC3 |
| by ARP- | | | | | |
| WASP | | | | | |
| Complex | | | | | |
| D-myo- | 1.34 | 144 | 8 | 0.06 | PTPRC, SET, PPP1CC, |
| inositol | | | | | PTPN11, PDCD1, PTPN13, |
| (1,4,5,6)- | | | | | NUDT16, PPP4C |
| Tetrakis- | | | | | |
| phosphate | | | | | |
| Biosynthesis | | | | | |
| D-myo- | 1.34 | 144 | 8 | 0.06 | PTPRC, SET, PPP1CC, |
| inositol | | | | | PTPN11, PDCD1, PTPN13, |
| (3,4,5,6)- | | | | | NUDT16, PPP4C |
| tetrakisphos phate | | | | | |
| Biosynthesis | | | | | |
| T Cell | 1.34 | 115 | 7 | 0.06 | CD247, PTPRC, CD3G, |
| Receptor | | | | | CALM1 |
| Signaling | | | | | (includes others), |
| | | | | | PTPN11, CTLA4, CD3D |
| Clathrin- | 1.34 | 207 | 10 | 0.05 | MYO6, UBB, SNX9, |
| mediated | | | | | ARPC1B, PTPN11, ACTB, |
| Endocytosis | | | | | ARPC2, ARPC3, CTTN, |
| Signaling | | | | | ACTG1 |
| Crosstalk | 1.32 | 89 | 6 | 0.07 | IFNG, HLA-A, ACTB, |
| between | | | | | HLA-B, CSF2, ACTG1 |
| Dendritic | | | | | |
| Cells and | | | | | |
| Natural | | | | | |
| Killer Cells | | | | | |
| Altered T | 1.31 | 90 | 6 | 0.07 | IL21, IFNG, CXCL13, |
| Cell and | | | | | HLA-A, HLA-B, CSF2 |
| B Cell | | | | | |
| Signaling in | | | | | |
| Rheumatoid | | | | | |
| Arthritis | | | | | |
| Phospholipase | 1.3 | 244 | 11 | 0.05 | CD247, GNG4, HMOX1, |
| C Signaling | | | | | CD3G, CALM1 |
| | | | | | (includes others), |
| | | | | | MYL6, RHOA, ITGA2, |
| | | | | | ITPR1, GNG5, CD3D |
| Polyamine | 1.3 | 22 | 3 | 0.14 | PPARG, OAZ1, CTNNB1 |
| Regulation | | | | | |
| in Colon | | | | | |
| Cancer | | | | | |

TABLE 10
Related to FIGS. 1, 3, 4, 10, and 11.
Gene signatures used for GSEA.

CADM1
CD1D
CD2
CD24
CD276
CD28
CD3D
CD3E
CD4
CD47
CD7
CLEC7A
CRTAM
EBI3
ELF4
GLMN
ICOSLG
IL12B
IL18
IL2
IL21
IL27
IL4
IL7
INS
JAG2
LAT
LAX1
LCK
NCK1
NCK2
NHEJ1
NLRC3
PTPRC
SART1
SFTPD
SIRPG
SIT1
SLA2
SOCS5
SPINK5
THY1
ZAP70
CD58
CLEC1
HLADRA
HLADRB1
SLAMF1
HLADPA1
HLADPB1

TABLE 11
Genes associated with cell programs that confer
superior functional properties of CD4+ Tfh-
like tumor infiltrating cells and CD4+ Tfh CTL cells.

| | Provision | Cell-cycle | |
|---|---|---|---|
| Cytotoxicity- | of CD8+ | regulation | Tfh-related |
| related | ‘help’ | (Proliferation) | (Proliferation) |
| GZMB | TNFRSF18 | STMN1 | MAF |
| KLRB | (GITR) | MAD2L1 | SH2D1A |
| FKBP1A | TNFRSF4 | SMC2 | (SAP) |
| SOD1 | (OX40) | NUSAP1 | PDCD1 |
| CCL3 | IFNG | TOP2A | BTLA |
| CCL4 | IL21 | | CD200 |
| ZEB2 | | | BCL6 |

TABLE 12

| | SEQ ID | |
|---|---|---|
| Description | NO: | Sequence |
| Human CD4 | 1 | ctctatcatttaagcacgactctgcagaaggaacaaagcaccctccccactgggctcctggttgcagagctccaag |
| variant 1 | | tcctcacacagatacgcctgtttgagaagcagcgggcaagaaagacgcaagcccagaggccctgccatttctgtg |
| (Genbank | | ggctcaggtccctactggctcaggcccctgcctccctcggcaaggccacaatgaaccggggagtcccttttaggc |
| Accession No. | | acttgcttctggtgctgcaactggcgctcctcccagcagccactcagggaaagaaagtggtgctgggcaaaaaag |
| NM_000616) | | gggatacagtggaactgacctgtacagcttcccagaagaagagcatacaattccactggaaaaactccaaccagat |
| | | aaagattctgggaaatcagggctccttcttaactaaaggtccatccaagctgaatgatcgcgctgactcaagaagaa |
| | | gcctttgggaccaaggaaactttcccctgatcatcaagaatcttaagatagaagactcagatacttacatctgtgaagt |
| | | ggaggaccagaaggaggaggtgcaattgctagtgttcggattgactgccaactctgacacccacctgcttcaggg |
| | | gcagagcctgaccctgaccttggagagcccccctggtagtagcccctcagtgcaatgtaggagtccaaggggtaa |
| | | aaacatacagggggggaagaccctctccgtgtctcagctggagctccaggatagtggcacctggacatgcactgt |
| | | cttgcagaaccagaagaaggtggagttcaaaatagacatcgtggtgctagctttccagaaggcctccagcatagtct |
| | | ataagaaagagggggaacaggtggagttctccttcccactcgcctttacagttgaaaagctgacgggcagtggcg |
| | | agctgtggtggcaggcggagagggcttcctcctccaagtcttggatcacctttgacctgaagaacaaggaagtgtct |
| | | gtaaaacgggttacccaggaccctaagctccagatgggcaagaagctcccgctccacctcaccctgccccaggc |
| | | cttgcctcagtatgctggctctggaaacctcaccctggcccttgaagcgaaaacaggaaagttgcatcaggaagtg |
| | | aacctggtggtgatgagagccactcagctccagaaaaatttgacctgtgaggtgtggggacccacctcccctaagc |
| | | tgatgctgagtttgaaactggagaacaaggaggcaaaggtctcgaagcgggagaaggcggtgtgggtgctgaac |
| | | cctgaggcggggatgtggcagtgtctgctgagtgactcgggacaggtcctgctggaatccaacatcaaggttctgc |
| | | ccacatggtccaccccggtgcagccaatggccctgattgtgctggggggcgtcgccggcctcctgcttttcattgg |
| | | gctaggcatcttcttctgtgtcaggtgccggcaccgaaggcgccaagcagagcggatgtctcagatcaagagactc |
| | | ctcagtgagaagaagacctgccagtgtcctcaccggtttcagaagacatgtagccccatttgaggcacgaggcca |
| | | ggcagatcccacttgcagcctccccaggtgtctgccccgcgtttcctgcctgcggaccagatgaatgtagcagatc |
| | | cccagcctctggcctcctgttcgcctcctctacaatttgccattgtttctcctgggttaggccccggcttcactggttga |
| | | gtgttgctctctagtttccagaggcttaatcacaccgtcctccacgccatttc0cttttccttcaagcctagcccttctctc |
| | | attatttctctctgaccctctccccactgctcatttggatcccaggggagtgttcagggccagccctggctggcatgga |
| | | gggtgaggctgggtgtctggaagcatggagcatgggactgttcttttacaagacaggaccctgggaccacagagg |
| | | gcaggaacttgcacaaaatcacacagccaagccagtcaaggatggatgcagatccaga0ggtttctggcagcca |
| | | gtacctcctgccccatgctgcccgcttctcaccctatgtgggtgg0gaccacagactcacatcctgaccttgcacaa |
| | | acagcccctctggacacagccccatgtacacggcctcaagggatgtctcacatcctctgtctatttgagacttagaaa |
| | | aatcctacaaggctggcagtgacagaactaagatgatcatctccagtttatagaccagaaccagagctcagagagg |
| | | ctagatgattgattaccaagtgccggactagcaagtgctggagtcgggactaacccaggtcccttgtcccaagttcc |
| | | actgctgcctcttgaatgcagggacaaatgccacacgg0ctctcaccagtggctagtggtgggtactcaatgtgtac |
| | | ttttgggttcacagaagcacagcacccatgggaagggtccatctcagagaatttacgagcagggatgaaggcctcc |
| | | ctgtctaaaatccctccttcatcccccgctggtggcagaatctgttaccagaggacaaagcctttggctcttctaatca |
| | | gagcgcaagctgggagcacaggcactgcaggagagaatgcccagtgaccagtcactgaccctgtgcagaacct |
| | | cctggaagcgagctttgctgggagagggggtagc0tagcctgagagggaaccctctaagggacctcaaaggtga |
| | | ttgtgccaggctctgcgcctgccccacaccctcccttaccctcctccagaccattcaggacacagggaaatcaggg |
| | | ttacaaatcttcttgatccacttctctcaggatcccctctcttcctacccttcctcaccacttccctcagtcccaactcctttt |
| | | ccctatttccttctcctcctgtctttaaagcctgcctcttccaggaagacccccctattgctgctggggctccccatttgc |
| | | ttactttgcatttgtgccca00ctctccacccctgctcccctgagctgaaataaaaatacaataaacttac |
| Human CD4 | 2 | MNRGVPFRHLLLVLQLALLPAATQGKKVVLGKKGDTVELTCTASQKKS |
| variant 1 | | IQFHWKNSNQIKILGNQGSFLTKGPSKLNDRADSRRSLWDQGNFPLIIKN |
| polypeptide | | LKIEDSDTYICEVEDQKEEVQLLVFGLTANSDTHLLQGQSLTLTLESPPGS |
| (Genbank | | SPSVQCRSPRGKNIQGGKTLSVSQLELQDSGTWTCTVLQNQKKVEFKIDI |
| Accession No. | | VVLAFQKASSIVYKKEGEQVEFSFPLAFTVEKLTGSGELWWQAERASSS |
| NP_000607.1) | | KSWITFDLKNKEVSVKRVTQDPKLQMGKKLPLHLTLPQALPQYAGSGN |
| | | LTLALEAKTGKLHQEVNLVVMRATQLQKNLTCEVWGPTSPKLMLSLKL |
| | | ENKEAKVSKREKAVWVLNPEAGMWQCLLSDSGQVLLESNIKVLPTWST |
| | | PVQPMALIVLGGVAGLLLFIGLGIFFCVRCRHRRRQAERMSQIKRLLSEK |
| | | KTCQCPHRFQKTCSPI |
| Human CD4 | 3 | ctctcttcatttaagcacgactctgcagaaggaacaaagcaccctccccactgggctcctggttgcagagctccaag |
| variant 2 | | tcctcacacagatacgcctgtttgagaagcagcgggcaagaaagacgcaagcccagaggtccatccaagctgaat |
| polynucleotide | | gatcgcgctgactcaagaagaagcctttgggaccaaggaaactttcccctgatcatcaagaatcttaagatagaaga |
| (Genbank | | ctcagatacttacatctgtgaagtggaggaccagaaggaggaggtgcaattgctagtgttcggattgactg0ccaac |
| Accession No. | | tctgacacccacctgcttcaggggcagagcctgaccctgaccttggagagcccccctggtagtagcccctcagtgc |
| NM_001195014) | | aatgtaggagtccaaggggtaaaaacatacagggggggaagaccctctccgtgtctcagctggagctccaggata |
| | | gtggcacctggacatgcactgtcttgcagaaccagaagaaggtggagttcaaaatagacatcgtggtgctagggtg |
| | | atgaatacgcctctaaggagtggaggccaaatggcttctgtggtccaggaatcctaaggacagcaagg0atcccct |
| | | gtggctgggctgctctgtgatggcttccgggaggagggaggtggcctgctgtaggaaaatgctgggtggaagaa |
| | | gggagagaaggctggagaggtaggaaggaactgaagtatctgaagtgacaaggtgggtgtctggactcgtcggg |
| | | tccccttccatctccctgctgcctccacatgccaaccccactcgtgcaccctcatcttcctatctcctcacccagggtct |
| | | ctcccttcccacctccagctttccagaaggcctccagcatagtctataagaaagagggggaacag0gtggagttct |
| | | ccttcccactcgcctttacagttgaaaagctgacgggcagtggcgagctgtggtggcaggcggagagggcttcctc |
| | | ctccaagtcttggatcacctttgacctgaagaac0aaggaagtgtctgtaaaacgggttacccaggaccctaagctc |
| | | cagatgggcaagaagctc0ccgctccacctcaccctgccccaggccttgcctcagtatgctggctctggaaacctc |
| | | accctggcccttgaagcgaaaacaggaaagttgcatcaggaagtgaacctggtggtgatgaga0gccactcagct |
| | | ccagaaaaatttgacctgtgaggtgtggggacccacctcccctaagctgatgctgagtttgaaactggagaacaag |
| | | gaggcaaaggtctcgaagcgggagaaggcggtgtgggtgctgaaccctgaggcggggatgtggcagtgtctgc |
| | | tgagtgactcgggacaggtcctgctggaatccaacatcaaggttctgcccacatggtccaccccggtgcagccaat |
| | | ggccctgattgtgctggggggcgtcgccggcctcctgcattcattgggctaggcatcacttc0tgtgtcaggtgcc |
| | | ggcaccgaaggcgccaagcagagcggatgtctcagatcaagagactcctcagtgagaagaagacctgccagtgt |
| | | cctcaccggatcagaagacatgtagccccatttgaggcacgaggccaggcagatcccacttgcagcctccccag |
| | | gtgtctgccccgcgtacctgcctgcggaccagatgaatgtagcagatccccagcctctggcctcctgacgcctcct |
| | | ctacaatagccattgatctcctgggttaggccccggcttcactggagagtgagctc0tctagatccagaggcttaat |
| | | cacaccgtcctccacgccataccattccacaagcctagcccactctcattatactctctgaccctctccccactgctc |
| | | ataggatcccaggggagtgacagggccagccctggctggcatggagggtgaggctgggtgtctggaagcatgg |
| | | agcatgggactgactatacaagacaggaccctgggaccacagagggcaggaacttgcac0aaaatcacacagc |
| | | caagccagtcaaggatggatgcagatccagaggtactggcagccag0tacctcctgccccatgctgcccgcttct |
| | | caccctatgtgggtgggaccacagactcacatcctgaccagcacaaacagcccctctggacacagccccatgtac |
| | | acggcctcaagggatgtctcacatcctctgtctatttgagacttagaaaaatcctacaaggctggcagtgacagaact |
| | | aagatgatcatctccagatatagaccagaaccagagctcagagaggctagatgattgattaccaagtgccggacta |
| | | gcaagtgctggagtcgggactaacccaggtcccagtccca0agaccactgctgcctcttgaatgcagggacaaat |
| | | gccacacggctctcaccagtggctagtggtgggtactcaatgtgtacttttgggttcacagaagcacagcacccatg |
| | | ggaagggtccatctcagagaatttacgagcagggatgaaggcctccctgtctaaaatccctccttcatcccccgctg |
| | | gtggcagaatctgaaccagaggacaaagccatggctcactaatcagagcgcaagctgggagcacaggcactgc |
| | | aggagagaatgcccagtgaccagtcactgaccctg0tgcagaacctcctggaagcgagctagctgggagaggg |
| | | ggtagctagcctgagagggaaccctctaagggacctcaaaggtgattgtgccaggctctgcgcctgccccacacc |
| | | ctcccttaccctcctccagaccattcaggacacagggaaatcagggaacaaatcacttgatccacactctcaggat |
| | | cccctctcttcctacccttcctcaccacttccctcagtcccaactccttttccctatttccttctcctcctgtctttaaagcct |
| | | gcctcttccaggaagaccccccta00ttgctgctggggctccccatttgcttactttgcatttgtgcccactctccacc |
| | | cctgctc0ccctgagctgaaataaaaatacaataaacttac |
| Human CD4 | 4 | MPTPLVHPHLPISSPRVSPFPPPAFQKASSIVYKKEGEQVEFSFPLAFTVEK |
| variant 2 | | LTGSGELWWQAERASSSKSWITFDLKNKEVSVKRVTQDPKLQMGKKLP |
| polypeptide | | LHLTLPQALPQYAGSGNLTLALEAKTGKLHQEVNLVVMRATQLQKNLT |
| (Genbank | | CEVWGPTSPKLMLSLKLENKEAKVSKREKAVWVLNPEAGMWQCLLSD |
| Accession No. | | SGQVLLESNIKVLPTWSTPVQPMALIVLGGVAGLLLFIGLGIFFCVRCRHR |
| NP_001181943.1) | | RRQAERMSQIKRLLSEKKTCQCPHRFQKTCSPI |
| Human CD4 | 5 | ctctcacatttaagcacgactctgcagaaggaacaaagcaccctccccactgggctcctggagcagagctccaag |
| variant 3 | | tcctcacacagatacgcctgtttgagaagcagcgggcaagaaagacgcaagcccagaggtccatccaagctgaat |
| polynucleotide | | gatcgcgctgactcaagaagaagcctttgggaccaaggaaactttcccctgatcatcaagaatcttaagatagaaga |
| (Genbank | | ctcagatacttacatctgtgaagtggaggaccagaaggaggaggtgcaattgctagtgacggatgtgagt0gggg |
| Accession No. | | cagtgactgccaactctgacacccacctgcttcaggggcagagcctgaccctgaccaggagagcccccctggta |
| NM_001195015) | | gtagcccctcagtgcaatgtaggagtccaaggggtaaaaacatacagggggggaagaccctctccgtgtctcagc |
| | | tggagctccaggatagtggcacctggacatgcactgtcagcagaaccagaagaaggtggagacaaaatagacat |
| | | cgtggtgctagcatccagaaggcctccagcatagtctataagaaagagggggaacaggtggagactcc0ttccc |
| | | actcgccatacagagaaaagctgacgggcagtggcgagctgtggtggcaggcggagagggcacctcctccaa |
| | | gtcaggatcaccatgacctgaagaacaaggaagtgtctgtaaaacgggttacccaggaccctaagctccagatgg |
| | | gcaagaagctcccgctccacctcaccctgccccaggccagcctcagtatgctggctctggaaacctcaccctggc |
| | | ccagaagcgaaaacaggaaagagcatcaggaagtgaacctggtggtgatgagagccactcagctc0cagaaaa |
| | | atagacctgtgaggtgtggggacccacctcccctaagctgatgctgagatgaaactggagaacaaggaggcaaa |
| | | ggtctcgaagcgggagaaggcggtgtgggtgctgaac0cctgaggcggggatgtggcagtgtctgctgagtgac |
| | | tcgggacaggtcctgctggaatcc0aacatcaaggactgcccacatggtccaccccggtgcagccaatggccct |
| | | gattgtgctggggggcgtcgccggcctcctgctatcattgggctaggcatcacttctgtgtcaggtgc0cggcacc |
| | | gaaggcgccaagcagagcggatgtctcagatcaagagactcctcagtgagaagaagacctgccagtgtcctcac |
| | | cggatcagaagacatgtagccccatttgaggcacgaggccaggcagatcccacttgcagcctccccaggtgtctg |
| | | ccccgcgtttcctgcctgcggaccagatgaatgtagcagatccccagcctctggcctcctgttcgcctcctctacaat |
| | | ttgccattgtttctcctgggttaggccccggcttcactggagagtgagctctctagatccag0aggcttaatcacacc |
| | | gtcctccacgccatttccttttccttcaagcctagcccttctctcattatttctctctgaccctctccccactgctcatttgg |
| | | atcccaggggagtgacagggccagccctggctggcatggagggtgaggctgggtgtctggaagcatggagcat |
| | | gggactgacattacaagacaggaccctgggaccacagagggcaggaacttgcacaaaatcacacagccaagcc |
| | | agtcaaggatggatgcagatccagaggtactggcagccagtacctcctgccc0catgctgcccgcactcacccta |
| | | tgtgggtgggaccacagactcacatcctgaccagcacaaacagcccctctggacacagccccatgtacacggcct |
| | | caagggatgtctcacatcctctgtctatttgagacttagaaaaatcctacaaggctggcagtgacagaactaagatga |
| | | tcatctccagatatagaccagaaccagagctcagagaggctagatgattgattaccaagtgc0cggactagcaagt |
| | | gctggagtcgggactaacccaggtcccttgtcccaagttccactgct0gcctcttgaatgcagggacaaatgccac |
| | | acggctctcaccagtggctagtggtgggtactcaatgtgtacttttgggttcacagaagcacagcacccatgggaag |
| | | ggtccatctcagagaatttacgagcagggatgaaggcctccctgtctaaaatccctccttcatcccccgctggtggc |
| | | agaatctgaaccagaggacaaagcctaggctcactaatcagagcgcaagctgggagcacaggcactgcagga |
| | | gagaatgcccagtgaccagtcactgaccctgtgcagaacctcc0tggaagcgagctagctgggagagggggta |
| | | gctagcctgagagggaaccctctaagggacctcaaaggtgattgtgccaggctctgcgcctgccccacaccctcc |
| | | cttaccctcctccagaccattcaggacacagggaaatcagggttacaaatcttcttgatccacttctctcaggatcccc |
| | | tctcttcctacccttcctcaccacttccctcagtcccaactccttttccctatttccttctcctcctgtctttaaagcctgcct |
| | | cttccaggaagacccccctattgctgctgggg0ctccccatttgcttactttgcatttgtgcccactctccacccctgct |
| | | cccctgagctgaaataaaaatacaataaacttac |
| Human CD4 | 6 | MGKKLPLHLTLPQALPQYAGSGNLTLALEAKTGKLHQEVNLVVMRATQ |
| variant 3 | | LQKNLTCEVWGPTSPKLMLSLKLENKEAKVSKREKAVWVLNPEAGMW |
| polypeptide | | QCLLSDSGQVLLESNIKVLPTWSTPVQPMALIVLGGVAGLLLFIGL GIFFC |
| (Genbank | | VRCRHRRRQAERMSQIKRLLSEKKTCQCPHRFQKTCSPI |
| Accession No. | | |
| NP_001181944.1) | | |
| Human CD4 | 7 | ctctcttcatttaagcacgactctgcagaaggaacaaagcaccctccccactgggctcctggttgcagagctccaag |
| variant 4 | | tcctcacacagatacgcctgtttgagaagcagcgggcaagaaagacgcaagcccagaggtccatccaagctgaat |
| polynucleotide | | gatcgcgctgactcaagaagaagcctttgggaccaaggaaactttcccctgatcatcaagaatcttaagatagaaga |
| (Genbank | | ctcagatacttacatctgtgaagtggaggaccagaaggaggaggtgcaattgctagtgttcggattgactg0ccaac |
| Accession No. | | tctgacacccacctgcttcaggggcagagcctgaccctgaccttggagagcccccctggtagtagcccctcagtgc |
| NM_001195016) | | aatgtaggagtccaaggggtaaaaacatacagggggggaagaccctctccgtgtctcagctggagctccaggata |
| | | gtggcacctggacatgcactgtcttgcagaaccagaagaaggtggagttcaaaatagacatcgtggtgctagctttc |
| | | cagaaggcctccagcatagtctataagaaagagggggaacaggtggagttctccttcccactcgcct0ttacagttg |
| | | aaaagctgacgggcagtggcgagctgtggtggcaggcggagagggcttcctcctccaagtcttggatcacctttg |
| | | acctgaagaacaaggaagtgtctgtaaaacgggttacccaggaccctaagctccagatgggcaagaagctcccg |
| | | ctccacctcaccctgccccaggccttgcctcagtatgctggctctggaaacctcaccctggcccttgaagcgaaaac |
| | | aggaaagttgcatcaggaagtgaacctggtggtgatgagagccactcagctccagaaaaatttga0cctgtgaggt |
| | | gtggggacccacctcccctaagctgatgctgagtttgaaactggagaacaaggaggcaaaggtctcgaagcggg |
| | | agaaggcggtgtgggtgctgaaccctgaggcgggga0tgtggcagtgtctgctgagtgactcgggacaggtcct |
| | | gctggaatccaacatcaaggttc0tgcccacatggtccaccccggtgcagccaatggccctgattgtgctggggg |
| | | gcgtcgccggcctcctgcttttcattgggctaggcatcttcttctgtgtcaggtgccggcaccgaaggc0gccaagc |
| | | agagcggatgtctcagatcaagagactcctcagtgagaagaagacctgccagtgtcctcaccggtttcagaagaca |
| | | tgtagccccatttgaggcacgaggccaggcagatcccacttgcagcctccccaggtgtctgccccgcgtttcctgc |
| | | ctgcggaccagatgaatgtagcagatccccagcctctggcctcctgttcgcctcctctacaatttgccattgtttctcct |
| | | gggttaggccccggcttcactggttgagtgttgctctctagtttccagaggcttaatcaca0ccgtcctccacgccatt |
| | | tccttttccttcaagcctagcccttctctcattatttctctctgaccctctccccactgctcatttggatcccaggggagtgt |
| | | tcagggccagccctggctggcatggagggtgaggctgggtgtctggaagcatggagcatgggactgttcttttaca |
| | | agacaggaccctgggaccacagagggcaggaacttgcacaaaatcacacagccaagccagtcaaggatggatg |
| | | cagatccagaggtttctggcagccagtacctcctgccccatgctgcccgct0tctcaccctatgtgggtgggaccac |
| | | agactcacatcctgaccttgcacaaacagcccctctggacacagccccatgtacacggcctcaagggatgtctcac |
| | | atcctctgtctatttgagacttagaaaaatcctacaaggctggcagtgacagaactaagatgatcatctccagtttatag |
| | | accagaaccagagctcagagaggctagatgattgattaccaagtgccggactagcaagt0gctggagtcgggact |
| | | aacccaggtcccttgtcccaagttccactgctgcctcttgaatgc0agggacaaatgccacacggctctcaccagtg |
| | | gctagtggtgggtactcaatgtgtacttttgggttcacagaagcacagcacccatgggaagggtccatctcagagaa |
| | | tttacgagcagggatgaaggcctccctgtctaaaatccctccttcatcccccgctggtggcagaatctgttaccagag |
| | | gacaaagcctttggctcttctaatcagagcgcaagctgggagcacaggcactgcaggagagaatgcccagtgacc |
| | | agtcactgaccctgtgcagaacctcctggaagcgagctt0tgctgggagagggggtagctagcctgagagggaa |
| | | ccctctaagggacctcaaaggtgattgtgccaggctctgcgcctgccccacaccctcccttaccctcctccagacca |
| | | ttcaggacacagggaaatcagggttacaaatcttcttgatccacttctctcaggatcccctctcttcctacccttcctca |
| | | ccacttccctcagtcccaactcdtttccctatttccttctcctcctgtctttaaagcctgcctcttccaggaagacccccc |
| | | tattgctgctggggctccccatttgct0tactttgcatttgtgcccactctccacccctgctcccctgagctgaaataaa |
| | | aatacaataaacttac |
| Human CD4 | 8 | MGKKLPLHLTLPQALPQYAGSGNLTLALEAKTGKLHQEVNLVVMRATQ |
| variant 4 | | LQKNLTCEVWGPTSPKLMLSLKLENKEAKVSKREKAVWVLNPEAGMW |
| polypeptide | | QCLLSDSGQVLLESNIKVLPTWSTPVQPMALIVLGGVAGLLLFIGLGIFFC |
| (Genbank | | VRCRHRRRQAERMSQIKRLLSEKKTCQCPHRFQKTCSPI |
| Accession No. | | |
| NP_001181945.1) | | |
| Human CD4 | 9 | ctctcttcatttaagcacgactctgcagaaggaacaaagcaccctccccactgggctcctggttgcagagctccaag |
| variant 5 | | tcctcacacagatacgcctgtttgagaagcagcgggcaagaaagacgcaagcccagaggtccatccaagctgaat |
| polynucleotide | | gatcgcgctgactcaagaagaagcctttgggaccaaggaaactttcccctgatcatcaagaatcttaagatagaaga |
| (Genbank | | ctcagatacttacatctgtgaagtggaggaccagaaggaggagtgactgccaactctgacacccacctgct0tcag |
| Accession No. | | gggcagagcctgaccctgaccttggagagcccccctggtagtagcccctcagtgcaatgtaggagtccaaggggt |
| NM_001195017) | | aaaaacatacagggggggaagaccctctccgtgtctcagctggagctccaggatagtggcacctggacatgcact |
| | | gtcttgcagaaccagaagaaggtggagttcaaaatagacatcgtggtgctagctttccagaaggcctccagcatagt |
| | | ctataagaaagagggggaacaggtggagttctccttcccactcgcctttacagttgaaaagctgacggg0cagtgg |
| | | cgagctgtggtggcaggcggagagggcttcctcctccaagtcttggatcacctttgacctgaagaacaaggaagtg |
| | | tctgtaaaacgggttacccaggaccctaagctccagatgggcaagaagctcccgctccacctcaccctgccccag |
| | | gccttgcctcagtatgctggctctggaaacctcaccctggcccttgaagcgaaaacaggaaagttgcatcaggaag |
| | | tgaacctggtggtgatgagagccactcagctccagaaaaatttgacctgtgaggtgtggggacccac0ctccccta |
| | | agctgatgctgagtttgaaactggagaacaaggaggcaaaggtctcgaagcgggagaaggcggtgtgggtgctg |
| | | aaccctgaggcggggatgtggcagtgtctgctgagtga0ctcgggacaggtcctgctggaatccaacatcaaggt |
| | | tctgcccacatggtccaccccggt0gcagccaatggccctgattgtgctggggggcgtcgccggcctcctgcttttc |
| | | attgggctaggcatcttcttctgtgtcaggtgccggcaccgaaggcgccaagcagagcggatgtctca0gatcaag |
| | | agactcctcagtgagaagaagacctgccagtgtcctcaccggtttcagaagacatgtagccccatttgaggcacga |
| | | ggccaggcagatcccacttgcagcctccccaggtgtctgccccgcgtttcctgcctgcggaccagatgaatgtagc |
| | | agatccccagcctctggcctcctgttcgcctcctctacaatttgccattgtttctcctgggttaggccccggcttcactg |
| | | gttgagtgttgctctctagtttccagaggcttaatcacaccgtcctccacgccatttcctt0ttccttcaagcctagccctt |
| | | ctctcattatttctctctgaccctctccccactgctcatttggatcccaggggagtgttcagggccagccctggctggc |
| | | atggagggtgaggctgggtgtctggaagcatggagcatgggactgttcttttacaagacaggaccctgggaccac |
| | | agagggcaggaacttgcacaaaatcacacagccaagccagtcaaggatggatgcagatccagaggtttctggca |
| | | gccagtacctcctgccccatgctgcccgcttctcaccctatgtgggtgggac0cacagactcacatcctgaccttgc |
| | | acaaacagcccctctggacacagccccatgtacacggcctcaagggatgtctcacatcctctgtctatttgagactta |
| | | gaaaaatcctacaaggctggcagtgacagaactaagatgatcatctccagtttatagaccagaaccagagctcaga |
| | | gaggctagatgattgattaccaagtgccggactagcaagtgctggagtcgggactaacccag0gtcccttgtccca |
| | | agttccactgctgcctcttgaatgcagggacaaatgccacacggctc0tcaccagtggctagtggtgggtactcaat |
| | | gtgtacttttgggttcacagaagcacagcacccatgggaagggtccatctcagagaatttacgagcagggatgaag |
| | | gcctccctgtctaaaatccctccttcatcccccgctggtggcagaatctgttaccagaggacaaagcctttggctcttc |
| | | taatcagagcgcaagctgggagcacaggcactgcaggagagaatgcccagtgaccagtcactgaccctgtgcag |
| | | aacctcctggaagcgagctttgctgggagagggggtagctag0cctgagagggaaccctctaagggacctcaaa |
| | | ggtgattgtgccaggctctgcgcctgccccacaccctcccttaccctcctccagaccattcaggacacagggaaat |
| | | cagggttacaaatcttcttgatccacttctctcaggatcccctctcttcctacccttcctcaccacttccctcagtcccaa |
| | | ctccttttccctatttccttctcctcctgtctttaaagcctgcctcttccaggaagacccccctattgctgctggggctccc |
| | | catttgcttactttgcatttgtgcccactc0tccacccctgctcccctgagctgaaataaaaatacaataaacttac |
| Human CD4 | 10 | MGKKLPLHLTLPQALPQYAGSGNLTLALEAKTGKLHQEVNLVVMRATQ |
| variant 5 | | LQKNLTCEVWGPTSPKLMLSLKLENKEAKVSKREKAVWVLNPEAGMW |
| polypeptide | | QCLLSDSGQVLLESNIKVLPTWSTPVQPMALIVLGGVAGLLLFIGLGIFFC |
| (Genbank | | VRCRHRRRQAERMSQIKRLLSEKKTCQCPHRFQKTCSPI |
| Accession No. | | |
| N_001181946.1) | | |
| Human | 11 | gagaagatgtttgaaaaaactgactctgctaatgagcctggactcagagctcaagtctgaactctacctccagacag |
| CXCL13 variant | | aatgaagttcatctcgacatctctgcttctcatgctgctggtcagcagcctctctccagtccaaggtgttctggaggtct |
| 1 polynucleotide | | attacacaagcttgaggtgtagatgtgtccaagagagctcagtctttatccctagacgcttcattgatcgaattcaaatc |
| (Genbank | | ttgccccgtgggaatggttgtccaagaaaagaaatcatagtctggaagaagaacaagtcaatt0gtgtgtgtggacc |
| Accession No. | | ctcaagctgaatggatacaaagaatgatggaagtattgagaaaaagaagttcttcaactctaccagttccagtgtttaa |
| NM_006419) | | gagaaagattccctgatgctgatatttccactaagaacacctgcattcttcccttatccctgctctggattttagttttgtg |
| | | cttagttaaatcttttccaggaaaaagaacttccccatacaaataagcatgagactatgtaaaaataaccttgcagaag |
| | | ctgatggggcaaactcaagcttcttcactcacagcaccctatataca0cttggagtttgcattcttattcatcagggag |
| | | gaaagtttctttgaaaatagttattcagttataagtaatacaggattattttgattatatacttgttgtttaatgtttaaaatttct |
| | | tagaaaacaatggaatgagaatttaagcctcaaatttgaacatgtggcttgaattaagaagaaaattatggcatatatta |
| | | aaagcaggcttctatgaaagactcaaaaagctgcctgggaggcagatggaacttgagcctgtcaagaggcaaag |
| | | gaatccatgtagtagatatcctctgctt0aaaaactcactacggaggagaattaagtcctacttttaaagaatttctttat |
| | | aaaatttactgtctaagattaatagcattcgaagatccccagacttcatagaatactcagggaaagca0tttaaagggt |
| | | gatgtacacatgtatcctttcacacatttgccttgacaaacttctttcac0tcacatctttttcactgactttttttgtggggg |
| | | gcggggccggggggactctggtatctaattctttaatgattcctataaatctaatgacattcaataaagttgagcaaac |
| | | attttact0taaaaaaaaaaaaaaaaaa |
| Human | 12 | mkfistslllmllvsslspvqgvlevyytslrcrcvqessvfiprrfidriqilprgngcprkeiivwkknksivcvd |
| CXCL13 variant | | pqaewiqrmmevlrkrssstlpvpvfkrkip |
| 1 polypeptide | | |
| (Genbank | | |
| Accession No. | | |
| NP_006410) | | |
| Human | 13 | acagcctggactcagagctcaagtctgaactctacctccagacagaatgaagttcatctcgacatctctgcttctcatg |
| CXCL13 variant | | ctgctggtcagcagcctctctccagtccaaggtgttctggaggtctattacacaagcttgaggtgtagatgtgtccaa |
| 2 polynucleotide | | gagagctcagtctttatccctagacgcttcattgatcgaattcaaatcttgccccgtgggaatggttgtccaagaaaag |
| (Genbank | | aaatcatagtctggaagaagaacaagtcaattgtgtgtgtggaccctcaagctgaatggataca0aagaatgatgga |
| Accession No. | | agtattgagaaaaagaagttcttcaactctaccagttccagtgtttaagagaaagattccctgatgctgatatttccact |
| NM_00137155) | | aagaacacctgcattcttcccttatccctgctctggattttagttttgtgcttagttaaatcttttccaggaaaaagaacttc |
| | | cccatacaaataagcatgagactatgtaaaaataaccttgcagaagctgatggggcaaactcaagcttcttcactcac |
| | | agcaccctatatacacttggagtttgcattcttattcatcagggagg0aaagtttctttgaaaatagttattcagttataag |
| | | taatacaggattattttgattatatacttgttgtttaatgtttaaaatttcttagaaaacaatggaatgagaatttaagcctca |
| | | aatttgaacatgtggcttgaattaagaagaaaattatggcatatattaaaagcaggcttctatgaaagactcaaaaagc |
| | | tgcctgggaggcagatggaacttgagcctgtcaagaggcaaaggaatccatgtagtagatatcctctgcttaaaaac |
| | | tcactacggaggagaattaagtccta0cttttaaagaatttctttataaaatttactgtctaagattaatagcattcgaaga |
| | | tccccagacttcatagaatactcagggaaagcatttaaagggtgatgtacacatgtatcctttca0cacatttgccttga |
| | | caaacttctttcactcacatattttcactgactttttttgtgggg0ggcggggccggggggactctggtatctaattcttt |
| | | aatgattcctataaatctaatgacattcaataaagttgagcaaacattttacttaa |
| Human | 14 | MKFISTSLLLMLLVSSLSPVQGVLEVYYTSLRCRCVQESSVFIPRRFIDRIQ |
| CXCL13 variant | | ILPRGNGCPRKEIIVWKKNKSIVCVDPQAEWIQRMMEVLRKRSSSTLPVP |
| 2 polypeptide | | VFKRKIP |
| (Genbank | | |
| Accession No. | | |
| NP_001358487.1) | | |
| Human CXCR5 | 15 | ctctcaacataagacagtgaccagtctggtgactcacagccggcacagccatgaactacccgctaacgctggaaat |
| variant 1 | | ggacctcgagaacctggaggacctgttctgggaactggacagattggacaactataacgacacctccctggtgga |
| polynucleotide | | aaatcatctctgccctgccacagaggggcccctcatggcctccttcaaggccgtgttcgtgcccgtggcctacagc |
| (Genbank | | ctcatcttcctcctgggcgtgatcggcaacgtcctggtgctggtgatcctggagcggcaccggcagacacgca0gt |
| Accession No. | | tccacggagaccttcctgttccacctggccgtggccgacctcctgctggtcttcatcttgccctttgccgtggccgag |
| NM_001716) | | ggctctgtgggctgggtcctggggaccttcctctgcaaaactgtgattgccctgcacaaagtcaacttctactgcagc |
| | | agcctgctcctggcctgcatcgccgtggaccgctacctggccattgtccacgccgtccatgcctaccgccaccgcc |
| | | gcctcctctccatccacatcacctgtgggaccatctggctggtgggcttcctccttgccttgccag0agattctcttcg |
| | | ccaaagtcagccaaggccatcacaacaactccctgccacgttgcaccttctcccaagagaaccaagcagaaacgc |
| | | atgcctggttcacctcccgattcctctaccatgtggcgggattcctgctgcccatgctggtgatgggctggtgctacgt |
| | | gggggtagtgcacaggttgcgccaggcccagcggcgccctcagcggcagaaggcagtcagggtggccatcct |
| | | ggtgacaagcatcttcttcctctgctggtcaccctaccacatcgtcatcttcctggacaccc0tggcgaggctgaagg |
| | | ccgtggacaatacctgcaagctgaatggctctctccccgtggccatcaccatgtgtgagttcctgggcctggcccac |
| | | tgctgcctcaaccccatgctctacactt0tcgccggcgtgaagttccgcagtgacctgtcgcggctcctgacgaagc |
| | | tgggctgtaccg0gccctgcctccctgtgccagctcttccctagctggcgcaggagcagtctctctgagtcagaga |
| | | atgccacctctctcaccacgttctaggtcccagtgtccccttttattgctgcttttc0cttggggcaggcagtgatgctg |
| | | gatgctccttccaacaggagctgggatcctaagggctcaccgtggctaagagtgtcctaggagtatcctcatttggg |
| | | gtagctagaggaaccaacccccatttctagaacatccctgccagctcttctgccggccctggggctaggctggagc |
| | | ccagggagcggaaagcagctcaaaggcacagtgaaggctgtccttacccatctgcacccccctgggctgagaga |
| | | acctcacgcacctcccatcctaatcatccaatgctcaagaaacaacttcta0cttctgcccttgccaacggagagcg |
| | | cctgcccctcccagaacacactccatcagcttaggggctgctgacctccacagcacccctctctcctcctgcccac |
| | | ctgtcaaacaaagccagaagctgagcaccaggggatgagtggaggaaaggctgaggaaaggccagctggcag |
| | | cagagtgtggccacggacaactcagtccctaaaaacacagacattctgccaggcccccaagcctgcagtcatctt |
| | | gaccaagcaggaagctcagactggagagacaggtagctgcccctggc0tctgaccgaaacagcgctgggtcca |
| | | ccccatgtcaccggatcctgggtggtctgcaggcagggctgactctaggtgcccaggaggccagccagtgacct |
| | | gaggaagcgtgaaggccgagaagcaagaaagaaacccgacagagggaagaaaagagcatcacccgaaccc |
| | | caaggagggagatggatcaatcaaacccggcggtcccctccgccaggcgagatggggtggggtggaga0actc |
| | | ctagggtggctgggtccaggggatgggaggttgtgggcattgatggggaaggaggc0tggcttgtcccctcctca |
| | | ctcccacccataagctatagacccgaggaaactcagagtcggaacggagaaaggtggactggaaggggcccgt |
| | | gggagtcatctcaaccatcccctccgtggcatcaccttaggcagggaagtgtaagaaacacactgaggcagggaa |
| | | gtccccaggccccaggaagccgtgccctgcccccgtgaggatgtcactcagatggaaccgcaggaagctgctcc |
| | | gtgcttgtttgctcacctggggtgtgggaggcccgtccggcagttctgggtgctcccta0ccacctccccagcctttg |
| | | atcaggtggggagtcagggacccctgcccagtcccactcaagccaagcagccaagctccagggaggccccact |
| | | ggggaaataacagctgtggctcacgtgagagtgtcacacggcaggacaacgaggaagccctaagacgtcccatt |
| | | ttctctgagtatctcctcgcaagctgggtaatcgatgggggagtctgaagcagatgcaaagaggcaagaggctgga |
| | | attgaattactattaataaaaaggcacctataaaacaggtcaatacagtaca0ggcagcacagagacccccggaa |
| | | caagcctaaaaattgtttcaaaataaaaaccaagaagatgtcttcacatattgtatttatatatttatatttatatatatattta |
| | | tataatggtacaaaatggctgggggtgtggccatggatggagggaagagtaggctggcctgtggcgtgggtggg |
| | | aggagaggggacggagagggcactcggcccgctgcaatctgacccctctctcctcagggcaggaaacacagag |
| | | tcagacagtttgggggggtcttgggccaggggtggagggctcaagg00gcacagggcccaggctgaggcagg |
| | | gcgggcaaagcgcctggcaggatgaagggcaagtgg0ccccccaaacacagaggccctggccatggaccctg |
| | | ggaggtgaccggggtgagtcaggggcctgagtcagccccagaggaagcgctggacctggccgatggtgggcc |
| | | gagaggacagcaccaggctgggagaagtggggcgagacccatgtattacagctgccagtgcaagaccaggcc |
| | | ctccaggccaggaaggctagggacgggtcctggtagaagacaccctgtctagaatggc0ccaggtcctggaggt |
| | | ggggcgcaaaaggcctcagccagggaactgccctgccacctcccgaggcaggaaaggaagtgagaaaagga |
| | | gaagatattactcctggggccaaagtagggggacaaacacccagtcgtatatggcttcagctctgaccaaaggcg |
| | | gatagggagctctcctgggtaggagcagggagccaagggggaggcagtggctgtgcctgggtgggcacagac |
| | | agatctggtacatggccctgagccctgggcagagggacaaccagccggtgagtgggcaggcag0agaggagg |
| | | cggcaggatgctglaccccgattccatcctcagggagtggagactggaggggaggtgcactgactcagatgaact |
| | | gactcccccacatgataagaagtaggtggcagcagcctctggaaaagtcagggccctggaggttacctggccca |
| | | gggctactacagccacaggccccagtggcaccatgccaccccaccatggctccactcaagggggccacacagc |
| | | caccgcctcacctcctaccacatcccaaactgggacaaaagacttcaagactggctaagat0gtagcagcagcg |
| | | gatgcccgggcatccaaagtggaaagccagggccccgtgtcaccggtgtgggcaaacacacatgcacgtgcac |
| | | acatgttctccctgaatcactcagcagcagacagg0ctgccgccctgggggtctcagccctgctagggctcacca |
| | | ggtggaagcctaggtggtctg0acctcagtttaggagtgggtcatttacgtcatcttaccatttggggacgagacag |
| | | gaatggtatcccttagggacccagagacactgcaaacagtgggtggccatgtagggctgcatgtc0cctgggtcc |
| | | aggggaatggagggagcaataacttgaagaaggggggaagggtttcttttatccttttttttttgtgtgacttctatcaa |
| | | aaca |
| Human CXCR5 | 16 | MNYPLTLEMDLENLEDLFWELDRLDNYNDTSLVENHLCPATEGPLMAS |
| variant 1 | | FKAVFVPVAYSLIFLLGVIGNVLVLVILERHRQTRSSTETFLFHLAVADLL |
| polypeptide | | LVFILPFAVAEGSVGWVLGTFLCKTVIALHKVNFYCSSLLLACIAVDRYL |
| (Genbank | | AIVHAVHAYRHRRLLSIHITCGTIWLVGFLLALPEILFAKVSQGHHNNSLP |
| Accession No. | | RCTFSQENQAETHAWFTSRFLYHVAGFLLPMLVMGWCYVGVVHRLRQ |
| NP_001707.1) | | AQRRPQRQKAVRVAILVTSIFFLCWSPYHIVIFLDTLARLKAVDNTCKLN |
| | | GSLPVAITMCEFLGLAHCCLNPMLYTFAGVKFRSDLSRLLTKLGCTGPAS |
| | | LCQLFPSWRRSSLSESENATSLTTF |
| Human CXCR5 | 17 | ccactctaaggaatgcggtccctttgacaggcgaaaaactgaagttggaaaagacaaagtgatttgttcaaaattga |
| variant 2 | | aatttgaaacttgacatttggtcagtgggccctatgtaggaaaaaacctccaagagagctagggttcctctcagaga |
| polynucleotide | | ggaaagacaggtccttaggtcctcaccctcccgtctccttgcccttgcagttctgggaactggacagattggacaact |
| (Genbank | | ataacgacacctccctggtggaaaatcatctctgccctgccacagaggggcccctcatggcctccttc0aaggccg |
| Accession No. | | tgttcgtgcccgtggcctacagcctcatcttcctcctgggcgtgatcggcaacgtcctggtgctggtgatcctggagc |
| NM_032966) | | ggcaccggcagacacgcagttccacggagaccttcctgttccacctggccgtggccgacctcctgctggtcttcat |
| | | cttgccctttgccgtggccgagggctctgtgggctgggtcctggggaccttcctctgcaaaactgtgattgccctgca |
| | | caaagtcaacttctactgcagcagcctgctcctggcctgcatcgccgtggaccgctacctg0gccattgtccacgcc |
| | | gtccatgcctaccgccaccgccgcctcctctccatccacatcacctgtgggaccatctggctggtgggcttcctcctt |
| | | gccttgccagagattctcttcgccaaagtcagccaaggccatcacaacaactccctgccacgttgcaccttctccca |
| | | agagaaccaagcagaaacgcatgcctggttcacctcccgattcctctaccatgtggcgggattcctgctgcccatgc |
| | | tggtgatgggctggtgctacgtgggggtagtgcacaggttgcgccaggcccag0cggcgccctcagcggcaga |
| | | aggcagtcagggtggccatcctggtgacaagcatcttcttcctctgctggtcaccctaccacatcgtcatcttcctgg |
| | | acaccctggcgaggctgaaggcc0gtggacaatacctgcaagctgaatggctctctccccgtggccatcaccatg |
| | | tgtgagttc0ctgggcctggcccactgctgcctcaaccccatgctctacactttcgccggcgtgaagttccgcagtg |
| | | acctgtcgcggctcctgacgaagctgggctgtaccggccctgcctccctgtgc0cagctcttccctagctggcgca |
| | | ggagcagtctctctgagtcagagaatgccacctctctcaccacgttctaggtcccagtgtccccttttattgctgcttttc |
| | | cttggggcaggcagtgatgctggatgctccttccaacaggagctgggatcctaagggctcaccgtggctaagagt |
| | | gtcctaggagtatcctcatttggggtagctagaggaaccaacccccatttctagaacatccctgccagctcttctgcc |
| | | ggccctggggctaggctggagcccagggagcggaaagcagctca0aaggcacagtgaaggctgtccttaccca |
| | | tctgcacccccctgggctgagagaacctcacgcacctcccatcctaatcatccaatgctcaagaaacaacttctactt |
| | | ctgcccttgccaacggagagcgcctgcccctcccagaacacactccatcagcttaggggctgctgacctccacag |
| | | cttcccctctctcctcctgcccacctgtcaaacaaagccagaagctgagcaccaggggatgagtggaggttaaggc |
| | | tgaggaaaggccagctggcagcagagtgtggccttcggacaac0tcagtccctaaaaacacagacattctgccag |
| | | gcccccaagcctgcagtcatcttgaccaagcaggaagctcagactggttgagttcaggtagctgcccctggctctg |
| | | accgaaacagcgctgggtccaccccatgtcaccggatcctgggtggtctgcaggcagggctgactctaggtgccc |
| | | ttggaggccagccagtgacctgaggaagcgtgaaggccgagaagcaagaaagaaaccc0gacagagggaag |
| | | aaaagagctttcttcccgaaccccaaggagggagatggatcaatcaaa0cccggcggtcccctccgccaggcga |
| | | gatggggtggggtggagaactcctagggtggctgggtccaggggatgggaggttgtgggcattgatggggaag |
| | | gaggctggcttgtcccctcctcactcccttcccataagctatagacccgaggaaactcagagtcggaacggagaaa |
| | | ggtggactggaaggggcccgtgggagtcatctcaaccatcccctccgtggcatcaccttaggcagggaagtgtaa |
| | | gaaacacactgaggcagggaagtccccaggccccaggaagccgtgccctgc0ccccgtgaggatgtcactcag |
| | | atggaaccgcaggaagctgctccgtgcttgtttgctcacctggggtgtgggaggcccgtccggcagttctgggtgc |
| | | tccctaccacctccccagcctttgatcaggtggggagtcagggacccctgcccttgtcccactcaagccaagcagc |
| | | caagctccttgggaggccccactggggaaataacagctgtggctcacgtgagagtgtcttcacggcaggacaacg |
| | | aggaagccctaagacgtcccttttttctctgagtatctcctcgcaagctggg0taatcgatgggggagtctgaagca |
| | | gatgcaaagaggcaagaggctggattttgaattttctttttaataaaaaggcacctataaaacaggtcaatacagtaca |
| | | ggcagcacagagacccccggaacaagcctaaaaattgtttcaaaataaaaaccaagaagatgtcttcacatattgta |
| | | aaaaaaaaaaaaaaaa |
| Human CXCR5 | 18 | MASFKAVFVPVAYSLIFLLGVIGNVLVLVILERHRQTRSSTETFLFHLAVA |
| variant 2 | | DLLLVFILPFAVAEGSVGWVLGTFLCKTVIALHKVNFYCSSLLLACIAVD |
| polypeptide | | RYLAIVHAVHAYRHRRLLSIHITCGTIWLVGFLLALPEILFAKVSQGHHN |
| (Genbank | | NSLPRCTFSQENQAETHAWFTSRFLYHVAGFLLPMLVMGWCYVGVVHR |
| Accession No. | | LRQAQRRPQRQKAVRVAILVTSIFFLCWSPYHIVIFLDTLARLKAVDNTC |
| NP_116743.1) | | KLNGSLPVAITMCEFLGLAHCCLNPMLYTFAGVKFRSDLSRLLTKLGCT |
| | | GPASLCQLFPSWRRSSLSESENATSLTTF |
| Human | 19 | agctccaaccagggcagccttcctgagaagatgcaaccaatcctgcttctgctggccttcctcctgctgcccagggc |
| Granzyme B | | agatgcaggggagatcatcgggggacatgaggccaagccccactcccgcccctacatggcttatcttatgatctgg |
| variant 1 | | gatcagaagtctctgaagaggtgcggtggcttcctgatacgagacgacttcgtgctgacagctgctcactgttgggg |
| polynucleotide | | aagctccataaatgtcaccttgggggcccacaatatcaaagaacaggagccgacccagcagtttatccct0gtgaa |
| (Genbank | | aagacccatcccccatccagcctataatcctaagaacttctccaacgacatcatgctactgcagctggagagaaag |
| Accession No. | | gccaagcggaccagagctgtgcagcccctcaggctacctagcaacaaggcccaggtgaagccagggcagacat |
| NM_004131) | | gcagtgtggccggctgggggcagacggcccccctgggaaaacactcacacacactacaagaggtgaagatgac |
| | | agtgcaggaagatcgaaagtgcgaatctgacttacgccattattacgacagtaccattgagttgtgcgtgggg0gac |
| | | ccagagattaaaaagacttcctttaagggggactctggaggccctcttgtgtgtaacaaggtggcccagggcattgt |
| | | ctcctatggacgaaacaatggcatgcctccacgagcctgcaccaaagtctcaagctttgtacactggataaagaaaa |
| | | ccatgaaacgctactaactacaggaagcaaactaagcccccgctgtaatgaaacaccttctctggagccaagtcca |
| | | gatttacactgggagaggtgccagcaactgaataaatacctcttagctgagtggaaaa |
| Human | 20 | MQPILLLLAFLLLPRADAGEIIGGHEAKPHSRPYMAYLMIWDQKSLKRC |
| Granzyme B | | GGFLIRDDFVLTAAHCWGSSINVTLGAHNIKEQEPTQQFIPVKRPIPHPAY |
| variant 1 | | NPKNFSNDIMLLQLERKAKRTRAVQPLRLPSNKAQVKPGQTCSVAGWG |
| polypeptide | | QTAPLGKHSHTLQEVKMTVQEDRKCESDLRHYYDSTIELCVGDPEIKKT |
| (Genbank | | SFKGDSGGPLVCNKVAQGIVSYGRNNGMPPRACTKVSSFVHWIKKTMK |
| Accession No. | | RY |
| NP_004122.2) | | |
| Human | 21 | agctccaaccagggcagccttcctgagaagatgcaaccaatcctgcttctgctggccttcctcctgctgcccagggc |
| Granzyme B | | agatgcagacttttccttcaggggagatcatcgggggacatgaggccaagccccactcccgcccctacatggctta |
| variant 2 | | tcttatgatctgggatcagaagtctctgaagaggtgcggtggcttcctgatacgagacgacttcgtgctgacagctgc |
| polynucleotide | | tcactgttggggaagctccataaatgtcaccttgggggcccacaatatcaaagaacaggagccgaccca0gcagtt |
| (Genbank | | tatccctgtgaaaagacccatcccccatccagcctataatcctaagaacttctccaacgacatcatgctactgcagct |
| Accession No. | | ggagagaaaggccaagcggaccagagctgtgcagcccctcaggctacctagcaacaaggcccaggtgaagcc |
| NM_001346011) | | agggcagacatgcagtgtggccggctgggggcagacggcccccctgggaaaacactcacacacactacaaga |
| | | ggtgaagatgacagtgcaggaagatcgaaagtgcgaatctgacttacgccattattacgacagtaccattga0gttg |
| | | tgcgtgggggacccagagattaaaaagacttcctttaagggggactctggaggccctcttgtgtgtaacaaggtgg |
| | | cccagggcattgtctcctatggacgaaacaatggcatgcctccacgagcctgcaccaaagtctcaagctttgtacac |
| | | tggataaagaaaaccatgaaacgctactaactacaggaagcaaactaagcccccgctgtaatgaaacaccttctct |
| | | ggagccaagtccagatttacactgggagaggtgccagcaactgaataaatacctcttagctgagtgg0aaaa |
| Human | 22 | MQTFPSGEIIGGHEAKPHSRPYMAYLMIWDQKSLKRCGGFLIRDDFVLT |
| Granzyme B | | AAHCWGSSINVTLGAHNIKEQEPTQQFIPVKRPIPHPAYNPKNFSNDIMLL |
| variant 2 | | QLERKAKRTRAVQPLRLPSNKAQVKPGQTCSVAGWGQTAPLGKHSHTL |
| polypeptide | | QEVKMTVQEDRKCESDLRHYYDSTIELCVGDPEIKKTSFKGDSGGPLVC |
| (Genbank | | NKVAQGIVSYGRNNGMPPRACTKVSSFVHWIKKTMKRY |
| Accession No. | | |
| NP_001332940.1) | | |
| Human | 23 | gtgggctcttgaaacccgagcatggcacagcacggggcgatgggcgcgtttcgggccctgtgcggcctggcgct |
| TNFRSF 18 | | gctgtgcgcgctcagcctgggtcagcgccccaccgggggtcccgggtgcggccctgggcgcctcctgcttggga |
| variant 1 | | cgggaacggacgcgcgctgctgccgggttcacacgacgcgctgctgccgcgattacccgggcgaggagtgctg |
| polynucleotide | | ttccgagtgggactgcatgtgtgtccagcctgaattccactgcggagacccttgctgcacgacctgccggcaccac |
| (Genbank | | cct0tgtcccccaggccagggggtacagtcccaggggaaattcagttttggcttccagtgtatcgactgtgcctcgg |
| Accession No. | | ggaccttctccgggggccacgaaggccactgcaaaccttggacagactgcacccagttcgggtttctcactgtgttc |
| NM_004195) | | cctgggaacaagacccacaacgctgtgtgcgtcccagggtccccgccggcagagccgcttgggtggctgaccgt |
| | | cgtcctcctggccgtggccgcctgcgtcctcctcctgacctcggcccagcttggactgcacatctggcagctgagg |
| | | 0agtcagtgcatgtggccccgagagacccagctgctgctggaggtgccgccgtcgaccgaagacgccagaagc |
| | | tgccagttccccgaggaagagcggggcgagcgatcggcagaggagaaggggcggctgggagacctgtgggt |
| | | gtgagcctggccgtcctccggggccaccgaccgcagccagcccctccccaggagctccccaggccgcagggg |
| | | ctctgcgttctgctctgggccgggccctgctcccctggcagcagaagtgggtgcaggaaggtggcagtgaccagc |
| | | gccctggacc0atgcagttcggcggccgcggctgggccctgcaggagggagagagagacacagtcatggccc |
| | | ccttcctcccttgctggccctgatggggtggggtcttaggacgggaggctgtgtccgtg0ggtgtgcagtgcccag |
| | | cacgggacccggctgcaggggaccttcaataaacacttgtccag0tga |
| Human | 24 | MAQHGAMGAFRALCGLALLCALSLGQRPTGGPGCGPGRLLLGTGTDAR |
| TNFRSF18 | | CCRVHTTRCCRDYPGEECCSEWDCMCVQPEFHCGDPCCTTCRHHPCPP |
| variant 1 | | GQGVQSQGKFSFGFQCIDCASGTFSGGHEGHCKPWTDCTQFGFLTVFPG |
| polypeptide | | NKTHNAVCVPGSPPAEPLGWLTVVLLAVAACVLLLTSAQLGLHIWQLRS |
| (Genbank | | QCMWPRETQLLLEVPPSTEDARSCQFPEEERGERSAEEKGRLGDLWV |
| Accession No. | | |
| NP_004186.1) | | |
| Human | 25 | gtgggctcttgaaacccgagcatggcacagcacggggcgatgggcgcgtttcgggccctgtgcggcctggcgct |
| TNFRSF18 | | gctgtgcgcgctcagcctgggtcagcgccccaccgggggtcccgggtgcggccctgggcgcctcctgcttggga |
| variant 2 | | cgggaacggacgcgcgctgctgccgggttcacacgacgcgctgctgccgcgattacccgggcgaggagtgctg |
| polynucleotide | | ttccgagtgggactgcatgtgtgtccagcctgaattccactgcggagacccttgctgcacgacctgccggcaccac |
| (Genbank | | cct0tgtcccccaggccagggggtacagtcccaggggaaattcagttttggcttccagtgtatcgactgtgcctcgg |
| Accession No. | | ggaccttctccgggggccacgaaggccactgcaaaccttggacagactgctgctggaggtgccgccgtcgaccg |
| NM_148901) | | aagacgccagaagctgccagttccccgaggaagagcggggcgagcgatcggcagaggagaaggggcggctg |
| | | ggagacctgtgggtgtgagcctggccgtcctccggggccaccgaccgcagccagcccctccccaggagctccc |
| | | caggccgca0ggggctctgcgttctgctctgggccgggccctgctcccctggcagcagaagtgggtgcaggaag |
| | | gtggcagtgaccagcgccctggaccatgcagttcggcggccgcggctgggccctgcaggagggagagagaga |
| | | cacagtcatggcccccttcctcccttgctggccctgatggggtggggtcttaggacgggaggctgtgtccgtgggt |
| | | gtgcagtgcccagcacgggacccggctgcaggggaccttcaataaacacttgtccagtga |
| Human | 26 | MAQHGAMGAFRALCGLALLCALSLGQRPTGGPGCGPGRLLLGTGTDAR |
| TNFRSF18 | | CCRVHTTRCCRDYPGEECCSEWDCMCVQPEFHCGDPCCTTCRHHPCPP |
| variant 2 | | GQGVQSQGKFSFGFQCIDCASGTFSGGHEGHCKPWTDCCWRCRRRPKT |
| polypeptide | | PEAASSPRKSGASDRQRRRGGWETCGCEPGRPPGPPTAASPSPGAPQAA |
| (Genbank | | GALRSALGRALLPWQQKWVQEGGSDQRPGPCSSAAAAGPCRRERETQS |
| Accession No. | | WPPSSLAGPDGVGS |
| NP_683699.1) | | |
| Human | 27 | gtgggctcttgaaacccgagcatggcacagcacggggcgatgggcgcgtttcgggccctgtgcggcctggcgct |
| TNFRSF18 | | gctgtgcgcgctcagcctgggtcagcgccccaccgggggtcccgggtgcggccctgggcgcctcctgcttggga |
| variant 3 | | cgggaacggacgcgcgctgctgccgggttcacacgacgcgctgctgccgcgattacccgggcgaggagtgctg |
| polynucleotide | | ttccgagtgggactgcatgtgtgtccagcctgaattccactgcggagacccttgctgcacgacctgccggcaccac |
| (Genbank | | cct0tgtcccccaggccagggggtacagtcccaggggaaattcagttttggcttccagtgtatcgactgtgcctcgg |
| Accession No. | | ggaccttctccgggggccacgaaggccactgcaaaccttggacagactgcacccagttcgggtttctcactgtgttc |
| NM_148902) | | cctgggaacaagacccacaacgctgtgtgcgtcccagggtccccgccggcagagccgcttgggtggctgaccgt |
| | | cgtcctcctggccgtggccgcctgcgtcctcctcctgacctcggcccagcttggactgcacatctggcagctgagg |
| | | 0aagacccagctgctgctggaggtgccgccgtcgaccgaagacgccagaagctgccagttccccgaggaagag |
| | | cggggcgagcgatcggcagaggagaaggggcggctgggagacctgtgggtgtgagcctggccgtcctccggg |
| | | gccaccgaccgcagccagcccctccccaggagctccccaggccgcaggggctctgcgttctgctctgggccgg |
| | | gccctgctcccctggcagcagaagtgggtgcaggaaggtggcagtgaccagcgccctggaccatgcagttcggc |
| | | ggccgcggc0tgggccctgcaggagggagagagagacacagtcatggcccccttcctcccttgctggccctgat |
| | | ggggtggggtcttaggacgggaggctgtgtccgtgggtgtgcagtgcccagcacgg0gacccggctgcagggg |
| | | accttcaataaacacttgtccagtga |
| Human | 28 | MAQHGAMGAFRALCGLALLCALSLGQRPTGGPGCGPGRLLLGTGTDAR |
| TNFRSF18 | | CCRVHTTRCCRDYPGEECCSEWDCMCVQPEFHCGDPCCTTCRHHPCPP |
| variant 3 | | GQGVQSQGKFSFGFQCIDCASGTFSGGHEGHCKPWTDCTQFGFLTVFPG |
| polypeptide | | NKTHNAVCVPGSPPAEPLGWLTVVLLAVAACVLLLTSAQLGLHIWQLR |
| (Genbank | | KTQLLLEVPPSTEDARSCQFPEEERGERSAEEKGRLGDLWV |
| Accession No. | | |
| NP_683700.1) | | |

All polynucleotide and amino acid sequences referenced by the Genbank accession numbers are incorporated by reference in their entirety.

TABLE 13

| Uniprot | Entry | Gene | Protein | Cross-reference |
|---|---|---|---|---|
| Entry | name | names | names | (RefSeq) |
| O43927 | CXCL13_HUMAN | CXCL13 BCA1 | CXCL13 | NP_006410.1; XP_006714126.1; |
| | | BLC SCYB13 | | |
| P32302 | CXCR5_HUMAN | CXCR5 BLR1 | CXCR5 | NP_001707.1 [P32302-1]; |
| | | MDR15 | | NP_116743.1 [P32302-2]; |
| P10144 | GRAB_HUMAN | GZMB CGL1 | GZMB | NP_004122.2; |
| | | CSPB CTLA1 | | |
| | | GRB | | |
| Q12918 | KLRB1_HUMAN | KLRB1 CLEC5B | KLRB | NP_002249.1; |
| | | NKRP1A | | |
| P62942 | FKB1A_HUMAN | FKBP1A FKBP1 | FKBP1A | NP_000792.1; NP_463460.1; |
| | | FKBP12 | | |
| P00441 | SODC_HUMAN | SOD1 | SOD1 | NP_000445.1; |
| P10147 | CCL3_HUMAN | CCL3 G0S19-1 | CCL3 | NP_002974.1; |
| | | MIP1A SCYA3 | | |
| P13236 | CCL4_HUMAN | CCL4 LAG1 | CCL4 | NP_002975.1; NP_996890.1; |
| | | MIP1B SCYA4 | | |
| O60315 | ZEB2_HUMAN | ZEB2 KIAA0569 | ZEB2 | NP_001165124.1 [O60315-2]; |
| | | SIP1 ZFHX1B | | NP_055610.1 [O60315-1]; |
| | | ZFX1B | | XP_006712944.1; XP_006712945.1; |
| | | HRIHFB2411 | | |
| Q9Y5U5 | TNR18_HUMAN | TNFRSF18 AITR | TNFRSF18 | NP_004186.1 [Q9Y5U5-1]; |
| | | GITR | (GITR) | NP_683699.1 [Q9Y5U5-2]; |
| | | UNQ319/PRO364 | | NP_683700.1 [Q9Y5U5-3]; |
| P43489 | TNR4_HUMAN | TNFRSF4 | TNFRSF4 | NP_003318.1; XP_016857721.1; |
| | | TXGP1L | (OX40) | |
| P01579 | IFNG_HUMAN | IFNG | IFNG | NP_000610.2; |
| Q9HBE4 | IL21_HUMAN | IL21 | IL21 | NP_001193935.1 [Q9HBE4-2]; |
| | | | | NP_068575.1 [Q9HBE4-1]; |
| P16949 | STMN1_HUMAN | STMN1 C1orf215 | STMN1 | NP_001138926.1 [P16949-2]; |
| | | LAP18 OP18 | | NP_005554.1 [P16949-1]; |
| | | | | NP_981944.1 [P16949-1]; |
| | | | | NP_981946.1 [P16949-1]; |
| Q13257 | MD2L1_HUMAN | MAD2L1 MAD2 | MAD2L1 | NP_002349.1 [Q13257-1]; |
| O95347 | SMC2_HUMAN | SMC2 CAPE | SMC2 | NP_001036015.1 [O95347-1]; |
| | | SMC2L1 | | NP_001036016.1 [O95347-1]; |
| | | PRO0324 | | NP_001252531.1 [O95347-1]; |
| | | | | NP_006435.2 [O95347-1]; |
| | | | | XP_006716996.1 [O95347-1]; |
| | | | | XP_011516450.1 [O95347-1]; |
| | | | | XP_011516451.1 [O95347-1]; |
| | | | | XP_011516455.1 [O95347-2]; |
| | | | | XP_016869695.1 [O95347-1]; |
| | | | | XP_016869696.1 [O95347-1]; |
| | | | | XP_016869697.1 [O95347-1]; |
| Q9BXS6 | NUSAP_HUMAN | NUSAP1 ANKT | NUSAP1 | NP_001230071.1 [Q9BXS6-3]; |
| | | BM-037 | | NP_001230072.1 [Q9BXS6-6]; |
| | | PRO0310 | | NP_001230073.1 [Q9BXS6-7]; |
| | | | | NP_001288065.1 [Q9BXS6-5]; |
| | | | | NP_057443.2 [Q9BXS6-1]; |
| | | | | NP_060924.4 [Q9BXS6-2]; |
| | | | | XP_005254487.1 [Q9BXS6-4]; |
| P11388 | TOP2A_HUMAN | TOP2A TOP2 | TOP2A | NP_001058.2 [P11388-1]; |
| O75444 | MAF_HUMAN | MAF | MAF | NP_001026974.1 [O75444-2]; |
| | | | | NP_005351.2 [O75444-1]; |
| O60880 | SH21A_HUMAN | SH2D1A DSHP | SH2D1A | NP_001108409.1 [O60880-4]; |
| | | SAP | | NP_002342.1 [O60880-1]; |
| Q15116 | PDCD1_HUMAN | PDCD1 PD1 | PDCD1 | NP_005009.2; |
| Q7Z6A9 | BTLA_HUMAN | BTLA | BTLA | NP_001078826.1 [Q7Z6A9-2]; |
| | | | | NP_861445.3 [Q7Z6A9-1]; |
| | | | | XP_016861237.1 [Q7Z6A9-1]; |
| P41217 | OX2G_HUMAN | CD200 MOX1 | CD200 | NP_001004196.2 [P41217-3]; |
| | | MOX2 My033 | | NP_001305757.1; |
| | | | | NP_005935.4 [P41217-2]; |
| | | | | XP_005247539.1; |
| P41182 | BCL6_HUMAN | BCL6 BCL5 | BCL6 | NP_001124317.1 [P41182-1]; |
| | | LAZ3 ZBTB27 | | NP_001128210.1 [P41182-2]; |
| | | ZNF51 | | NP_001697.2 [P41182-1]; |
| | | | | XP_005247751.1 [P41182-1]; |
| | | | | XP_011511364.1 [P41182-2]; |

All polynucleotide and amino acid sequences referenced by the Genbank accession numbers are incorporated by reference in their entirety.

1. An engineered T-follicular helper (Tfh)-like tumor-infiltrating cell engineered to modulate expression of the surface markers CD4, CXCL13 and CXCR5 or one or more proteins selected from MAF, SH2D1A (SAP), PDCD1, BTLA, CD200, and BCL6.

2. The cell of claim 1, wherein the cell is engineered to express the surface markers CD4 and CXCL13 and lack the surface marker CXCR5.

3. The cell of claim 1 wherein the cell is further engineered to express GZMB.

4. The cell of claim 1, wherein the cell is a Tfh-like tumor-infiltrating cell that activates a CD8⁺ CTL response.

5. The cell of claim 4, wherein the CD8⁺ CTL response is activated in a tumor or tumor microenvironment.

6. The cell of claim 1, wherein the cell is a Tfh-like tumor-infiltrating cell that activates a CD8⁺ TRM response.

7. The cell of claim 6, wherein the CD8⁺ TRM response is activated in a tumor or tumor microenvironment.

8.-9. (canceled)

10. An isolated T-follicular helper (Tfh)-like tumor-infiltrating cell expressing the surface markers CD4 and CXCL13 and lacking the surface marker CXCR5.

11. The cell of claim 10, wherein the cell is a cytotoxic Tfh-like tumor-infiltrating cell expressing GZMB.

12. The cell of claim 1, wherein the cell is engineered to increase expression and/or function of one or more of: TNFRSF18, TNFRSF4, IFNG, Granzyme B and/or IL21 in the cell.

13. The cell of claim 1, wherein the cell is engineered to expresses an antigen binding domain that binds at least one tumor antigen.

14. The cell of claim 13, wherein the tumor antigen comprises any one of: a CD19, a disialoganglioside-GD2, a c-mesenchymal-epithelial transition (c-Met), a mesothelin, a ROR1, an EGFRvIII, an ephrin type-A receptor 2 (EphA2), an interleukin (IL)-13r alpha 2, an EGFR V111, a PSMA, an EpCAM, a GD3, a fucosyl GM1, a PSCA, a PLAC1, a sarcoma breakpoint, a Wilms Tumor 1 antigen or a combination thereof.

15. The cell of claim 1, wherein the cell is engineered to express or expresses an antigen binding domain that binds at least one antigen.

16. The cell of claim 15, wherein the antigen is selected from a neo-antigen, tumor-associated antigen, viral antigen, bacterial antigen, and parasitic antigen.

17. The cell of claim 1, wherein the cell further comprises a suicide gene.

18. The cell of claim 1, wherein the cell further comprises a chimeric antigen receptor (CAR).

19. The cell of claim 18, wherein the chimeric antigen receptor (CAR) comprises: (a) an antigen binding domain; (b) a hinge domain; (c) a transmembrane domain; (d) and an intracellular domain and optionally a CD3 zeta signaling domain.

20. (canceled)

21. The cell of claim 19, wherein the antigen binding domain of the CAR binds a tumor antigen.

22.-55. (canceled)

56. A method of determining whether a subject will respond to a treatment for cancer comprising measuring the amount of one or more of: CD4⁺CXCL13⁺CXCR5⁻ Tfh-like tumor-infiltrating cell, and/or CD4⁺CXCL13⁺CXCR5′GZMB⁺ cytotoxic Tfh-like tumor-infiltrating cell in a sample isolated from the subject, wherein higher amounts of the cells indicates that the subject is likely to respond to the treatment and lower amounts of the cells indicates that the subject is not likely to respond to the treatment.

57.-62. (canceled)

63. A method of treating cancer in a subject comprising administering to the subject an effective amount of the cell of claim 1.

64.-70. (canceled)
